(19) United States
(12) Patent Application Publication
(10) Pub. No.: US 2006/0009478 A1
(43) Pub. Date: Jan. 12, 2006

(54) Methods for the treatment of back pain

(76) Inventors: Nadav Friedmann, Lafayette, CA (US); Remi Barbier, San Francisco, CA (US); Grant L. Schoenhard, San Carlos, CA (US)

Correspondence Address: MCANDREWS HELD & MALLOY, LTD, 500 WEST MADISON STREET, SUITE 3400, CHICAGO, IL, 60661, US

(21) Appl. No.: 11/089,283

(22) Filed: Mar. 23, 2005

Related U.S. Application Data
(63) Continuation-in-part of application No. 10/966,703, filed on Oct. 15, 2004.
(60) Provisional application No. 60/511,841, filed on Oct. 15, 2003.
(60) Provisional application No. 60/566,189, filed on Apr. 27, 2004.

(51) Int. Cl. A61K 31/485

(52) U.S. Cl. 514/282

(57) ABSTRACT

Methods and materials, including novel compositions, dosage forms and methods of administration, useful for treating back pain using opioid antagonists, including combinations of opioid antagonists and opioid agonists. Methods and materials comprising opioid antagonists or combinations opioid antagonists and agonists may optionally include one or more additional therapeutic agents.

### CROSS REFERENCE TO RELATED APPLICATIONS

This application claims the priority of U.S. Patent Application No. 60/511,841, filed Oct. 15, 2003 (provisional) and U.S. Patent Application No. 60/566,189, filed Apr. 27, 2004 (provisional), and is a continuation-in-part of U.S. patent application Ser. No. 10/966,703, filed Oct. 15, 2004 (nonprovisional). The applications cited above are hereby incorporated by reference in their entirety.

### FIELD OF THE INVENTION

The present invention relates to methods and materials useful for the treatment of back pain, including low back pain, using opioid antagonists, including combinations of opioid antagonists and opioid agonists. The methods and materials provide human subjects with an alleviation of back pain and with an attenuation of one or more adverse effects, including withdrawal, dependence, tolerance, or addiction. The methods and materials additionally or alternatively provide human subjects with an alleviation of back pain and with an attenuation of one or more opioid-related adverse effects such as pruritis, somnolence or constipation. Methods and materials comprising opioid antagonists or combinations of opioid antagonists and agonists are provided and may optionally include one or more additional therapeutic agents.

### BACKGROUND OF THE INVENTION

Approximately one in six U.S. adults (16%) suffered from back pain every single day during the preceding month, according to a recent national survey by the North American Spine Society (NASS). NASS also stated that eighty percent of adults will suffer from back pain at some point during their lives, and people suffering from back pain are dealing with the issue an average of 14 days per month. According to the Cleveland Clinic Health System, eighty to ninety percent of the population in the United States will suffer from back pain during their lifetime, and back pain is the second most common reason for doctor visits.

Back pain can arise from many causes, including disc herniation, osteoarthritis, spinal stenosis, spondylolisthesis, and ankylosing spondylitis. Back pain can also arise from infection, cancer, fracturem, and nonspinal causes. Low back pain is a very common disorder among adults. Most low back pain is nonspecific; it has no known cause and generally cannot be given a precise pathoanatomical diagnosis. According to Snook, J. Occup Rehabil., 14(4):243-53 (2004), there are many different treatments for nonspecific low back pain, but most of them are ineffective.

Many cases of back pain result from weakness of the back muscles. This includes back pain associated with stress, when the back muscles often go into spasm. The intervertebral disks are subjected to different types of stress, and degeneration of the disks can give rise to chronic back pain as the muscles supporting the disks go into spasm. The degenerating disk can itself become inflamed and may cause mechanical pain. The lower part of the back (the lumbar region) is the most vulnerable area of the back, and lower back pain is the most common occurrence of back pain. This is largely due to the fact the lower part of the spine bears the entire weight of the upper body and is bent, twisted and flexed during everyday activities more than any other part of the spine. Pain associated with damage to the supportive soft tissues (muscles, tendons and ligaments), and the intervertebral disks themselves, is caused by the inflammatory process.

Inflammation arises in connection with many chronic conditions, including, for example, arthritic conditions such as osteoarthritis (OA). Osteoarthritis is a degenerative joint disease, characterized by the breakdown of the joint's cartilage. Cartilage breakdown causes bones to rub against each other, causing pain and/or loss of movement. Osteoarthritis can range from very mild to very severe and can affect the back, the neck, the hands and weight-bearing joints such as knees, hips, and feet.

Another chronic condition with which inflammation is associated is rheumatoid arthritis (RA). Rheumatoid arthritis involves inflammation in the lining of the joints and/or other internal organs. Rheumatoid arthritis typically affects many different joints. It is typically chronic, but can be a disease of flare-ups. Rheumatoid arthritis is a systemic disease that affects the entire body including the back and is one of the most common forms of arthritis.

Another chronic condition with which inflammation is associated is back pain, particularly lower back pain. Lower back pain affects approximately two-thirds of the U.S. adult population, leads to significant increases in physician office visits, and has a significant effect on disability.

Oxycodone, morphine and oxymorphone have been used in clinical studies of patients with chronic back pain. Oxycodone controlled release and oxycodone immediate release compositions have been used in clinical studies of patients with stable, chronic moderate-to-severe low back pain as described by Hale et al., Clin. J. Pain, 15, 179-183 (1999). A morphine sulfate extended-release product identified as AVINZA® (Ligand Pharmaceuticals Incorporated, San Diego, Calif., USA) has been approved for once daily administration and is indicated for relief of moderate to severe pain requiring continuous around-the-clock opioid therapy for an extended period of time. An oxymorphone extended release composition has been used in clinical studies of ambulatory patients with moderate-to-severe chronic low back pain as described by Hale et al., Clin. J. Pain, 6(1), 21-28 (2005).

Crain and Shen in U.S. Pat. Nos. 5,472,943; 5,512,578 reissued as RE 36,457; 5,580,876; 5,767,125; 6,096,756; and 6,362,194 as well as U.S. Patent Application Publication No. 20020094947 A1 (the disclosures of which are incorporated herein by reference) describe methods and compositions of opioids for selectively enhancing the analgesic potency of a bimodally-acting opioid agonist and simultaneously attenuating anti-analgesia, hyperalgesia, hyperexcitability, physical dependence and/or tolerance effects associated with the administration of the bimodally-acting opioid agonist, by administering to a subject an analgesic or sub-analgesic amount of a bimodally-acting opioid agonist and an amount of an excitatory opioid receptor antagonist effective to enhance the analgesic potency of the bimodally-acting opioid agonist and attenuate the anti-analgesia, hyperalgesia, hyperexcitability, physical dependence and/or tolerance effects of the bimodally-acting opioid agonist. Also disclosed are methods and compositions of opioids for treating pain in a subject by administering to the subject an analgesic or sub-analgesic amount of a bimodally-acting opioid agonist and an amount of an excitatory opioid receptor antagonist effective to enhance the analgesic potency of the bimodally-acting opioid agonist and simultaneously attenuate anti-analgesia, hyperalgesia, hyperexcitability, physical dependence and/or tolerance effects of the bimodally-acting opioid agonist.

U.S. Patent Application Publication Nos. 20010006967 A1 and 20020094947 A1 (the disclosures of which are incorporated herein by reference) describe a method for selectively enhancing the analgesic potency of a bimodally-acting opioid agonist such as tramadol and simultaneously attenuating anti-analgesia, hyperalgesia, hyperexcitability, physical dependence and/or tolerance effects associated with the administration of the bimodally-acting opioid agonist. Disclosed are methods and compositions of tramadol in analgesic or sub-analgesic amounts and an opioid antagonist such as naltrexone or nalmefene.

U.S. Patent Application Publication No. 20010018413 A1 and U.S. Pat. No. 6,737,400 (published as U.S. Patent Application No. 20020173466 A1) (the disclosures of which are incorporated herein by reference) describe a method for treating a subject with irritable bowel syndrome (“IBS”) with an opioid antagonist. Disclosed are materials and methods for long-term administration of an opioid receptor antagonist at an appropriately low dose which will selectively antagonize excitatory opioid receptor functions, but not inhibitory opioid receptor functions, in myenteric neurons in the intestinal tract as well as in neurons of the central nervous system (“CNS”). The administration of the opioid receptor antagonist at a low dose reduces abdominal pain and stool frequency. Also disclosed are compositions for treating a subject with IBS, which comprise an effective dose of an opioid receptor antagonist, and a pharmaceutically acceptable carrier.

U.S. Patent Application Publication No. 2002013776 A1 (the disclosure of which is incorporated herein by reference) describes a method for increasing analgesic potency of a bimodally-acting opioid agonist in a subject, by inhibiting GM1-ganglioside in nociceptive neurons. The publication describes methods for treating pain, including methods for treating chronic pain, in a subject in need of treatment thereof. Additionally, a method is described for treating tolerance to or an addiction to a bimodally-acting opioid agonist in a subject in need of treatment thereof. A pharmaceutical composition of analgesic agents and a pharmaceutically-acceptable carrier is described.

International Publication No. WO 01/085150 (International PCT/US01/14644) (the disclosure of which is incorporated herein by reference) describes novel compositions and methods for enhancing potency or reducing adverse side effects of opioid agonists in humans, including with an opioid agonist and an opioid antagonist to differentially dose a human subject so as to either enhance analgesic potency without attenuating an adverse side effect of the agonist, or alternatively maintain the analgesic potency of the agonist while attenuating an adverse side effect of the agonist. Also described are novel opioid compositions and methods for the gender-based dosing of men and women.

U.S. Patent Application Publication No. 20030191147 A1 (the disclosure of which is incorporated herein by reference) describes novel dosage forms, pharmaceutical compositions, kits, and methods of administration of an opioid antagonist, including in an amount of at least about 0.0001 mg to about or less than about 1.0 mg, including from about 0.0001 mg to less than about 0.5 mg. Disclosed are solid oral dosage forms comprising an opioid antagonist and another active ingredient, such as an opioid agonist. Also disclosed are immediate release oral dosage forms and concurrent release dosage forms comprising an opioid antagonist and another active ingredient.

Although a variety of therapeutic agents have been used for treating pain and/or inflammation, including chronic pain and/or inflammation the treatment is often still ineffective. In particular, back pain is often poorly managed or controlled even by the chronic administration of such agents. This may be due to the loss of potency of the agent and/or the development of side effects associated with chronic treatment with the agent.

### SUMMARY OF THE INVENTION

The present disclosure provides methods for the treatment of back pain. Methods and materials are described which provide human subjects with alleviation of back pain, including low back pain. Methods and materials are described which provide treatment for back pain, including chronic back pain, and including wherein the back pain is moderate or severe. Methods and materials are described which comprise opioid antagonists or combinations of opioid antagonists and agonists and may optionally include one or more additional therapeutic agents.

In one aspect, the present invention provides methods for treating back pain in a human subject comprising administering to the subject an opioid agonist and an opioid antagonist wherein back pain is alleviated and wherein the amount of the antagonist or the amount of the agonist and the amount of the antagonist together is effective to attenuate withdrawal.

In another aspect, the present invention provides methods for treating back pain in a human subject comprising administering to the subject an opioid agonist and an opioid antagonist, wherein back pain is alleviated and wherein the amount of the antagonist is effective to attenuate one or more of the adverse effects commonly associated with opioids (e.g., opioid-related or opioid associated adverse effects, such as, for example, skin and subcutaneous tissue disorders (e.g., pruritus), gastrointestinal disorders (e.g., constipation, nausea, vomiting), nervous system disorders (e.g., dizziness, somnolence), or respiratory depression.

In yet another aspect, the present disclosure provides methods for treating back pain in a human subject comprising administering to the subject an opioid agonist and an opioid antagonist, wherein back pain is alleviated and wherein the amount of the antagonist or the amount of agonist and an amount of antagonist together is effective to attenuate one or more adverse effects, such as tolerance, dependence or addiction.

In another aspect, the present invention provides methods for treating back pain in a human subject by administering to the subject an opioid antagonist, wherein the amount of the antagonist is effective for enhancing the potency of an opioid agonist to alleviate back pain and for attenuating withdrawal.

Potency may refer to the strength of a drug or drug treatment in producing desired effects, for example, improved pain relief, improved pain control, reduced stiffness, and/or improved physical function. Potency also may refer to the effectiveness or efficacy of a drug treatment in eliciting desired effects, for example, improved pain relief, improved pain control, reduced stiffness, and/or improved physical function. For example, enhanced potency may refer to the lowering of a dose in achieving desired effects or to an increased therapeutic benefit including that not previously seen. Enhanced potency may be seen where a subject titrates at a lower dose of opioid agonist, or obtains an acceptable level of pain relief at a lower dose, wherein the lower dose is a lower daily dose or a lower cumulative dose over a period. In therapeutics, for example, potency may refer to the relative pharmacological activity of a compound or a composition.

In another aspect, the present invention provides methods for treating back pain in a human subject by administering to the subject an opioid antagonist, wherein the amount of the antagonist is effective for enhancing the potency of the opioid agonist to alleviate back pain and for attenuating one or more opioid-related adverse effects, such as, for example, skin and subcutaneous tissue disorders (e.g., pruritus), gastrointestinal disorders (e.g., constipation, nausea, vomiting), or nervous system disorders (e.g., dizziness, somnolence), or respiratory depression.

In yet another aspect, the present invention provides methods for treating back pain in a human subject by administering to the subject an opioid antagonist, wherein the amount of the antagonist is effective for enhancing the potency of an opioid agonist to alleviate back pain and for attenuating one or more adverse effects, such as tolerance, dependence, or addiction.

In yet another aspect, the present invention provides methods for the treatment of back pain, including chronic back pain, and the inflammation associated with the back pain.

For any of the methods described herein, including the foregoing methods: the back pain may be associated with a neck, upper back, middle back or lower back of the subject, the back pain may be chronic, and/or the back pain may be moderate or severe; the agonist, the antagonist, or both the agonist and the antagonist may be administered no more than twice in a 24-hour period, or no more than once in a 24-hour period; the amount of the antagonist may be 0.004 mg or less in a 24-hour period, or 0.002 mg or less in a 24-hour period; the antagonist, the agonist, or both the antagonist and the agonist may be administered in an oral dosage form, including a solid oral dosage form or a liquid oral dosage form; the agonist may be codeine, hydromorphone, meperidine, morphine, oxycodone, oxymorphone, propoxyphene, hydrocodone, pentazocine, fentanyl, sufentanyl, methadone, tramadol, or dihydrocodeine; the antagonist may be naltrexone, nalmefene, or naloxone; the mode of administration of the agonist, the antagonist, or both, may be oral, intravenous, intrathecal, epidural, intramuscular, subcutaneous, perineural, intradermal, topical, or transcutaneous; the amount of the agonist may be from about 2.5 mg to about 160 mg; the amount of the antagonist may be from about 0.0001 mg to about 0.004 mg; the amount of the antagonist may be 0.001 or 0.002 mg or less, 0.0001 mg or 0.0002 mg or less, 0.00001 mg or 0.00002 mg or less, including wherein the amount is administered one-time, two-times, three-times, or four-times per day, preferably two-times per day; the amount of the agonist may be about 2.5 mg, about 5 mg, about 10 mg, about 15 mg, about 20 mg, about 30 mg, about 40 mg, about 80 mg, about 160 mg, or about 320 mg; the amount of the antagonist administered may be at least about 1250 fold less than the amount of the agonist administered; the amount of the antagonist administered may be at most about 1,600,000 fold less than the amount of the agonist administered; the amount of the antagonist administered to the subject may be no more than about 80.4 μgs, about 40.2 μgs, about 20 μgs, about 10 μgs, about 5 μgs, about 2.5 μgs, about 1.2 μgs, about 0.6 μg about 0.3 μg or about 0.12 μg; the amount of the antagonist administered to the subject may be at least about 0.0002 μg about 0.1 μg about 0.2 μg about 0.4 μg about 0.8 μg about 1.6 μg about 3.3 μg or about 6.6 μg; the amount of the antagonist administered may be effective to enhance the potency of the agonist for alleviating back pain, including wherein the enhanced potency is measured by administering a lower dose to achieve alleviation of back pain.

Novel compositions, dosage forms, kits, and other materials are described which comprise an opioid antagonist for use in or with the foregoing methods including wherein the amount of the antagonist is effective for attenuating withdrawal, or for attenuating one or more opioid-related adverse effects, or for attenuating tolerance, dependence, or addiction, and including compositions, dosage forms, kits, and other materials with an opioid agonist and an opioid antagonist, including wherein the amount of the agonist and the amount of the antagonist together are effective for alleviating back pain.

In yet another aspect, the present disclosure provides methods and materials for dosing an opioid antagonist administered to a human subject having back pain. An amount of an opioid antagonist and an amount of an opioid agonist are administered to the subject, and back pain is assessed. A level of the opioid antagonist or a surrogate of the opioid antagonist in a sample from the subject is measured. The amount of the opioid antagonist or the amount of the opioid agonist to the subject is adjusted based on the measured level.

In another aspect, the present disclosure provides methods and materials for dosing an opioid antagonist administered to a human subject having back pain. An amount of an opioid antagonist and an amount of an opioid agonist are administered to the subject, and back pain is assessed. The amount of the opioid antagonist administered to the subject is adjusted if back pain is not alleviated to a desired extent and/or if one or more adverse effects are not alleviated to a desired extent.

In another aspect, the present disclosure provides methods and materials for dosing an opioid antagonist administered to a human subject having back pain. An amount of an opioid antagonist and an amount of an opioid agonist are administered to the subject, and back pain is assessed. A level of the opioid antagonist or a surrogate of the opioid antagonist in a sample from the subject is measured. The amount of the opioid antagonist administered to the subject is adjusted if the measured level is outside a (e.g., above or below) predetermined range.

In another aspect, the present disclosure provides methods and materials for determining the amount of an opioid antagonist or opioid agonist to be administered to a human subject and back pain is assessed. A level of the opioid antagonist or a surrogate of the opioid antagonist in a sample obtained from the human subject is measured. For example, the level of 6β-naltrexol can be measured as a surrogate. The 6β-naltrexol level (e.g., the concentration of 6β-naltrexol in a plasma sample) can be a surrogate marker for assessing back pain. On the basis of the measured level, the amount of the opioid antagonist or the amount of the opioid agonist for administration to the human subject is adjusted.

In another aspect, the present disclosure provides methods and materials for reducing the level of a biomarker in a human subject having back pain wherein a composition comprising an opioid antagonist and optionally an opioid agonist is administered to the human subject.

In yet another aspect, the present disclosure provides methods and materials for monitoring the response of a human subject being treated for back pain by administering an opioid antagonist and optionally an opioid agonist. The level of one or more one biomarker(s) in a first sample from the subject is determined prior to treatment with the opioid antagonist and optionally the opioid agonist. The level of the biomarker in at least a second sample from the subject is determined subsequent to the initial treatment with the opioid antagonist and optionally the opioid agonist. The level of the biomarker in the second sample is compared with the level of the biomarker in the first sample. A change in the level of the biomarker in the second sample compared to the level of the biomarker in the first sample indicates the effectiveness of the treatment.

Novel compositions, dosage forms, kits, and other materials are described which comprise an opioid antagonist for use in or with the foregoing methods including wherein the amount of the antagonist is effective for enhancing the potency of an opioid agonist for alleviating back pain, and including compositions, dosage forms, kits, and other materials with an opioid agonist and an opioid antagonist, including wherein the amount of the agonist and the amount of the antagonist together are effective for alleviating back pain.

Thus, the present disclosure provides methods and materials comprising opioid antagonists, including opioid agonists and antagonists, that provide pain relief (including enhanced potency of the opioid agonist), with an improvement in side effect profile, even with chronic administration including as compared with methods and materials without opioid antagonists. Advantages of methods and materials of the disclosure include enhanced and prolonged analgesia, attenuation of one or more adverse effects, including, for example, opioid-related adverse effects, such as, for example, skin or subcutaneous tissue disorders (e.g., pruritus), or including, for example, one or more adverse effects selected from withdrawal, dependence, tolerance, or addiction. Additional advantages include, for example, attenuation of one or more symptoms or signs of withdrawal or dependence, prevention of tolerance or continued protection against tolerance even with chronic administration, reversal of opioid agonist-induced hyperalgesia, prevention of physical dependence or withdrawal, decreased rewarding/euphoric side effect, or decreased potential for relapse/addiction.

### BRIEF DESCRIPTION OF THE FIGURES

FIG. 1 shows plasma concentrations (mean±SEM) of oxycodone (ng/mL) in the three treatment groups from the clinical study conducted as described in Example 1: oxycodone QID represented as red circles (∘); the combination drug of oxycodone and naltrexone QID represented as green triangles (Δ); and the combination drug of oxycodone and naltrexone BID represented as pink squares (□).

FIG. 2 shows plasma concentrations (mean±SEM) of oxymorphone (ng/mL) in the three treatment groups from the clinical study conducted as described in Example 1: oxycodone QID represented as the bar having diagonal lines; the combination drug of oxycodone and naltrexone QID represented as the bar having diamonds; the combination drug of oxycodone and naltrexone BID represented as the darker bar having polka dots.

FIG. 3 shows plasma concentrations (median±quartiles) of oxycodone (ng/mL) after the final dose in the three treatment groups from the clinical study conducted as described in Example 1.

FIG. 4 shows log-transformed plasma concentrations (median±quartiles) of oxycodone after the final dose in the three treatment groups from the clinical study conducted as described in Example 1.

FIG. 5 shows log-transformed plasma concentrations (median±quartiles) of oxymorphone after the final dose in the three treatment groups from the clinical study conducted as described in Example 1.

FIG. 6 shows dose-normalized plasma concentrations (mean±SEM) of oxycodone (ng/mL) in the three treatment groups from the clinical study conducted as described in Example 1: oxycodone QID represented as red circles (∘); the combination drug of oxycodone and naltrexone QID represented as green triangles (Δ); and the combination drug of oxycodone and naltrexone BID represented as pink squares (□).

FIG. 7 shows plasma concentrations (mean±SEM) of 6β-naltrexol (μg/mL) for two of the treatment groups from the clinical study conducted as described in Example 1: the combination drug of oxycodone and naltrexone QID represented as the bar having diamonds; the combination drug of oxycodone and naltrexone BID represented as the darker bar having dots.

FIG. 8 shows efficacy measures versus oxycodone concentrations after the final dose for the three treatment from the clinical study conducted as described in Example 1: oxycodone QID represented as black circles; the combination drug of oxycodone and naltrexone BID represented as red squares; the combination drug of oxycodone and naltrexone QID represented as green diamonds.

FIG. 9 shows efficacy measures versus oxymorphone concentrations after the final dose for the three treatment groups from the clinical study conducted as described in Example 1: oxycodone QID represented as black circles; the combination drug of oxycodone and naltrexone BID represented as red squares; the combination drug of oxycodone and naltrexone QID represented as green diamonds.

FIG. 10 shows efficacy measures versus 6β-naltrexol concentrations after the final dose for two of the treatment groups from the clinical study conducted as described in Example 1: the combination drug of oxycodone and naltrexone BID represented as black circles; the combination drug of oxycodone and naltrexone QID represented as red squares.

FIG. 11 shows the percent change in pain intensity reported by some of the subjects in Table 23 vs. 6β-naltrexol plasma concentrations measured for those subjects, as described in Example 3.

FIG. 12 shows the percent change in pain intensity reported by subjects in Table 23 who received the BID dosing regimen vs. 6β-naltrexol plasma concentrations measured for those subjects as described in Example 3.

FIG. 13 shows steps in a process for the preparation of dosage forms of opioid agonist and opioid antagonist.

### DETAILED DESCRIPTION OF THE INVENTION

The present disclosure provides methods and materials, including novel compositions, dosage forms and methods of administration, useful for the treatment of back pain using opioid antagonists, including combinations of opioid antagonists and opioid agonists. The methods and materials provide human subjects with alleviation of back pain and attenuation of one or more adverse effects of the opioid agonist. The back pain may be chronic back pain. The back pain may be moderate or severe. The back pain may be nociceptive, neuropathic or mixed in origin. The methods and materials alleviate back pain and attenuate (e.g., ameliorate, alleviate, reduce, diminish, block, inhibit or prevent) one or more adverse effects selected from withdrawal, dependence, tolerance or addiction. The methods and materials alleviate back pain and attenuate (e.g., ameliorate, alleviate, reduce, diminish, block, inhibit or prevent) one or more opioid-related adverse effects, such as, for example, skin and subcutaneous tissue disorders (e.g., pruritus), gastrointestinal disorders (e.g., constipation, nausea, vomiting), or nervous system disorders (e.g., dizziness, somnolence). Methods and materials provided comprise opioid antagonists, including combinations opioid antagonists and agonists and may optionally include one or more additional therapeutic agents.

The present methods and materials are useful for the treatment of back pain, including but not limited to chronic back pain, acute back pain, low back pain, acute low back pain, chronic low back pain, neck pain, upper back pain, middle back pain, cancerous back pain, non-cancerous back pain, arthritic back pain, non-arthritic back pain, nociceptive back pain, neuropathic back pain, radicular back pain, referred back pain, mechanical back pain, and sciatic back pain.

The present methods and materials are useful for the treatment of inflammation associated with a chronic condition, including chronic back pain.

The present methods and materials are useful for the treatment of back pain, including chronic back pain that is associated with or mediated by the peripheral or central nervous system.

Chronic back pain is pain that persists beyond acute pain, for example for more than 3 months, or beyond the time that normal healing occurs. It is often progressive and the cause can be difficult to determine. Chronic back pain can include pain from or associated with cancer. Acute or short-term low back pain generally lasts from a few days to a few weeks. Acute or chronic back pain may be the result of surgery, including failed back surgery, or trauma, for example, trauma to the lower back or the result of a disorder such as arthritis. Acute or chronic pain may be the result of pathogenic mechanisms that are nociceptive, neuropathic or mixed in origin. Often, however, the acute or chronic back pain has no known cause.

Back pain can be described as radicular back pain, which involves an inflamed nerve root, and referred back pain, which involves a musculoskeletal sprain or strain. Neuropathic back pain generally involves damage to nerve tissue. Nociceptive back pain generally involves an injury or disease outside the nervous system. Some people experience mixed pain, which is a combination of neuropathic and nociceptive back pain. Mechanical back pain is aggravated by movement and worsened by coughing. Mechanical back pain is typical of a herniated disc or stress fracture. For patients with this condition, forward movements of the spine usually cause pain. In addition, posture, coughing, sneezing, and movement can all influence pain coming from the spine. When acute back pain is severe and travels down both legs, it could be caused by lumbar disc disease. Back pain also includes sciatic back pain (or sciatica). Sciatica refers to pain that begins in the hip and buttocks and continues down the leg. The term sciatica generally indicates that the sciatic nerve, which travels from the lower back through the buttocks and into the leg, is thought to be the cause of the pain in this condition. True sciatica is a condition that occurs when a herniated lumbar disc compresses one of the contributing roots of the sciatic nerve.

Back pain can arise from many different causes, such as muscle strains or other muscle injury or disease, rupture or other damage to one or more discs, spinal stenosis, arthritis, spondylolisthesis, and osteoporosis. Muscle strains are a common cause of low back pain. A ruptured intervertebral disc, also called a herniated disc, is another common cause of back pain. Discogenic back pain is thought to be a common cause of low back pain. Discogenic back pain is the result of damage to the intervertabral disc, but without disc herniation. Spinal stenosis refers to constriction of the spinal canal as people age. It may be due in part to arthritis and other conditions. If the spinal canal becomes too tight, back pain can be the result. Arthritis can affect any joint in the body, including the small joints of the spine. Arthritis of the spine can cause back pain with movement. Spondylolisthesis can cause back pain because adjacent vertebra become unstable and begin to “slip.” The most common cause of spondylolisthesis is due to degenerative changes causing loss of the normal stabilizing structures of the spinal column. Osteoporosis can cause a number of orthopedic problems and generalized discomfort. Back pain from osteoporosis is most commonly related to compression fractures of the vertebra. Osteoporosis causes weak bones and can lead to these fractures.

Back pain can originate from spinal compression, degeneration or injury, muscle trauma or irritation, or another non-traumatic event. Low back pain can also begin in other regions of the body and eventually attack the muscles or other structures in the lower back. Sometimes low back pain can even begin in the nerves or nervous system. Other origins for low back pain are surgery, postneural difficulties, congenital disorders, trauma, infections, degenerative disorders, inflammatory diseases, circulatory disorders, failed surgery, or other causes.

Back pain can result from nerve or muscle irritation, bone lesions, or other causes. Most low back pain follows injury or trauma to the back, but pain may also be caused by degenerative conditions such as arthritis or disc disease, osteoporosis or other bone diseases, viral infections, irritation to joints and discs, or congenital abnormalities in the spine. Obesity, smoking, weight gain during pregnancy, stress, poor physical condition, posture inappropriate for the activity being performed, and poor sleeping position also may contribute to low back pain. Additionally, scar tissue created when the injured back heals itself does not have the strength or flexibility of normal tissue. Buildup of scar tissue from repeated injuries eventually weakens the back and can lead to more serious injury.

As discussed in detail herein, back pain can be alleviated using opioid agonists. The present methods and materials comprise opioid antagonists and are useful for the treatment of back pain and provide human subjects with alleviation of back pain and attenuation of one or more adverse effects, such as withdrawal, dependence, tolerance or addiction.

Adverse effects of opioids can include withdrawal, dependence, tolerance or addiction. Physical dependence, tolerance and addiction have been defined by the American Academy of Pain Medicine, the American Pain Society, and the American Society of Addiction Medicine. American Pain Society, “Definitions Related to the Use of Opioids for the Treatment of Pain” (2001). Physical dependence is described as a state of adaptation that is manifested by a drug class specific withdrawal syndrome that can be produced by abrupt cessation, rapid dose reduction, decreasing blood level of the drug, and/or administration of an antagonist. Tolerance is described as a state of adaptation in which exposure to a drug induces changes that result in a diminution of one or more of the drug's effects over time. Addiction is described as a primary, chronic, neurobiologic disease, with genetic, psychosocial, and environmental factions influencing its development and manifestations. It is characterized by behaviors that include one or more of the following: impaired control over drug use, compulsive use, continued use despite harm, and craving.

Physical dependence on and/or tolerance to prescribed drugs are predicatable adverse effects that often occur with the persistent use of certain medications, such as opioids. Physical dependence may develop with chronic use of many classes of medications. When drugs that induce physical dependence are no longer needed, they should be carefully tapered while monitoring clinical symptoms to avoid withdrawal phenomena and such effects as rebound hyperalgesia. At times, anxiety and sweating can be seen in patients who are dependent on sedative drugs, such as alcohol or benzodiazepines, and who continue taking these drugs. This is usually an indication of development of tolerance, though the symptoms may be due to a return of the symptoms of an underlying anxiety disorder, due to the development of a new anxiety disorder related to drug use, or due to true withdrawal symptoms.

Tolerance may occur to both the desired and undesired effects of drugs, and may develop at different rates for different effects. For example, in the case of opioids, tolerance usually develops more slowly to analgesia than to respiratory depression, and tolerance to the constipating effects may not occur at all. Tolerance to the analgesic effects of opioids is considered to be an undesirable or adverse effect. It is variable in occurrence but is not generally absolute; thus, no upper limit to dosage of pure opioid agonists can be established.

Symptoms and signs of withdrawal include hyperalgesia, increased heart rate, increased respiratory rate, or increased pupilary diameter. Symptoms and signs of withdrawal also include muscle cramps, yawning, upset stomach, runny nose, watery eyes, abdominal cramps, chills/gooseflesh, or clammy/damp skin, all of which can be determined measured objectively by a trained observer and/or subjectively by the human subject. Symptoms and signs of withdrawal also include symptoms of the Short Opiate Withdrawal Scale, namely the symptoms of feeling sick, stomach cramps, muscle spasms/twitching, feelings of coldness, heart pounding, muscular tension, aches and pains, yawning, runny eyes, or insomnia/problems sleeping (e.g., insomnia and/or problems sleeping). The SOWS assessment is described in Gossop, “The Development of a Short Opiate Withdrawal Scale (SOWS).” Addictive Behaviors, Vol. 15, p. 487-490, 1990 (incorporated by reference herein), which states “The scale has shown criterion validity in that the scores are elevated during the acute phase of withdrawal and gradually return to normal, baseline after levels after detoxification, and good discriminative efficiency is shown by the scale's capacity to differentiate between addicts during withdrawal phase and post withdrawal.”

Attenuation of withdrawal can be measured by attenuation of hyperalgesia, increased heart rate, increased respiratory rate, increased pupilary diameter, muscle cramps, yawning, upset stomach, runny nose, watery eyes, abdominal cramps, chills/gooseflesh, or clammy/damp skin. Attenuation of withdrawal can also be measured on the Short Opiate Withdrawal Scale (SOWS). Attenuation of withdrawal can also be measured by attenuation of one or more of the symptoms of the Short Opiate Withdrawal Scale, namely the symptoms of feeling sick, stomach cramps, muscle spasms/twitching, feelings of coldness, heart pounding, muscular tension, aches and pains, yawning, runny eyes, or insomnia/problems sleeping.

Attenuation of withdrawal indicates attenuation of physical dependence, since lesser withdrawal indicates lesser physical dependence. Symptoms and signs of physical dependence on an opioid include the symptoms and signs of withdrawal when the patient discontinues taking the opioid. Another symptom or sign of physical dependence is the production of an abstinence syndrome, or its symptoms or signs, by the administration of a sufficiently high dose of opioid antagonist. Symptoms and signs of an abstinence syndrome include anxiety, irritability, chills and hot flashes, joint pain, salivation, lacrimation, rhinorrhea, diaphoresis, piloerection, nausea, vomiting, abdominal cramps, diarrhea, or sleep disturbances/insomnia.

Symptoms and signs of tolerance include a need to increase dose to maintain pain relief, a decrease in duration of pain relief for a given dose, anxiety, or sweating.

Symptoms and signs of addiction include impaired control over opioid agonist, compulsive use of opioid agonist, continued use of opioid agonist despite harm to the subject, or craving of opioid agonist.

The present disclosure provides methods and materials for alleviating back pain and attenuating one or more of the adverse effects and/or symptoms or signs of the adverse effects described above. The present disclosure provides methods and materials for treating back pain by administering to a human subject with back pain an opioid antagonist or an opioid agonist with an opioid antagonist. Methods and materials provided are effective for the treatment of back pain, including chronic back pain, with attenuation of adverse effects. Methods and materials provided are effective for the treatment of moderate, moderate-to-severe back pain or severe back pain. For example, the amount of an opioid antagonist is an amount effective for maintaining or enhancing the potency of an opioid agonist for alleviating the back pain. The pain intensity is thereby alleviated (e.g., ameliorated, attenuated, reduced, diminished, blocked, inhibited or prevented).

The present methods and materials are effective for the treatment of chronic back pain. In the present methods and materials, the antagonist, the agonist, or both the antagonist and the agonist can be administered chronically. For example, the antagonist, the agonist, or both the antagonist and the agonist can be administered for at least six weeks, for at least twelve weeks, or for a longer period.

In the treatment of back pain, including chronic back pain, back pain from inflammation associated with a chronic condition, or back pain associated with an arthritic condition, the antagonist, the agonist, or both the opioid agonist and the opioid antagonist can be administered for at least one week, alternatively for at least two weeks, for at least three weeks, for at least six weeks, for at least twelve weeks, or for a longer period. The antagonist, the agonist, or both the opioid agonist and the opioid antagonist can be administered at least once daily for at least one week, alternatively two weeks, alternatively three weeks, alternatively six weeks, alternatively twelve weeks, alternatively chronically.

The method for treating back pain, including chronic back pain, or back pain from inflammation associated with a chronic condition, or back pain associated with an arthritic condition, may comprise administering the opioid antagonist or each of the opioid agonist and the opioid antagonist no more than twice daily for at least one week, alternatively two weeks, alternatively three weeks, alternatively six weeks, alternatively twelve weeks, alternatively chronically. In the treatment of back pain, an opioid agonist, an opioid antagonist, or the combination of an opioid agonist and an opioid antagonist can each be administered at least once daily or twice daily, alternatively no more than twice daily. The method for treating back pain may comprise administering to the subject a daily amount of the opioid antagonist that is 0.004 mg or less, alternatively 0.002 mg or less.

An effective amount to alleviate (e.g., ameliorate, attenuate, reduce, diminish, block, inhibit or prevent) back pain refers to an amount of opioid antagonist or combination of opioid agonist and antagonist with or without one or more additional therapeutic agents which elicits alleviation (e.g., amelioration, attenuation, reduction, diminishment, blockage, inhibition or prevention) of back pain upon administration to a subject (e.g., patient) in need thereof. The amount of the opioid agonist, the opioid antagonist, or another therapeutic agent can refer to the weight of the salt or the weight of the free base of such agonist, antagonist or agent.

An amount of opioid antagonist that enhances the potency of an opioid agonist to alleviate back pain, is the amount that when added to an analgesic or subanalgesic amount of agonist results upon administration in a greater alleviation (e.g., amelioration, attenuation, reduction, diminishment, blockage, inhibition or prevention) of back pain, than the alleviation of back pain resulting from administration of that agonist alone (i.e., without that amount of antagonist).

An amount of opioid antagonist that enhances the potency of an endogenous opioid agonist is the amount that when administered alone or with opioid agonist or another therapeutic agent, results in a greater alleviation (e.g., amelioration, attenuation, reduction, diminishment, blockage, inhibition or prevention) of at least one sign or symptom of pain than the alleviation of that sign or symptom without that amount of antagonist.

In the compositions for use in methods according to the present disclosure, the agonist may be present in its original form or in the form of a pharmaceutically acceptable salt. The agonists for use in methods according to the present disclosure include: alfentanil, allylprodine, alphaprodine, anileridine, apomorphine, apocodeine, benzylmorphine, bezitramide, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, narceine, nicomorphine, norlevorphanol, normethadone, nalorphine, normorphine, norpipanone, ohmefentanyl, opium, oxycodone, oxymorphone, papaveretum, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, salts thereof, mixtures of any of the foregoing, mixed mu-agonists/antagonists, mu-antagonist combinations, or others known to those skilled in the art. Preferred agonists for use in methods according to the present disclosure are morphine, hydrocodone, oxycodone, codeine, fentanyl (and its relatives), hydromorphone, meperidine, methadone, oxymorphone, propoxyphene or tramadol, or mixtures thereof. Particularly preferred contemplated agonists are morphine, hydrocodone, oxycodone or tramadol. Opioid agonists include exogenous or endogenous opioids. Endogenous opioid agonists include endorphin, beta-endorphin, enkephalin, met-enkephalin, dynorphin, orphanin FQ, neuropeptide FF, nociceptin, endomorphin, endormorphin-1, endormorphin-2.

The agonist may be present in an amount that is analgesic or subanalgesic (e.g., non-analgesic) in the human subject. The agonist is administered in dosage forms containing from about 0.1 to about 300 mg of agonist, alternatively from about 2.5 to about 160 mg of agonist. For example, an agonist (including but not limited to oxycodone) can be administered in dosage forms containing about 1 mg, alternatively about 2.5 mg, alternatively about 5 mg, alternatively about 7.5 mg, alternatively about 10 mg, alternatively about 15 mg, alternatively about 20 mg, alternatively about 30 mg, alternatively about 40 mg, alternatively about 60 mg, alternatively about 80 mg, alternatively about 5 mg, alternatively about 160 mg, alternatively about 320 mg of opioid agonist. Alternatively, the agonist can be administered in dosage forms containing a dose of opioid agonist that is equivalent to the foregoing doses of oxycodone hydrochloride. Equivalent doses of one opioid agonist (such as morphine, hydromorphone, fertanyl, or others can be easily calculated from a stated dose of another opioid agonist (such as oxycodone) using a conversion factor. For example, conversion factors are available from the American Pain Society's website http://www.talaria.org/calculatorJ20.html as Drug Conversion Calculator Version 2.0, from the PDR Electronic Library (2002), and from Goodman and Gilman, supra (see, e.g., Tables 23.6 at page 606 of 10^{th} Edition. The Opioid Conversion Chart set forth in Example 6 below can also be used to determine an equivalent dose of an opioid agonist based on a stated dose of another opioid agonist. For example, where dose amounts of oxycocodone hydrochloride are set forth in the present disclsosure, an equianalgesic dose amount of another opioid agonist may be used. For example, for dose amounts of oxycodone such as 1 mg, 2.5 mg, 5 mg, 7.5 mg, 10 mg, 15 mg, 20 mg, 30 mg, 40 mg, 60 mg, 80 mg, 160 mg, and 320 mg, the following dose amounts of the following opioid agonists may be used:

Codeine Phosphate: 6 mg, 15 mg, 30 mg, 45 mg, 60 mg, 90 mg, 120 mg, 180 mg, 240 mg, 360 mg, 480 mg, 960 mg, and 1920 mg. For example, an oral dose of codeine of 130 mg (q3-4 hr) is an approximate equianalgesic oral dose of 30 mg (q-3-4 hr) of oxycodone.

Hydrocodone Bitartrate: 1 mg, 2.5 mg, 5 mg, 7.5 mg, 10 mg, 15 mg, 20 mg, 30 mg, 40 mg, 60 mg, 80 mg, 160 mg, and 320 mg. For example, an oral dose of hydrocodone of 30 mg (q3-4 hrs) is an approximately equianalgesic oral dose of 30 mg (q3-4 hr) of oxycodone.

Propoxyphene HCl: 9 mg, 22.5 mg, 45 mg, 67.5 mg, 90 mg, 135 mg, 180 mg, 270 mg, 360 mg, 540 mg, 720 mg, 1440 mg, and 2880 mg. For example, an oral dose of propoxyphene of 130 mg (q3-4 hrs) is an approximately equianalgesic oral dose of 30 mg (q3-4 hr) of oxycodone.

Propoxyphene Napsylate: 15 mg, 37.5 mg, 75 mg, 112.5 mg, 150 mg, 225 mg, 300 mg, 450 mg, 600 mg, 900 mg, 1200 mg, 2400 mg, and 4800 mg.

Meperidine HCl: 15 mg, 37.5 mg, 75 mg, 112.5 mg, 150 mg, 225 mg, 300 mg, 450 mg, 600 mg, 900 mg, 1200 mg, 2400 mg, and 4800 mg. For example, an oral dose of meperidine of 300 mg (q3-4 hrs) is an approximately equianalgesic oral dose of 30 mg (q3-4 hr) of oxycodone.

Morphine Sulfate: 1 mg, 2.5 mg, 5 mg, 7.5 mg, 10 mg, 15 mg, 20 mg, 30 mg, 40 mg, 60 mg, 80 mg, 160 mg, and 320 mg. For example, an oral dose of morphine of 30 mg (q3-4 hrs) is an approximately equianalgesic oral dose of 30 mg (q3-4 hr) of oxycodone.

Methadone HCl: 0.333 mg, 0.8325 mg, 1.665 mg, 2.4975 mg, 3.33 mg, 4.995 mg, 6.66 mg, 9.09 mg, 13.32 mg, 19.98 mg, 26.64 mg, 53.28 mg, and 106.56 mg. For example, an oral dose of methadone of 20 mg (q3-4 hrs) is an approximately equianalgesic oral dose of 30 mg (q3-4 hr) of oxycodone.

Levorphanol Tartrate: 0.065 mg, 0.1625 mg, 0.325 mg, 0.4875 mg, 0.065 mg, 0.975 mg, 1.3 mg, 1.95 mg, 2.6 mg, 3.9 mg, 5.2 mg, 10.4 mg, and 20.8 mg. For example, an oral dose of levorphanol of 4 mg (q3-4 hrs) is an approximately equianalgesic oral dose of 30 mg (q3-4 hr) of oxycodone.

Hydromorphone HCl: 0.25 mg, 0.625 mg, 1.25 mg, 1.875 mg, 2.5 mg, 3.75 mg, 5 mg, 7.5 mg, 10 mg, 15 mg, 20 mg, 40 mg, and 80 mg. For example, an oral dose of hydromorphone of 7.5 mg (q3-4 hrs) is an approximately equianalgesic oral dose of 30 mg (q3-4 hr) of oxycodone.

Butorphanol tartrate: 0.1 mg, 0.25 mg, 0.5 mg, 0.75 mg, 1 mg, 1.5 mg, 2 mg, 3 mg, 4 mg, 6 mg, 8 mg, 16 mg, and 32 mg.

Pentazocine HCl: 8.35 mg, 20.875 mg, 41.75 mg, 62.625 mg, 83.5 mg, 125.25 mg, 167 mg, 250.5 mg, 334 mg, 501 mg, 668 mg, 1336 mg, and 2672 mg.

Fentanyl: transdermal doses of 0.025 mg/hr, 0.05 mg/hr, 0.075 mg/hr, and 0.100 mg/hr. A transdermal dose of fentanyl (e.g., DURAGESIC®) of 0.025 mg/hr, 0.050 mg/hr, 0.075 mg/hr, 0.100 mg/hr, 0.125 mg/hr, 0.150 mg/hr, 0.175 mg/hr, 0.20 mg/hr, 0.225 mg/hr, 0.250 mg/hr, 0.275 mg/hr, or 0.300 mg/hr are equivalent to an oxycodone dose of 45-134 mg/day, 135-224 mg/day, 225-314 mg/day, 315-404 mg/day, 405-494 mg/day, 495-584 mg/day, 585-674 mg/day, 675-764 mg/day, 765-854 mg/day, 855-944 mg/day, 945-1034 mg/day, or 1035-1124 mg/day, respectively.

The agonist, in conjunction with antagonist, is included in the dosage form in an amount sufficient to produce the desired effect upon the process or condition of pain, including inflammatory pain, such as alleviation (e.g., amelioration, attenuation, reduction, diminishment, blockage, inhibition or prevention) of at least one symptom of pain, including inflammatory pain. Symptoms and signs include, for example, pain (including chronic pain), stiffness or difficulty in physical function.

Preferred opioid agonists for the present methods and compositions treating back pain include codeine, hydromorphone, meperidine, morphine, oxycodone, oxymorphone, propoxyphene, hydrocodone, pentazocine, fentanyl, sufentanyl, methadone, tramadol, and dihydrocodeine.

Preferred combinations of an opioid antagonist and opioid agonist in the present compositions are naltrexone and oxycodone; naltrexone and oxymorphone; naltrexone and hydrocodone; naltrexone and hydromorphone; naltrexone and morphine; nalmefene and oxycodone; nalmefene and oxymorphone; nalmefene and hydrocodone; nalmefene and hydromorphone; nalmefene and morphine; naloxone and oxycodone; naloxone and oxymorphone; naloxone and hydrocodone; naloxone and hydromorphone; and naloxone and morphine, respectively.

The more preferred combinations of an opioid antagonist and opioid agonist in the present compositions are naltrexone and oxycodone; naltrexone and oxymorphone; naltrexone and hydrocodone; naltrexone and hydromorphone; naltrexone and morphine; nalmefene and oxycodone; nalmefene and oxymorphone; nalmefene and hydrocodone; nalmefene and hydromorphone; and nalmefene and morphine, respectively.

The most preferred combinations of an opioid antagonist and opioid agonist in the present compositions are naltrexone and oxycodone; naltrexone and oxymorphone; naltrexone and hydrocodone; naltrexone and hydromorphone; and naltrexone and morphine, respectively.

The present disclosure also provides methods and materials, including novel compositions, dosage forms and methods of administration, useful for the treatment of back pain, including where the back pain is from, arthritic conditions, inflammation associated with a chronic condition or chronic pain, using opioid antagonists, including combinations of opioid antagonists and opioid agonists. The present methods and materials provide human subjects with alleviation of one or more symptoms or signs of the arthritic condition, inflammation associated with a chronic condition or chronic pain, including, for example, alleviation of pain, alleviation of stiffness and/or improvement of physical function. The present methods and materials comprise opioid antagonists or combinations of opioid antagonists and agonists may optionally include one or more additional therapeutic agents.

In one aspect, the amount of the antagonist is effective for enhancing the potency of an opioid agonist for alleviating one or more symptoms or signs associated with the arthritic condition inflammation, or chronic condition, or the amount of the agonist and the amount of the antagonist together are effective for alleviating one or more symptoms or signs associated with the arthritic condition, inflammation, or chronic condition.

In another aspect, the amount of the antagonist is effective for enhancing the potency of an opioid agonist for inhibiting progression of the arthritic condition, inflammation, or chronic condition, or the amount of the agonist and the amount of the antagonist together are effective for inhibiting progression of the arthritic condition, inflammation, or chronic condition.

In another aspect, the amount of the antagonist is effective for enhancing the potency of an opioid agonist for reversing damage associated with the arthritic condition, inflammation, or chronic condition, or the amount of the agonist and the amount of the antagonist together are effective for reversing damage due to the arthritic condition, inflammation or chronic condition.

In another aspect, the present disclosure provides methods and materials for treating chronic pain by administering to a human subject with chronic pain an opioid antagonist, wherein the amount of the opioid antagonist is effective for enhancing the potency of an opioid agonist to attenuate the chronic pain, or the amount of the agonist and the amount of the antagonist together are effective to attenuate the chronic pain. Chronic pain may result from various abnormal or compromised states (e.g., diseased), including but not limited to osteoarthritis, rheumatoid arthritis, psoriatic arthritis, back pain, cancer, injury or trauma.

One or more symptoms and signs of arthritic conditions, inflammation associated chronic conditions or chronic pain are alleviated (e.g., ameliorated, attenuated, reduced, diminished, blocked, inhibited or prevented), by the present methods and materials, for example, as measured by an alleviation (e.g., amelioration, attenuation, reduction, diminishment, blockage, inhibition or prevention) of pain, stiffness, or difficulty in physical function.

Symptoms and signs of arthritic conditions and inflammation resulting from chronic conditions, are alleviated (e.g., ameliorated, attenuated, reduced, diminished, blocked, inhibited or prevented), by the present methods and materials, for example, as measured by an alleviation (e.g., amelioration, attenuation, reduction, diminishment, blockage, inhibition or prevention) of pain, stiffness, and/or difficulty in physical function.

Thus, the present disclosure provides methods and materials comprising opioid antagonists, including opioid agonists and antagonists, that provide greater pain relief, better pain control, improved function, with no change in side effect profile, even with chronic administration including as compared with methods and materials without opioid antagonists. Advantages of the present methods and materials include enhanced and prolonged analgesia, prevention of tolerance and continued protection against tolerance even with chronic administration, reversal of opioid agonists-induced hyperalgesia, prevention of physical dependence or withdrawal, decreased rewarding/euphoric side effect, and/or decreased potential for relapse/addiction.

The present disclosure provides methods and materials for treating back pain, including back pain from arthritic conditions and/or inflammation associated with chronic conditions in a human subject by administering to the subject an opioid antagonist or an opioid agonist with an opioid antagonist. For example, the amount of an opioid antagonist is effective to enhance the potency of an opioid agonist for alleviating one or more symptoms or signs associated with an arthritic condition or inflammation associated with a chronic condition, for example, symptoms or signs such as pain, stiffness or difficulty in physical function.

The present disclosure provides methods and materials for inhibiting progression of an arthritic condition or inflammation associated with chronic conditions in a human subject including wherein the arthritic condition or inflammation associated with chronic conditions is associated with back pain by administering to the subject an opioid antagonist or an opioid agonist with an opioid antagonist. For example, the amount of an opioid antagonist is an amount effective for enhancing the potency of an opioid agonist for inhibiting progression of the arthritic condition or chronic conditions associated with inflammation. The present disclosure thus provides methods and materials for inhibiting the change or progression in a subject from a normal or uncompromised state (e.g., healthy) to an abnormal or compromised state (e.g., diseased), as indicated, for example, by a symptom or sign associated with an arthritic condition, inflammation from a chronic condition or chronic pain. The progression of an arthritic condition or inflammation associated with a chronic condition can be measured by a variety of methods, including by radiography, by measuring levels of cytokines and/or by measuring B cell and T cell subtype ratios.

The present disclosure provides methods and materials for reversing damage associated with an arthritic condition or inflammation associated with chronic conditions in a human subject including wherein the arthritic condition or inflammation associated with chronic conditions is associated with back pain comprising administering to the subject an opioid antagonist or an opioid agonist with an opioid antagonist. For example, the amount of an opioid antagonist is an amount effective for enhancing the potency of an opioid agonist for reversing damage due to the arthritic condition or inflammation associated with chronic conditions. The present disclosure thus provides methods and materials for reversing the change or progression in a subject from a normal or uncompromised state to an abnormal or compromised state as indicated, for example, by a symptom or sign associated with an arthritic condition, inflammation from a chronic condition or chronic pain. The progression of the arthritic condition or inflammation associated with chronic conditions can be measured by a variety of methods, including by radiography, by measuring levels of cytokines and/or by measuring B cell and T cell subtype ratios.

The present disclosure provides compositions that comprise an opioid antagonist (e.g., an excitatory opioid receptor antagonist). Such compositions additionally preferentially comprise an opioid agonist (e.g., a bimodally-acting opioid agonist), and optionally a pharmaceutically acceptable carrier or excipient for administration to a subject, preferably a human, in need thereof. Such compositions optionally comprise an additional therapeutic agent.

It is contemplated that the present methods and compositions may be employed for the treatment of inflammation associated with chronic conditions (including inhibiting progression of and/or reversing damage associated with inflammation), including the chronic conditions associated with inflammation in and around joints, muscles, bursae, tendons, vertebrae, or fibrous tissue. Such methods and compositions provide reduced pain, reduced stiffness and/or improved physical function.

It is also contemplated that the present methods and compositions may be employed for the treatment of back pain from chronic conditions (including inhibiting progression of and/or reversing damage associated with chronic conditions). Chronic conditions include, for example, arthritic conditions such as osteoarthritis, rheumatoid arthritis, and psoriatic arthritis. For example, the present methods and compositions may be used to treat one or more symptoms or signs of osteoarthritis of the joint, (such as a hip or knee) or the back (for example, the lower back). Chronic conditions also include, for example, conditions associated with or resulting from pain such as chronic pain, including pain associated with or arising from cancer, from infection or from the nervous system (e.g., neurogenic pain such as peripheral neurogenic pain following pressure upon or stretching of a peripheral nerve or root or having its origin in stroke, multiple sclerosis or trauma, including of the spinal cord). Chronic conditions also include, for example, conditions associated with or arising from psychogenic pain (e.g., pain not due to past disease or injury or visible sign of damage inside or outside the nervous system).

The present methods and compositions may also be employed for the treatment of back pain from other arthritic conditions, including gout and spondylarthropathris (including ankylosing spondylitis, Reiter's syndrome, psoriatic arthropathy, enterapathric spondylitis, juvenile arthropathy or juvenile ankylosing spondylitis, and reactive arthropathy). The present methods and compositions may be used for the treatment of back pain from infectious or post-infectious arthritis (including gonoccocal arthritis, tuberculous arthritis, viral arthritis, fungal arthritis, syphlitic arthritis, and Lyme disease).

Additionally, the present methods and compositions may be used for the treatment of arthritis associated with various syndromes, diseases, and conditions, such as arthritis associated with vasculitic syndrome, arthritis associated with polyarteritis nodosa, arthritis associated with hypersensitivity vasculitis, arthritis associated with Luegenec's granulomatosis, arthritis associated with polymyalgin rheumatica, and arthritis associated with joint cell arteritis. Other preferred indications contemplated for employing the compositions and methods herein include calcium crystal deposition arthropathies (such as pseudo gout), non-articular rheumatism (such as bursitis, tenosynomitis, epicondylitis, carpal tunnel syndrome, and repetitive use injuries), neuropathic joint disease, hemarthrosis, Henoch-Schonlein Purpura, hypertrophic osteoarthropathy, and multicentric reticulohistiocytosis. Other preferred indications contemplated for employing the compositions and methods herein include arthritic conditions associated with surcoilosis, hemochromatosis, sickle cell disease and other hemoglobinopathries, hyperlipo proteineimia, hypogammaglobulinemia, hyperparathyroidism, acromegaly, familial Mediterranean fever, Behat's Disease, lupus (including systemic lupus erythrematosis), hemophilia, and relapsing polychondritis.

The methods and compositions for treating back pain, including back pain from arthritic conditions, inflammation associated with chronic conditions or chronic pain alleviate (e.g., ameliorate, attenuate, reduce, diminish, block, inhibit or prevent) at least one symptom or sign of an arthritic condition, inflammation associated with a chronic condition, or chronic pain. For example, the methods and compositions may alleviate one or more of pain intensity, stiffness, or difficulty in physical functions. The methods and compositions may attenuate one or more symptoms or signs of an arthritic condition, inflammation associated with a chronic condition, or chronic pain, wherein the sign or symptom after administration of the composition is ameliorated as compared to the sign or symptom before administration of the composition.

The present disclosure relates to compositions, dosage forms, and kits with an opioid antagonist, including an opioid antagonist in combination with an opioid agonist, wherein the amount of the antagonist enhances the potency of an opioid agonist or wherein the amounts of the agonist and the amount of the antagonist together are effective to alleviate (e.g., ameliorate, attenuate, reduce, diminish, block, inhibit or prevent) one or more symptoms or signs of an arthritic condition, inflammation associated with a chronic condition, or chronic pain. The disclosure further relates to methods for administering to human subjects such compositions, dosage forms, and kits. Optionally, the present methods and materials may further comprise administering a pharmaceutically acceptable carrier or excipient for administration to the subject, preferably a human, in need thereof. Further, optimally, the present methods and materials may comprise an additional therapeutic agent.

The present disclosure also provides methods for treating a subject with pain from an arthritic condition or inflammation associated with a chronic condition, comprising administering an amount of opioid antagonist effective to enhance the pain-alleviating potency of an opioid agonist, including an endogenous opioid agonist and optionally a pharmaceutically acceptable carrier or excipient for administration to the subject, preferably a human, in need thereof, whereby the pain is alleviated. Such methods optionally include additionally administering an opioid agonist, and in such methods, the amount of antagonist is effective to enhance the pain-alleviating potency of the administered agonist.

Back pain can be associated with an arthritic condition or inflammation associated with chronic conditions. The present disclosure also provides methods and materials for treating back pain, including where the back pain is from an arthritic condition or inflammation associated with chronic conditions. The methods comprise administering to a human subject an amount of an opioid antagonist or the combination of an opioid agonist and an opioid antagonist that is effective to enhance potency of the agonist and/or to alleviate one or more symptoms or signs of an arthritic condition or inflammation associated with a chronic condition, including for example, as measured by a suitable index, scale or measure. The attenuation of one or more symptoms or signs of an arthritic condition or of inflammation associated with a chronic condition may be measured on the WOMAC Osteoarthritis Index or one of its subscales (in other words, the pain, stiffness, or physical function subscales of the WOMAC Osteoarthritis Index). Any suitable version of the WOMAC OA Index may be used, including, for example, Version 3.0 or Version 3.1. Any suitable scale may be used as well. The WOMAC OA Index is available in Likert and Visual Analog scaled formats, either of which may be employed in the present methods. WOMAC values can be considered as surrogate markers for the diagnosis, prognosis, monitoring or treatment of an arthritic condition, inflammation from a chronic condition, and/or chronic pain. The WOMAC values represent a subjective surrogate marker. Alternatively or additionally, the attenuation of one or more symptoms or signs may be measured on another suitable index, scale or measure, such the Australian/Canadian (AUSCAN) Osteoarthritis Hand Index or the Osteoarthritis Global Index (OGI). The AUSCAN 3.1 Index and User Guide are currently available from http://www.womac.org/contact/index.cfm, as are the WOMAC 3.1 Osteoarthritis Index and User Guide. Another suitable measure of attenuation is the Definition of Improvement in Rheumatoid Arthritis described in Felson et al., Arthritis & Rheumatism 38:727-735 (1995) incorporated herein by reference. This measure, which also may be designated as the ACR (American College of Rheumatology) 20 improvement, is a composite defined as both improvement of 20% in the number of tender and number of swollen joints, and a 20% improvement in three of the following five: patient global, physician global, patient pain, patient function assessment, and C-reactive protein (CRP). Another suitable measure is described by Paulus et al., Arthritis & Rheumatism 33:477-484 (1990) which is incorporated herein by reference. Paulus et al. provides a definition of improvement based on a set of measures that discriminate between active second-line drug treatment and placebo. These include a 20% improvement in morning stiffness, erythrocyte sedimentation rate (ESR), joint tenderness score, and joint swelling score and improvement by at least 2 grades on a 5-grade scale (or from grade 2 to grade 1) for patient and physician global assessments of current disease severity. Current disease severity can be measured in a variety of ways, including patient or physician global assessments, patient or physician assessments of joint tenderness, joint swelling stiffness, pain, or physical function, cytokine levels, B-cell or T-cell subtype ratios, erythrocyte sedimentation rate (ESR), or C-reactive protein. Suitable measures of attenuation of one or more symptoms or signs, of inhibiting the progression of an arthritic condition or chronic condition, or of reversing tissue or cellular damage include measuring current disease severity. Other indexes, definitions, measures, or scales may also be used for measuring attenuation of one or more symptoms or signs, inhibition of progression, or reversal of tissue or cellular damage.

The present disclosure provides methods and materials for alleviating pain associated with arthritic conditions or inflammation associated with chronic conditions. For example, the amount of an opioid antagonist or the combination of an opioid agonist and an opioid antagonist may be effective to enhance the potency of the agonist and/or to attenuate (e.g., ameliorate, alleviate, reduce, diminish, block, inhibit or prevent) (1) the pain felt by the subject when walking on a flat surface; (2) the pain felt by the subject when going up or down stairs; (3) the pain felt by the subject at night while in bed; (4) the pain felt by the subject that disturbs the sleep of the subject; (5) the pain felt by the subject while sitting or lying down; and/or (6) the pain felt by the subject while standing.

Alternatively or additionally, the present disclosure provides methods and materials for alleviating stiffness associated with arthritic conditions or inflammation associated with chronic conditions. For example, the amount of an opioid antagonist or the combination of an opioid agonist and an opioid antagonist may be effective to enhance the potency of the agonist and/or to attenuate (e.g., ameliorate, alleviate, reduce, diminish, block, inhibit or prevent) (1) the severity of the stiffness felt by the patient after the subject first woke up in the morning; (2) the severity of the stiffness felt by the subject after sitting or lying down later in the day; and/or (3) the severity of the stiffness felt by the subject while resting later in the day.

Alternatively or additionally, the present disclosure provides methods and materials for alleviating difficulty in physical function associated with arthritic conditions or inflammation associated with chronic conditions. For example, the amount of an opioid antagonist or the combination of an opioid agonist and an opioid antagonist may be effective to enhance the potency of the agonist and/or to attenuate (e.g., ameliorate, alleviate, reduce, diminish, block, inhibit or prevent) (1) the difficulty had by the subject when going down stairs; (2) the difficulty had by the human subject when going up stairs; (3) the difficulty had by the subject when getting up from a sitting position; (4) the difficulty had by the subject while standing; (5) the difficulty had by the subject when bending to the floor; (6) the difficulty had by the patient when walking on a flat surface; (7) the difficulty had by the human subject when getting in or out of a car or bus; (8) the difficulty had by the subject while going shopping; (9) the difficulty had by the patient when getting out of bed; (10) the difficulty had by the subject when putting on socks, or panty hose or stockings; (11) the difficulty had by the subject while lying in bed; (12) the difficulty had by the subject when getting in or out of the bathtub; (13) the difficulty had by the subject while sitting; (14) the difficulty had by the patient when getting on or off the toilet; (15) the difficulty had by the subject while doing heavy household chores; and/or (16) the difficulty had by the subject while doing light household chores.

Biomarkers have been identified, as described herein, that are useful in methods and materials for the treatment of back pain, including where the back pain is from an arthritic condition, inflammation from a chronic condition and/or chronic pain, including pain from an arthritic condition or inflammation. A biomarker is a molecular entity, for example, a biochemical in the body, which has a molecular feature that makes it useful for diagnosis, prognosis, monitoring or treatment of a subject, including, for example, measuring progress of disease or effects of treatment. Biomarkers can include inflammatory biomarkers. An inflammatory biomarker can be any suitable biomarker known or recognized as being related to an inflammatory condition, including but not limited to: pro-inflammatory or anti-inflammatory, such as cytokines, interleukin-1 through 17, including interleukin-1α(IL1a), interleukin-1β(IL1b), IL2, IL4, IL5, IL6, IL8, IL10, IL13, tumor necrosis factor alpha (TNFα), GM-CSF, interferon gamma (IFN-γ); markers of systemic inflammation, including, for example, CRP; certain cellular adhesion molecules such as e-selectin, integrins, ICAM-1, ICAM-3, BL-CAM, LFA-2, VCAM-1, NCAM, PECAM, and neopterin; and B61; leukotriene, thromboxane, isoprostane, serum amyloid A protein, fibrinectin, fibrinogen, leptin, prostaglandin E2, serum procalcitonin, soluble TNF receptor 2 (sTNFr2), erythrocyte sedimentation rate, erythema; elevated white blood count (WBC), including percent and total granulocytes (polymorphonuclear leukocytes) monocytes, lymphocytes and eosinophils; and increased erythrocyte sedimentation rate. Further biomarkers of an inflammatory condition may include decreased levels of pre-albumin and albumin.

A sample that contains or may contain a biomarker can be obtained, including a biological sample. Biological sample refers to a sample obtained from an organism (e.g., a human subject) or from components (e.g., cells, tissues or fluids) of an organism. The sample can be a body fluid, tissue, or cell, including, but not limited to, blood, plasma, serum, blood cells (e.g., white cells), tissue or biopsy samples (e.g., tumor biopsy), urine, saliva, tears, sputum, synovial fluid, cerebrospinal fluid, peritoneal fluid, and pleural fluid, or cells therefrom. An exemplary sample is a plasma sample. Biological samples can also include sections of fluids, tissues or cells such as frozen sections taken for histological purposes.

Samples can be analyzed for the presence of biomarkers by a variety of methods. Candidate biomarkers in such samples can include cytokines (e.g., objective biomarkers). Measurement of cytokines can be carried out in a number of ways known to those with skill in the art. Methods are available which can detect cytokines individually using traditional ELISA techniques (for example, Quantikine kits, available from R&D Systems, Minneapolis, Minn.), or several cytokines can be detected simultaneously, using liquid or solid based array systems. For example, Luminex (Austin, Tex.) has developed a liquid array system based on microspheres, wherein the spheres contain a mixture of two fluorophors. The ratio of the two dyes within the mix is precisely controlled, and gives a unique spectral signature to 100 different species of the microbeads. Each of these 100 different species is then coated with known and unique capture reagents, capable of interacting with molecules of interest within a complex mixture such as serum, plasma or cell culture supernatant. These binder molecules can be entities such as antibodies, oligonucleotides, peptides and receptors. A reporter molecule, specific for the analyte molecule of interest, is then used to quantitate binding. The Luminex system requires a specific detector that uses microfluidics to detect individually labeled beads.

Various kits are available for use with this Luminex technology, including the Biosource International (Camarillo, Calif., www.biosource.com) human cytokine ten-plex antibody bead kit. This kit measures members of two classes of cytokines, the TH1/TH2 and the inflammatory cytokines. The TH1/TH2 set includes IL-2, -4, -5, -10, INFγ while the inflammatory set is IL-1β, IL-6, IL-8, GMOCSF, and TNF α. Linco (St. Charles, Mo., www.lincoresearch.com) makes 13, 21, or 22-plex kits for cytokine measurement. The 22-plex kit can simultaneously measure IL-1α, IL-1β, IL-2, -4, -5, -6, -7, -8, -10, -12p70, -13, -15, -17, Eotaxin, G-CSF, GM-CSF, IFNγ, IP-10, MCP-1, MIP-1α, TNFαand RANTES. Another vendor, R & D Systems (Minneapolis, Minn., www.rndsystems.com) makes a kit for the detection of twelve cytokines, including INFγ, bFGF, GM-CSF, G-CSF, IL-2, -4, -5, -6, -8, -10, -17, IL-1β, IL-1α, IL-1ra, TNFα, VEGF, ENA-78, MIP-1, MCP-1, RANTES, and Tpo. Upstate (Charlottesville, Va., www.upstate.com) sells a variety of cytokine detection kits for use with the Luminex system that can detect up to 22 cytokines including IL-1 α, IL-1 β, IL-2, -3, -4, -5, -6, -7, -8, -10, -12(p40), -12(p70), -13, -15, IP-10, Eotaxin, IFNγ, GM-CSF, MCP-1, MIP-1a, RANTES, and TNFα. Qiagen (Valencia, Calif., www.qiagen.com) sells a kit capable of detecting 11 analytes at once, including Eotaxin, MCP-1, RANTES, GM-CSF, INFγ, IL-1α, IL-1β, IL-2, -4, -5, -6, -8, 10, -12p70, and IL-13. Finally, BIORAD (Hercules, Calif., www.biorad.com) sells kits that can detect up to 17 cytokines at once, including: IL-1 β, IL-2, -4, -5, -6, -7, -8, -10, -12p70, -13, -17, G-CSF, GM-CSF, INFγ, MCP-1, MIP-10, and TNFα. In addition, there are other vendors which have similar kits available for purchase for use with the Luminex system.

Other liquid array systems are available for detection of cytokines such as the CBA System developed by BD Bioscience/Pharmingen (Franklin Lakes, N.J., www.bdbiosciences.com). The CBA system also uses coated beads for detection of analytes. The beads are coated with binding molecules, and bound analyte is detected in a ‘sandwich’ assay using a phycoerytherin labeled antibody specific for that analyte in a standard flow cytometer. BD Bioscience/Pharmingen sells kits for detecting several (1-7) analytes at once and examples of these kits are the human TH1/TH2 kit that measures IL-2, -4, -6, 10, TNFαand INFγ, or the human inflammation kit which measures IL1β, IL6, IL8, IL10, TNFαand IL12p70. Bender MedSystems (Vienna, Austria, www.bendermedsystems.com) has developed a product line, the FlowCytomix system, for use with flow cytometer that consists of microbeads coated with antibodies which will interact with various cytokines. The beads are of varying sizes and have unique spectral qualities due to varying amounts of an internal fluorescent dye, and these properties allow the identification of each type of beads within a mixture of beads. Bender MedSystems's multicytokine kit measures several cytokines at once, and those to choose from include INFγ, IL1β, IL-2, -4, -5, -6, -8, -12, MCP-1, TNFα. Bender also sells a TH1/TH2 kit which measures human IL-1β, IL-2, -4, -5, -6, -8, -10, TNFα, TNFβ and INFγ simultaneously.

In addition to the fluid based systems discussed above, methodologies are available for measuring several cytokines at once in solid based array systems. For example, mini array ELISA systems have been used which measure seven different cytokines, TNF-α, IFNα, IFNγ, IL-1α, IL-1β, IL-6, and IL-10 (see Moody et al, BioTechniques 31:186-194 (July 2001)). Biochips have been developed for cytokine measurement (see Huang et al, CANCER RESEARCH 62, 2806-2812, May 15, 2002) wherein 43 cytokines can be detected including GM-CSF, G-CSF, IL-1α, IL-1β, IL-2, -3, -4, -5, 6, -8, -10, -12, -13, TNFα and VEGF. Array systems on glass slides have been developed (Tam et al. Journal of Immunological Methods 261: 157-165 (2002)), for example, capable of measuring eight cytokines including INFγ, IL-2, -4, -5, -6, -10 and -13 and TNFα), or rolling circle amplified-antibody arrays which can measure up to 75 cytokines simultaneously (Schweitzer et al, Nature Biotechnology 20: 359-365 (2002)) including IL-1α, IL-1β, IL-2, -4, -5, -6, -8, 10, -12, TNFα, RANTES and VEGF.

Other array systems, capable of acting either as fluid- or solid-based systems, are available from Pointilliste (Mountain View, Calif., http://www.pointilliste.com). This flexible technology is comprised of self assembling arrays in which the user is able to specifically select the analytes they wish to study. A reporter molecule, specific for the analyte molecule of interest, is then used to quantitate binding. As used herein, the measurements are done on a solid support where capture antibody arrays are applied to a ‘canvas’, wherein each canvas contains up to 96 arrays, and each array may contain up to 625 addressable spots. In this way, each canvas may contain up to 14 million unique, addressable molecules. Anti-cytokine arrays can be prepared in this system, making use of paired antibodies sets such as for example, Cytosets, available from BioSource International. A commercial human Th1/Th2 cytokine canvas is available from Pointilliste and was used as described in Example 4.

One or more cytokines can be employed as biomarkers for treatment using methods and materials as described herein. For example, one or more cytokines can be employed as a biomarker for treatment of an arthritic condition, inflammation associated with a chronic condition, and/or chronic pain, including pain from an arthritic condition or inflammation. One or more cytokines can be used as a biomarker of the existence or extent (e.g., diagnosis, prognosis, monitoring) of an arthritic condition, of inflammation associated with a chronic condition, and/or of chronic pain, including pain from arthritic conditions or inflammation. Alternatively or additionally, one or more cytokines can be used as a biomarker to assess the treatment of an arthritic condition, inflammation associated with a chronic condition, and/or chronic pain, including pain from an arthritic condition or inflammation. Examples of cytokines contemplated for such use as biomarkers include IL1α, IL1β, IL2, IL4, IL5, IL6, IL10, IL13, GM-CSF, interferon-γ and TNFα. Preferably, the cytokines TNFα, IL6, IL4, and/or are used as biomarkers.

Cytokines can be measured as biomarkers before, during and/or after the administration of an opioid agonist, an opioid antagonist, or a combination of an opioid antagonist and opioid agonist. When cytokines are to be employed as biomarkers for a subject, one or more cytokine levels for that subject are measured. Cytokines can be employed as biomarkers, for example, for monitoring, diagnosing, prognosing and/or treating the subject, including but not limited to selecting dose amounts and/or dosing regimens of an opioid antagonist alone or in combination with an opioid agonist.

Level(s) of one or more cytokines, for example, plasma levels, can be measured in a subject at risk for, or seeking, for example, diagnosis, prognosis, monitoring and/or treatment of, or reporting, one or more signs or symptoms of back pain from an arthritic condition or inflammation associated with a chronic condition, and/or chronic back pain, including chronic back pain from an arthritic condition or inflammation. For example, depending on the measured cytokine level(s), an appropriate treatment can be selected and administered. The measured cytokine level(s) can be used to determine whether and how much opioid agonist and/or opioid antagonist are administered. Furthermore, for example, the dose amount and/or dosing regimen of an opioid agonist, an opioid antagonist, or a combination of an opioid antagonist and opioid agonist can be selected based upon the measured cytokine level(s). For example, if one or more of the measured cytokine levels is above a value, a physician can choose to treat a subject by administering an opioid agonist, an opioid antagonist, or a combination of opioid antagonist and opioid agonist. The value can be a predetermined value or a value determined at the time of or after measurement of the cytokine level(s). As another example, a physician can select a higher or lower amount of opioid agonist and/or a higher or lower amount of antagonist for administration. As yet another example, a more frequent or less frequent dosing regimen can be selected based on the measured cytokine level(s). For example, if the level of cytokines are higher than desired, an opioid antagonist can be dosed more frequently, or if the level of cytokines are lower than desired, an opioid antagonist can be dosed less frequently.

Level(s) of one or more cytokines, for example, plasma levels, can be measured for a subject who has already received or who is receiving treatment for back pain from an arthritic condition or inflammation associated with a chronic condition, and/or chronic back pain, including chronic back pain from an arthritic condition or inflammation. The measured cytokine level(s) can be used to determine whether appropriate amounts and regimens have been or are being employed for treating the subject. For example, level(s) of one or more cytokines can be measured in a subject receiving treatment for back pain from an arthritic condition or inflammation associated with a chronic condition, and/or chronic back pain, including chronic back pain from an arthritic condition or inflammation. If the one or more of the measured cytokine levels is above a value, the treatment can be adjusted by administering a greater or lesser amount of an opioid agonist, an opioid antagonist, or a combination of opioid antagonist and opioid agonist and/or by altering the dosing regimen. The value can be a predetermined value or a value determined at the time of or after measurement of the cytokine level(s).

Concentrations of cytokines, for example, plasma concentrations, can be used as biomarkers in adjusting the administration of an opioid antagonist to a subject. A single cytokine concentration can be selected to evaluate whether a subject is in need of treatment. As an example, if a subject has a plasma concentration of TNFα which is higher than about 0.08 ng/ml, alternatively higher than 0.2 ng/ml, the subject is administered more opioid antagonist and/or more opioid agonist, by administering higher dose amounts and/or by administering on a more frequent dosing regimen. As another example, if the subject has a plasma concentration of TNFα which is about 0.08 ng/ml or lower, alternatively lower than 0.2 ng/ml, either the administration of opioid antagonist is not changed, or the subject is administered less opioid antagonist and/or less opioid agonist, by administering lower dose amounts and/or by administering on a less frequent dosing regimen. As another example, if a subject has a plasma concentration of IL4 which is higher than about 0.23 ng/ml, the subject is administered more opioid antagonist and/or more opioid agonist, by administering higher dose amounts and/or by administering on a more frequent dosing regimen. As another example, if the subject has a plasma concentration of IL4 which is about 0.23 ng/ml or lower, either the administration of opioid antagonist is not changed, or the subject is administered less opioid antagonist and/or less opioid agonist, by administering lower dose amounts and/or by administering on a less frequent dosing regimen. As another example, if a subject has a plasma concentration of IL6 which is higher than about 0.18 ng/ml, the subject is administered more opioid antagonist and/or more opioid agonist, by administering higher dose amounts and/or by administering on a more frequent dosing regimen. As another example, if the subject has a plasma concentration of IL6 which is about 0.18 ng/ml or lower, either the administration of opioid antagonist is not changed, or the subject is administered less opioid antagonist and/or less opioid agonist, by administering lower dose amounts and/or by administering on a less frequent dosing regimen.

One or more cytokine concentrations can be used as biomarkers in adjusting the administration of an opioid antagonist to a subject. For example, one or more of the concentrations of IL1α, IL1β, IL2, IL4, IL5, IL6, IL10, IL13, GM-CSF, interferon-γ and TNFα can be used to determine or adjust the treatment of back pain from an arthritic condition or inflammation associated with a chronic condition, and/or chronic back pain, including chronic back pain from an arthritic condition or inflammation.

Concentrations of agonist, antagonists, surrogates such as 6β-naltrexol and/or biomarkers such as cytokines can be useful in methods and materials for the treatment of back pain, including where the back pain is from an arthritic condition, inflammation from a chronic condition and/or chronic pain, including pain from an arthritic condition or inflammation. Such concentrations are particularly useful where a relationship is known between the concentration and an effect on the subject. For example, where the relationship between the effect of an opioid antagonist and a concentration of an agonist, antagonist surrogate or a biomarker is known, preferred and/or suitable ranges for the combined use of an opioid antagonist with an opioid agonist can be selected for seeking a desired effect. The plasma concentration-effect relationship of low dose of an opioid antagonist when administered with an opioid agonist has been represented for the first time by the Emax composite model:
E=[Emax1(Cpⁿ¹)/EC51ⁿ¹+Cpⁿ¹]+[Emax2(Cpⁿ²)/EC52ⁿ²+Cpⁿ²]
where the respective Emax values represent maximum effect for a given drug; EC51 and EC52 represent the potencies, for the drug notated as either 1 or 2, respectively (in other words, EC51 is not the concentration having 51% of the maximal effect, but rather EC51 is the concentration having a particular potency (e.g. 50% of the maximal effect for Effect No. 1); the respective values for C are the concentrations of drugs notated as 1 or 2, and the values of n₁, and n₂ that correspond to the sigmoidicity factors that are associated with particular EC values. In the Emax composite model, “+” is used to indicate absolute values; sometimes it is shown as a “−” which reflects a negative second term.

The Emax composite model is a recognized composite model for PK/PD data analysis set forth, for example, in Gabrielsson et al., PHARMACOKINETIC/PHARMACODYNAMIC DATA ANALYSIS: CONCEPTS AND APPLICATIONS, pp. 191-193 and 801-808 (2000), and the computer command files provided with the reference and described, including with examples of the computer printouts on pages 801-808, all of which is incorporated by reference herein. However, it is believed that the Emax composite model has not previously been utilized for the analysis of PK data from administering low doses of opioid antagonists such as naltrexone for enhancing the potency of opioid agonists such as oxycodone, as described herein. From the plasma concentration-effect data obtained and described in Example 3, it is contemplated that the opioid antagonist, at lower plasma concentrations, is impacting the total effect (percent change in pain intensity), primarily as described by the terms of the equation denoted with a 2.

The recognition of the applicability and utility of a composite model as shown above enables the selection of preferred and/or suitable ranges for the combined use of an opioid antagonist with an opioid agonist as described herein. The composite model provides the relative contribution of an opioid antagonist with respect to enhancing pain relief, for example, as measured by a reduction in pain intensity. The effective percentage decrease in pain intensity, E, has been found to be described by a relatively wide scope of preferred plasma concentrations by the Emax composite model, as described in Example 3 and as shown in the data and Figures described herein.

Opioids refer to compounds or compositions, including metabolites of the compounds or compositions, that bind to specific opioid receptors and have agonist (activation) or antagonist (inactivation) effects at the opioid receptors.

Inhibitory opioid receptors refer to opioid receptors that mediate inhibitory opioid receptor functions, such as analgesia.

Opioid receptor agonist or opioid agonist refers to an opioid compound or composition, including any active metabolite of such compound or composition, that binds to and activates opioid receptors on neurons that mediate pain.

An opioid receptor antagonist or opioid antagonist refers to an opioid compound or composition, including any active metabolite of such compound or composition, for example, that binds to and blocks opioid receptors on neurons that mediate pain. An opioid antagonist attenuates (e.g., blocks, inhibits, prevents, or competes with) the action of an opioid agonist.

Pharmaceutically acceptable refers to those compounds, materials, compositions, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio.

Pharmaceutically acceptable salts refer to derivatives of the disclosed compounds wherein the compounds are modified by making at least one acid or base salt thereof, and includes inorganic and organic salts.

An analgesic amount of opioid agonist refers to an amount of the opioid agonist which causes analgesia in a patient administered the opioid receptor agonist alone, and includes standard doses of the agonist which are typically administered to cause analgesia (e.g. mg doses).

A subanalgesic amount of opioid agonist refers to an amount which does not cause analgesia in a patient administered the opioid receptor agonist alone, but when used in combination with a potentiating or enhancing amount of opioid antagonist, results in analgesia.

An effective antagonistic amount of opioid agonist refers to an amount that effectively attenuates (e.g. ameliorates, reduces, diminishes, blocks, inhibits, prevents, or competes with) the analgesic activity of an opioid agonist.

A therapeutically effective amount of a composition refers to an amount that elicits alleviation (e.g., amelioration, attenuation, reduction, diminishment, blockage, inhibition or prevention) of at least one sign or symptom of an arthritic condition, inflammation associated with a chronic condition, or chronic pain upon administration to a patient in need thereof.

The antagonist in the present compositions may be present in its original form or in the form of a pharmaceutically acceptable salt. The antagonists in the present compositions include: naltrexone, naloxone, nalmefene, methylnaltrexone, methiodide, nalorphine, naloxonazine, nalide, nalmexone, nalorphine dinicotinate, naltrindole (NTI), naltrindole isothiocyanate, (NTII), naltriben (NTB), nor-binaltorphimine (nor-BNI), b-funaltrexamine (b-FNA), BNTX, cyprodime, ICI-174,864, LY117413, MR2266, or an opioid antagonist having the same pentacyclic nucleus as nalmefene, naltrexone, levorphanol, meptazinol, dezocine, or their pharmacologically effective esters or salts. Preferred opioid antagonists include naltrexone, nalmefene, naloxone, or mixtures thereof. Particularly preferred is nalmefene or naltrexone.

In general, for compositions, dosage forms, kits and methods according to the present disclosure, an opioid antagonist is provided in an amount from about 1 fg to about 1.0 mg or from about 1 fg to about 1 μg including where the amount is provided by administration 1, 2, 3, or 4 times per day. Alternatively, the opioid antagonist is provided in an amount from at least about 0.000001 mg to about or less than about 0.5 or 1.0 mg, 0.00001 mg to about or less than about 0.5 or 1.0 mg, 0.0001 mg to about or less than about 0.5 or 1.0 mg, or at least about 0.001 mg to about or less than about 0.5 or 1.0 mg, or at least about 0.01 mg to about or less than about 0.5 or 1.0 mg, or at least about 0.1 mg to about or less than about 0.5 or 1.0 mg. Preferred ranges of opioid antagonists also include: from about 0.000001 mg to less than 0.2 mg; from about 0.00001 mg to less than 0.2 mg; from about 0.0001 mg to less than 0.2 mg; from about 0.001 mg to less than 0.2 mg; from about 0.01 mg to less than 0.2 mg; or from about 0.1 mg to less than 0.2 mg. Additional preferred ranges of opioid antagonists include: from about 0.0001 mg to about 0.1 mg; from about 0.001 mg to about 0.1 mg; from about 0.01 mg to about 0.1 mg; from about 0.001 mg to about 0.1 mg; from about 0.001 mg to about 0.01 mg; or from about 0.01 mg to about 0.1 mg.

In a preferred dosage form, the maximum amount of antagonist is 1 mg, alternatively less than 1 mg, alternatively 0.99 mg, alternatively 0.98 mg, alternatively 0.97 mg, alternatively 0.96 mg, alternatively 0.95 mg, alternatively 0.94 mg, alternatively 0.93 mg, alternatively 0.92 mg, alternatively 0.91 mg, alternatively 0.90 mg, alternatively 0.89 mg, alternatively 0.88 mg, alternatively 0.87 mg, alternatively 0.86 mg, alternatively 0.85 mg, alternatively 0.84 mg, alternatively 0.83 mg, alternatively 0.82 mg, alternatively 0.81 mg, alternatively 0.80 mg, alternatively 0.79 mg, alternatively 0.78 mg, alternatively 0.77 mg, alternatively 0.76 mg, alternatively 0.75 mg, alternatively 0.74 mg, alternatively 0.73 mg, alternatively 0.72 mg, alternatively 0.71 mg, alternatively 0.70 mg, alternatively 0.69 mg, alternatively 0.68 mg, alternatively 0.67 mg, alternatively 0.66 mg, alternatively 0.65 mg, alternatively 0.64 mg, alternatively 0.63 mg, alternatively 0.62 mg, alternatively 0.61 mg, alternatively 0.60 mg, alternatively 0.59 mg, alternatively 0.58 mg, alternatively 0.57 mg, alternatively 0.56 mg, alternatively 0.55 mg, alternatively 0.54 mg, alternatively 0.53 mg, alternatively 0.52 mg, alternatively 0.51 mg, alternatively 0.50 mg.

Additionally, the maximum amount of antagonist in the dosage form is less than 0.5 mg, alternatively 0.49 mg, alternatively 0.48 mg, alternatively 0.47 mg, alternatively 0.46 mg, alternatively 0.45 mg, alternatively 0.44 mg, alternatively 0.43 mg, alternatively 0.42 mg, alternatively 0.41 mg, alternatively 0.40 mg, alternatively 0.39 mg, alternatively 0.38 mg, alternatively 0.37 mg, alternatively 0.36 mg, alternatively 0.35 mg, alternatively 0.34 mg, alternatively 0.33 mg, alternatively 0.32 mg, alternatively 0.31 mg, alternatively 0.30 mg, alternatively 0.29 mg, alternatively 0.28 mg, alternatively 0.27 mg, alternatively 0.26 mg, alternatively 0.25 mg, alternatively 0.24 mg, alternatively 0.23 mg, alternatively 0.22 mg, alternatively 0.21 mg, alternatively 0.20 mg, alternatively 0.19 mg, alternatively 0.18 mg, alternatively 0.17 mg, alternatively 0.16 mg, alternatively 0.15 mg, alternatively 0.14 mg, alternatively 0.13 mg, alternatively 0.12 mg, alternatively 0.11 mg, alternatively 0.10 mg, alternatively 0.09 mg, alternatively 0.08 mg, alternatively 0.07 mg, alternatively 0.06 mg, alternatively 0.05 mg, alternatively 0.04 mg, alternatively 0.03 mg, alternatively 0.02 mg, alternatively 0.01 mg, alternatively 0.009 mg, alternatively 0.008 mg, alternatively 0.007 mg, alternatively 0.006 mg, alternatively 0.005 mg, alternatively 0.004 mg, alternatively 0.003 mg, alternatively 0.002 mg, alternatively 0.001 mg, alternatively 0.0009 mg, alternatively 0.0008 mg, alternatively 0.0007 mg, alternatively 0.0006 mg, alternatively 0.0005 mg, alternatively 0.0004 mg, alternatively 0.0003 mg, alternatively 0.0002 mg.

The minimum amount of antagonist in the dosage form is 0.0001 mg, alternatively 0.0002 mg, alternatively 0.0003 mg, alternatively 0.0004 mg, alternatively 0.0005 mg, 0.0006 mg, alternatively 0.0007 mg, alternatively 0.0008 mg, alternatively 0.0009 mg, alternatively 0.001 mg, alternatively 0.002 mg, alternatively 0.003 mg, alternatively 0.004 mg, alternatively 0.005 mg, alternatively 0.006 mg, alternatively 0.007 mg, alternatively 0.008 mg, alternatively 0.009 mg, alternatively 0.01 mg, alternatively 0.011 mg, alternatively 0.012 mg, alternatively 0.013 mg, alternatively 0.014 mg, alternatively 0.015 mg, alternatively 0.016 mg, alternatively 0.017 mg, alternatively 0.018 mg, alternatively 0.019 mg, alternatively 0.02 mg, alternatively 0.021 mg, alternatively 0.022 mg, alternatively 0.023 mg, alternatively 0.024 mg, alternatively 0.025 mg, alternatively 0.026 mg, alternatively 0.027 mg, alternatively 0.028 mg, alternatively 0.029 mg, alternatively 0.03 mg, alternatively 0.031 mg, alternatively 0.032 mg, alternatively 0.033 mg, alternatively 0.034 mg, alternatively 0.035 mg, alternatively 0.036 mg, alternatively 0.037 mg, alternatively 0.038 mg, alternatively 0.039 mg, alternatively 0.04 mg, alternatively 0.041 mg, alternatively 0.042 mg, alternatively 0.043 mg, alternatively 0.044 mg, alternatively 0.045 mg, alternatively 0.046 mg, alternatively 0.047 mg, alternatively 0.048 mg, alternatively 0.049 mg, alternatively 0.05 mg, alternatively 0.051 mg, alternatively 0.052 mg, alternatively 0.053 mg, alternatively 0.054 mg, alternatively 0.055 mg, alternatively 0.056 mg, alternatively 0.057 mg, alternatively 0.058 mg, alternatively 0.059 mg, alternatively 0.06 mg, alternatively 0.061 mg, alternatively 0.062 mg, alternatively 0.063 mg, alternatively 0.064 mg, alternatively 0.065 mg, alternatively 0.066 mg, alternatively 0.067 mg, alternatively 0.068 mg, alternatively 0.069 mg, alternatively 0.07 mg, alternatively 0.071 mg, alternatively 0.072 mg, alternatively 0.073 mg, alternatively 0.074 mg, alternatively 0.075 mg, alternatively 0.076 mg, alternatively 0.077 mg, alternatively 0.078 mg, alternatively 0.079 mg, alternatively 0.08 mg, alternatively 0.081 mg, alternatively 0.082 mg, alternatively 0.083 mg, alternatively 0.084 mg, alternatively 0.085 mg, alternatively 0.086 mg, alternatively 0.087 mg, alternatively 0.088 mg, alternatively 0.089 mg, alternatively 0.09 mg, alternatively 0.091 mg, alternatively 0.092 mg, alternatively 0.093 mg, alternatively 0.094 mg, alternatively 0.095 mg, alternatively 0.096 mg, alternatively 0.097 mg, alternatively 0.098 mg, alternatively 0.099 mg, alternatively 0.1 mg, alternatively 0.11 mg, alternatively 0.12 mg, alternatively 0.13 mg, alternatively 0.14 mg, 0.15 mg, alternatively 0.16 mg, alternatively 0.17 mg, alternatively 0.18 mg, alternatively 0.19 mg, alternatively 0.2 mg, alternatively 0.21 mg, alternatively 0.22 mg, alternatively 0.23 mg, alternatively 0.24 mg, alternatively 0.25 mg, alternatively 0.26 mg, alternatively 0.27 mg, alternatively 0.28 mg, alternatively 0.29 mg, alternatively 0.3 mg, alternatively 0.31 mg, alternatively 0.32 mg, alternatively 0.33 mg, alternatively 0.34 mg, alternatively 0.35 mg, alternatively 0.36 mg, alternatively 0.37 mg, alternatively 0.38 mg, alternatively 0.39 mg alternatively 0.40 mg, alternatively 0.41 mg, alternatively 0.42 mg, alternatively 0.43 mg, alternatively 0.44 mg, alternatively 0.45 mg, alternatively 0.46 mg, alternatively 0.47 mg, alternatively 0.48 mg, alternatively 0.49 mg, alternatively 0.5 mg, alternatively 0.51 mg, alternatively 0.52 mg, alternatively 0.53 mg, alternatively 0.54 mg, alternatively 0.55 mg, alternatively 0.56 mg, alternatively 0.57 mg, alternatively 0.58 mg, alternatively 0.59 mg, alternatively 0.6 mg, alternatively 0.61 mg, alternatively 0.62 mg, alternatively 0.63 mg, alternatively 0.64 mg, alternatively 0.65 mg, alternatively 0.66 mg, alternatively 0.67 mg, alternatively 0.68 mg, alternatively 0.69 mg, alternatively 0.7 mg, alternatively 0.71 mg, alternatively 0.72 mg, alternatively 0.73 mg, alternatively 0.74 mg, alternatively 0.75 mg, alternatively 0.76 mg, alternatively 0.77 mg, alternatively 0.78 mg, alternatively 0.79 mg, alternatively 0.8 mg, alternatively 0.81 mg, alternatively 0.82 mg, alternatively 0.83 mg, alternatively 0.84 mg, alternatively 0.85 mg, alternatively 0.86 mg, alternatively 0.87 mg, alternatively 0.88 mg, alternatively 0.89 mg, alternatively 0.9 mg, alternatively 0.91 mg, alternatively 0.92 mg, alternatively 0.93 mg, alternatively 0.94 mg, alternatively 0.95 mg, alternatively 0.96 mg, alternatively 0.97 mg, alternatively 0.98 mg, alternatively 0.99 mg.

In a more preferred dosage form, the maximum amount of antagonist is less than 0.0020 mg, alternatively 0.0019 mg, alternatively 0.0018 mg, alternatively 0.0017 mg, alternatively 0.0016 mg, alternatively 0.0015 mg, alternatively 0.0014 mg, alternatively 0.0013 mg, alternatively 0.0012 mg, alternatively 0.0011 mg, alternatively 0.0010 mg, alternatively 0.0009 mg, alternatively 0.0008 mg, alternatively 0.0007 mg, alternatively 0.0006 mg, alternatively 0.0005 mg, alternatively 0.0004 mg, alternatively 0.0003 mg, alternatively 0.0002 mg, alternatively 0.0001 mg.

In a more preferred dosage form, the minimum amount of antagonist in the preferred dosage form is 0.0001 mg, alternatively 0.0002 mg, alternatively 0.0003 mg, alternatively 0.0004 mg, alternatively 0.0005 mg, alternatively 0.0006 mg, alternatively 0.0007 mg, alternatively 0.0008 mg, alternatively 0.0009 mg, alternatively 0.0010 mg, alternatively 0.0011 mg, alternatively 0.0012 mg, alternatively 0.0013 mg, alternatively 0.0014 mg, alternatively 0.0015 mg, alternatively 0.0016 mg, alternatively 0.0017 mg, alternatively 0.0018 mg, alternatively 0.0019 mg, alternatively 0.002 mg.

Any minimum amount and any maximum amount of antagonist in the dosage form, as specified above, may be combined to define a range of amounts, providing that the minimum selected is equal to or less than the maximum selected.

The amount of an opioid antagonist in the compositions for use in methods according to the present disclosure effective to enhance the potency of an opioid agonist can be less than an effective antagonistic amount. The effective amount of an opioid antagonist in the present compositions can be about 0.002 mg. The effective amount of an opioid antagonist in the present compositions can be less than 0.002 mg. The effective amount of an opioid antagonist in the present compositions can be about 0.001 mg. The effective amount of an opioid antagonist in the present compositions can be less than 0.001 mg. The effective amount of an opioid antagonist in the present compositions can be more than 0.0001 mg. The effective amount of an opioid antagonist in the present compositions can be about 0.0001 mg. The effective amount of an opioid antagonist in the present compositions can be about 0.00001 mg. The effective amount of an opioid antagonist in the present compositions can be less than 0.00001 mg. The effective amount of an opioid antagonist in the present compositions can be more than 0.00001 mg. The effective amount of an opioid antagonist in the present compositions can be about 0.000001 mg. The effective amount of an opioid antagonist in the present compositions can be less than 0.000001 mg. The effective amount of an opioid antagonist in the present compositions can be more than 0.000001 mg.

Any of the foregoing effective amounts may be administered one time per day, alternatively two times per day, alternatively three times per day, alternatively four times per day, preferably two times per day. Alternatively any of the following effective amounts may be divided over a series of dosages within one day or other relevant time period. For example, the effective amount may be divided into one, two, three or four doses administered over the day or other time period. Preferred effective amounts of an opioid antagonist include a total daily dose from about 0.00002 mg to about 0.002 mg, wherein the total daily dose is divided into 1, 2, 3, or 4 doses. For example, where the dose is administered two times per day, the opioid antagonist in preferably in an amount from about 0.00001 mg to about 0.001 mg in each of the two doses. Alternatively, where the dose is administered one time per day, the opioid antagonist in an amount from about 0.00002 mg to about 0.002 mg in the dose. Alternatively, where the dose is administered four times per day, the opioid antagonist in an amount from about 0.000005 mg to about 0.0005 mg in each of the four doses.

The amount of antagonist in a dosage form may be less than an effective amount to antagonize an exogenous or endogenous agonist, but such an amount is effective to enhance the pain-enhancing potency, including the inflammatory pain-enhancing potency, of the agonist and optionally but preferably is effective to attenuate an adverse effect of the agonist, for example, tolerance, withdrawal, dependence and/or addiction. Alternatively, the opioid agonist can be administered, in either a combined dosage form with the antagonist or in a separate dosage form. The present disclosure also provides an immediate release solid oral dosage form comprising one or more pharmaceutical excipients, a dose of an opioid agonist and a low dose of an opioid antagonist, wherein the opioid agonist and opioid antagonist are release concurrently when placed in an aqueous environment. The opioid antagonist and opioid agonist can be formulated as immediate release, (IR), controlled release (CR) and/or sustained released (SR) formulations. Formulations can have components that are combinations of IR and/or CR and/or SR components.

The combination dosage forms of the present compositions can be formulated to provide a concurrent release of the opioid antagonist in combination with opioid agonist and/or other therapeutic agent generally throughout at least a majority of the delivery profile for the formulation. As used herein, the terms “concurrent release” and “released concurrently” mean that the agonist and antagonist are released in in vitro dissolution assays in an overlapping manner. The respective beginnings of release of each agent can but need not necessarily be simultaneous. Concurrent release will occur when the majority of the release of the first agent overlap a majority of release of the second agent. A desired portion of each active pharmaceutical ingredient may be released within a desired time. The desired portions may be, for example, 5%, 50% or 90%, or some other percentage between 1% and 100%. The desired time may be in minutes or hours, for example, 10 minutes, 20 minutes, 30 minutes, or 45 minutes, or some other time. The desired portion and the desired time may be varied by the inclusion of formulants for the controlled release or sustained release of any therapeutic agent(s).

The optimum amounts of the opioid antagonist administered in combination with an opioid agonist or other therapeutic agent will of course depend upon the particular antagonist and agonist or other agent used, the excipient chosen, the route of administration, and/or the pharmacokinetic properties of the patient being treated. Effective administration levels of antagonist and agonist or other agent will vary upon the state and circumstances of the patient being treated. As those skilled in the art will recognize, many factors that modify the action of an active ingredient will be taken into account by a treating physician, such as the age, body weight, sex, diet, and condition of the patient, the lapse of time between the condition or injury and the administration of the present compositions, and the administration technique. A person of ordinary skill in the art will be able to ascertain the optimal dosage for a given set of conditions in view of the disclosure herein.

The opioid agonist and/or antagonist can be present in the present compositions as an acid, base, pharmaceutically acceptable salt, or a combination thereof. The pharmaceutically acceptable salt embraces inorganic or organic salts. Examples of pharmaceutically acceptable salts include, but are not limited to, mineral or organic acid salts. The pharmaceutically acceptable salts include the conventional non-toxic salts made, for example, from non-toxic inorganic or organic acids. For example, such conventional non-toxic salts include those derived from inorganic acids such as hydrochloric, hydrobromic, sulfuric, sulfonic, sulfamic, phosphoric, nitric and others known to those skilled in the art; and the salts prepared from organic acids such as amino acids, acetic, propionic, succinic, glycolic, stearic, lactic, malic, malonic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, glucuronic, and other acids. Other pharmaceutically acceptable salts and variants include mucates, phosphate (dibasic), phosphate (monobasic), acetate trihydrate, bi(heptafluorobutyrate), bi(methylcarbamate), bi(pentafluoropropionate), mesylate, bi(pyridine-3-carboxylate), bi(trifluoroacetate), bitartrate, chlorhydrate, and sulfate pentahydrate. An oxide, though not usually referred to by chemists as a salt, is also a “pharmaceutically acceptable salt” for the present purpose. For acidic compounds, the salt may include an amine-based (primary, secondary, tertiary or quaternary amine) counter ion, an alkali metal cation, or a metal cation. Lists of suitable salts are found in texts such as Remington's Pharmaceutical Sciences, 18^{th} Ed. (Alfonso R. Gennaro, ed.; Mack Publishing Company, Easton, Pa., 1990); Remington: the Science and Practice of Pharmacy 19^{th} Ed.(Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are all incorporated herein by reference. Additional representative salts include hydrobromide, hydrochloride, mucate, succinate, n-oxide, sulfate, malonate, acetate, phosphate dibasic, phosphate monobasic, acetate trihydrate, bi(heplafluorobutyrate), maleate, bi(methylcarbamate), bi(pentafluoropropionate), mesylate, bi(pyridine-3-carboxylate), bi(trifluoroacetate), bitartrate, chlorhydrate, fumarate, and sulfate pentahydrate.

The methods may further comprise administering to the subject another therapeutic agent, for example, non-steroidal anti-inflammatory drug agents or local anesthetic and/or analgesic agents, muscle relaxants, antidiarrheal agents such as loperamide, TNF-α antagonists, corticosteroids, disease-modifying anti-rheumatic drugs (DMARDs), anticonvulsant agents, tricyclic antidepressant agents, anti-dynorphin agents, glutamate receptor antagonist agents. In particularly, it is specifically completed that, in addition to the opioid agonist and the opioid antagonist, the subject may be administered TNF-α antagonists, P38 inhibitors, and cytokines inhibitors (including but not limited to IL-2, IL-6, IL-8, and GM-CSF). The opioid agonist, the opioid antagonist, and other therapeutic agent may be administered to the subject in a combined dosage form.

An NSAID refers to a non-steroidal anti-inflammatory drug and includes anti-inflammatory drugs such as aspirin, members of the cycloxgenease I, II and III inhibitors, and includes naproxen sodium, diclofenac and misoprostol, valdecoxib, diclofenac, celecoxib, sulindac, oxaprozin, diflunisal, piroxicam, indomethacin, meloxicam, ibuprofen, naproxen, mefenamic acid, nabumetone, ketorolac, choline or magnesium salicylates, rofecoxib, tolmetin sodium, phenylbutazone, oxyphenbutzone, meclofenamate sodium or diflusenal.

In an embodiment, the present compositions further comprise at least one non-narcotic analgesic, such as a nonsteroidal anti-inflammatory agent (NSAID). Representative nonsteroidal anti-inflammatory agents include aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxican, sulindac, tolmetin, and zomepirac. Currently marketed NSAIDs include Celebrex®, Vioxx®, Anaprox®, Arthrotec®, Bextra®, Cataflam®, Clinoril®, DayPro®, Dolobid®, Feldene®, Indocin®, Mobic®, Motrin®, Negprelen®, Naprosyn®, Ponstel®, Relafen®, Toradol®.

In an embodiment, the present compositions may further comprise an analgesic, antipyretic, and/or anti-inflammatory therapeutic agent. For example, the composition may further comprise one or more of aspirin, sodium salicylate, choline magnesium trisalicylate, salsalate, diflunisal, sulfasalazine, olsalazine, acetaminophen (paracetamol), for example Tylenol PM, indomethacin, sulindac, tolmetin, diclofenac, ketorolac, ibuprofen, naproxen, flurbiprofen, ketoprofen, fenoprofen, oxaprozin, mefenamic acid, meclofenamic acid, piroxicam, meloxicam, nabumetone, refecoxib, celecoxib, etodolac, and nimesulide. For example, the present compositions may comprise an opioid antagonist (such as naltrexone), an opioid agonist (such as oxycodone or hydrocodone), and an analgesic (such as acetaminophen).

In an embodiment, the present compositions may further comprise, at least one mscle relaxant. Representative muscle relaxants include acetylcholine inhibitors such as Botox, neuromuscular blocking agents such as rocuronium (ZEMURON®), succinylcholine (ANECTINE, others), d-tubocurarine, atracurium (TRACRIUM), doxacurium (NUROMAX), mivacurium (MIVACRON), pancuronium (PAVULON), pipecuronium (ARDUAN), rapacuronium (RAPLON), and vecuronium (NORCURON), and skeletal muscle relaxants & combinatives such as dantrolene (Dantrium), cyclobenzaprine (Flexeril®), orphenadrine (Norflex®), carisoprodol (Soma®), and diazepam (Valium®).

With regard to dosage levels, the non-narcotic analgesic is present in a inflammatory pain-alleviating amount or an amount that is not pain-alleviating alone but is pain-alleviating in combination with an opioid agonist and opioid antagonist according to the present disclosure. This amount is at a level corresponding to the generally recommended adult human dosages for a particular non-narcotic analgesic. The effective inflammatory pain-alleviating amount of the opioid antagonist and the opioid agonist can be present at a level that potentiates the inflammatory pain-alleviating effectiveness of the non-narcotic analgesic. Specific dosage levels for the non-narcotic analgesic that can be used herein as given, inter alia, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosures of all are incorporated herein by reference. As is well known to one of ordinary skill in the art, there can be a wide variation in the dosage level of the non-narcotic analgesic, wherein the dosage level depends to a large extent on the specific non-narcotic analgesic being administered. These amounts can be determined for a particular drug combination in accordance with the present disclosure by employing routine experimental testing.

In an embodiment, the present compositions further comprise at least one inhibitor of TNF-α. Inhibitors of TNF-α may also be designated TNF-α antagonists. TNF-α antagonists are compounds which are capable of, directly or indirectly, counteracting, reducing or inhibiting the biological activity of TNF-α, or the activation of receptors therefore. Tumor necrosis factor (TNF) is a key proinflammatory cytokine released by a number of cell types, particularly activated macrophages and monocytes. Additional details regarding the manufacture and use of TNF-α antagonists are available in U.S. Patent Application Publication No. U.S. 2003/0157061 A1, which is incorporated herein by reference.

Preferred TNF-α antagonists for the present disclosure include ENBREL® (etanercept) from Wyeth-Ayerst Laboratories/Immunex; REMICADE®, infiximab, which is an anti-TNF chimeric Mab (Centocor; Johnson & Johnson); anti-TNF-α, D2E7 human Mab (Cambridge antibody Technology); CDP-870, which is a PEGylated antibody fragment (Celltech); CDP-571; Humicade, which is a humanized Mab described in U.S. Pat. No. 5,994,510 (Celltech); PEGylated soluble TNF-α Receptor-1 (Amgen); TBP-1, which is a TNF binding protein (Ares Serono); PASSTNF-alpha®, which is an anti-TNF-α polyclonal antibody (Verigen); AGT-1, which is a mixture of three anti-cytokine antibodies to IFN-alpha, IFN-gamma, and TNF (Advanced Biotherapy Concepts); TENEFUSE®, ienercept, which is a TNFR-Ig fusion protein (Roche); CytoTAB®(Protherics); TACE, which is a small molecule TNF-α converting enzyme inhibitor (Immunex); small molecule TNF mRNA synthesis inhibitor (Nereus); PEGylated p75TNFR Fc mutein (Immunex); and TNF-α antisense inhibitor.

With regard to dosage levels, the TNF-α antagonist is present at an amount effective to inhibit progression or reduce damage from an arthritic condition or a chronic condition associated with inflammation. Alternatively, the TNF-α antagonist is present in an amount that is not effective to inhibit progression or reduce damage alone but is effective to inhibit progression or reduce damage in combination with an opioid agonist and opioid antagonist according to the present disclosure. This amount is at a level corresponding to the generally recommended adult human dosages for a particular TNF-α antagonist. The effective pain-alleviating amount of the opioid antagonist and the opioid agonist can be present at a level that potentiates the effectiveness of a TNF-α antagonist. Specific dosage levels for TNF-α antagonists that can be used herein as given, inter alia, are included, for example, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosure of all are incorporated herein by reference. As is well known to one of ordinary skill in the art, there can be a wide variation in the dosage level of the TNF-α antagonist, wherein the dosage level depends to a large extent on the specific TNF-α antagonist being administered. These amounts can be determined for a particular drug combination, in accordance with the present disclosure, by employing routine experimental testing.

In an embodiment, the present compositions further comprise at least one anti-rheumatic drug. Anti-rheumatic drugs include those referred to as Disease-modifying antirheumatic drugs (DMARDs). Anti-rheumatic drugs include methotrexate (RHEUMATREX, TREXALL), leflunomide (ARAVA), D-Penicillamine, sulfasalazine, gold therapy, minocycline, azathioprine, hydroxychloroquine (PLAQUENIL) and other antimalarials, cyclosporine and biologic agents. Biologic response modifiers, often referred to as biologic agents or simply biologics, are designed to either inhibit or supplement immune system components called cytokines. Cytokines play a role in either fueling or suppressing the inflammation that causes damage in RA and some other diseases. The four biologics currently approved for RA all work by inhibiting inflammatory cytokines. Adalimumab (HUMIRA), etanercept (ENBREL) and infliximab (REMICADE) work to inhibit a cytokine called tumor necrosis factor (TNF). Anakinra (KINERET) blocks the action of the cytokine interleukin-1 (IL-1).

With regard to dosage levels, the anti-rheumatic drug is present at an amount that attenuates a symptom or sign of rheumatism or an amount that does not attenuate such a symptom or sign alone but does attenuate such a symptom or sign in combination with an opioid agonist and opioid antagonist according to the present disclosure. This amount is at a level corresponding to the generally recommended adult human dosages for a particular anti-rheumatic drug. The effective amount of the opioid antagonist and the opioid agonist can be present at a level that potentiates the effectiveness of the anti-rheumatic drug. Specific dosage levels for anti-rheumatic drugs that can be used herein as given, inter alia, are included, for example, in the “Physicians' Desk Reference”, 2003 Edition (Medical Economics Data Production Company, Montvale, N.J.) as well as in other reference works.

In an embodiment, the present compositions further comprise at least one anticonvulsant or anti-epileptic agent. Any therapeutically effective anticonvulsant may be used according to the present disclosure. For extensive listings of anticonvulsants, see, e.g., Goodman and Gilman's “The Pharmaceutical Basis Of Therapeutics”, 8th ed., McGraw-Hill, Inc. (1990), pp. 436-462, and “Remington's Pharmaceutical Sciences”, 17th ed., Mack Publishing Company (1985), pp. 1075-1083 (the disclosures of which are incorporated herein by reference). Representative anticonvulsants that can be used herein include lamotrigine, gabapentin, valproic acid, topiramate, famotodine, phenobarbital, diphenylhydantoin, phenyloin, mephenyloin, ethotoin, mephobarbital, primidone, carbamazepine, ethosuximide, methsuximide, phensuximide, trimethadione, benzodiazepine, phenacemide, acetazolamide, progabide, clonazepam, divalproex sodium, magnesium sulfate injection, metharbital, paramethadione, phenyloin sodium, valproate sodium, clobazam, sulthiame, dilantin, diphenylan and L-5-hydroxytryptophan. Currently marketed anticonvulant/anti-epileptic drugs include Keppra®, Lamictol®, Neurontin®, Tegretol®, Carbatrol®, Topiramate®, Trileptal®, and Zonegran®.

With regard to dosage levels, the anticonvulsant is present at a pain-alleviating amount or an amount that is not pain-alleviating alone but is pain-alleviating in combination with an opioid agonist and opioid antagonist according to the present disclosure. This amount is at a level corresponding to the generally recommended adult human dosages for a particular anticonvulsant. The effective pain-alleviating amount of the opioid antagonist and the opioid agonist can be present at a level that potentiates the pain-alleviating effectiveness of the anticonvulsant. Specific dosage levels for anticonvulsants that can be used herein as given, inter alia, are included, for example, in the “Physicians' Desk Reference”, 2003 Edition (Medical Economics Data Production Company, Montvale, N.J.) as well as in other reference works including Goodman and Gilman's “The Pharmaceutical Basis of Therapeutics” and “Remington's Pharmaceutical Sciences,” the disclosure of all are incorporated herein by reference. As is well known to one of ordinary skill in the art, there can be a wide variation in the dosage level of the anticonvulsant, wherein the dosage level depends to a large extent on the specific anticonvulsant being administered. These amounts can be determined for a particular drug combination, in accordance with this present disclosure, by employing routine experimental testing.

The compositions presented herein may be compounded, for example, with the usual non-toxic, pharmaceutically acceptable excipients, carriers, diluents or other adjuvants. The choice of adjuvants will depend upon the active ingredients employed, the physical form of the composition, the route of administration, and other factors.

The excipients, binders, carriers, and diluents which can be used include water, glucose, lactose, natural sugars such as sucrose, glucose, or corn sweeteners, sorbitol, natural and synthetic gums such as gum acacia, tragacanth, sodium alginate, and gum arabic, gelatin, mannitol, starches such as starch paste, corn starch, or potato starch, magnesium trisilicate, talc, keratin, colloidal silica, urea, stearic acid, magnesium stearate, dibasic calcium phosphate, crystalline cellulose, methyl cellulose, carboxymethyl cellulose, polyethylene glycol, waxes, glycerin, and saline solution, among others.

Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

The dosage forms can also comprise one or more acidifying agents, adsorbents, alkalizing agents, antiadherents, antioxidants, binders, buffering agents, colorants, complexing agents, diluents or fillers, direct compression excipients, disintegrants, flavorants, fragrances, glidants, lubricants, opaquants, plasticizers, polishing agents, preservatives, sweetening agents, or other ingredients known for use in pharmaceutical preparations.

Acidifying agents are a compound used to provide an acidic medium for product stability. Such compounds include, by way of example and without limitation, acetic acid, amino acid, citric acid, fumaric acid and other alpha hydroxy acids, hydrochloric acid, ascorbic acid, nitric acid, phosphoric acid, and others known to those skilled in the art.

Adsorbents are agents capable of holding other molecules onto their surface by physical or chemical (chemisorption) means. Such compounds include, by way of example and without limitation, powdered and activated charcoal, zeolites, and other materials known to one of ordinary skill in the art.

Alkalizing agent are compounds used to provide an alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those skilled in the art.

Antiadherent are agents that prevents the sticking of solid dosage formulation ingredients to punches and dies in a tableting machine during production. Such compounds include, by way of example and without limitation, magnesium stearate, talc, calcium stearate, glyceryl behenate, PEG, hydrogenated vegetable oil, mineral oil, stearic acid and other materials known to one of ordinary skill in the art.

Antioxidants are agents which inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art.

Binders are substances used to cause adhesion of powder particles in solid dosage formulations. Such compounds include, by way of example and without limitation, acacia, alginic acid, carboxymethylcellulose sodium, poly(vinylpyrrolidone), compressible sugar (e.g., NuTab), ethylcellulose, hydroxypropyl methylcellulose, gelatin, liquid glucose, methylcellulose, povidone and pregelatinized starch and other materials known to one of ordinary skill in the art.

When needed, binders may also be included in the dosage forms. Exemplary binders include acacia, tragacanth, gelatin, starch, cellulose materials such as methyl cellulose, HPMC, HPC, HEC and sodium carboxy methyl cellulose, alginic acids and salts thereof, polyethylene glycol, guar gum, polysaccharide, bentonites, sugars, invert sugars, poloxamers (PLURONIC™F68, PLURONIC™ F127), collagen, albumin, gelatin, cellulosics in nonaqueous solvents, combinations thereof and others known to those skilled in the art. Other binders include, for example, polypropylene glycol, polyoxyethylene—polypropylene copolymer, polyethylene ester, polyethylene sorbitan ester, polyethylene oxide, combinations thereof and other materials known to one of ordinary skill in the art.

Buffering agents are compounds used to resist changes in pH upon dilution or addition of acid or alkali. Such compounds include, by way of example and without limitation, potassium metaphosphate, potassium phosphate, monobasic sodium acetate and sodium citrate anhydrous and dihydrate and other materials known to one of ordinary skill in the art.

Sweetening agents are compounds used to impart sweetness to a preparation. Such compounds include, by way of example and without limitation, aspartame, dextrose, glycerin, mannitol, saccharin sodium, sorbitol, sucrose, and other materials known to one of ordinary skill in the art.

Diluents or fillers are inert substances used to create the desired bulk, flow properties, and compression characteristics in the preparation of solid dosage forms. Such compounds include, by way of example and without limitation, dibasic calcium phosphate, kaolin, lactose, dextrose, magnesium carbonate, sucrose, mannitol, microcrystalline cellulose, powdered cellulose, precipitated calcium carbonate, calcium sulfate, sorbitol, and starch and other materials known to one of ordinary skill in the art.

Direct compression excipients are compounds used in compressed solid dosage forms. Such compounds include, by way of example and without limitation, dibasic calcium phosphate (e.g., Ditab) and other materials known to one of ordinary skill in the art.

Disintegrants are compounds used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starches thereof, sweeteners, clays such as bentonite, low substituted hydroxypropyl cellulose, microcrystalline cellulose (e.g., Avicel), methyl cellulose, carboxymethylcellulose calcium, sodium carboxymethylcellulose, alginic acid, sodium alginate, cellulose polyacrilin potassium (e.g., Amberlite), alginates, sodium starch glycolate, gums, agar, guar, locust bean, karaya, xanthan, pectin, tragacanth, agar, bentonite, and other materials known to one of ordinary skill in the art.

Glidants are agents used in solid dosage formulations to promote flowability of the solid mass. Such compounds include, by way of example and without limitation, colloidal silica, cornstarch, talc, calcium silicate, magnesium silicate, colloidal silicon, tribasic calcium phosphate, silicon hydrogel and other materials known to one of ordinary skill in the art.

Lubricants are substances used in solid dosage formulations to reduce friction during compression. Such compounds include, by way of example and without limitation, sodium oleate, sodium stearate, calcium stearate, zinc stearate, magnesium stearate, polyethylene glycol, talc, mineral oil, stearic acid, sodium benzoate, sodium acetate, sodium chloride, and other materials known to one of ordinary skill in the art.

Opaquants are compounds used to render a coating opaque. An opaquant may be used alone or in combination with a colorant. Such compounds include, by way of example and without limitation, titanium dioxide, talc and other materials known to one of ordinary skill in the art.

Polishing agents are compounds used to impart an attractive sheen to solid dosage forms. Such compounds include, by way of example and without limitation, carnauba wax, white wax and other materials known to one of ordinary skill in the art.

Colorants are compounds used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, ferric oxide, other FD&C dyes and natural coloring agents such as grape skin extract, beet red powder, beta-carotene, annato, carmine, turmeric, paprika, and other materials known to one of ordinary skill in the art. The amount of coloring agent used will vary as desired.

Flavorants are compounds used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors will be present in any amount as desired by those skilled in the art. Particularly contemplated flavors are the grape and cherry flavors and citrus flavors such as orange.

Complexing agents include for example EDTA disodium or its other salts and other agents known to one of ordinary skill in the art.

Exemplary fragrances include those generally accepted as FD&C grade.

Exemplary preservatives include materials that inhibit bacterial growth, such as Nipagin, Nipasol, alcohol, antimicrobial agents, benzoic acid, sodium benzoate, benzyl alcohol, sorbic acid, parabens, isopropyl alcohol and others known to one of ordinary skill in the art.

Solid dosage forms of the present methods and materials can also employ one or more surface active agents or cosolvents that improve wetting or disintegration of the core and/or layer of the solid dosage form.

Plasticizers can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co. The PEG based plasticizers are available commercially or can be made by a variety of methods, such as disclosed in Poly(ethylene glycol) Chemistry. Biotechnical and Biomedical Applications (J. M. Harris, Ed.; Plenum Press, NY) the disclosure of which is hereby incorporated by reference.

Solid dosage forms of the present methods and materials can also include oils, for example, fixed oils, such as peanut oil, sesame oil, cottonseed oil, corn oil and olive oil; fatty acids, such as oleic acid, stearic acid and isostearic acid; and fatty acid esters, such as ethyl oleate, isopropyl myristate, fatty acid glycerides and acetylated fatty acid glycerides. It can also be mixed with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent.

Soaps and synthetic detergents may be employed as surfactants and as vehicles for the solid pharmaceutical compositions. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl beta-aminopropionates and 2-alkylimidazoline quaternary ammonium salts; and others known to one of ordinary skill in the art; and mixtures thereof.

A water soluble coat or layer can be formed to surround a solid dosage form or a portion thereof. The water soluble coat or layer can either be inert or drug-containing. Such a coat or layer will generally comprise an inert and non-toxic material which is at least partially, and optionally substantially completely, soluble or erodible in an environment of use. Selection of suitable materials will depend upon the desired behavior of the dosage form. A rapidly dissolving coat or layer will be soluble in the buccal cavity and/or upper GI tract, such as the stomach, duodenum, jejunum or upper small intestines. Exemplary materials are disclosed in U.S. Pat. No. 4,576,604 to Guittard et al. and U.S. Pat. No. 4,673,405 to Guittard et al., and U.S. Pat. No. 6,004,582 to Faour et al. and the text Pharmaceutical Dosage Forms: Tablets Volume I, 2^{nd} Edition. (A. Lieberman. ed. 1989, Marcel Dekker, Inc.), the disclosures of which are hereby incorporated by reference. In some embodiments, the rapidly dissolving coat or layer will be soluble in saliva, gastric juices, or acidic fluids.

Materials which are suitable for making the water soluble coat or layer include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; croscarmellose sodium; other cellulose polymers such as sodium carboxymethylcellulose; and other materials known to those skilled in the art. Other lamina-forming materials that can be used for this purpose include poly(vinyl alcohol), poly(ethylene oxide), gelatin, glucose and saccharides. The water soluble coating can comprise other pharmaceutical excipients that may or may not alter the way in which the water soluble coating behaves. The artisan of ordinary skill will recognize that the above-noted materials include film-forming polymers.

A water soluble coat or layer can also comprise hydroxypropyl methylcellulose, which is supplied by Dow under its Methocel E-15 trademark. The materials can be prepared in solutions having different concentrations of polymer according to the desired solution viscosity. For example, a 2% W/V aqueous solution of Methocel™ E-15 has a viscosity of about 13-18 cps at 20° C.

For transcutaneous or transdermal administration, the compounds may be combined with skin penetration enhancers such as propylene glycol, polyethylene glycol, isopropanol, ethanol, oleic acid, N-methylpyrrolidone, or others known to those skilled in the art, which increase the permeability of the skin to the compounds, and permit the compounds to penetrate through the skin and into the bloodstream. The compound/enhancer compositions also may be combined additionally with a polymeric substance such as ethylcellulose, hydroxypropyl cellulose, ethylene/vinylacetate, or others known to those skilled in the art, to provide the composition in gel form, which can be dissolved in solvent such as methylene chloride, evaporated to the desired viscosity, and then applied to backing material to provide a patch.

For intravenous, intramuscular, subcutaneous, intrathecal, epidural, perineural or intradermal administration, the active ingredients may be combined with a sterile aqueous solution. The solution may be isotonic with the blood of the recipient. Such formulations may be prepared by dissolving one or more solid active ingredients in water containing physiologically compatible substances such as sodium chloride, glycine, or others known to those skilled in the art, and/or having a buffered pH compatible with physiological conditions to produce an aqueous solution, and/or rendering the solution sterile. The formulations may be present in unit dose containers such as sealed ampoules or vials.

For topical (e.g., dermal or subdermal) or depot administration, the active ingredients may be formulated with oils such as cottonseed, hydrogenated castor oil and mineral oil; short chain alcohols as chlorobutanol and benzyl alcohol; also including polyethylene glycols, polysorbates; polymers such as sucrose acetate isobutyrate, caboxymethocellusose and acrylates; buffers such as dihydrogen phosphate; salts such as sodium chloride and calcium phosphate; and other ingredients included but not exclusive to povidone, lactose monohydrate, magnesium stearate, myristyo-gamma-picolinium; and water.

A solid dosage form of the present methods and materials can be coated with a finish coat as is commonly done in the art to provide the desired shine, color, taste or other aesthetic characteristics. Materials suitable for preparing the finish coat are well known in the art and found in the disclosures of many of the references cited and incorporated by reference herein.

Various other components, in some cases not otherwise listed above, can be added to the present formulation for optimization of a desired active agent release profile including, by way of example and without limitation, glycerylmonostearate, nylon, cellulose acetate butyrate, d,l-poly(lactic acid), 1,6-hexanediamine, diethylenetriamine, starches, derivatized starches, acetylated monoglycerides, gelatin coacervates, poly (styrene—maleic acid) copolymer, glycowax, castor wax, stearyl alcohol, glycerol palmitostearate, poly(ethylene), poly(vinyl acetate), poly(vinyl chloride), 1,3-butylene-glycoldimethacrylate, ethyleneglycol-dimethacrylate and methacrylate hydrogels.

The compositions for use in the methods of the present disclosure can be formulated in capsules, tablets, caplets, or pills. Such capsules, tablets, caplets, or pills of the present inflammatory pain-alleviating compositions can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Similarly, the carrier or diluent may include any sustained release material known in the art, such as glyceryl monostearate or glyceryl distearate, alone or mixed with a wax. The formulations of the present methods and materials may be formulated so as to provide quick, sustained, or delayed release of the active ingredient after administration to the patient by employing procedures well known in the art.

Controlled release or sustained-release dosage forms, as well as immediate release dosage forms are specifically contemplated. Controlled release or sustained release as well as immediate release compositions in liquid forms in which a therapeutic agent may be incorporated for administration orally or by injection are also contemplated.

The pharmaceutical compositions or dosage forms of the present methods and materials may be used in the form of a pharmaceutical preparation which contains one or more opioid antagonists in combination with one or more opioid agonists.

It has previously been discovered that some opioid antagonists undesirably bind significantly to certain pharmaceutical excipients, including during the preparation of dosage forms of antagonist of 1 mg or less, 0.5 mg or less or less, 0.1 mg or less, 0.01 mg or less, or 0.001 mg or less. Those pharmaceutical excipients generally cause an incomplete amount of the opioid antagonist to be released from a dosage form, within a particular time allotted for release. For example, when naltrexone hydrochloride in solution was mixed with croscarmellose sodium in suspension, the croscarmellose sodium bound more than 90% of the naltrexone hydrochloride. Accordingly, opioid antagonists must be tested with pharmaceutical excipients, so as to ensure that the excipient does not bind the opioid antagonist to a significant degree. Excipients, for example, binders, disintegrants, glidants, lubricants, or acidifiers, as well as process conditions, such as pH, should be selected with this in mind.

The compositions present herein for alleviating the symptoms or signs of arthritic conditions, chronic conditions associated with inflammation or chronic pain can be administered from about one time daily to about six times daily, two times daily to about four times daily, or one time daily to about two times daily.

Pain-alleviating compositions, including inflammatory pain-alleviating compositions, presented herein preferably comprise at least one colloidal dispersion system, additive or preservative, diluent, binder, plasticizer, or slow release agent.

It should be understood that compounds used in the art of pharmaceutical formulation generally serve a variety of functions or purposes. Thus, whether a compound named herein is mentioned only once or is used to define more than one term herein, its purpose or function should not be construed as being limited solely to the named purpose(s) or function(s).

The present pain-alleviating compositions, including inflammatory pain-alleviating compositions, may be in admixture with an organic or inorganic carrier or excipient suitable for administration in enteral or parenteral applications, such as orally, topically, transdermally, by inhalation spray, rectally, by subcutaneous, intravenous, intramuscular, subcutaneous, intrathecal, epidural, perineural, intradermal, intraocular injection or infusion techniques. Preferably, such compositions are in the form of a topical, intravenous, intrathecal, epidural, perineural, or oral formulation. More preferably, such compositions are in the form of an intrathecal, epidural or perineural formulation. Even more preferably, such compositions are in the form of an intravenous formulation. Most preferably, such compositions are in the form of an oral formulation.

The present methods and materials are additionally advantageous because they can be used to maintain or enhance (e.g., increase) analgesic potency of the opioid agonists without substantially increasing the adverse side effects in humans associated with that dose of agonist, including attenuating one or more adverse effects. For example, the present methods and compositions may be employed in human subjects without significant increases, including attenuating in incidents of eye disorders, gastrointestinal disorders (such as upper abdominal pain, constipation, diarrhea, nausea, and vomiting), general disorders and conditions (such as lethargy), nervous system disorders (such as dizziness, headache, sedation, and sommolence), psychiatric disorders (such as euphoric mood), and skin and subcutaneous tissue disorders (such as pruritus). For compositions and methods as described that enhance analgesic potency of the opioid agonist, it is advantageous that adverse side effects are not increased with that enhanced e.g., increased) potency.

The following examples are provided for illustrative purposes and are not to be construed to limit the scope of the claims in any manner whatsoever. For example, Example 6 describes studies using methods and materials comprising opioid antagonists, including combinations of opioid agonists and antagonists, for the treatment of back pain. Examples 1-5 describe correlative studies.

### EXAMPLE 1

A.

In a clinical study, the effects of an exemplary opioid agonist oxycodone in combination with an exemplary opioid antagonist naltrexone were evaluated in subjects with moderate to severe chronic pain due to an exemplary arthritic condition osteoarthritis of the hip or knee.

A clinical study was designed as follows: (1) to evaluate the efficacy and safety of combinations of oxycodone (oxy) and naltrexone (ntx) administered twice daily and four times daily relative to oxycodone administered four times daily while maintaining the same total daily oxycodone dose, and (2) to evaluate the frequency and severity of opioid withdrawal in patients who received combinations of oxycodone and naltrexone compared to those patients who received oxycodone.

A multicenter, randomized, double-blind, active- and placebo-controlled, dose escalation, clinical study was designed and conducted. The study evaluated the efficacy and safety of an oral formulation of oxycodone and naltrexone relative to oxycodone over a 3-week period in patients with chronic pain due to osteoarthritis of the hip or knee. A total of 360 patients were enrolled into four treatment groups: two groups for combinations of oxycodone and naltrexone, a group for oxycodone alone, and a group for placebo. During a 4- to 7-day washout period, patients stopped taking all of their pain medication other than acetaminophen (500 mg every 4-6 hours PRN (a maximum of 5 caplets per day)).

A daily diary was to be utilized to record overall pain intensity (PI) and other signs and symptoms. The patient was enrolled in the study if: (1) the mean value of the diary PI over the last 2 days of the 4- to 7-day baseline period was ≧5; (2) the confirmatory PI obtained at the baseline clinic visit was also ≧5; and, (3) the patient met all inclusion/exclusion criteria. Baseline functional assessments were conducted with the SF-12 Health Survey as shown in Table 1 and the Western Ontario and MacMaster Universities Osteoarthritis Index (WOMAC) as shown in Table 2 below before the initiation of study medication.

TABLE 1
The SF-12v2 ™ Health Survey

Instructions for Completing the Questionnaire
Please answer every question. Some questions may look like others, but each one is
different. Please take the time to read and answer each question carefully
by filling in the bubble that best represents your response.
EXAMPLE
This is for your review. Do not answer this question. The questionnaire begins with the
section Your Health in General below.
For each question you will be asked to fill in a bubble in each line:
1. How strongly do you agree or disagree with each of the following statements?

| | | Strongly | | | | Strongly |
|---|---|---|---|---|---|---|
| | | agree | Agree | Uncertain | Disagree | disagree |
| a) | I enjoy listening to | ◯ | ● | ◯ | ◯ | ◯ |
| | music. | | | | | |
| b) | I enjoy reading | ● | ◯ | ◯ | ◯ | ◯ |
| | magazines. | | | | | |

Please begin answering the questions now.

Your Health in General

1. In general, would you say your health is:

| Excellent | Very good | Good | Fair | Poor | |
|---|---|---|---|---|---|
| ◯₁ | ◯₂ | ◯₃ | ◯₄ | ◯₅ | GH1 |

2. The following questions are about activities you might do during a typical day.
Does your health now limit you in these activities? If so, how much?

| | | Yes, limited | Yes, limited | No, not limited | |
|---|---|---|---|---|---|
| | | a lot | a little | at all | |
| a) | Moderate activities, | ◯₁ | ◯₂ | ◯₃ | PF02 |
| | such as moving a | | | | |
| | table, pushing a | | | | |
| | vacuum cleaner, | | | | |
| | bowling, or | | | | |
| | playing golf | | | | |
| b) | Climbing several | ◯₁ | ◯₂ | ◯₃ | PF04 |
| | flights of stairs | | | | |

3. During the past week, how much of the time have you had any of the following
problems with your work or other regular daily activities as a result of your
physical health?

| | | All of | Most of | Some of | A little of | None of | |
|---|---|---|---|---|---|---|---|
| | | the time | the time | the time | the time | the time | |
| a) | Accomplished less | ◯₁ | ◯₂ | ◯₃ | ◯₄ | ◯₅ | RP2 |
| | than you would like | | | | | | |
| b) | Were limited in the | ◯₁ | ◯₂ | ◯₃ | ◯₄ | ◯₅ | RP3 |
| | kind of work or | | | | | | |
| | other activities | | | | | | |

4. During the past week, how much of the time have you had any of the following
problems with your work or other regular daily activities as a result of any
emotional problems (such as feeling depressed or anxious)?

| | | All of | Most of | Some of | A little of | None of | |
|---|---|---|---|---|---|---|---|
| | | the time | the time | the time | the time | the time | |
| a) | Accomplished less | ◯₁ | ◯₂ | ◯₃ | ◯₄ | ◯₅ | RE2 |
| | than you would like | | | | | | |
| b) | Did work or | ◯₁ | ◯₂ | ◯₃ | ◯₄ | ◯₅ | RE3 |
| | other activities less | | | | | | |
| | carefully than usual | | | | | | |

5. During the past week, how much did pain interfere with your normal work
(including both work outside the home and housework)?

| Not at all | A little bit | Moderately | Quite a bit | Extremely | |
|---|---|---|---|---|---|
| ◯₁ | ◯₂ | ◯₃ | ◯₄ | ◯₅ | BP2 |

6. These questions are about bow you feel and how things have been with you
during the past week. For each question, please give the one answer that comes
closest to the way you have been feeling. How much of the time during the past week . . .

| | | All of | Most of | Some of | A little of | None of | |
|---|---|---|---|---|---|---|---|
| | | the time | the time | the time | the time | the time | |
| a) | have you felt calm | ◯₁ | ◯₂ | ◯₃ | ◯₄ | ◯₅ | MH3 |
| | and peaceful? | | | | | | |
| b) | did you have a lot | ◯₁ | ◯₂ | ◯₃ | ◯₄ | ◯₅ | VT2 |
| | of energy? | | | | | | |
| c) | have you felt | ◯₁ | ◯₂ | ◯₃ | ◯₄ | ◯₅ | MH4 |
| | downhearted and | | | | | | |
| | depressed? | | | | | | |

7. During the past week, how much of the time has your physical health or emotional
problems interfered with your social activities (like visiting friends, relatives, etc.)?

| All of | Most of | Some of | A little | None of | |
|---|---|---|---|---|---|
| the time | the time | the time | of the time | the time | |
| ◯₁ | ◯₂ | ◯₃ | ◯₄ | ◯₅ | SF2 |

THANK YOU FOR COMPLETING THIS QUESTIONNAIRE!

TABLE 2
WOMAC OSTEOARTHRITIS INDEX

Directions: Please refer to the instructions provided to you for completion
of the following questions.

Section A
PAIN

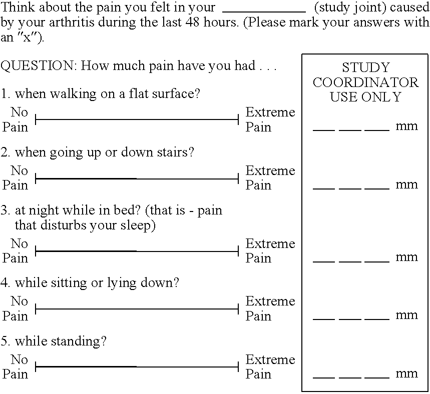

Section B
STIFFNESS

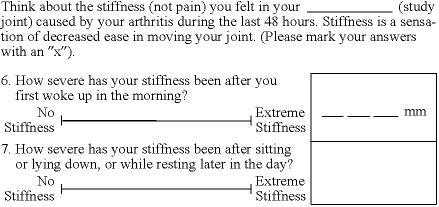

Section C
DIFFICULTY PERFORMING DIALY ACTIVITIES

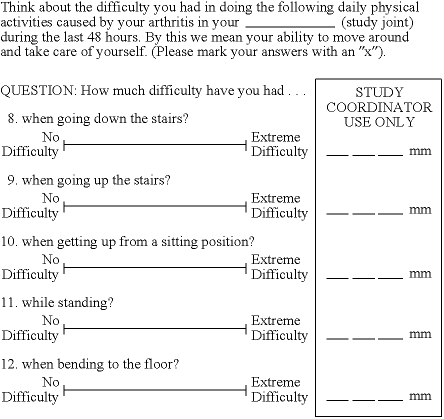

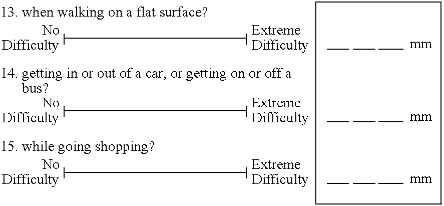

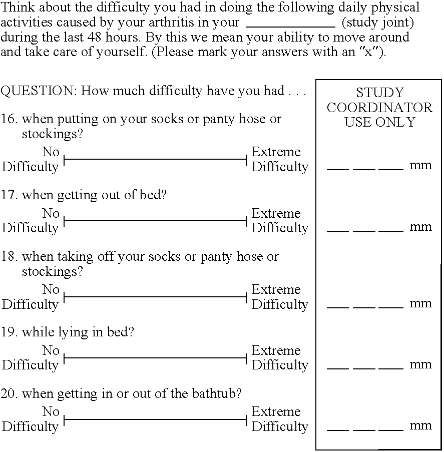

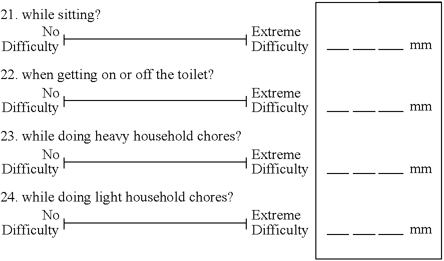

Patients were randomly assigned to one of the four treatment groups as shown in Table 3.

TABLE 3

| Treatment | Week 1 | | Week 2 | Week 3 |
|---|---|---|---|---|
| Group | (Days 1-3) | (Days 4-8) | (Days 1-8) | (Days 1-8) |
| A | (OXY 5 mg + | (OXY 10 mg + | (OXY 15 mg + | (OXY 20 mg + |
| | NTX 0.001 mg) | NTX 0.001 mg) | NTX 0.001 mg) | NTX 0.001 mg) |
| | BID | BID | BID | |
| B | (OXY 2.5 mg + | (OXY 5 mg + | (OXY 7.5 mg + | (OXY 10 mg + |
| | NTX 0.001 mg) | NTX 0.001 mg) | NTX 0.001 mg) | NTX 0.001 mg) |
| | QID | QID | QID | QID |
| C | OXY 2.5 mg QID | OXY 5 mg QID | OXY 7.5 mg QID | OXY 10 mg QID |
| D | Placebo QID | Placebo QID | Placebo QID | Placebo QID |

The demographics of the four groups was balanced across the groups as shown in Table 4.

TABLE 4

| | | | Oxycodone | Oxycodone |
|---|---|---|---|---|
| | | | Plus | Plus |
| | | Oxycodone | Naltrexone | Naltrexone |
| | Placebo | QID | QID | BID |
| ITT Population | 51 | 102 | 104 | 103 |
| Female | 69% | 70% | 69% | 69% |
| Male | 31% | 30% | 31% | 31% |
| Age | 56.0 | 53.5 | 53.6 | 55.1 |
| Weight (kg) | 90.7 | 93.3 | 96.0 | 94.6 |

All treatment groups were scheduled for QID dosing to protect the double-blind study design as shown in Table 5.

TABLE 5

| Treatment | QID Daily Dosing Scheme* | | | |
|---|---|---|---|---|
| Group | Upon Waking | Noon | Afternoon | Bedtime |
| A | Oxycodone and | Placebo | Oxycodone and | Placebo |
| | Naltrexone | | Naltrexone | |
| B | Oxycodone and | Oxycodone | Oxycodone and | Oxycodone |
| | Naltrexone | and | Naltrexone | and |
| | | Naltrexone | | Naltrexone |
| C | Oxycodone | Oxycodone | Oxycodone | Oxycodone |
| D | Placebo | Placebo | Placebo | Placebo |

*Doses were to be taken 30-60 minutes before meals and at least 4 hours apart. On Day 1 (Week 1 only), patients were to receive three doses of study drug (noon, afternoon and bedtime).

During the 3-week treatment period, patients recorded their PI every 24 hours in their daily diary immediately before their bedtime dose. In addition, patients recorded adverse events and date/time of taking the study medication in the daily diary. Patients returned to the clinic on Week 2, Day 1; Week 3, Day 1 and for End of Treatment assessments (±one day) by the investigator. At each clinic visit, the investigator also collected, additional data, including quality of analgesia, pain control, the SF-12 Health Survey, the WOMAC Osteoarthritis Index and a global assessment of study medication. Patients were required to return for a post-treatment follow-up visit approximately one week after the final dose of study medication (±two days).

Safety was evaluated by vital signs (blood pressure, heart rate, respiratory rate and temperature), physical examinations, EKGs, clinical laboratory tests, adverse events, opioid toxicity assessments and the assessment of opiate withdrawal symptoms using the Short Opiate Withdrawal Scale (SOWS) as shown in Table 6 below.

TABLE 6
Short Opiate Withdrawal Scale
Please put a check mark in the appropriate box for each of the
following conditions in the last 24 hours:

| | | | | |
|---|---|---|---|---|
| Description | None | Mild | Moderate | Severe |

Feeling Sick
Stomach Cramps
Muscle Spasms/Twitching
Feelings of Coldness
Heart Pounding
Muscular Tension
Aches and Pains
Yawning
Runny Eyes
Insomnia/Problems Sleeping

Note: This table shows the 10 items of SOWS and the format in which it is administered.

The Study Population was three hundred sixty-two (360) patients with moderate to severe chronic pain due to osteoarthritis of the hip or knee. According to the study design described above, there were to be about 100 patients each in the oxycodone and naltrexone BID, oxycodone and naltrexone QID and oxycodone alone treatment groups; and about 50 patients in the placebo group.

Inclusion criteria were as follows:
(1) Males and females who were ≧18 and ≧70 years of age;
(2) Females who were postmenopausal, physically incapable of childbearing, or practicing an acceptable method of birth control. Acceptable methods of birth control included surgical sterilization, hormonal contraceptives, or double-barrier methods (condom or diaphragm with a spermicidal agent or IUD). If practicing an acceptable method of birth control, a negative urine pregnancy test result was obtained at the Screening Visit;
(3) Patient was ambulatory;
(4) Patient had moderate to severe pain in one or more hip or knee joint(s) caused by osteoarthritis for at least three months prior to the Screening Visit;
(5) Patient had moderate to severe pain in the hip or knee joint(s) while taking one or more oral analgesic medication(s) (e.g., NSAIDs, COX-2 inhibitors, tramadol, opioid) in the past one month prior to the Screening Visit;
(6) Patient had a pain intensity score of ≧5 on an 11-point numerical scale at the Screening Visit;
(7) Patient had a mean daily diary overall pain intensity (taken immediately before their bedtime dose of acetaminophen) of ≧5 on an 11-point numerical scale during the last two days of the 4- to 7-day washout period and a confirmatory pain intensity level of ≧5 on an 11-point numerical scale measured at the clinic at Visit 2;
(8) Patient was able to understand and cooperate with study procedures, and has signed a written informed consent prior to entering the study; and
(9) Patient agreed to refrain from taking any pain medications other than study drug during the 3-week treatment period. (Aspirin (up to 325 mg/day) was permitted for cardiovascular prophylaxis if at a stable dose one month prior to the Screening Visit.)

Exclusion criteria for subjects were as follows:
(1) Patient had received a daily opioid dose equivalent (if applicable) of oxycodone >20 mg for two or more days within four weeks prior to the Screening Visit (as calculated by the Drug Conversion Calculator Version 2.0, American Pain Study);
(2) Patient had received an opioid within 72 hours of the Screening Visit;
(3) Patient weighed more than 300 lbs or less than 100 lbs;
(4) Patient had major surgery within three months prior to the Screening Visit or had surgery planned for this joint during the proposed study period;
(5) Patient had received oral or parenteral corticosteroid therapy within one month prior to the Screening Visit;
(6) Patient had received an intraarticular injection of hyaluronic acid within nine months prior to the Screening Visit;
(7) Patient had received any epidural or intrathecal infusion of any analgesic medication(s) within one month prior to the Screening Visit;
(8) Patient was pregnant or breast-feeding;
(9) Patient had a history of severe hepatic or renal impairment;
(10) Patient had acute hepatitis;
(11) Patient had a known allergy or significant reaction to any of the study medications;
(12) Patient had severe impairment of pulmonary function, hypercarbia, hypoxia, significant chronic obstructive airways disease or cor pulmonale, acute or severe bronchial asthma, sleep apnea syndrome or respiratory depression;
(13) Patient had or is suspected of having paralytic ileus, acute abdomen (serious abdominal pain requiring emergency surgery), or delayed gastric emptying;
(14) Patient had chronic biliary tract disease, chronic pancreatitis, or inflammatory bowel disorders;
(15) Patient had untreated myxedema, untreated hypothyroidism, elevated intracranial pressure, severe anemia, adrenocortical insufficiency, hypotension or hypovolemia;
(16) Patient had other diseases significant enough, in the opinion of the Investigator, to pose a risk for the administration of study drug or that will interfere with pain assessments;
(17) Patient had started or stopped monoamine oxidase inhibitors, tricyclic antidepressant drugs, serotonin reuptake inhibitors, glucosamine/chondroitin, or St. John's Wort within four weeks prior to receiving study medication. A constant dose for longer than four weeks is acceptable;
(18) Patient was receiving high doses of sedatives, hypnotics or tranquilizers;
(19) Patient was receiving phenothiazines or other agents that compromise vasomotor tone;
(20) Patient had a history of alcohol or drug abuse;
(21) Patient had previously received the investigational study drug of oxycodone and naltrexone;
(22) Patient had participated in another investigational drug trial or therapeutic trial within 30 days of the Screening Visit; or
(23) Patient had taken analgesic medication (other than acetaminophen—up to 5 caplets per day) during the 4- to 7-day washout period prior to randomization.

The physical descriptions of the drugs used for the study were as follows. For the 4-to 7-day washout period, a container of acetaminophen (500 mg caplets) was dispensed at the Screening Visit in a sufficient quantity for dosing up to five caplets per day. The investigational drug supplies were in tablet dosage forms containing oxycodone HCl and naltrexone HCl, oxycodone HCl or placebo. All of the tablet dosage forms were indistinguishable from one another to facilitate blinding. The tablets were round (approximately 7 mm diameter), biconvex and had a pale yellow color coating. The investigational drug supplies were dispensed in these weekly kits.

The study procedures were as follows. Prior to any study-related activities, written informed consent was signed and dated by the patient. Clinical examinations were performed that comprised the standard-of-care evaluations routinely performed as part of ongoing care for patients with moderate to severe chronic pain due to osteoarthritis of the hip or knee. Pain assessments were performed by assessing: (1) Pain Intensity, (2) Quality of Analgesia, (3) Pain Control, and (4) Global Assessment of Study Medication.

Pain Intensity was assessed by prompting the patient with the question, “How would you rate your overall pain intensity at this time?”, and the PI score was recorded in the clinic. Pain Intensity was also assessed by prompting the patient with the question, “How would you rate your overall pain intensity during the past 24 hours?”, and a daily PI diary score was recorded by the patient at bedtime. For both Pain Intensity prompts, the response was scored on an 11-point numerical scale (0=no pain and 10=severe pain).

Quality of Analgesia was assessed weekly at clinic visits. The patient was prompted with the question, “How would you rate the quality of your pain relief at this time?”, and responses were selected from poor, fair, good, very good, and excellent.

Pain Control was also assessed weekly at clinic visits. The patient was prompted with the question, “During the past week, how would you describe your pain control during the course of each day?” Responses were selected from: Pain was controlled for (1) a few hours or less each day; (2) several hours each day; (3) most of each day; and (4) throughout each day.

Global Assessment of Study Medication was also assessed weekly at clinic visits. The patient was prompted with the question, “How would you rate the study medication you received this past week? (Please consider the quality of your pain relief, your side effects, your activity level, your mood and sense of well-being, etc. in this evaluation.)”. Responses were selected from poor, fair, good, very good, and excellent.

Additionally, functional assessments were conducted with the SF-12 Health Survey (see Table 1) and the WOMAC Osteoarthritis Index (see Table 2).

Safety procedures included vital signs (blood pressure, respiratory rate, heart rate and temperature), physical examinations, EKGs, clinical laboratory tests, adverse events, opioid toxicity assessments and the assessment of opiate withdrawal symptoms using the SOWS (see Table 6). The opioid toxicity assessment included: (A) CNS review by assessing for (1) confusion, altered mental state, (2) excessive drowsiness, lethargy, stupor, (3) slurred speech (new onset), (4) respiratory, (5) hypoventilation, shortness of breath, apnea, (6) hypoxia, hypercarbia; and (b) cardiac review by assessing for bradycardia, hypotension, and shock. If patients experienced any of these or other symptoms that, in the principal investigator's opinion, would pose a significant risk if additional opioid doses were administered, doses were not escalated on Week 2, Day 1 or Week 3, Day 1.

At the first visit, pre-enrollment screening was performed. The following assessments were conducted at Visit 1, four to seven days prior to enrollment in the study: (1) written informed consent, (2) clinic PI, (3) review inclusion and exclusion criteria, (4) detailed medical history including concomitant medications taken one month prior to the screening visit, (5) complete physical examination including height, weight and vital signs, (6) EKG (QTc interval only), (7) blood samples for clinical laboratory tests (hematology and chemistry), (8) urine sample for clinical laboratory tests, (drug screening and urinalysis), (9) urine pregnancy test for all women of childbearing potential, and (10) dispense acetaminophen, take-home diary and provide an appointment card for the next visit. The study nurse thoroughly reviewed each section of the diary with the patient. The diary issued at Visit 1 was to be used by the patient to record the following information at bedtime immediately before the patient's dose of acetaminophen was taken: (a) overall PI in the past 24 hours, (b) signs and symptoms, and (c) date/time of each acetaminophen dose.

The second visit was on the first day of the first treatment week of the study. The patients returned to the study center four to seven days after the Screening Visit for completion of the pre-dose assessments. This visit included (1) reviewing the take-home diary from the past four to seven days; (2) collecting the bottle of acetaminophen and performing accountability; (3) a baseline clinic PI rating, (4) reviewing inclusion and exclusion criteria. This assessment also included verifying that (a) the mean daily overall pain intensity score collected in the diary over the last two days of the 4- to 7-day washout period was ≧5 (on a scale of 0 to 10) while off all analgesic medications (except acetaminophen as directed); (b) the clinic PI at this visit measured ≧5 (on a scale of 0 to 10); and (c) checking that the clinical laboratory tests results from the screening visit were without significant clinical abnormalities, that the urine pregnancy test was negative (if required), and that the urine drug screen was negative.

Patients meeting the study entry criteria were randomly assigned to one of the four treatment groups, and were assigned a randomization number and study medication kit number. The following assessments were then conducted: (1) a brief (interim) medical history; (2) vital signs; (3) blood sample for PK analysis (procedures for collection, storage and shipping of PK samples were provided under separate cover); (4) review and record concomitant medications; (5) SF-12 Health Survey; and (6) WOMAC Osteoarthritis Index.

Once these assessments and procedures were completed, the study medication kit was dispensed for Week 1 (Study Days 1-8). Patients were instructed to take up to three doses of study medication on this day (noon, afternoon and at bedtime). In addition, patients were instructed to take their Day 8 ‘waking’ dose from this medication kit. The patients received their take-home daily diaries and were provided with an appointment card for the next visit. The study nurse thoroughly reviewed each section of the diary with the patient. The daily diary issued at Visit 2 was used to record the following information at Bedtime immediately prior to dosing: (1) overall PI in the past 24 hours; (2) Date and time of each dose of study medication taken; and (3) adverse events.

Patients were contacted by telephone on the evenings of Days 3 and 4 of Treatment Week 1. On Day 3, patients were contacted to determine whether the dose should be escalated on the morning of Day 4. On Day 4, patients were contacted to determine whether patients were tolerating the higher dose. The telephone visits were also used to check for adverse events, compliance and concomitant medications and to remind the patients to complete the daily diary and bring it to the next visit.

Patients returned to the study center for their third visit on Week 2, Day 1 (±one day) for the following:
(1) opioid toxicity assessment;
(2) review take-home diary;
(3) record new/changed adverse events and concomitant medications;
(4) collect study medication from Week 1 (Study Days 1-8) and account for used/unused supplies;
(5) vital signs;
(6) blood sample for PK analysis;
(7) quality of analgesia;
(8) pain control;
(9) global assessment of study medication;
(10) SF-12 Health Survey;
(11) WOMAC Osteoarthritis Index; and
(12) dispense take-home daily diary and study medication kit for Week 2 (Study Days 1-8).

Patients were instructed to take up to three doses of study medication on this day (noon, afternoon and at bedtime). In addition, patients were instructed to take their Day 8 ‘waking’ dose from this medication kit. At the conclusion of this visit, the patient was given an appointment card for the next study visit.

Patients were contacted by telephone on the evening of Day 1 of Treatment Week 2 to determine whether they are tolerating the higher dose; to check for adverse events, compliance and concomitant medications; and to remind them to complete the daily diary and bring it to the next visit.

Patients returned to the study center for their fourth visit on Week 3, Day 1 (±one day) for the following:
(1) opioid toxicity assessment;
(2) review take-home diary;
(3) record new/changed adverse events and concomitant medications;
(4) collect study medication from Week 2 (Study Days 1-8) and account for used/unused supplies;
(5) vital signs;
(6) blood sample for pk analysis;
(7) quality of analgesia;
(8) pain control;
(9) global assessment of study medication;
(10) SF-12 Health Survey;
(11) WOMAC Osteoarthritis Index; and
(12) dispense take-home daily diary and study medication for Week 3 (Study Days 1-8).

Patients were instructed to take up to three doses of study medication on this day (noon, afternoon and at bedtime). In addition, patients were instructed to take their Day 8 ‘waking’ dose from this medication kit. At the conclusion of this visit, the patient was given an appointment card for the next study visit.

Patients were contacted by telephone on the evening of Day 1 of Treatment Week 3 to determine whether they are tolerating the higher dose; to check for adverse events, compliance and concomitant medications; and to remind them to complete the daily diary and bring it to the next visit.

Patients returned to the study center for their fifth (End of Treatment) visit on Week 3, Day 8 (±one day) for the following:
(1) review take-home diary;
(2) record new/changed adverse events and concomitant medications;
(3) collect study medication from Week 3 (Study Days 1-8) and account for used/unused supplies;
(4) complete physical examination and vital signs;
(5) EKG (QTc interval only);
(5) blood samples for clinical laboratory tests (hematology and chemistry);
(6) urine sample for clinical laboratory tests (urinalysis);
(7) blood sample for PK analysis;
(8) quality of analgesia;
(9) pain control;
(10) global assessment of study medication;
(11) SF-12 Health Survey;
(12) WOMAC Osteoarthritis Index;
(13) SOWS; and
(14) dispense take-home daily diary (SOWS and adverse event log) for follow-up period.

Blood samples that were taken during the study at various patient visits were used for a variety of analyses including clinical laboratory tests, PK analysis (see, e.g., Example 3) and cytokine analysis (see, e.g., Example 4).

At the conclusion of this visit, prior to departing the center, the patient was given an appointment card for the next study visit. Patients were instructed to contact the study center immediately if they experienced severe signs and symptoms of opioid withdrawal.

The study center contacted patients before noon once daily (for four days after the last dose of study medication) to monitor for symptoms of opioid withdrawal. On each telephone call, the study center verified that the SOWS have been completed each day (in the morning) by the patients. In addition, there was a check for adverse events and concomitant medications. If necessary, a clinic visit was required for those patients with clinically significant withdrawal symptoms.

Patients returned to the study center approximately one week (±two days) after the last dose of study medication for a post-treatment follow-up visit. At this visit, the following assessments were completed:
(1) review take-home diary; and
(2) record new/changed adverse events and concomitant medications.

Patients could choose to discontinue study drug or study participation at any time, for any reason, specified or unspecified, and without prejudice. If a patient chose to discontinue study drug early, the investigator requested that the patient return to the clinic within 24 hours of stopping the study medication and complete the end-of-treatment assessments described above, as well as the opioid withdrawal monitoring period described above. The investigator also requested that the patient remain in the study for the post-treatment follow-up visit.

For randomization and blinding of the study, the randomization was stratified on patient sex; it was not stratified on investigator. The randomization schedule was generated using a permuted blocks algorithm. The study randomization was unblinded only after all study patients completed therapy and the database was finalized and locked.

The primary analysis population for both efficacy and safety included all patients who took study medication. In the event that a patient was randomized incorrectly or was otherwise administered the incorrect study drug, the patient was to be analyzed according to the study drug actually received.

For the efficacy analysis, endpoints were represented and analyzed by week. Missing efficacy data was imputed across weeks using the last-observation-carried-forward (LOCF) method. Thus, the primary procedure for the analysis of efficacy data was based on a LOCF approach.

The daily pain intensity ratings were summarized as follows. For each week, the pain intensity recorded on the last two full days of dosing within the week, restricted to Day 5 or later, was averaged. If only a single observation was available, it was used; otherwise, the endpoint was not defined. The pain intensity averages were represented as both (1) a change from baseline and (2) a percent change from baseline. The baseline value was defined as the average pain intensity over the last two values recorded during the baseline period; if necessary, a single value was used.

The global assessment, quality of analgesia, and pain control, recorded at the end of each week, were summarized in terms of category proportions.

The SF-12 evaluations, recorded at baseline and at the end of each week, were scored as described in Ware et al., “SF-12: How to score the SF-12 physical and mental health summary scales.” QualityMetric Inc., Lincoln, R.I., and the Health Assessment Lab, Boston, Mass. (3d Ed. 1998), which is incorporated by reference herein. The summarization and analysis of the WOMAC Osteoarthritis Index were specified in the Statistical Analysis Plan per the WOMAC User Guide, which is obtainable at the WOMAC organization website www.womac.org/contact/index.cfm and incorporated by reference herein.

For primary analysis of data, the primary efficacy endpoint was the percent change from baseline in pain intensity at Week 3. Percent change in pain intensity was analyzed using ANOVA methods. The ANOVA model included factors for treatment, sex, and their interaction. Additional covariates could be added to the model for exploratory purposes. Pairwise treatment group comparisons were made using contrasts within the ANOVA framework. Testing employed Type III sums of squares. If the assumptions of the parametric tests were not valid, non-parametric tests were used.

For secondary analysis of data, pain intensity changes, SF-12, and WOMAC were analyzed with the ANOVA methods as described above. Within treatment arms, weeks were compared in pairwise fashion using paired-sample methods. The global assessments, quality of analgesia ratings, and pain control was analyzed at each week, globally and in pairwise fashion, using the Cocharan-Mantel-Haenszel row mean scores (CMH-RMS) test, using equally spaced scores. An “observed data” analysis, without any data imputation, was conducted on pain intensity changes, global assessments, quality of analgesia ratings, pain control, SF-12, and WOMAC using the same analysis methods described previously.

Adverse events reported were mapped to preferred terms and organ systems using the MedDRA mapping system. Adverse events were associated with weeks according to their onset date. The number and percentage of patients reporting each event were summarized by treatment group and week.

Treatment groups were examined for differences in the incidence and severity of selected opioid-associated adverse events, including constipation, dizziness, somnolence, headache, pruritus, nausea, vomiting, urinary retention, and bradypnoea. The homogeneity of response between males was investigated descriptively.

Each of the SOWS assessments (Gossop, “The Development of a Short Opiate Withdrawal Scale (SOWS).” Addictive Behaviors, Vol. 15, p. 487-490, 1990 (incorporated by reference herein)) on Days 1 through 4 of opioid withdrawal monitoring was reduced to an average symptom score and was summarized in terms of changes from baseline, which was defined as the in-clinic assessment at the end of treatment visit (Week 3, Day 8).

The study's sensitivity was broadly assessed by calculating the power of the Wilcoxon test to detect a pairwise treatment difference in an underlying normally distributed endpoint where the two treatment group means differ by one-half a standard deviation. Under these assumptions, the statistical power of a 2-sided Wilcoxon test was:

| | |
|---|---|
| | Power of a Pairwise |
| Analyzable Sample | Treatment Comparison |

| Size per Group | 0.05-level | 0.025-level |
|---|---|---|
| 100 | 92% | 87% |
| 90 | 89% | 83% |
| 80 | 85% | 78% |
| 70 | 80% | 72% |

Results were obtained using nQuery Advisor®, version 4.0 (Statistical Solutions Ltd., Boston, Mass.).

One efficacy endpoint for this study was percent change in pain intensity from baseline to Week 3. In general, a dose response relationship was observed. That is, greater reductions in mean PI occurred as the dose increased in all active treatment groups. The greatest reduction occurred in the oxycodone plus naltrexone BID combination treatment group. The mean percent change in pain intensity from baseline to Week 3 was 39.2% for this BID group. This was both clinically and statistically significant when compared to the other treatment groups. Tables 7A, 7B, and 7C show averages for actual values for Pain Intensity at each of Weeks 1, 2 and 3, based on the Intent to Treat Population using the LOCF method and Table 7D shows the baseline values.

TABLE 7A
PAIN INTENSITY - BY WEEK¹
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 1 | | | | | |
| N | 46 | 93 | 96 | 84 | 319 |
| MEAN | 6.5 | 6.1 | 6.3 | 5.5 | 6.1 |
| STANDARD DEVIATION | 2.10 | 2.17 | 2.14 | 2.11 | 2.16 |
| MINIMUM | 2.5 | 1.0 | 1.0 | 0.0 | 0.0 |
| MEDIAN | 6.5 | 6.0 | 6.8 | 5.5 | 6.0 |
| MAXIMUM | 10.0 | 10.0 | 10.0 | 10.0 | 10.0 |
| MODEL P-VALUES² | | | | | |
| TREATMENT | 0.053 | | | | |
| SEX | 0.727 | | | | |
| TREATMENT + SEX | 0.860 | | | | |
| PAIRWISE COMPARISON P-VALUES² | | | | | |
| PLACEBO QID | | 0.225 | 0.456 | 0.013 | |
| OXY QID | | | 0.561 | 0.108 | |
| OXY + NTX QID | | | | 0.030 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

TABLE 7B
PAIN INTENSITY - BY WEEK¹
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 2 | | | | | |
| N | 46 | 93 | 96 | 84 | 319 |
| MEAN | 6.2 | 5.8 | 6.0 | 5.0 | 5.7 |
| STANDARD DEVIATION | 2.47 | 2.25 | 2.20 | 2.21 | 2.29 |
| MINIMUM | 1.0 | 0.0 | 1.0 | 0.0 | 0.0 |
| MEDIAN | 7.0 | 6.0 | 6.0 | 5.0 | 6.0 |
| MAXIMUM | 10.0 | 10.0 | 10.0 | 10.0 | 10.0 |
| MODEL P-VALUES² | | | | | |
| TREATMENT | 0.007 | | | | |
| SEX | 0.972 | | | | |
| TREATMENT + SEX | 0.795 | | | | |
| PAIRWISE COMPARISON P-VALUES² | | | | | |
| PLACEBO QID | | 0.148 | 0.463 | 0.002 | |
| OXY QID | | | 0.375 | 0.046 | |
| OXY + NTX QID | | | | 0.004 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

TABLE 7C
PAIN INTENSITY - BY WEEK¹
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 3 | | | | | |
| N | 46 | 93 | 96 | 84 | 319 |
| MEAN | 6.1 | 5.6 | 5.7 | 4.5 | 5.4 |
| STANDARD DEVIATION | 2.79 | 2.33 | 2.43 | 2.44 | 2.51 |
| MINIMUM | 0.0 | 0.0 | 0.0 | 0.0 | 0.0 |
| MEDIAN | 7.0 | 5.5 | 6.0 | 4.3 | 5.5 |
| MAXIMUM | 10.0 | 10.0 | 10.0 | 10.0 | 10.0 |
| MODEL P-VALUES² | | | | | |
| TREATMENT | <0.001 | | | | |
| SEX | 0.416 | | | | |
| TREATMENT + SEX | 0.348 | | | | |
| PAIRWISE COMPARISON P-VALUES² | | | | | |
| PLACEBO QID | | 0.092 | 0.193 | <0.001 | |
| OXY QID | | | 0.630 | 0.009 | |
| OXY + NTX QID | | | | 0.002 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

TABLE 7D
PAIN INTENSITY - BY WEEK¹
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| BASELINE | | | | | |
| N | 50 | 102 | 102 | 101 | 355 |
| MEAN | 7.7 | 7.4 | 7.7 | 7.6 | 7.6 |
| STANDARD DEVIATION | 1.29 | 1.30 | 1.41 | 1.37 | 1.35 |
| MINIMUM | 5.0 | 5.0 | 5.0 | 4.5 | 4.5 |
| MEDIAN | 8.0 | 7.0 | 8.0 | 7.5 | 7.5 |
| MAXIMUM | 10.0 | 10.0 | 10.0 | 10.0 | 10.0 |
| MODEL P-VALUES² | | | | | |
| TREATMENT | 0.482 | | | | |
| SEX | 0.018 | | | | |
| TREATMENT + SEX | 0.876 | | | | |
| PAIRWISE COMPARISON P-VALUES² | | | | | |
| PLACEBO QID | | 0.220 | 0.915 | 0.568 | |
| OXY QID | | | 0.165 | 0.415 | |
| OXY + NTX QID | | | | 0.564 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

Tables 8A, 8B, and 8C for males and 8E, 8F and 8G for females show averages for actual values for Pain Intensity at Weeks 1, 2 and 3, respectively. Tables 8D and 8H show baseline values for males and females, respectively.

TABLE 8A
PAIN INTENSITY - BY WEEK¹ AND SEX
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 15) | (N = 31) | (N = 32) | (N = 32) | (N = 110) |
| ACTUAL VALUE AT WEEK 1 | | | | | |
| N | 14 | 29 | 29 | 25 | 97 |
| MEAN | 6.7 | 5.9 | 6.1 | 5.5 | 6.0 |
| STANDARD DEVIATION | 1.81 | 2.18 | 2.07 | 1.68 | 1.98 |
| MINIMUM | 3.0 | 1.5 | 1.0 | 2.0 | 1.0 |
| MEDIAN | 7.3 | 6.0 | 6.5 | 5.5 | 6.0 |
| MAXIMUM | 9.0 | 10.0 | 10.0 | 8.0 | 10.0 |
| MODEL P-VALUES² | | | | | |
| TREATMENT | 0.325 | | | | |
| PAIRWISE COMPARISON P-VALUES² | | | | | |
| PLACEBO QID | | 0.217 | 0.332 | 0.069 | |
| OXY QID | | | 0.741 | 0.445 | |
| OXY + NTX QID | | | | 0.280 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8B
PAIN INTENSITY - BY WEEK¹ AND SEX
ANALYSIS POPULATION: INTENT TO TREAT POPULATION
MALE

| | PLACEBO | | | OXY + NTX | |
|---|---|---|---|---|---|
| | QID | OXY QID | OXY + NTX QID | BID | TOTAL |
| | (N = 15) | (N = 31) | (N = 32) | (N = 32) | (N = 110) |
| ACTUAL VALUE AT WEEK 2 | | | | | |
| N | 14 | 29 | 29 | 25 | 97 |
| MEAN | 6.6 | 5.5 | 6.0 | 4.9 | 5.7 |
| STANDARD DEVIATION | 2.04 | 2.36 | 2.00 | 1.97 | 2.16 |
| MINIMUM | 3.0 | 0.0 | 1.0 | 1.0 | 0.0 |
| MEDIAN | 7.3 | 5.5 | 6.0 | 5.0 | 6.0 |
| MAXIMUM | 9.0 | 10.0 | 10.0 | 9.0 | 10.0 |
| MODEL P-VALUES² | | | | | |
| TREATMENT | 0.084 | | | | |
| PAIRWISE COMPARISON P-VALUES² | | | | | |
| PLACEBO QID | | 0.117 | 0.366 | 0.017 | |
| OXY QID | | | 0.404 | 0.287 | |
| OXY + NTX QID | | | | 0.064 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8C
PAIN INTENSITY - BY WEEK¹ AND SEX
ANALYSIS POPULATION: INTENT TO TREAT POPULATION
MALE

| | PLACEBO | | | OXY + NTX | |
|---|---|---|---|---|---|
| | QID | OXY QID | OXY + NTX QID | BID | TOTAL |
| | (N = 15) | (N = 31) | (N = 32) | (N = 32) | (N = 110) |
| ACTUAL VALUE AT WEEK 3 | | | | | |
| N | 14 | 29 | 29 | 25 | 97 |
| MEAN | 6.6 | 5.0 | 5.6 | 4.0 | 5.2 |
| STANDARD DEVIATION | 2.30 | 2.45 | 2.05 | 2.40 | 2.42 |
| MINIMUM | 2.5 | 0.0 | 1.0 | 0.5 | 0.0 |
| MEDIAN | 7.3 | 4.5 | 6.0 | 4.0 | 5.0 |
| MAXIMUM | 9.5 | 10.0 | 10.0 | 8.5 | 10.0 |
| MODEL P-VALUES² | | | | | |
| TREATMENT | 0.007 | | | | |
| PAIRWISE COMPARISON P-VALUES² | | | | | |
| PLACEBO QID | | 0.034 | 0.149 | <0.001 | |
| OXY QID | | | 0.395 | 0.110 | |
| OXY + NTX QID | | | | 0.017 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8D
PAIN INTENSITY - BY WEEK¹ AND SEX
ANALYSIS POPULATION: INTENT TO TREAT POPULATION
MALE

| | PLACEBO | | | OXY + NTX | |
|---|---|---|---|---|---|
| | QID | OXY QID | OXY + NTX QID | BID | TOTAL |
| | (N = 15) | (N = 31) | (N = 32) | (N = 32) | (N = 110) |
| BASELINE | | | | | |
| N | 15 | 33 | 32 | 32 | 110 |
| MEAN | 7.4 | 7.2 | 7.3 | 7.3 | 7.3 |
| STANDARD DEVIATION | 1.03 | 1.11 | 1.35 | 1.19 | 1.18 |
| MINIMUM | 5.5 | 5.0 | 5.0 | 5.0 | 5.0 |
| MEDIAN | 7.5 | 7.0 | 7.5 | 7.0 | 7.0 |
| MAXIMUM | 9.0 | 10.0 | 10.0 | 10.0 | 10.0 |
| MODEL P-VALUES² | | | | | |
| TREATMENT | 0.952 | | | | |
| PAIRWISE COMPARISON P-VALUES² | | | | | |
| PLACEBO QID | | 0.583 | 0.812 | 0.780 | |
| OXY QID | | | 0.697 | 0.736 | |
| OXY + NTX QID | | | | 0.959 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8E
PAIN INTENSITY - BY WEEK¹ AND SEX
ANALYSIS POPULATION: INTENT TO TREAT POPULATION
FEMALE

| | PLACEBO | | | OXY + NTX | |
|---|---|---|---|---|---|
| | QID | OXY QID | OXY + NTX QID | BID | TOTAL |
| | (N = 35) | (N = 71) | (N = 70) | (N = 70) | (N = 246) |
| ACTUAL VALUE AT WEEK 1 | | | | | |
| N | 32 | 64 | 67 | 59 | 222 |
| MEAN | 6.4 | 6.2 | 6.4 | 5.5 | 6.1 |
| STANDARD DEVIATION | 2.24 | 2.18 | 2.18 | 2.28 | 2.23 |
| MINIMUM | 2.5 | 1.0 | 1.0 | 0.0 | 0.0 |
| MEDIAN | 6.5 | 6.3 | 7.0 | 5.5 | 6.0 |
| MAXIMUM | 10.0 | 10.0 | 10.0 | 10.0 | 10.0 |
| MODEL P-VALUES² | | | | | |
| TREATMENT | 0.081 | | | | |
| PAIRWISE COMPARISON P-VALUES² | | | | | |
| PLACEBO QID | | 0.649 | 0.995 | 0.055 | |
| OXY QID | | | 0.567 | 0.073 | |
| OXY + NTX QID | | | | 0.018 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8F
PAIN INTENSITY - BY WEEK¹ AND SEX
ANALYSIS POPULATION: INTENT TO TREAT POPULATION
FEMALE

| | PLACEBO | | | OXY + NTX | |
|---|---|---|---|---|---|
| | QID | OXY QID | OXY + NTX QID | BID | TOTAL |
| | (N = 35) | (N = 71) | (N = 70) | (N = 70) | (N = 246) |
| ACTUAL VALUE AT WEEK 2 | | | | | |
| N | 32 | 64 | 67 | 59 | 222 |
| MEAN | 6.1 | 5.9 | 6.0 | 5.0 | 5.7 |
| STANDARD DEVIATION | 2.64 | 2.20 | 2.29 | 2.32 | 2.35 |
| MINIMUM | 1.0 | 0.0 | 1.5 | 0.0 | 0.0 |
| MEDIAN | 6.5 | 6.3 | 6.0 | 5.0 | 5.8 |
| MAXIMUM | 10.0 | 10.0 | 10.0 | 10.0 | 10.0 |
| MODEL P-VALUES² | | | | | |
| TREATMENT | 0.052 | | | | |
| PAIRWISE COMPARISON P-VALUES² | | | | | |
| PLACEBO QID | | 0.699 | 0.960 | 0.039 | |
| OXY QID | | | 0.676 | 0.040 | |
| OXY + NTX QID | | | | 0.013 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8G
PAIN INTENSITY - BY WEEK¹ AND SEX
ANALYSIS POPULATION: INTENT TO TREAT POPULATION
FEMALE

| | PLACEBO | | | OXY + NTX | |
|---|---|---|---|---|---|
| | QID | OXY QID | OXY + NTX QID | BID | TOTAL |
| | (N = 35) | (N = 71) | (N = 70) | (N = 70) | (N = 246) |
| ACTUAL VALUE AT WEEK 3 | | | | | |
| N | 32 | 64 | 67 | 59 | 222 |
| MEAN | 5.9 | 5.9 | 5.7 | 4.8 | 5.5 |
| STANDARD DEVIATION | 2.98 | 2.25 | 2.59 | 2.44 | 2.54 |
| MINIMUM | 0.0 | 0.0 | 0.0 | 0.0 | 0.0 |
| MEDIAN | 6.0 | 6.0 | 6.0 | 5.0 | 5.5 |
| MAXIMUM | 10.0 | 10.0 | 10.0 | 10.0 | 10.0 |
| MODEL P-VALUES² | | | | | |
| TREATMENT | 0.057 | | | | |
| PAIRWISE COMPARISON P-VALUES² | | | | | |
| PLACEBO QID | | 0.977 | 0.769 | 0.044 | |
| OXY QID | | | 0.744 | 0.016 | |
| OXY + NTX QID | | | | 0.034 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

TABLE 8H
PAIN INTENSITY - BY WEEK¹ AND SEX
ANALYSIS POPULATION: INTENT TO TREAT POPULATION
FEMALE

| | PLACEBO | | | OXY + NTX | |
|---|---|---|---|---|---|
| | QID | OXY QID | OXY + NTX QID | BID | TOTAL |
| | (N = 35) | (N = 71) | (N = 70) | (N = 70) | (N = 246) |
| BASELINE | | | | | |
| N | 35 | 71 | 70 | 69 | 245 |
| MEAN | 7.9 | 7.4 | 7.9 | 7.7 | 7.7 |
| STANDARD DEVIATION | 1.38 | 1.38 | 1.41 | 1.43 | 1.41 |
| MINIMUM | 5.0 | 5.0 | 5.0 | 4.5 | 4.5 |
| MEDIAN | 8.0 | 7.5 | 8.0 | 7.5 | 8.0 |
| MAXIMUM | 10.0 | 10.0 | 10.0 | 10.0 | 10.0 |
| MODEL P-VALUES² | | | | | |
| TREATMENT | 0.248 | | | | |
| PAIRWISE COMPARISON P-VALUES² | | | | | |
| PLACEBO QID | | 0.155 | 0.902 | 0.530 | |
| OXY QID | | | 0.058 | 0.332 | |
| OXY + NTX QID | | | | 0.358 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT AS THE EFFECT.

Tables 9A, 9B and 9C show the percent change from baseline PI scores at Weeks 1, 2 and 3, respectively.

TABLE 9A
PAIN INTENSITY - BY WEEK¹
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| PERCENT CHANGE FROM BASELINE TO WEEK 1 | | | | | |
| N | 46 | 93 | 96 | 83 | 318 |
| MEAN | −16.6 | −18.4 | −17.8 | −26.7 | −20.1 |
| STANDARD DEVIATION | 21.86 | 25.09 | 26.27 | 25.80 | 25.40 |
| MINIMUM | −75.0 | −83.3 | −83.3 | −100.0 | −100.0 |
| MEDIAN | −12.9 | −16.7 | −15.5 | −25.0 | −17.6 |
| MAXIMUM | 33.3 | 50.0 | 66.7 | 16.7 | 66.7 |
| MODEL P-VALUES² | | | | | |
| TREATMENT | 0.142 | | | | |
| SEX | 0.224 | | | | |
| TREATMENT + SEX | 0.751 | | | | |
| PAIRWISE COMPARISON P-VALUES² | | | | | |
| PLACEBO QID | | 0.496 | 0.612 | 0.044 | |
| OXY QID | | | 0.827 | 0.099 | |
| OXY + NTX QID | | | | 0.062 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

TABLE 9B
PAIN INTENSITY - BY WEEK¹
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| PERCENT CHANGE FROM BASELINE TO WEEK 2 | | | | | |
| N | 46 | 93 | 96 | 83 | 318 |
| MEAN | −20.4 | −22.9 | −21.2 | −33.9 | −24.9 |
| STANDARD DEVIATION | 28.55 | 27.52 | 28.17 | 27.24 | 28.19 |
| MINIMUM | −84.6 | −100.0 | −83.3 | −100.0 | −100.0 |
| MEDIAN | −13.8 | −20.0 | −20.0 | −33.3 | −21.4 |
| MAXIMUM | 58.3 | 42.9 | 66.7 | 12.5 | 66.7 |
| MODEL P-VALUES² | | | | | |
| TREATMENT | 0.013 | | | | |
| SEX | 0.232 | | | | |
| TREATMENT + SEX | 0.622 | | | | |
| PAIRWISE COMPARISON P-VALUES² | | | | | |
| PLACEBO QID | | 0.341 | 0.718 | 0.007 | |
| OXY QID | | | 0.462 | 0.031 | |
| OXY + NTX QID | | | | 0.004 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

TABLE 9C
PAIN INTENSITY - BY WEEK¹
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| PERCENT CHANGE FROM BASELINE TO WEEK 3 | | | | | |
| N | 46 | 93 | 96 | 83 | 318 |
| MEAN | −21.5 | −24.6 | −26.0 | −39.2 | −28.4 |
| STANDARD DEVIATION | 31.66 | 29.22 | 31.16 | 32.51 | 31.89 |
| MINIMUM | −100.0 | −100.0 | −100.0 | −100.0 | −100.0 |
| MEDIAN | −17.6 | −20.0 | −23.5 | −40.0 | −25.0 |
| MAXIMUM | 58.3 | 42.9 | 66.7 | 55.6 | 66.7 |
| MODEL P-VALUES² | | | | | |
| TREATMENT | 0.002 | | | | |
| SEX | 0.855 | | | | |
| TREATMENT + SEX | 0.221 | | | | |
| PAIRWISE COMPARISON P-VALUES² | | | | | |
| PLACEBO QID | | 0.219 | 0.282 | <0.001 | |
| OXY QID | | | 0.848 | 0.006 | |
| OXY + NTX QID | | | | 0.003 | |

NOTE:
DATA IMPUTED USING THE LAST-OBSERVATION-CARRIED-FORWARD METHOD.
¹USING AVERAGE OF LAST TWO DAYS WITHIN EACH DOSING WEEK.
²P-VALUES FROM ANOVA MODEL WITH TREATMENT, SEX, AND TREATMENT + SEX INTERACTION AS EFFECTS.

Another efficacy endpoint for this study was assessments of quality of analgesia and the results are shown in Tables 10A, 10B and 10C. for Weeks 1, 2 and 3, respectively. The oxycodone plus naltrexone BID treatment group show a consistent and greater improvement in the quality of analgesia at each of Weeks 1 and 2 (see Tables 10A and 10B). At Week 3, oxycodone plus naltrexone QID and oxycodone plus naltrexone BID were significantly better than placebo as shown in Table 10C. A quality of analgesia assessment at Week 3 of very good or excellent was reported by 12.0% of patients treated with placebo, 19.6% of patients treated with oxycodone alone QID, 10.6% of patients with oxycodone plus naltrexone QID, and 33.3% of patients treated with oxycodone plus naltrexone BID.

TABLE 10A
QUALITY OF ANALGESIA - BY WEEK
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 50) | (N = 102) | (N = 102) | (N = 102) | (N = 356) |
| WEEK 1 | | | | | |
| EXCELLENT (4) | 1 (2.0%) | 3 (2.9%) | 3 (2.9%) | 3 (2.9%) | 10 (2.8%) |
| VERY GOOD (3) | 5 (10.0%) | 8 (7.8%) | 13 (12.7%) | 14 (13.7%) | 40 (11.2%) |
| GOOD (2) | 13 (26.0%) | 18 (37.3%) | 26 (25.5%) | 29 (28.4%) | 106 (29.8%) |
| FAIR (1) | 13 (26.0%) | 31 (30.4%) | 33 (32.4%) | 34 (33.3%) | 111 (31.2%) |
| POOR (0) | 17 (34.0%) | 18 (17.6%) | 25 (24.5%) | 21 (20.6%) | 81 (22.8%) |
| MISSING | 1 (2.0%) | 4 (3.9%) | 2 (2.0%) | 1 (1.0%) | 8 (2.2%) |
| OVERALL P-VALUE¹ | 0.446 | | | | |
| PAIRWISE COMPARISON P-VALUES² | | | | | |
| PLACEBO QID | | 0.125 | 0.353 | 0.162 | |
| OXY QID | | | 0.501 | 0.925 | |
| OXY + NTX QID | | | | 0.572 | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 10B
QUALITY OF ANALGESIA - BY WEEK
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 50) | (N = 102) | (N = 102) | (N = 102) | (N = 356) |
| WEEK 2 | | | | | |
| EXCELLENT (4) | 2 (4.0%) | 4 (3.9%) | 4 (3.9%) | 6 (5.9%) | 16 (4.5%) |
| VERY GOOD (3) | 8 (16.0%) | 16 (15.7%) | 13 (12.7%) | 28 (27.5%) | 65 (18.3%) |
| GOOD (2) | 15 (30.0%) | 30 (29.4%) | 37 (36.3%) | 21 (20.6%) | 103 (28.9%) |
| FAIR (1) | 6 (12.0%) | 29 (28.4%) | 24 (23.5%) | 28 (27.5%) | 87 (24.4%) |
| POOR (0) | 18 (36.0%) | 19 (18.6%) | 22 (21.6%) | 18 (17.6%) | 77 (21.6%) |
| MISSING | 1 (2.0%) | 4 (3.9%) | 2 (2.0%) | 1 (1.0%) | 8 (2.2%) |
| OVERALL P-VALUE¹ | 0.254 | | | | |
| PAIRWISE COMPARISON P-VALUES² | | | | | |
| PLACEBO QID | | 0.391 | 0.478 | 0.081 | |
| OXY QID | | | 0.841 | 0.221 | |
| OXY + NTX QID | | | | 0.155 | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 10C
QUALITY OF ANALGESIA - BY WEEK
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 50) | (N = 102) | (N = 102) | (N = 102) | (N = 356) |
| WEEK 3 | | | | | |
| EXCELLENT (4) | 0 (0.0%) | 5 (4.9%) | 3 (2.9%) | 8 (7.8%) | 16 (4.5%) |
| VERY GOOD (3) | 6 (12.0%) | 15 (14.7%) | 17 (16.7%) | 26 (25.5%) | 64 (18.0%) |
| GOOD (2) | 14 (28.0%) | 32 (31.4%) | 34 (33.3%) | 22 (21.6%) | 102 (28.7%) |
| FAIR (1) | 11 (22.0%) | 23 (22.5%) | 27 (26.5%) | 28 (27.5%) | 89 (25.0%) |
| POOR (0) | 18 (36.0%) | 23 (22.5%) | 19 (18.6%) | 17 (16.7%) | 77 (21.6%) |
| MISSING | 1 (2.0%) | 4 (3.9%) | 2 (2.0%) | 1 (1.0%) | 8 (2.2%) |
| OVERALL P-VALUE¹ | 0.017 | | | | |
| PAIRWISE COMPARISON P-VALUES² | | | | | |
| PLACEBO QID | | 0.052 | 0.028 | 0.002 | |
| OXY QID | | | 0.855 | 0.139 | |
| OXY + NTX QID | | | | 0.173 | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

Another efficacy endpoint for this study was a global assessment and the results are shown in Tables 11A, 11B and 11C for Weeks 1, 2 and 3, respectively. At Week 3, oxycodone plus naltrexone QID and oxycodone plus naltrexone BID were statistically significantly better than placebo (in pairwise comparisons) as shown in Table 11C. A global assessment of very good or excellent at Week 3 was reported by 16.0% of patients treated with oxycodone plus naltrexone QID, 19.6% of patients treated with oxycodone alone QID, 22.5% of patients with oxycodone plus naltrexone QID, and 30.4% of patients treated with oxycodone plus naltrexone BID. Tables 11A, 11B and 11C also show the p value vs. placebo calculated for the scores from the global assessment for Weeks 1, 2 and 3, respectively, which were determined using the Cochran-Mantel-Haenszel row mean scores (CMH-RMS) test, using equally spaced scores. Thus, the results in Table 11C generally show a population shift from patient responses of poor and fair toward patient responses of very good and excellent when comparing the placebo group to the oxycodone alone QID, oxycodone plus naltrexone QID and oxycodone plus naltrexone BID treatment groups. Larger percentages of patients in the oxycodone plus naltrexone BID treatment group gave responses of very good or excellent.

TABLE 11A
GLOBAL ASSESSMENT - BY WEEK
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 50) | (N = 102) | (N = 102) | (N = 102) | (N = 356) |
| WEEK 1 | | | | | |
| EXCELLENT (4) | 0 (0.0%) | 3 (2.9%) | 5 (4.9%) | 2 (2.0%) | 10 (2.8%) |
| VERY GOOD (3) | 8 (16.0%) | 12 (11.8%) | 17 (16.7%) | 18 (17.6%) | 55 (15.4%) |
| GOOD (2) | 11 (22.0%) | 30 (29.4%) | 27 (26.5%) | 31 (30.4%) | 99 (27.8%) |
| FAIR (1) | 14 (28.0%) | 37 (36.3%) | 25 (24.5%) | 26 (25.5%) | 102 (28.7%) |
| POOR (0) | 16 (32.0%) | 16 (15.7%) | 26 (25.5%) | 24 (23.5%) | 82 (23.0%) |
| MISSING | 1 (2.0%) | 4 (3.9%) | 2 (2.0%) | 1 (1.0%) | 8 (2.2%) |
| OVERALL P-VALUE¹ | 0.487 | | | | |
| PAIRWISE COMPARISON P-VALUES² | | | | | |
| PLACEBO QID | | 0.160 | 0.174 | 0.173 | |
| OXY QID | | | 0.896 | 0.970 | |
| OXY + NTX QID | | | | 0.927 | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 11B
GLOBAL ASSESSMENT - BY WEEK
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 50) | (N = 102) | (N = 102) | (N = 102) | (N = 356) |
| WEEK 2 | | | | | |
| EXCELLENT (4) | 0 (0.0%) | 6 (5.9%) | 4 (3.9%) | 6 (5.9%) | 16 (4.5%) |
| VERY GOOD (3) | 10 (20.0%) | 14 (33.7%) | 14 (13.7%) | 20 (19.6%) | 58 (16.3%) |
| GOOD (2) | 13 (26.0%) | 13 (32.4%) | 38 (37.3%) | 28 (27.5%) | 112 (31.5%) |
| FAIR (1) | 7 (14.0%) | 29 (28.4%) | 22 (21.6%) | 23 (22.5%) | 81 (22.8%) |
| POOR (0) | 19 (38.0%) | 16 (15.7%) | 22 (21.6%) | 24 (23.5%) | 81 (22.8%) |
| MISSING | 1 (2.0%) | 4 (3.9%) | 2 (2.0%) | 1 (1.0%) | 9 (2.2%) |
| OVERALL P-VALUE¹ | 0.319 | | | | |
| PAIRWISE COMPARISON P-VALUES² | | | | | |
| PLACEBO QID | | 0.074 | 0.166 | 0.120 | |
| OXY QID | | | 0.597 | 0.860 | |
| OXY + NTX QID | | | | 0.742 | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 11C
GLOBAL ASSESSMENT - BY WEEK
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 50) | (N = 102) | (N = 102) | (N = 102) | (N = 356) |
| WEEK 3 | | | | | |
| EXCELLENT (4) | 1 (2.0%) | 6 (5.9%) | 5 (4.9%) | 9 (8.8%) | 21 (5.9%) |
| VERY GOOD (3) | 7 (14.0%) | 14 (13.7%) | 18 (17.6%) | 22 (21.6%) | 61 (17.1%) |
| GOOD (2) | 13 (26.0%) | 36 (35.3%) | 34 (33.3%) | 23 (22.5%) | 106 (29.8%) |
| FAIR (1) | 9 (18.0%) | 18 (17.6%) | 21 (20.6%) | 22 (21.6%) | 70 (19.7%) |
| POOR (0) | 19 (38.0%) | 24 (23.5%) | 22 (21.6%) | 25 (24.5%) | 90 (25.3%) |
| MISSING | 1 (2.0%) | 4 (3.9%) | 2 (2.0%) | 1 (1.0%) | 8 (2.2%) |
| OVERALL P-VALUE¹ | 0.165 | | | | |
| PAIRWISE COMPARISON P-VALUES² | | | | | |
| PLACEBO QID | | 0.078 | 0.048 | 0.039 | |
| OXY QID | | | 0.818 | 0.604 | |
| OXY + NTX QID | | | | 0.760 | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

Another efficacy endpoint for this study was an assessment of pain control and the results are shown in Tables 12A, 12B and 12C for Weeks 1, 2 and 3, respectively. From Week 1 to Weeks 2 and 3, there were significant changes within treatment group (indicating better control throughout the day) in the pain control assessments for the oxycodone plus naltrexone BID treatment group. There were also statistically significant changes for oxycodone plus naltrexone QID from Week 1 to Week 3 and for placebo from Week 1 to Week 3. As shown in Tables 12A, 12B and 12C, patients in the oxycodone plus naltrexone BID treatment group showed improved pain control when compared to placebo at each week of treatment. Tables 12A, 12B and 12C also show the p value vs. placebo calculated for the scores from Pain Control, which were determined using the Cochran-Mantel-Haenszel row mean scores (CMH-RMS) test, using equally spaced scores.

TABLE 12A
PAIN CONTROL - BY WEEK
ANALYSIS POPULATION: INTENT TO TREAT POPULATION

| | PLACEBO | | OXY + NTX | OXY + NTX | |
|---|---|---|---|---|---|
| | QID | OXY QID | QID | BID | TOTAL |
| | (N = 50) | (N = 102) | (N = 102) | (N = 102) | (N = 356) |
| WEEK 1 | | | | | |
| PAIN CONTROLLED THROUGHOUT EACH DAY (4) | 2 (4.0%) | 10 (9.8%) | 5 (4.9%) | 5 (4.9%) | 22 (6.2%) |
| PAIN CONTROLLED MOST OF EACH DAY (3) | 8 (16.0%) | 17 (16.7%) | 20 (19.6%) | 23 (22.5%) | 68 (19.1%) |
| PAIN CONTROLLED SEVERAL HRS EACH DAY (2) | 8 (16.0%) | 29 (28.4%) | 31 (30.4%) | 29 (28.4%) | 97 (27.2%) |
| PAIN CONTROLLED A FEW HRS/LESS EACH DAY (1) | 31 (62.0%) | 42 (41.2%) | 44 (43.1%) | 44 (43.1%) | 161 (45.2%) |
| MISSING | 1 (2.0%) | 4 (3.9%) | 2 (2.0%) | 1 (1.0%) | 8 (2.2%) |
| OVERALL P-VALUE¹ | 0.227 | | | | |
| PAIRWISE COMPARISON P-VALUES² | | | | | |
| PLACEBO QID | | 0.051 | 0.120 | 0.084 | |
| OXY QID | | | 0.514 | 0.673 | |
| OXY + NTX QID | | | | 0.810 | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 12B
PAIN CONTROL - BY WEEK
ANALYSIS POPULATION: INTENT TO TREAT POPULATION

| | PLACEBO | | OXY + NTX | OXY + NTX | |
|---|---|---|---|---|---|
| | QID | OXY QID | QID | BID | TOTAL |
| | (N = 50) | (N = 102) | (N = 102) | (N = 102) | (N = 356) |
| WEEK 2 | | | | | |
| PAIN CONTROLLED THROUGHOUT EACH DAY (4) | 2 (4.0%) | 10 (9.8%) | 6 (5.9%) | 9 (8.8%) | 27 (7.6%) |
| PAIN CONTROLLED MOST OF EACH DAY (3) | 11 (22.0%) | 26 (25.5%) | 29 (28.4%) | 33 (32.4%) | 99 (27.8%) |
| PAIN CONTROLLED SEVERAL HRS EACH DAY (2) | 11 (22.0%) | 26 (25.5%) | 26 (25.5%) | 18 (17.6%) | 81 (22.8%) |
| PAIN CONTROLLED A FEW HRS/LESS EACH DAY (1) | 25 (50.0%) | 36 (35.3%) | 39 (38.2%) | 41 (40.2%) | 141 (39.6%) |
| MISSING | 1 (2.0%) | 4 (3.9%) | 2 (2.0%) | 1 (1.0%) | 8 (2.2%) |
| OVERALL P-VALUE¹ | 0.299 | | | | |
| PAIRWISE COMPARISON P-VALUES² | | | | | |
| PLACEBO QID | | 0.080 | 0.179 | 0.087 | |
| OXY QID | | | 0.560 | 0.983 | |
| OXY + NTX QID | | | | 0.577 | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

TABLE 12C
PAIN CONTROL - BY WEEK
ANALYSIS POPULATION: INTENT TO TREAT POPULATION

| | PLACEBO | | OXY + NTX | OXY + NTX | |
|---|---|---|---|---|---|
| | QID | OXY QID | QID | BID | TOTAL |
| | (N = 50) | (N = 102) | (N = 102) | (N = 102) | (N = 356) |
| WEEK 3 | | | | | |
| PAIN CONTROLLED THROUGHOUT EACH DAY (4) | 3 (6.0%) | 8 (7.8%) | 7 (6.9%) | 12 (21.8%) | 30 (8.4%) |
| PAIN CONTROLLED MOST OF EACH DAY (3) | 10 (20.0%) | 27 (26.5%) | 31 (30.4%) | 32 (31.4%) | 100 (28.1%) |
| PAIN CONTROLLED SEVERAL HRS EACH DAY (2) | 11 (22.0%) | 28 (27.5%) | 22 (21.6%) | 16 (17.6%) | 79 (22.2%) |
| PAIN CONTROLLED A FEW HRS/LESS EACH DAY (1) | 25 (50.0%) | 35 (34.3%) | 40 (39.2%) | 39 (38.2%) | 139 (39.0%) |
| MISSING | 1 (2.0%) | 4 (3.9%) | 2 (2.0%) | 1 (1.0%) | 8 (2.2%) |
| OVERALL P-VALUE¹ | 0.259 | | | | |
| PAIRWISE COMPARISON P-VALUES² | | | | | |
| PLACEBO QID | | 0.123 | 0.177 | 0.055 | |
| OXY QID | | | 0.822 | 0.553 | |
| OXY + NTX QID | | | | 0.419 | |

NOTE:
DATA IMPUTED USING THE LAST-OBSERVATION-CARRIED-FORWARD METHOD.
¹COCHRAN-MANTEL-HAENSZEL (ROW MEAN SCORES) TEST ACROSS TREATMENT GROUPS USING EQUALLY SPACED SCORES.
²COCHRAN-MANTEL-HAENSZEL (ROW MEAN SCORES) TEST BETWEEN TREATMENT GROUPS USING EQUALLY SPACED SCORES.

Another efficacy endpoint for this study was a functional assessment using WOMAC, including its three subscales for pain, stiffness and physical function. The actual values from the WOMAC pain subscale are shown in Tables 13A, 13B and 13C for Weeks 1, 2 and 3, respectively and Table 13D shows the baseline values. Greater improvements (% change from baseline to Week 3) were observed with BID administration of oxycodone plus naltrexone in all categories (pain, stiffness, or physical function). Oxycodone plus naltrexone BID was statistically significantly better than placebo at Weeks 2 and 3 as measured by the WOMAC pain subscale, stiffness subscale, physical function subscale and total score, as shown below in Tables 13A, 13B and 13C (pain), 14A, 14B and 14C (stiffness), 15A, 15B and 15C (physical function) and 16A, 16B and 16C (total score). In each case, the A, B and C tables show the values at Weeks 1, 2 and 3, respectively and the D tables show the baseline values.

TABLE 13A
WOMAC OSTEOARTHRITIS PAIN SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 1 | | | | | |
| N | 50 | 98 | 100 | 100 | 348 |
| MEAN | 311.1 | 263.2 | 292.5 | 271.9 | 281.0 |
| STANDARD DEVIATION | 121.29 | 124.90 | 107.04 | 119.53 | 118.60 |
| MINIMUM | 37.0 | 3.0 | 28.0 | 0.0 | 0.0 |
| MEDIAN | 320.0 | 279.0 | 312.0 | 274.0 | 297.0 |
| MAXIMUM | 500.0 | 483.0 | 492.0 | 488.0 | 500.0 |
| MODEL P-VALUES¹ | | | | | |
| TREATMENT | 0.079 | | | | |
| SEX | 0.380 | | | | |
| TREATMENT + SEX | 0.856 | | | | |
| PAIRWISE COMPARISON P-VALUES¹ | | | | | |
| PLACEBO QID | — | 0.029 | 0.531 | 0.088 | |
| OXY QID | — | — | 0.053 | 0.544 | |
| OXY + NTX QID | — | — | — | 0.179 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 13B
WOMAC OSTEOARTHRITIS PAIN SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 2 | | | | | |
| N | 50 | 98 | 100 | 101 | 349 |
| MEAN | 285.7 | 245.0 | 257.2 | 237.7 | 252.2 |
| STANDARD DEVIATION | 144.01 | 131.13 | 120.08 | 133.58 | 131.10 |
| MINIMUM | 14.0 | 0.0 | 22.0 | 0.0 | 0.0 |
| MEDIAN | 295.5 | 227.5 | 272.0 | 226.0 | 257.5 |
| MAXIMUM | 500.0 | 482.0 | 492.0 | 494.0 | 500.0 |
| MODEL P-VALUES¹ | | | | | |
| TREATMENT | 0.107 | | | | |
| SEX | 0.559 | | | | |
| TREATMENT + SEX | 0.631 | | | | |
| PAIRWISE COMPARISON P-VALUES¹ | | | | | |
| PLACEBO QID | — | 0.064 | 0.230 | 0.020 | |
| OXY QID | — | — | 0.415 | 0.568 | |
| OXY + NTX QID | — | — | — | 0.161 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 13C
WOMAC OSTEOARTHRITIS PAIN SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 3 | | | | | |
| N | 50 | 98 | 100 | 101 | 349 |
| MEAN | 269.1 | 238.7 | 245.3 | 225.7 | 241.2 |
| STANDARD DEVIATION | 160.58 | 135.11 | 131.06 | 139.11 | 139.12 |
| MINIMUM | 4.0 | 0.0 | 0.0 | 0.0 | 0.0 |
| MEDIAN | 316.5 | 225.0 | 280.0 | 211.0 | 244.0 |
| MAXIMUM | 500.0 | 488.0 | 492.0 | 495.0 | 500.0 |
| MODEL P-VALUES¹ | | | | | |
| TREATMENT | 0.172 | | | | |
| SEX | 0.155 | | | | |
| TREATMENT + SEX | 0.439 | | | | |
| PAIRWISE COMPARISON P-VALUES¹ | | | | | |
| PLACEBO QID | — | 0.154 | 0.354 | 0.035 | |
| OXY QID | — | — | 0.531 | 0.409 | |
| OXY + NTX QID | — | — | — | 0.142 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 13D
WOMAC OSTEOARTHRITIS PAIN SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| BASELINE | | | | | |
| N | 50 | 102 | 102 | 101 | 355 |
| MEAN | 355.1 | 327.9 | 342.9 | 342.7 | 340.3 |
| STANDARD DEVIATION | 80.68 | 90.62 | 71.40 | 80.97 | 81.56 |
| MINIMUM | 134.0 | 25.0 | 148.0 | 86.0 | 25.0 |
| MEDIAN | 363.5 | 338.0 | 340.0 | 350.0 | 347.0 |
| MAXIMUM | 489.0 | 500.0 | 482.0 | 490.0 | 500.0 |
| MODEL P-VALUES¹ | | | | | |
| TREATMENT | 0.371 | | | | |
| SEX | 0.021 | | | | |
| TREATMENT + SEX | 0.925 | | | | |
| PAIRWISE COMPARISON P-VALUES¹ | | | | | |
| PLACEBO QID | — | 0.110 | 0.586 | 0.481 | |
| OXY QID | — | — | 0.190 | 0.267 | |
| OXY + NTX QID | — | — | — | 0.842 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 14A
WOMAC OSTEOARTHRITIS STIFFNESS SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 1 | | | | | |
| N | 49 | 98 | 100 | 99 | 346 |
| MEAN | 131.0 | 120.3 | 127.4 | 119.0 | 123.5 |
| STANDARD DEVIATION | 50.41 | 51.28 | 44.29 | 51.32 | 49.24 |
| MINIMUM | 8.0 | 2.0 | 6.0 | 0.0 | 0.0 |
| MEDIAN | 139.0 | 129.5 | 131.0 | 128.0 | 130.5 |
| MAXIMUM | 199.0 | 200.0 | 197.0 | 200.0 | 200.0 |
| MODEL P-VALUES¹ | | | | | |
| TREATMENT | 0.274 | | | | |
| SEX | 0.290 | | | | |
| TREATMENT + SEX | 0.656 | | | | |
| PAIRWISE COMPARISON P-VALUES¹ | | | | | |
| PLACEBO QID | — | 0.192 | 0.858 | 0.174 | |
| OXY QID | — | — | 0.163 | 0.954 | |
| OXY + NTX QID | — | — | — | 0.142 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 14B
WOMAC OSTEOARTHRITIS STIFFNESS SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 2 | | | | | |
| N | 50 | 98 | 100 | 100 | 348 |
| MEAN | 128.3 | 111.4 | 115.8 | 106.5 | 113.7 |
| STANDARD DEVIATION | 55.95 | 56.29 | 48.60 | 55.44 | 54.10 |
| MINIMUM | 8.0 | 0.0 | 8.0 | 0.0 | 0.0 |
| MEDIAN | 142.5 | 119.5 | 125.0 | 113.5 | 121.0 |
| MAXIMUM | 200.0 | 199.0 | 197.0 | 200.0 | 200.0 |
| MODEL P-VALUES¹ | | | | | |
| TREATMENT | 0.098 | | | | |
| SEX | 0.249 | | | | |
| TREATMENT + SEX | 0.745 | | | | |
| PAIRWISE COMPARISON P-VALUES¹ | | | | | |
| PLACEBO QID | — | 0.061 | 0.238 | 0.019 | |
| OXY QID | — | — | 0.383 | 0.569 | |
| OXY + NTX QID | — | — | — | 0.145 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 14C
WOMAC OSTEOARTHRITIS STIFFNESS SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 3 | | | | | |
| N | 50 | 98 | 100 | 100 | 348 |
| MEAN | 119.8 | 106.3 | 110.7 | 101.1 | 208.0 |
| STANDARD DEVIATION | 64.12 | 57.99 | 53.42 | 57.66 | 57.62 |
| MINIMUM | 4.0 | 0.0 | 0.0 | 0.0 | 0.0 |
| MEDIAN | 139.5 | 109.0 | 117.5 | 113.5 | 117.5 |
| MAXIMUM | 200.0 | 200.0 | 197.0 | 200.0 | 200.0 |
| MODEL P-VALUES¹ | | | | | |
| TREATMENT | 0.148 | | | | |
| SEX | 0.068 | | | | |
| TREATMENT + SEX | 0.396 | | | | |
| PAIRWISE COMPARISON P-VALUES¹ | | | | | |
| PLACEBO QID | — | 0.143 | 0.496 | 0.042 | |
| OXY QID | — | — | 0.328 | 0.491 | |
| OXY + NTX QID | — | — | — | 0.092 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 14D
WOMAC OSTEOARTHRITIS STIFFNESS SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| BASELINE | | | | | |
| N | 50 | 102 | 102 | 101 | 355 |
| MEAN | 149.7 | 141.8 | 149.3 | 141.4 | 145.0 |
| STANDARD DEVIATION | 41.75 | 37.28 | 33.50 | 39.35 | 37.56 |
| MINIMUM | 13.0 | 32.0 | 41.0 | 10.0 | 10.0 |
| MEDIAN | 158.5 | 145.5 | 152.0 | 147.0 | 148.0 |
| MAXIMUM | 200.0 | 200.0 | 197.0 | 200.0 | 200.0 |
| MODEL P-VALUES¹ | | | | | |
| TREATMENT | 0.221 | | | | |
| SEX | 0.075 | | | | |
| TREATMENT + SEX | 0.688 | | | | |
| PAIRWISE COMPARISON P-VALUES¹ | | | | | |
| PLACEBO QID | — | 0.266 | 0.993 | 0.149 | |
| OXY QID | — | — | 0.170 | 0.686 | |
| OXY + NTX QID | — | — | — | 0.075 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 15A
WOMAC OSTEOARTHRITIS PHYSICAL FUNCTION SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 1 | | | | | |
| N | 50 | 98 | 100 | 100 | 348 |
| MEAN | 1034.4 | 891.8 | 998.7 | 932.9 | 954.8 |
| STANDARD DEVIATION | 431.11 | 423.35 | 378.56 | 413.37 | 410.74 |
| MINIMUM | 123.0 | 15.0 | 149.0 | 52.0 | 15.0 |
| MEDIAN | 1098.0 | 930.5 | 1098.5 | 955.5 | 1018.5 |
| MAXIMUM | 1700.0 | 1669.0 | 1658.0 | 1642.0 | 1700.0 |
| MODEL P-VALUES[1] | | | | | |
| TREATMENT | 0.085 | | | | |
| SEX | 0.622 | | | | |
| TREATMENT + SEX | 0.771 | | | | |
| PAIRWISE COMPARISON P-VALUES¹ | | | | | |
| PLACEBO QID | — | 0.034 | 0.640 | 0.136 | |
| OXY QID | — | — | 0.040 | 0.424 | |
| OXY + NTX QID | — | — | — | 0.204 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 15B
WOMAC OSTEOARTHRITIS PHYSICAL FUNCTION SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 2 | | | | | |
| N | 50 | 98 | 100 | 101 | 349 |
| MEAN | 985.0 | 847.6 | 910.6 | 834.4 | 881.5 |
| STANDARD DEVIATION | 461.89 | 451.42 | 410.69 | 439.16 | 439.31 |
| MINIMUM | 57.4 | 16.0 | 54.0 | 17.0 | 16.0 |
| MEDIAN | 1038.0 | 880.0 | 979.5 | 799.0 | 888.0 |
| MAXIMUM | 1700.0 | 1669.0 | 1658.0 | 1646.0 | 1700.0 |
| MODEL P-VALUES¹ | | | | | |
| TREATMENT | 0.099 | | | | |
| SEX | 0.457 | | | | |
| TREATMENT + SEX | 0.455 | | | | |
| PAIRWISE COMPARISON P-VALUES¹ | | | | | |
| PLACEBO QID | — | 0.070 | 0.471 | 0.036 | |
| OXY QID | — | — | 0.176 | 0.738 | |
| OXY + NTX QID | — | — | — | 0.088 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 15C
WOMAC OSTEOARTHRITIS PHYSICAL FUNCTION SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 3 | | | | | |
| N | 50 | 98 | 100 | 101 | 349 |
| MEAN | 939.9 | 820.0 | 869.0 | 801.9 | 846.0 |
| STANDARD DEVIATION | 534.35 | 468.67 | 452.25 | 458.64 | 471.41 |
| MINIMUM | 9.6 | 5.0 | 0.0 | 17.0 | 0.0 |
| MEDIAN | 1004.0 | 867.0 | 969.5 | 776.0 | 890.0 |
| MAXIMUM | 1700.0 | 1680.0 | 1662.0 | 1671.0 | 1700.0 |
| MODEL P-VALUES¹ | | | | | |
| TREATMENT | 0.176 | | | | |
| SEX | 0.238 | | | | |
| TREATMENT + SEX | 0.380 | | | | |
| PAIRWISE COMPARISON P-VALUES¹ | | | | | |
| PLACEBO QID | — | 0.131 | 0.489 | 0.052 | |
| OXY QID | — | — | 0.307 | 0.604 | |
| OXY + NTX QID | — | — | — | 0.120 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 15D
WOMAC OSTEOARTHRITIS PHYSICAL FUNCTION SUBSCALE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| BASELINE | | | | | |
| N | 49 | 101 | 102 | 101 | 353 |
| MEAN | 1146.7 | 1101.7 | 1147.8 | 1142.9 | 1133.1 |
| STANDARD DEVIATION | 341.34 | 324.42 | 302.25 | 304.85 | 314.33 |
| MINIMUM | 208.0 | 208.0 | 199.0 | 273.0 | 199.0 |
| MEDIAN | 1180.0 | 1151.0 | 1187.5 | 1210.0 | 1187.0 |
| MAXIMUM | 1688.0 | 1700.0 | 1659.0 | 1650.0 | 1700.0 |
| MODEL P-VALUES¹ | | | | | |
| TREATMENT | 0.837 | | | | |
| SEX | 0.020 | | | | |
| TREATMENT + SEX | 0.936 | | | | |
| PAIRWISE COMPARISON P-VALUES¹ | | | | | |
| PLACEBO QID | — | 0.474 | 0.929 | 0.864 | |
| OXY QID | — | — | 0.435 | 0.498 | |
| OXY + NTX QID | — | — | — | 0.918 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 16A
WOMAC OSTEOARTHRITIS TOTAL SCORE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 1 | | | | | |
| N | 49 | 98 | 100 | 99 | 346 |
| MEAN | 1468.6 | 1275.3 | 1418.5 | 1316.8 | 1355.9 |
| STANDARD DEVIATION | 592.38 | 582.28 | 511.30 | 565.78 | 561.54 |
| MINIMUM | 214.0 | 20.0 | 227.0 | 55.0 | 20.0 |
| MEDIAN | 1539.0 | 1312.5 | 1552.5 | 1323.0 | 1428.0 |
| MAXIMUM | 2398.0 | 2347.0 | 2340.0 | 2328.0 | 2398.0 |
| MODEL P-VALUES¹ | | | | | |
| TREATMENT | 0.085 | | | | |
| SEX | 0.576 | | | | |
| TREATMENT + SEX | 0.782 | | | | |
| PAIRWISE COMPARISON P-VALUES¹ | | | | | |
| PLACEBO QID | — | 0.039 | 0.664 | 0.119 | |
| OXY QID | — | — | 0.042 | 0.516 | |
| OXY + NTX QID | — | — | — | 0.162 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 16B
WOMAC OSTEOARTHRITIS TOTAL SCORE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 2 | | | | | |
| N | 50 | 98 | 100 | 100 | 348 |
| MEAN | 1399.0 | 1204.0 | 1283.0 | 1170.3 | 1245.0 |
| STANDARD DEVIATION | 649.08 | 626.06 | 557.71 | 611.83 | 608.70 |
| MINIMUM | 81.4 | 20.0 | 99.0 | 55.0 | 20.0 |
| MEDIAN | 1483.0 | 1222.5 | 1309.0 | 1137.5 | 1257.0 |
| MAXIMUM | 2400.0 | 2347.0 | 2346.0 | 2338.0 | 2400.0 |
| MODEL P-VALUES¹ | | | | | |
| TREATMENT | 0.082 | | | | |
| SEX | 0.463 | | | | |
| TREATMENT + SEX | 0.546 | | | | |
| PAIRWISE COMPARISON P-VALUES¹ | | | | | |
| PLACEBO QID | — | 0.061 | 0.372 | 0.023 | |
| OXY QID | — | — | 0.222 | 0.635 | |
| OXY + NTX QID | — | — | — | 0.087 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 16C
WOMAC OSTEOARTHRITIS TOTAL SCORE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| ACTUAL VALUE AT WEEK 3 | | | | | |
| N | 50 | 98 | 100 | 100 | 348 |
| MEAN | 1328.8 | 1164.9 | 1225.0 | 1121.0 | 1193.1 |
| STANDARD DEVIATION | 750.67 | 651.16 | 624.27 | 638.73 | 656.01 |
| MINIMUM | 17.6 | 6.0 | 0.0 | 55.0 | 0.0 |
| MEDIAN | 1468.0 | 1191.0 | 1312.0 | 1143.0 | 1231.5 |
| MAXIMUM | 2400.0 | 2368.0 | 2348.0 | 2364.0 | 2400.0 |
| MODEL P-VALUES¹ | | | | | |
| TREATMENT | 0.149 | | | | |
| SEX | 0.201 | | | | |
| TREATMENT + SEX | 0.410 | | | | |
| PAIRWISE COMPARISON P-VALUES¹ | | | | | |
| PLACEBO QID | — | 0.129 | 0.451 | 0.039 | |
| OXY QID | — | — | 0.341 | 0.511 | |
| OXY + NTX QID | — | — | — | 0.204 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

TABLE 16D
WOMAC OSTEOARTHRITIS TOTAL SCORE - BY WEEK
ANALYSIS POPULATION: INTENT TO TREAT POPULATION

| | PLACEBO | | | OXY + NTX | |
|---|---|---|---|---|---|
| | QID | OXY QID | OXY + NTX QID | BID | TOTAL |
| | (N = 50) | (N = 102) | (N = 102) | (N = 102) | (N = 356) |
| BASELINE | | | | | |
| N | 49 | 101 | 102 | 101 | 353 |
| MEAN | 1650.2 | 1569.6 | 1640.1 | 1627.0 | 1617.6 |
| STANDARD DEVIATION | 445.37 | 432.36 | 382.02 | 397.20 | 409.67 |
| MINIMUM | 421.0 | 328.0 | 570.4 | 397.0 | 328.0 |
| MEDIAN | 1677.0 | 1640.0 | 1659.0 | 1703.0 | 1662.0 |
| MAXIMUM | 2377.0 | 2400.0 | 2330.0 | 2273.0 | 2400.0 |
| MODEL P-VALUES¹ | | | | | |
| TREATMENT | 0.704 | | | | |
| SEX | 0.017 | | | | |
| TREATMENT + SEX | 0.949 | | | | |
| PAIRWISE COMPARISON P-VALUES¹ | | | | | |
| PLACEBO QID | — | 0.330 | 0.867 | 0.694 | |
| OXY QID | — | — | 0.315 | 0.469 | |
| OXY + NTX QID | — | — | — | 0.778 | |

NOTE:
DATA IMPUTED USING THE LAST-OBSERVATION-CARRIED-FORWARD METHOD.
¹P-VALUES FROM ANOVA MODEL WITH TREATMENT, SEX, AND TREATMENT + SEX INTERACTION AS EFFECTS.

The overall incidences of adverse events in all three active treatment groups were generally comparable, and the numerical differences observed are shown in the following tables. The most frequent adverse events (AEs) reported were those commonly associated with opioid medications: dizziness, constipation, dry mouth, nausea, vomiting, somnolence, and pruritis. Table 17 shows adverse events experienced by >5% of the patients during Weeks 1, 2, or 3 of treatment, based on the Intent To Treat Population.

TABLE 17
Adverse Events
Number (%) of Patients

| | | | Oxycodone Plus | Oxycodone Plus |
|---|---|---|---|---|
| Adverse Event | Placebo | Oxycodone QID | Naltrexone QID | Naltrexone BID |
| | | Days 1-3: 10 mg OXY/d | Days 1-3: 10 mg OXY/d | Days 1-3: 10 mg OXY/d |
| Week 1, | Placebo QID | Days 4-8: 20 mg OXY/d | Days 4-8: 20 mg OXY/d | Days 4-8: 20 mg OXY/d |
| Dose N | 51 | 102 | 104 | 103 |
| Constipation | 2 (3.9) | 13 (12.7) | 5 (4.8) | 11 (10.7) |
| Dry Mouth | 0 (0.0) | 6 (5.9) | 6 (5.8) | 7 (6.8) |
| Nausea | 4 (7.8) | 24 (23.5) | 12 (11.5) | 28 (27.2) |
| Vomiting | 2 (3.9) | 4 (3.9) | 2 (1.9) | 8 (7.8) |
| Fatigue | 2 (3.9) | 3 (2.9) | 7 (6.7) | 3 (2.9) |
| Dizziness | 0 (0.0) | 17 (16.7) | 16 (15.4) | 31 (30.1) |
| Headache | 7 (13.7) | 17 (16.7) | 13 (12.5) | 12 (11.7) |
| Sommolence | 2 (3.9) | 16 (15.7) | 12 (11.5) | 12 (11.7) |
| Pruritus | 2 (3.9) | 8 (7.8) | 3 (2.9) | 10 (9.7) |
| Week 2, | Placebo QID | 30 mg OXY/d | 30 mg OXY/d | 30 mg OXY/d |
| Dose N | 45 | 85 | 84 | 74 |
| Constipation | 0 (0.0) | 6 (7.1) | 15 (17.9) | 9 (12.2) |
| Dry Mouth | 0 (0.0) | 4 (4.7) | 2 (2.4) | 5 (6.8) |
| Nausea | 1 (2.2) | 12 (14.1) | 3 (3.6) | 10 (13.5) |
| Vomiting | 0 (0.0) | 3 (3.5) | 1 (1.2) | 4 (5.4) |
| Fatigue | 3 (6.7) | 0 (0.0) | 1 (1.2) | 1 (1.4) |
| Dizziness | 0 (0.0) | 10 (11.8) | 10 (11.9) | 7 (9.5) |
| Headache | 3 (6.7) | 3 (3.5) | 5 (6.0) | 3 (4.1) |
| Sommolence | 1 (2.2) | 6 (7.1) | 6 (7.1) | 5 (6.8) |
| Pruritus | 0 (0.0) | 5 (5.9) | 3 (3.6) | 5 (6.8) |
| Week 3, | Placebo QID | 40 mg OXY/d | 40 mg OXY/d | 40 mg OXY/d |
| Dose N | 42 | 75 | 74 | 61 |
| Constipation | 1 (2.4) | 2 (2.7) | 9 (12.2) | 3 (4.9) |
| Dry Mouth | 0 (0.0) | 0 (0.0) | 3 (4.1) | 4 (6.6) |
| Nausea | 2 (4.8) | 8 (10.7) | 8 (10.8) | 6 (9.8) |
| Vomiting | 1 (2.4) | 9 (12.0) | 2 (2.7) | 2 (3.3) |
| Dizziness | 0 (0.0) | 4 (5.3) | 6 (8.1) | 2 (3.3) |
| Headache | 3 (7.1) | 6 (8.0) | 1 (1.4) | 3 (4.9) |
| Sommolence | 1 (2.4) | 2 (2.7) | 8 (10.8) | 5 (8.2) |
| Pruritus | 0 (0.0) | 3 (4.0) | 1 (1.4) | 7 (11.5) |

Seventy-nine of the 360 patients who received study drug-discontinued treatment because of adverse events (0 placebo; 29 oxycodone alone QID; 18 oxycodone plus naltrexone QID and 32 oxycodone plus naltrexone BID). Oxycodone plus naltrexone QID had the lowest AE discontinuation rate among the 3-active treatment group while oxycodone plus naltrexone BID and oxycodone alone QID were comparable. Adverse events that resulted in the discontinuation of treatment in two patients or more in any treatment group are shown in Table 18 below, based on the Intent to Treat Population. Most of the adverse events that resulted in treatment discontinuation are commonly associated with the use of opioid analgesics, e.g., nausea, vomiting, constipation, dizziness and somnolence.

TABLE 18
Selected Adverse Events
Number (%) of Patients

| | | Oxycodone | Oxycodone Plus | Oxycodone Plus |
|---|---|---|---|---|
| System Organ Class | Placebo | QID | Naltrexone QID | Naltrexone BID |
| Adverse Event | N = 51 | N = 102 | N = 104 | N = 103 |
| Any adverse event | 0 (0.0) | 29 (28.4) | 18 (17.3) | 32 (31.1) |
| Eye disorders | 0 (0.0) | 0 (0.0) | 2 (1.8) | 0 (0.0) |
| Gastrointestinal disorders | 0 (0.0) | 13 (12.7) | 7 (6.7) | 17 (16.5) |
| Upper abdominal pain | 0 (0.0) | 0 (0.0) | 0 (0.0) | 2 (1.9) |
| Constipation | 0 (0.0) | 1 (1.0) | 0 (0.0) | 2 (1.9) |
| Diarrhea | 0 (0.0) | 0 (0.0) | 2 (1.9) | 0 (0.0) |
| Nausea | 0 (0.0) | 7 (6.9) | 4 (3.8) | 12 (11.7) |
| Vomiting | 0 (0.0) | 4 (3.9) | 1 (1.0) | 3 (2.9) |
| General disorders and | 0 (0.0) | 2 (2.0) | 0 (0.0) | 2 (1.9) |
| administration site | | | | |
| conditions | | | | |
| Lethargy | 0 (0.0) | 0 (0.0) | 0 (0.0) | 2 (1.9) |
| Nervous system disorders | 0 (0.0) | 8 (7.8) | 11 (10.6) | 14 (13.6) |
| Dizziness | 0 (0.0) | 1 (1.0) | 5 (4.8) | 9 (8.7) |
| Headache | 0 (0.0) | 2 (2.0) | 2 (1.9) | 0 (0.0) |
| Sedation | 0 (0.0) | 2 (2.0) | 0 (0.0) | 0 (0.0) |
| Sommolence | 0 (0.0) | 2 (2.0) | 5 (4.8) | 5 (4.9) |
| Psychiatric disorders | 0 (0.0) | 1 (1.0) | 3 (2.9) | 0 (0.0) |
| Euphoric mood | 0 (0.0) | 0 (0.0) | 2 (1.9) | 0 (0.0) |
| Skin and subcutaneous | 0 (0.0) | 3 (2.9) | 1 (1.0) | 2 (1.9) |
| tissue disorders | | | | |
| Pruritus | 0 (0.0) | 1 (1.0) | 1 (1.0) | 2 (1.9) |

Serious adverse events (SAEs) were reported for five patients. All of the serious adverse events were unrelated to treatment with study medication.

The study was also designed to investigate potential opioid withdrawal effects when patients stopped study drug without tapering at the end of treatment. The Short Opioid Withdrawal Scale (SOWS) (see Table 6 above), originally used for collecting withdrawal data from heroin addicts, was used to assess opioid withdrawal in this study. Although there were statistically significant differences between treatment groups, the differences were considered clinically insignificant because both the mean SOWS changes and the differences of their changes were of small magnitude. The lack of clinically significant opioid withdrawal in this study is attributable to the relatively low opioid doses and short treatment duration. Opioid withdrawal was not reported as an adverse event in any of the treatment groups.

In summary, in this study oxycodone plus naltrexone BID was shown to be a safe and effective treatment for patients with chronic pain and with osteoarthritis of the hip or knee. Oxycodone plus naltrexone BID provided statistically and clinically significant reductions in pain intensity compared to oxycodone alone QID when the same total daily dose of oxycodone was administered. The overall incidence of opioid-related adverse events was comparable in the oxycodone plus naltrexone and oxycodone alone treatment groups and no clinically meaningful effects on vital signs, laboratory safety tests or QTc interval changes were noted in the oxycodone plus naltrexone or oxycodone alone treatment groups. Oxycodone plus naltrexone BID provided better daily pain control to that of oxycodone alone QID. Oxycodone plus naltrexone BID showed greater improvements in all categories of the WOMAC Osteoarthritis Index (pain, stiffness and physical function) when compared to the other active treatment groups.

B.

An additional clinical study was designed substantially the same as that described in Part A, with differences indicated below.

The clinical study was designed as follows: (1) to evaluate the efficacy and safety of combinations of oxycodone (oxy) and naltrexone (ntx) when compared to oxycodone, (2) to evaluate the efficacy and safety of combinations of oxycodone (oxy) and naltrexone (ntx) administered when compared to naltrexone, and (3) to compare the quality of life measures (WOMAC and SF-12) between treatment groups.

A multicenter, randomized, double-blind, active- and placebo-controlled, clinical study was designed and is conducted. The study evaluates the efficacy and safety of an oral formulation of oxycodone and naltrexone relative to oxycodone and to naltrexone over a 12-week fixed-dose period following one week of titration (instead of a three week period). A total of 750 patients (instead of 360 patents) with chronic pain due to osteoarthritis of the hip or knee are enrolled into six (instead of four) treatment groups: three groups for combinations of oxycodone and naltrexone, a group for oxycodone alone, a group for naltrexone alone and a group for placebo.

Patients are randomly assigned to one of the six treatment groups as shown in Table 19.

TABLE 19

| Treatment | | Weeks 1-12 |
|---|---|---|
| Group | Titration | (Fixed-Dose) |
| A | (OXY 10 mg + NTX | (OXY 20 mg + NTX |
| | 0.001 mg) BID | 0.001 mg) BID |
| B | (OXY 5 mg + NTX | (OXY 10 mg + NTX |
| | 0.001 mg) QID | 0.001 mg) QID |
| C | (OXY 10 mg + NTX | (OXY 10 mg + NTX |
| | 0.001 mg) BID | 0.001 mg) BID |
| D | OXY 5 mg QID | OXY 10 mg QID |
| E | NTX 0.001 mg BID | NTX 0.001 mg BID |
| F | Placebo QID | Placebo QID |

All treatment groups are scheduled for QID dosing to protect the double-blind study design as shown in Table 20.

TABLE 20

| Treatment | QID Daily Dosing Scheme* | | | |
|---|---|---|---|---|
| Group | Upon Waking | Noon | Afternoon | Bedtime |
| A | (OXY + NTX) | Placebo | (OXY + NTX) | Placebo |
| B | (OXY + NTX) | (OXY + | (OXY + NTX) | (OXY + NTX) |
| | | NTX) | | |
| C | (OXY + NTX) | Placebo | (OXY + NTX) | Placebo |
| D | OXY | OXY | OXY | OXY |
| E | NTX | Placebo | NTX | Placebo |
| F | Placebo | Placebo | Placebo | Placebo |

*Doses must be taken 30-60 minutes before meals and at least 4 hours apart.

Patients return to the clinic for weekly visits (±one day) for the first five weeks and then at 2-week (+two days) intervals for the remainder of the study (instead of the visit schedule described in Part A). At each clinic visit, quality of analgesia, pain control, and a global assessment of study medication are collected as described above. The SF-12 Health Survey and the WOMAC Osteoarthritis Index are collected monthly (instead of at each clinic visit).

Safety is evaluated as described in Part A.

The Study Population is seven hundred fifty (750) patients with moderate to severe chronic pain due to osteoarthritis of the hip or knee. According to the study design described above, there are 150 patients each in the oxycodone and naltrexone BID, oxycodone and naltrexone QID and oxycodone alone treatment groups; and 75 patients each in the naltrexone and placebo groups.

The inclusion criteria are essentially the same as described above in Part A, with the following exceptions:
Patient agrees to refrain from taking any pain medications other than study drug during the 13-week treatment period, rather than the shorter treatment period of the clinical study of Part A. (Aspirin [up to 325 mg/day] is permitted for cardiovascular prophylaxis if at a stable dose one month prior to the Screening Visit.); and
Patient is able to ambulate for a specified distance (at least 100 meters).

The exclusion criteria are essentially the same as described above in Part A, with the exceptions listed below. Additional exclusion criteria are as follows:
(a) Patient has a positive urine drug screen at the Baseline/Titration Visit NOT caused by any therapeutic medication permitted during the study;
(b) Patient has pain in the hip(s) or knee(s) caused by conditions other than osteoarthritis, e.g., malignancy, gout, inflammatory disease such as rheumatoid arthritis, trauma, fibromyalgia, bony fracture, or infection;
(c) Patient has a history of cardiac disease (such as coronary artery disease, cardiomyopathy, congestive heart disease, valvular disease, arrythmia, etc.), angina, myocardiac infarct (MI), cerebral aneurysm, cerebral vascular accident (CVA), transient ischemic event (TIA), inadequately controlled hypertension, or health condition(s) which poses significant health risk in the event of opioid withdrawal;
(d) Patient has started or stopped physical therapy, transcutaneous electrical nerve stimulation, chiropractic, osteopathic, acupuncture, or other complementary treatment within the past four weeks or is expected to undergo any changes in these therapies in the duration of the study;
(e) Patient has a psychiatric illness or medical illness/condition, and/or abnormal diagnostic finding, that, in the opinion of the investigator, would interfere with the completion of the study, confound the results of the study, or pose risk to the patient;
(f) Patient has a history of the following neoplastic disease: leukemia, lymphoma, or myeloproliferative disease, metastatic cancer. In patients with treated localized malignancies, the decision to exclude is made by the Sponsor on individual cases; and
(g) Patient has AST, ALT, GGT, or alkaline phosphatase >2 times the upper limit of normal; hematocrit <30%; or creatinine >1.8 at the Screening Visit. The following exclusion criteria in the clinical study describe of Part A were modified or omitted for the present clinical study:
(a) Acute hepatitis is not included in the exclusion factors;
(b) History of severe hepatic or renal impairment is not included in the exclusion factors;
(c) “Patient had other diseases significant enough, in the opinion of the Investigator, to pose a risk for the administration of study drug or that will interfere with pain assessments,” is not included in the exclusion factors;
(d) Patient has a history of gastric, biliary, or small intestine surgery, or any other diseases that cause clinical malabsorption is an exclusion factor (rather than “Patient had chronic biliary tract disease, chronic pancreatitis, or inflammatory bowel disorders”); and
(e) Patient's history of alcohol or drug abuse is within the past 5 years;

The physical descriptions of the drugs used for the study are as follows. For the washout period, acetaminophen is dispensed as described in Part A. The investigational drug supplies are in tablet dosage forms containing oxycodone HCl and naltrexone HCl, oxycodone HCl, naltrexone or placebo. All of the tablet dosage forms are indistinguishable from one another to facilitate blinding. Tablets are arranged on each blister card by Study Day and contain four doses per day. Each blister card also contains three days of extra study drug to allow for flexibility in planning return clinic visits. The extra study drug must remain intact within its original packaging so that it may be returned during each clinic visit. The investigational drug supplies are dispensed in these kits.

Safety procedures include those described in Part A. The opioid toxicity assessment includes: (A) CNS review by assessing for (1) confusion, altered mental state, (2) excessive drowsiness, lethargy, stupor, (3) slurred speech (new onset), (B) respiratory review by assessing for (1) hypoventilation, shortness of breath, apnea, (2) decreased respiratory rate (<8) or cyanosis; and (3) cardiac review by assessing for heart rate <60, hypotension. If patients must be terminated from the study, the Early Drug Termination assessments and opioid withdrawal monitoring (as needed) are performed as discussed below.

At the first visit, pre-enrollment screening is performed as described in Part A.

The second visit is on the first day of the first titration period of the study. The patients returned to the study center four to seven days after the Screening Visit for completion of the pre-dose assessments. This visit included (1) obtaining a urine sample for drug screening using a rapid drug screen kit (BioChek iCup™ Drug Screen). If positive for any drugs not caused by any therapeutic medication permitted during the study, no further assessments are made. Patient cannot continue in the study; (2) reviewing the take-home diary from the past four to seven days; (3) collect bottle of acetaminophen and perform accountability, (4) baseline clinic PI rating; and (5) reviewing inclusion and exclusion criteria. This assessment also includes verifying that (a) the mean daily overall pain intensity score collected in the diary over the last two days of the 4- to 7-day washout period is ≧5 (on a scale of 0 to 10) while off all analgesic medications (except acetaminophen as directed); (b) the clinic PI at this visit measures ≧5 (on a scale of 0 to 10); and (c) checking that the clinical laboratory tests results from the screening visit are without significant clinical abnormalities, and that the urine pregnancy test is negative (if required).

Patients meeting the study entry criteria are randomly assigned to one of the six treatment groups, and are assigned a randomization number and study medication kit number. The following assessments are then conducted: (1) a brief (interim) medical history; (2) vital signs; (3) review and record concomitant medications; (4) SF-12 Health Survey; and (5) WOMAC Osteoarthritis Index.

Once these assessments and procedures are completed, the study medication kit is dispensed for the titration period. The patients received their take-home daily diaries and are provided with an appointment card for the next visit. The study nurse thoroughly reviewed each section of the diary with the patient. The daily diary issued at Visit 2 is used to record the following information at bedtime immediately prior to dosing: (1) overall PI in the past 24 hours; and (2) adverse events.

Patients return to the study center at the end of titration (±one day) for the following:
(1) opioid toxicity assessment;
(2) review take-home diary (including overall daily bedtime PI and opioid-related adverse events);
(3) record new/changed adverse events and concomitant medications;
(4) collect study medication from previous week and account for used/unused supplies;
(5) vital signs;
(6) quality of analgesia;
(7) pain control;
(8) global assessment of study medication;
(9) dispense take-home daily diary; and
(10) dispense one blister card of study medication (by telephoning IVRS).

At the conclusion of this visit, the patient is given an appointment card for the next study visit.

Patients return to the study center at weekly intervals (±one day) for 4 weeks (Visits 4-7) and at the end of Weeks 6, 8, and 10 (±two days) (Visits 8-10) for the assessments:
(1) opioid toxicity assessment;
(2) review take-home diary (including overall daily bedtime PI and opioid-related adverse events);
(3) record new/changed adverse events and concomitant medications;
(4) collect study medication from previous week and account for used/unused supplies;
(5) vital signs;
(6) quality of analgesia;
(7) pain control;
(8) global assessment of study medication;
(9) SF-12 Health Survey;
(10) WOMAC Osteoarthritis Index;
(11) dispense take-home daily diary;
(12) dispense one blister card of study medication (by telephoning IVRS). Two blister cards are dispensed at the End of Week 4 Visit.

At the conclusion of each visit, the patient is given an appointment card for the next study visit.

Patients return to the study center at either the end of Week 12 (±two days) or after early drug termination for the same End of Treatment assessments described above except that a blood sample for PK analysis is not taken, and SOWS is only performed if the subject is on the study drug ≧4 weeks.

At the conclusion of this visit, prior to departing the center, the patient is given an appointment card for the next study visit. Patients are instructed to contact the study center immediately if they experience severe signs and symptoms of opioid withdrawal.

For four days after the last dose of study medication, the study center contacts patients as described in Part A to monitor for symptoms of opioid withdrawal.

Patients return to the study center approximately one week (±two days) after the last dose of study medication for a post-treatment follow-up visit (Visit 12). At this visit, the following assessments are completed:
(1) review take-home diary; and
(2) record new/changed adverse events and concomitant medications.

Patients may choose to discontinue study drug or study participation at any time, for any reason, specified or unspecified, and without prejudice. If a patient chooses to discontinue study drug early, the investigator must request that the patient return to the clinic within 24 hours of stopping the study medication and complete the end of study assessments described above. For patients who have been on study medication for >4 weeks, Day 1 of the opioid withdrawal monitoring period begins 24 hours after the last dose of study medication. The investigator can request that the patient remain in the study for the post-treatment follow-up visit. Study drug assigned to patients who discontinue early may not be reassigned.

The primary analysis population is the intent-to-treat (ITT) population. The ITT population will consist of all patients who take study medication and are used for both efficacy and safety analyses. In the event that a patient is randomized incorrectly or is administered the incorrect study medication, the patient is analyzed according to the study drug actually received. Additional analysis populations may be defined as appropriate based on the actual study experience.

Demographic variables and patient characteristics are summarized descriptively by treatment group and overall. Demographic variables will include age, weight, height, gender, and race/ethnicity. Baseline characteristics includes the PI score recorded in the clinic and baseline values of efficacy variables. Baseline and post-baseline patient characteristics includes study drug administration and prior and concomitant medications.

The following endpoints are summarized and analyzed:
(1) Daily diary PI score—The daily PI assessments are analyzed as weekly values as follows. For each post-baseline week, the PI recorded during the last two days of dosing within the week are averaged. Baseline PI is defined as the average PI recorded during the two days immediately prior to the Baseline/Titration visit;
(2) Quality of analgesia—assessed and analyzed by clinic visit (weekly for the first five weeks and biweekly thereafter);
(3) Pain control—assessed and analyzed by clinic visit (weekly for the first five weeks and biweekly thereafter);
(4) Global assessment of study medication—assessed and analyzed by clinic visit (weekly for the first five weeks and biweekly thereafter);
(5) SF-12 Health Survey—assessed and analyzed monthly; scored as described in the documentation (e.g., Ware et al., 1998); and
(6) WOMAC Osteoarthritis Index-assessed and analyzed monthly; calculated per the WOMAC User Guide.
   Missing efficacy data is imputed using the last-observation-carried-forward (LOCF) approach. If the number of patients per center is small, centers may be pooled for analysis, or omitted from statistical models. Unless otherwise indicated, all testing of statistical hypotheses is two-sided, and a difference resulting in a p-value of less than or equal to 0.05 is considered statistically significant.

For primary analysis of data, the primary efficacy endpoint is the percent change from baseline to Visit 11 (Week 12 or early drug termination) in average daily PI. An analysis of covariance (ANCOVA) model is employed, as described below. The pairwise treatment comparison that is of primary interest is treatment group A ([OXY 20 mg+NTX 0.001 mg] during the fixed-dose period) vs. treatment group D (OXY 10 mg QID during the fixed-dose period).

For secondary analysis of data, the average daily PI, SF-12 Health Survey, and WOMAC Osteoarthritis Index is analyzed in terms of the values themselves as well as in terms of change and percent change from baseline. These variables are summarized descriptively by treatment group and by sex. Treatments are compared globally and in pairwise fashion at each time point using an analysis of covariance (ANCOVA) model that includes treatment, center, and sex as factors and baseline value as a covariate. Potential interactions are assessed by also fitting a model with the same main effects and with the treatment by center, treatment by sex, and treatment by baseline interaction terms. In addition, pairwise t-tests are used to compare each post-baseline time point to each prior time point, within treatment group, overall and by sex.

The quality of analgesia, pain control, and global assessment of study medication are summarized descriptively by treatment group, overall and by sex. Treatments are compared at each time point using the Cochran-Mantel-Haenszel row mean scores test, using equally spaced scores, stratified by sex. Cochran-Mantel-Haenszel row mean scores tests will also be used to compare each post-baseline time point to each prior time point, within treatment group, overall and by sex.

Sensitivity analyses are carried out to determine the extent to which the statistical analysis results are influenced by the choice to impute missing observations using LOCF. The primary analysis is repeated using one or more alternative imputation methods (e.g., imputing data following withdrawal depending on the reason for withdrawal) and using an appropriate longitudinal analysis technique such as repeated measures mixed-effects analysis of variance. In addition, an “observed data” analysis, without any data imputation, is conducted on selected endpoints using the same analysis methods described previously.

Adverse events are reported and examined as described in Part A. Change from baseline is summarized descriptively for vital signs and QTc interval. Laboratory data is summarized descriptively on the original scale, change from baseline, and in terms of the normal range.

### EXAMPLE 2

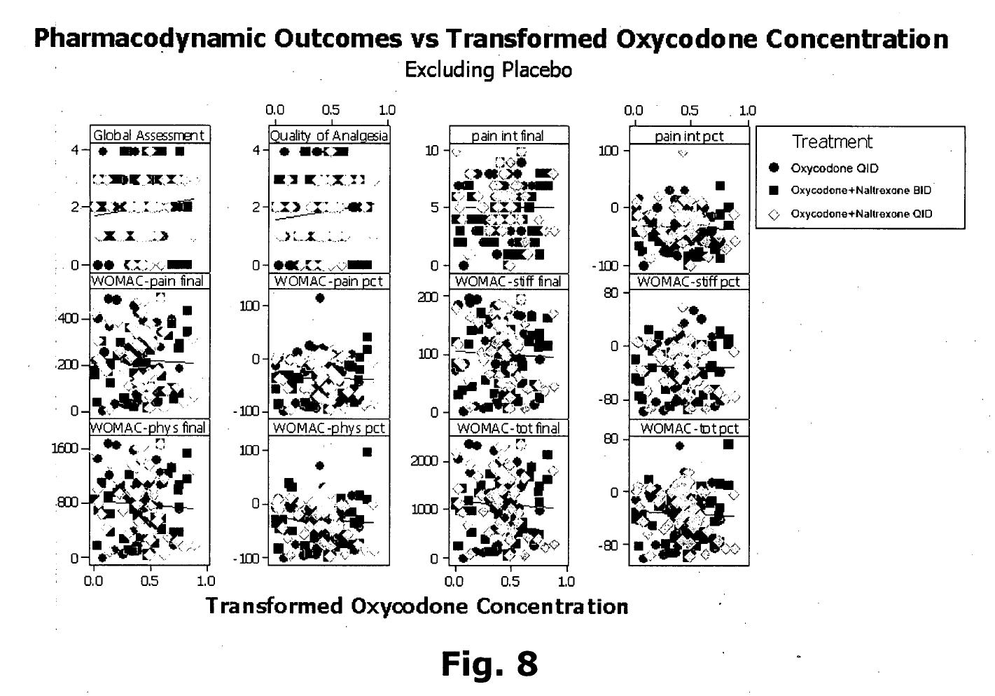

A clinical study was conducted as described in Example 1 wherein safety and analgesic effects of oxycodone or a combination of oxycodone and naltrexone were measured in patients with chronic pain as described in Example 1. Plasma concentrations of the administered drugs and their major metabolites were measured to determine: (1) oxycodone absorption from the combination drug of oxycodone and naltrexone; (2) dose proportionality of plasma concentrations of oxycodone and oxymorphone from the combination drug of oxycodone and naltrexone; (3) achievement of steady state of plasma concentrations of oxycodone, oxymorphone and 6β-naltrexol from the combination drug of oxycodone and naltrexone; and (4) consistency of the half-life and clearance of oxycodone over the course of the study. The relationships between clinical outcomes and the plasma concentrations of oxycodone, oxymorphone, and 6β-naltrexol were plotted for each treatment as shown in FIGS. 8 to 10.

Patients with moderate-to-severe pain due to osteoarthritis of the hip or knee were randomly assigned to one of four treatment groups as shown in Table 3 of Example 1. Plasma samples were obtained for each patient at the beginning of Weeks 1, 2, 3 and at the end of dosing during Week 3.

Patients for inclusion in the bioanalytical analyses (24/sex/treatment arm) were randomly selected from those who completed all three weeks of treatment in each of the three active treatment arms. Plasma samples randomly selected from each of those treatment arms were analyzed for oxycodone, oxymorphone, noroxycodone, naltrexone and 6β-naltrexol by validated coupled solid phase extraction LC-MS/MS methods.

For the analysis of linearity and dose proportionality, linear equation coefficients were obtained by averaging patient-specific slopes and intercepts obtained by within-patient least squares regression. This was done to account for the correlation among the repeated measurements due to the patient's contributing data at each dose level. The resulting slopes among treatment groups were compared by one-way analysis of variance (ANOVA), and a one-sample t-test assessed the common slope's difference from zero. A measure of deviation from linearity was constructed as the difference between the concentration at the middle dose versus the average of the concentrations at the lower and higher doses. Due to equal spacing of doses, this measure has expectation zero under the hypothesis of linearity. As above, ANOVA and t-tests were used to assess linearity.

The relationship between oxycodone plasma concentration and various outcome measures were assessed by Pearson correlation coefficients and associated p-values. For these analyses, the plasma concentration data were log-transformed in order to achieve approximately Gaussian distributions. Oxycodone plasma concentrations (ignoring time of blood draw) in active treatment arms were compared by one-way ANOVA. Regression analyses combined with F-tests on the extra sums of squares assessed whether profiles and correlations differed among active treatment arms. P-values were computed without adjustment for multiple testing. Similar analyses were conducted on the oxymorphone and 6β-naltrexol plasma concentrations.

The oxycodone and oxymorphone plasma concentration data showed a skewed distribution commonly seen in pharmacokinetic data. The base-10 log transformation reduced the skewness on the right tail (larger concentrations) but introduced skewness on the lower tail. To achieve symmetry, modified log transformations were used. Symmetry was achieved using the following modified log transformations:
Transformed oxycodone value=log(original value+10)−log(10)
Transformed oxymorphone value=log(original value+0.1)−log(0.1)
Transformed 6β-naltrexol value=log(original value).

The translations by −log(10) and −log(0.1) were used so that concentrations of zero on the original scale would be transformed to zero. The transformation for 6β-naltrexol did not require a translation to achieve an approximately Gaussian distribution.

In addition to summary statistics and graphs, box-and-whisker plots were used to summarize the distribution of variables. Those figures depict either the minimum value in the data or selected outliers at the lower end, the quartile (25th percentile), the median, the upper quartile (75th percentile) and the maximum or selected outliers at the upper end.

Statistical analyses except extra sum of squares analyses were performed using MINTAB®, release 14.1 (Minitab Inc., 2003). The extra sum of squares analyses were calculated using Microsoft Excel, with MINTAB® sum of squares input.

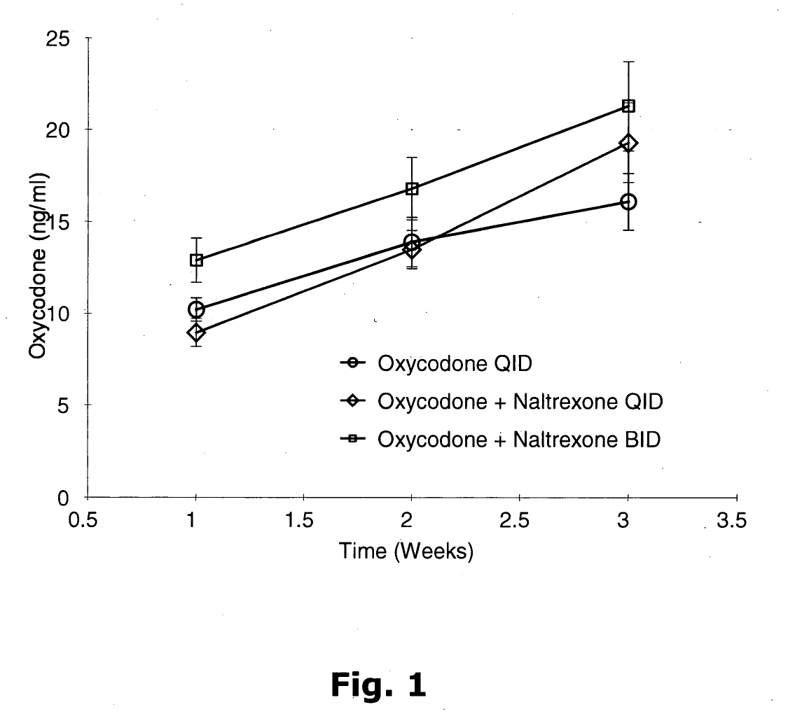

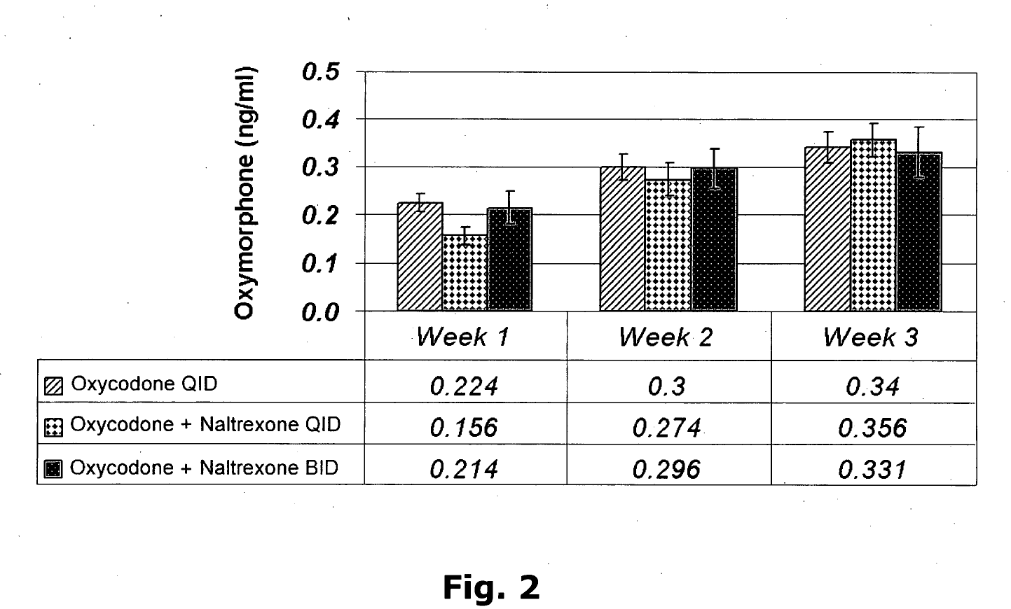

As shown in FIGS. 1 and 2, the mean plasma concentrations of oxycodone and oxymorphone from each drug increased linearly with increasing dose levels over the course of the study. Oxycodone dose levels increase during the course of the study; the oxycodone dose per dose of the combination drug of oxycodone and naltrexone BID was 5 mg (days 1-3), 10 mg (days 4-8), 15 mg (week 2) and 20 mg (week 3). The total daily dose of oxycodone was equal in active treatment arms; i.e., the oxycodone dose in individual doses of the combination drug of oxycodone and naltrexone QID or oxycodone QID was half that of the combination drug of oxycodone and naltrexone BID. The relationship between the oxycodone plasma concentrations and the amount of oxycodone in the dosage form was estimated as: plasma concentration of oxycodone (ng/mL)=2.28+0.4223(dose of oxycodone, mg/day). The slope was statistically significant (p<0.001, t-test) and slopes did not differ significantly among treatments (p=0.258, ANOVA). The test for deviation from linearity was not statistically significant (p=0.787, t-test). The relationship between the oxymorphone plasma concentrations and the amount of oxycodone in the dosage form was estimated to be: plasma concentration of oxymorphone (ng/mL)=0.0607+0.007153(dose of oxycodone, mg/day). The slope was statistically significant (p<0.001, t-test) and the slopes did not differ significantly among treatments (p=0.163, ANOVA). The outcome for the test for deviation from linearity was (p=0.056, t-test). The linear equation coefficients were obtained by averaging patient-specific slopes and intercepts obtained by within-patient least squares regression.

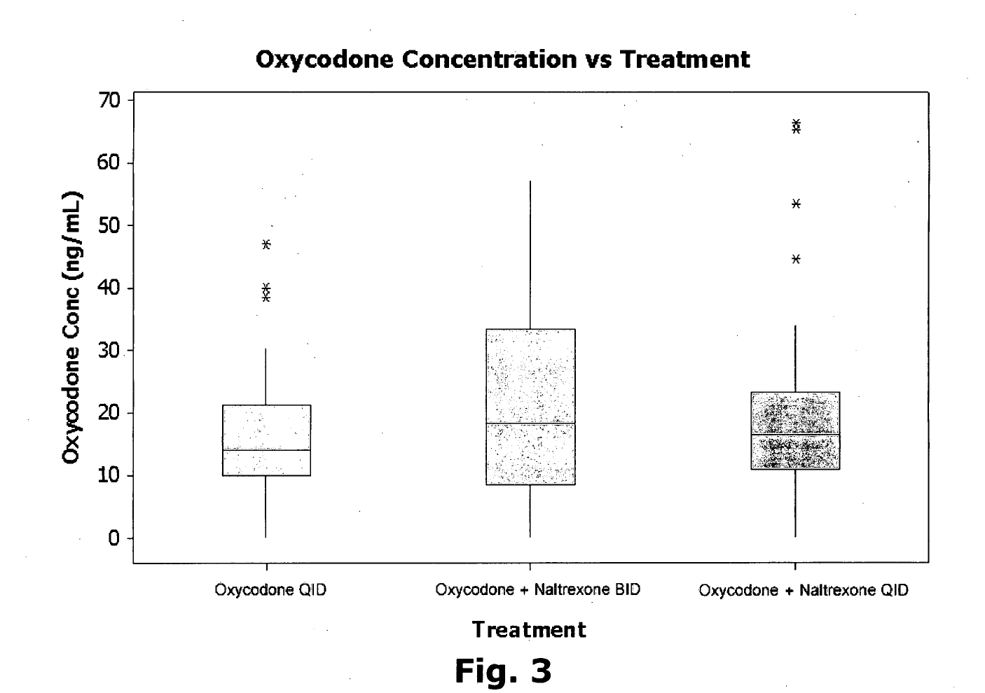

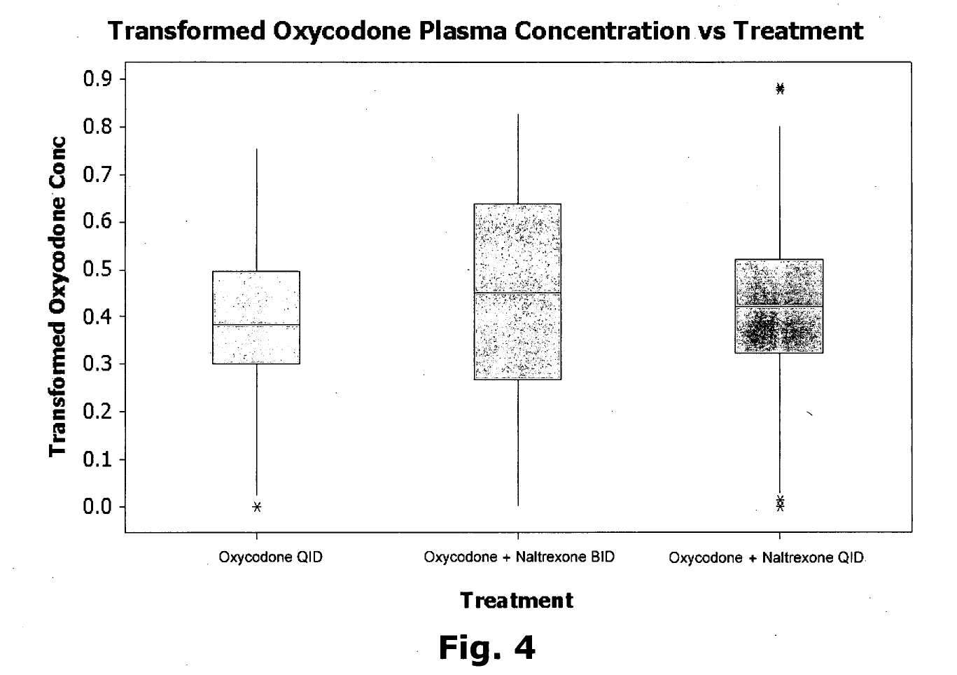

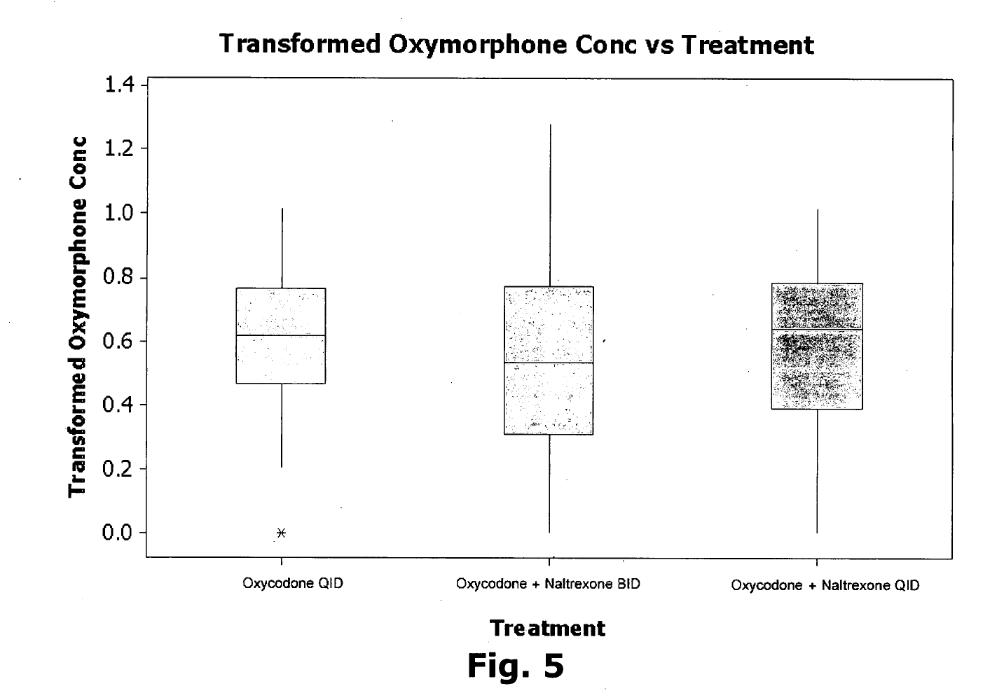

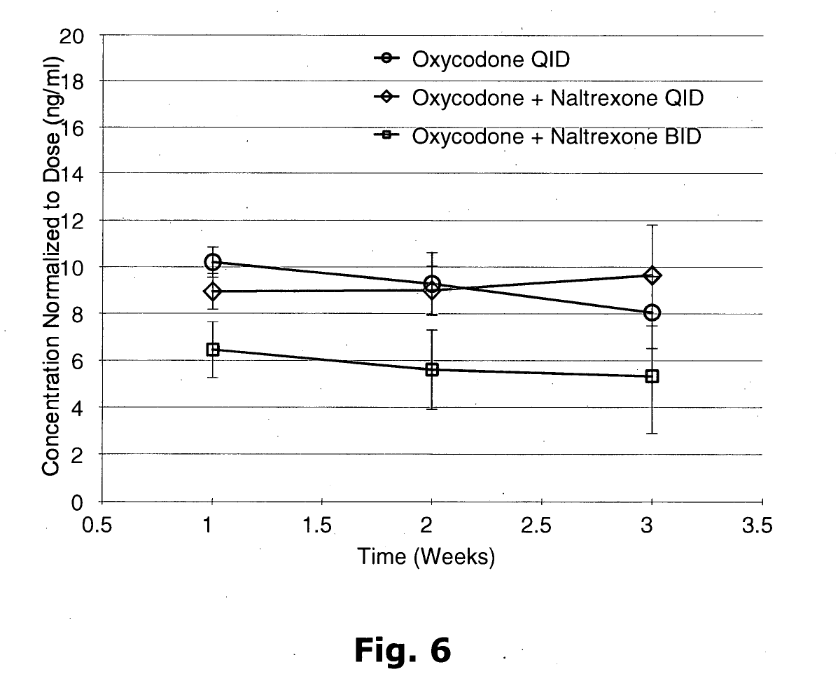

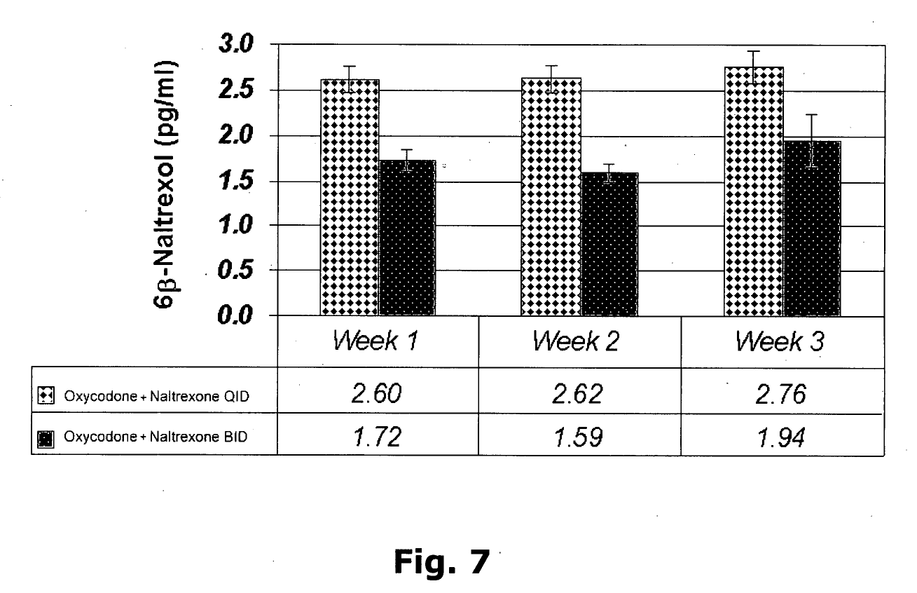

FIG. 3 shows the box-and-whisker plots of the plasma concentrations for oxycodone for each treatment group. FIG. 4 shows the box-and-whisker plots of the transformed plasma concentrations for oxycodone for each treatment group. The results in FIGS. 3 and 4 show that there was no statistical difference (ANOVA, p=0.492) among active treatment groups for oxycodone plasma concentrations. As shown in FIG. 3, the median plasma concentrations of oxycodone are not different following the final dose for each treatment group (* indicates outlying value). As shown in FIG. 4, the median log-transformed plasma concentrations of oxycodone are not different following the final dose for each treatment group (* indicates outlying value). FIG. 5 shows the box-and-whisker plots of the transformed plasma concentrations of oxymorphone for each treatment group. The median log-transformed plasma concentrations of oxymorphone are not different following the final dose for each treatment arm (* indicates outlying value). As shown in FIG. 6, the plasma concentrations of oxycodone and oxymorphone normalized by dose remained constant throughout the study, irrespective of treatment group, suggesting that steady state is achieved and maintained. As shown in FIG. 7, the plasma concentrations of 6β-naltrexol, the major metabolite of naltrexone and a marker for its concentration, remained constant throughout the study (sampled at the end of each treatment week). Maintenance of constant concentrations of 6β-naltrexol, the major metabolite of naltrexone, suggests that naltrexone reached steady state by the end of Week 1 regardless of a 2-fold difference in dose and dosing frequency between treatments.

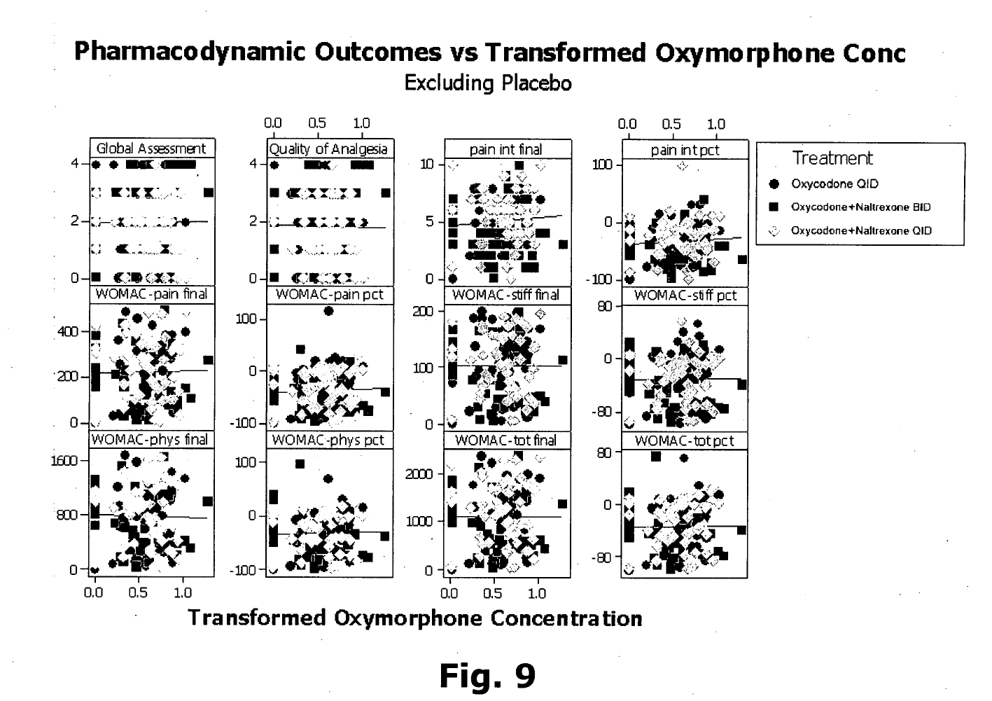

There was no statistically significant difference among treatment groups for any of the correlations between measures of clinical efficacy versus plasma concentrations of oxycodone or oxymorphone (p≧0.193). For the efficacy measurements shown in FIG. 8, the plasma concentrations of oxycodone correlated only with global assessment and quality of analgesia. The lines in each plot panel of FIG. 8 are least square fits. For the efficacy measurements shown in FIG. 9, there was no correlation between the plasma concentrations of oxymorphone and those efficacy measurements. The lines in each plot panel of FIG. 9 are least square fits.

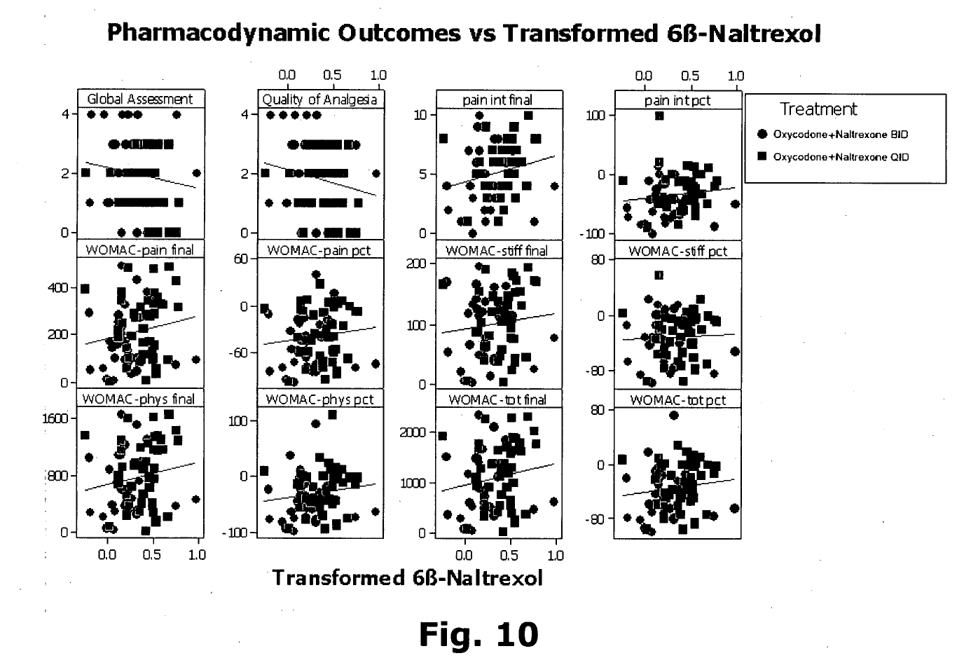

The mean 6β-naltrexol plasma concentration in the BID group is statistically different from that in the QID group (p<0.001, t-test of log-transformed plasma concentrations). There was also a significant difference between the two groups in pain intensity reduction. In addition to these group differences, the relationship between 6β-naltrexol concentrations and clinical outcomes can be observed in the individual patients, with lower plasma concentrations of 6β-naltrexol corresponding to greater clinical efficacy (e.g., pain relief) as shown in FIG. 10. The lines in each plot panel of FIG. 10 are least square fits.

As shown in the above-described figures, the similarity of oxycodone and oxymorphone plasma concentrations after administration of the combination drug of oxycodone and naltrexone BID versus oxycodone QID indicates that the absorption of oxycodone from the combination drug of oxycodone and naltrexone was similar to absorption from oxycodone alone. The plasma concentrations of oxycodone and oxymorphone increased linearly with dose, demonstrating that the exposure to oxycodone from the combination drug of oxycodone and naltrexone is proportional to dose. Maintenance of dose proportionality of oxycodone and oxymorphone throughout the study suggests that steady state was achieved during each dose interval. The consistency of dose proportionality also indicates that the oxycodone elimination half-life did not change during the course of the study. Likewise, the uniform concentrations of 6β-naltrexol, the major metabolite of naltrexone, suggest that naltrexone had attained steady state by the end of Week 1 for both doses and dosing frequencies.

Plasma concentrations of oxycodone and its metabolites, as well as the major metabolite of naltrexone (6β-naltrexol) showed stable pharmacokinetic parameters indicating that the dosage regimens for the combination drug of oxycodone and naltrexone are predictable and easy to manage. Comparisons of the BID and QID dosing regimens for the combination drug of oxycodone and naltrexone showed good correlation between 6β-naltrexol concentration and statistically significant reduction in pain intensity and the percentage change in pain intensity.

The dissimilar clinical response in the presence of similar oxycodone exposures suggests that the naltrexone/6β-naltrexol concentrations are important in determining the threshold for clinical efficacy.

The studies described in this Example show that oxycodone and naltrexone were well-absorbed from the combination drug of oxycodone and naltrexone and that plasma concentrations of oxycodone, oxymorphone and 6β-naltrexol from the combination drug of oxycodone and naltrexone increased directly proportional to the dose and reached steady state over each dosing interval. Clearances and apparent half-lives of oxycodone, oxymorphone and 6β-naltrexol from the combination drug of oxycodone and naltrexone did not change over the course of the study and dose and dosage regimen for the combination drug of oxycodone and naltrexone BID resulted in significantly greater clinical efficacy compared to the QID regimen in reduction in pain intensity or percentage change in pain intensity. Plasma concentrations of oxycodone and oxymorphone did not correlate with greater pain relief and the lowest dose of naltrexone (from the administration of the combination drug of oxycodone and naltrexone BID) utilized in this study resulting in the lowest plasma concentrations of 6β-naltrexol, as a measure of naltrexone plasma concentrations, corresponded to greater pain relief.

### EXAMPLE 3

Data were obtained from a clinical study conducted as described in Examples 1 and 2. Plasma concentrations of the administered drugs and their major metabolites were measured by validated solid phase extraction coupled HPLC-MS/MS. As described in this Example, pharmacokinetic/pharmacodynamic (PK/PD) analyses, including novel applications of modeling analysis, provide novel methods and materials for treating chronic pain, including but not limited to novel dosage forms and methods of administration.

As described in Example 2, the oxycodone and oxymorphone plasma concentration data showed a skewed distribution commonly seen in pharmacokinetic (PK) data. To achieve symmetry, modified log transformations were used as described in Example 2. As noted in Example 2, 6β-naltrexol plasma concentrations did not require transformation to achieve an approximately Gaussian distribution. Table 21 shows the correspondence between the transformed and original scales (where “a” indicates beyond range of observed data).

TABLE 21
Data Transformations of Oxycodone and Oxymorphone Concentrations

| | Original | Original |
|---|---|---|
| | Oxycodone | Oxymorphone |
| | Concentration | Concentration |
| Transformed Value | (ng/ml) | (ng/ml) |
| 0.0 | 0.0 | 0.00 |
| 0.1 | 2.6 | 0.03 |
| 0.2 | 5.8 | 0.06 |
| 0.3 | 10.0 | 0.10 |
| 0.4 | 15.1 | 0.15 |
| 0.5 | 21.6 | 0.22 |
| 0.6 | 29.8 | 0.30 |
| 0.7 | 40.1 | 0.40 |
| 0.8 | 53.1 | 0.53 |
| 0.9 | 69.4 | 0.69 |
| 1.0 | A | 0.90 |
| 1.1 | A | 1.16 |
| 1.2 | A | 1.48 |
| 1.3 | A | 1.90 |

Pharmacodynamic outcome measures as described in Example 2 and FIGS. 8-10 were paired with the appropriate analyte plasma concentrations for PK/PD analyses. The plasma concentrations of oxycodone, oxymorphone, naltrexone and 6β naltrexol were measured. It has been observed that plasma concentrations of naltrexone are about one-tenth the plasma concentrations of 6β-naltrexol in the same plasma samples. Accordingly, 6β-naltrexol plasma concentrations are useful as indicators of naltrexone plasma concentrations and to identify preferred plasma concentrations of naltrexone.

Included in the PK/PD analytes were oxycodone and oxymorphone plasma concentrations individually paired with: pain intensity at final visit; pain intensity percent change from baseline at final visit; patient's global assessment at final visit; quality of analgesia at final visit; WOMAC-pain at final visit; WOMAC-pain percent change from baseline at final visit; WOMAC-stiffness at final visit; WOMAC-stiffness percent change from baseline at final visit; WOMAC-physical function at final visit; WOMAC-physical function percent change from baseline at final visit; WOMAC-total score at final visit; and WOMAC-total score percent change from baseline at final visit. Among the subjects from whom blood was drawn for determination of plasma concentrations, the times from the last dose of the study drug administered to the blood draw were recorded. Times are centered on the hour (e.g., hour 4 covers times from 3.5 up to but excluding 4.5 hours). Table 22 summarizes the times from last dose to blood draw by treatment group for those subjects with plasma concentration data. There was no significant difference among the three treatment groups in time from last dose to blood draw.

TABLE 22
Number of Patients with Plasma Concentration Data by Hour and
Treatment

| | | | Oxycodone + | Oxycodone + |
|---|---|---|---|---|
| | | Oxycodone | Naltrexone | Naltrexone |
| Hour | Total | QID | BID | QID |
| 1 | 19 | 7 | 5 | 7 |
| 2 | 31 | 7 | 8 | 16 |
| 3 | 29 | 7 | 13 | 9 |
| 4 | 24 | 11 | 6 | 7 |
| 5 | 15 | 6 | 5 | 4 |
| 6 | 8 | 4 | 2 | 2 |
| >=6.5 | 10 | 3 | 5 | 2 |
| All | 136 | 45 | 44 | 47 |

As described in Example 2, for the analysis of linearity and dose proportionality, linear equation coefficients were obtained by averaging patient-specific slopes and intercepts obtained by within-patient least squares regression. This was done to account for the correlation among the repeated measurements due to the patient's contributing data at each dose level. The resulting slopes among treatment groups were compared by one-way analysis of variance ANOVA), and a one-sample t-test assessed the common slope's difference from zero. A measure of deviation from linearity was constructed as the difference between the concentration at the middle dose versus the average of the concentrations at the lower and higher doses. Due to equal spacing of doses, this measure has expectation zero under the hypothesis of linearity. As above, ANOVA and t-tests were used to assess linearity.

As also described in Example 2, the relationship between oxycodone plasma concentration and various outcome measures were assessed by Pearson correlation coefficients and associated p-values. For these analyses, the plasma concentration data were log-transformed in order to achieve approximately Gaussian distributions. Oxycodone plasma concentrations (ignoring time of blood draw) in active treatment arms were compared by one-way ANOVA. Regression analyses combined with F-tests on the extra sums of squares assessed whether profiles and correlations differed among active treatment arms. P-values were computed without adjustment for multiple testing. Similar analyses were conducted on the oxymorphone plasma concentrations.

A Kruskal-Wallis test was used to compare active treatment arms with respect to time from last dose to blood draw. The main PK assessment used linear regression analysis to fit the time-concentration profiles. One-way analysis of variance (ANOVA) was used to compare active treatment arms with respect to average oxycodone and oxymorphone plasma concentration (ignoring time of blood draw). Pearson correlation coefficients and associated p-values were used to describe the relationship between plasma concentration versus the outcome measures. Regression analyses combined with F-tests on the extra sums of squares were used to assess whether the time-concentration profiles and correlations differed among the three active treatment arms. P-values were computed and reported without adjustment for multiple testing. As described in Example 2, statistical analyses except extra sum of squares analyses were performed using MINITAB®, release 14.1. The extra sum of squares analyses were calculated using Microsoft Excel, with Minitab sums of squares as input.

Statistically significant correlations (r=0.21, p=0.005) were observed between transformed oxycodone concentration and each of the measures Patient's Global Assessment and Quality of Analgesia (which are pharmacodynamic data), at the final visit. Correlations for the other outcome measures were close to zero and not statistically significant. The calculation of these correlation coefficients included placebo data with imputed oxycodone concentrations of 0.0. In an analysis that excluded the placebo patients, the correlations were smaller and less significant. Table 23 lists the correlation coefficients. There was no statistically significant difference among treatment arms in these plasma concentration and measures of efficacy relationships.

TABLE 23
Correlation Coefficients vs Transformed Oxycodone Concentration

| Pharmacodynamic outcome | All | Oxycodone | | | All except |
|---|---|---|---|---|---|
| measure¹ | Patients | QID | Oxycodone + Naltrexone BID | Oxycodone + Naltrexone QID | placebo |
| Pain Intensity at final visit | −0.084 | −0.014 | 0.143 | −0.057 | 0.010 |
| | 0.263 | 0.927 | 0.353 | 0.703 | 0.912 |
| Paint intensity percent | −0.102 | −0.085 | 0.080 | −0.084 | −0.044 |
| change | 0.176 | 0.579 | 0.605 | 0.575 | 0.612 |
| Global assessment at final | 0.209 | 0.174 | 0.055 | 0.147 | 0.124 |
| visit | 0.005 | 0.254 | 0.722 | 0.323 | 0.150 |
| Quality of analgesia at final | 0.211 | 0.207 | −0.009 | 0.145 | 0.114 |
| visit | 0.005 | 0.172 | 0.954 | 0.332 | 0.185 |
| WOMAC-Pain at final visit | −0.070 | −0.049 | 0.192 | −0.230 | −0.044 |
| | 0.356 | 0.748 | 0.213 | 0.120 | 0.611 |
| WOMAC-Pain percent | −0.057 | −0.040 | 0.196 | −0.237 | −0.044 |
| change | 0.451 | 0.793 | 0.203 | 0.109 | 0.612 |
| WOMAC-Stiffness at final | −0.070 | −0.030 | 0.088 | −0.151 | −0.038 |
| visit | 0.352 | 0.844 | 0.573 | 0.312 | 0.665 |
| WOMAC-Stiffness percent | −0.042 | 0.102 | 0.078 | −0.208 | −0.015 |
| change | 0.576 | 0.506 | 0.618 | 0.162 | 0.865 |
| WOMAC-Physical | −0.071 | −0.036 | 0.123 | −0.216 | −0.055 |
| functioning at final visit | 0.345 | 0.815 | 0.433 | 0.145 | 0.529 |
| WOMAC-Physical | −0.060 | 0.015 | 0.089 | −0.211 | −0.044 |
| functioning percent change | 0.426 | 0.924 | 0.570 | 0.155 | 0.615 |
| WOMAC-Total score at | −0.072 | −0.038 | 0.137 | −0.215 | −0.052 |
| final visit | 0.343 | 0.802 | 0.381 | 0.147 | 0.551 |
| WOMAC-Total score | −0.062 | 0.010 | 0.125 | −0.249 | −0.044 |
| percent change | 0.417 | 0.948 | 0.425 | 0.091 | 0.614 |

¹For each pharmacodynamic outcome measure, the first row of data displays correlation coefficients, and the second row displays corresponding p-values.

The concentration time course of oxymorphone was well modeled by a straight line. Separate regression lines were also fit for each treatment group. Observed differences in slope are not statistically significant. Overall, ignoring time of blood draw, there was no statistically significant difference among active treatment arms in transformed oxymorphone plasma concentration. None of the correlations between oxymorphone and outcome measures was statistically significant, as shown in Table 24. There was no statistically significant difference among treatment arms in these PK/PD relationships.

TABLE 24
Correlation Coefficients vs Transformed Oxymorphone Concentration

| Pharmacodynamic outcome | All | Oxycodone | Oxycodone + Naltrexone | Oxycodone + Naltrexone | All except |
|---|---|---|---|---|---|
| measure¹ | Patients | QID | BID | QID | placebo |
| Pain Intensity at final visit | −0.044 | 0.293 | −0.095 | 0.021 | 0.079 |
| | 0.560 | 0.051 | 0.540 | 0.889 | 0.359 |
| Paint intensity percent change | −0.028 | 0.228 | −0.099 | 0.061 | 0.076 |
| | 0.709 | 0.131 | 0.521 | 0.685 | 0.379 |
| Global assessment at final visit | 0.140 | −0.126 | 0.297 | −0.171 | 0.011 |
| | 0.063 | 0.410 | 0.051 | 0.251 | 0.898 |
| Quality of analgesia at final visit | 0.130 | −0.126 | 0.230 | −0.144 | −0.015 |
| | 0.085 | 0.409 | 0.134 | 0.333 | 0.863 |
| WOMAC-Pain at final visit | −0.028 | 0.148 | −0.041 | −0.074 | 0.026 |
| | 0.711 | 0.332 | 0.789 | 0.623 | 0.766 |
| WOMAC-Pain percent change | 0.007 | 0.153 | −0.036 | −0.002 | 0.060 |
| | 0.930 | 0.317 | 0.817 | 0.991 | 0.491 |
| WOMAC-Stiffness at final visit | −0.045 | 0.155 | −0.141 | −0.018 | 0.005 |
| | 0.553 | 0.308 | 0.366 | 0.905 | 0.953 |
| WOMAC-Stiffness percent | −0.026 | 0.215 | −0.170 | 0.002 | 0.012 |
| change | 0.730 | 0.157 | 0.275 | 0.989 | 0.891 |
| WOMAC-Physical functioning at | −0.045 | 0.139 | −0.146 | −0.051 | −0.011 |
| final visit | 0.548 | 0.364 | 0.349 | 0.734 | 0.896 |
| WOMAC-Physical functioning | −0.017 | 0.184 | −0.221 | 0.139 | 0.026 |
| percent change | 0.818 | 0.227 | 0.154 | 0.350 | 0.765 |
| WOMAC-Total score at final visit | −0.043 | 0.143 | −0.129 | −0.053 | −0.003 |
| | 0.574 | 0.347 | 0.411 | 0.723 | 0.973 |
| WOMAC-Total score percent | −0.021 | 0.184 | −0.174 | 0.059 | 0.023 |
| change | 0.782 | 0.227 | 0.264 | 0.692 | 0.794 |

¹For each pharmacodynamic outcome measure, the first row of data displays correlation coefficients and the second row displays corresponding p-values.

Pharmacokinetic and pharmacodynamic data (e.g., percentage change in pain intensity) associated with the administration of oxycodone and naltrexone in clinical studies as described above were evaluated to identify desirable parameters involving dosage forms comprising naltrexone. Table 25 shows 6β-naltrexol plasma concentrations from the randomly selected samples for the subjects receiving oxycodone and naltrexone. Table 25 also shows pain intensity measurements for those subjects, including pain intensity baseline, final pain intensity, and the percent change in pain intensity. As discussed in more detail below, the percent change in pain intensity was the drug effect used in a modeling analysis of plasma concentration vs. drug effect.

TABLE 25
PK/PD Data For Combination Drug of Oxycodone and Naltrexone
In QID and BID Dosing Regimens

| | | | Time from | Pain Int | Pain Int | | |
|---|---|---|---|---|---|---|---|
| Subject | Treatment | Sex | Last Dose | Base | Final | Pain Int Percent | 6βNaltrexol |
| 004-0530 | QID | F | 2.22 | 9 | 7 | −22.222 | 3.29 |
| 004-0560 | BID | F | 2.67 | 8 | 1 | −87.5 | 5.67 |
| 004-0741 | QID | F | 4.25 | 10 | 8 | −20 | 3.25 |
| 006-0015 | QID | M | 4.2 | 8 | 7 | −12.5 | 2.47 |
| 006-0554 | BID | F | 2.42 | 7 | 1 | −85.714 | 0.89 |
| 006-0592 | QID | F | 0.5 | 8 | 4 | −50 | 4.69 |
| 006-0700 | QID | F | 3.05 | 9 | 6 | −33.333 | 3.16 |
| 006-0705 | BID | F | 2.52 | 9 | 8 | −11.111 | 2.24 |
| 006-0724 | BID | F | 2.53 | 8 | 6 | −25 | |
| 006-0727 | QID | F | 4.33 | 9 | 5 | −44.444 | 2.95 |
| 007-0011 | BID | M | 2.25 | 7 | 2 | −71.429 | 1.57 |
| 007-0045 | BID | M | 1.5 | 8 | 4 | −50 | |
| 007-0088 | QID | M | 3.92 | 7 | 7 | 0 | 3.58 |
| 007-0097 | BID | M | 2.33 | 6 | 2 | −66.667 | |
| 007-0520 | QID | F | 1.83 | 7 | 6 | −14.288 | |
| 007-0650 | BID | F | 0.97 | 9 | 4 | −55.556 | 0.61 |
| 009-0003 | QID | M | 1.92 | 9 | 8 | −11.111 | 0.56 |
| 009-0022 | BID | M | 2.08 | 6 | 1 | −83.333 | 1.64 |
| 009-0025 | QID | M | 0.67 | 9 | 6 | −33.333 | 4.07 |
| 009-0049 | QID | M | 0.67 | 9 | 8 | −11.111 | 5.98 |
| 009-0058 | QID | M | 1.33 | 10 | 8 | −20 | 5.63 |
| 009-0612 | BID | F | 4.58 | 8 | 3 | −62.5 | 1.2 |
| 010-0030 | QID | M | 1.83 | 7 | 8 | 14.286 | 2.86 |
| 010-0033 | BID | M | 3.58 | 7 | 4 | −42.857 | 1.6 |
| 010-0501 | BID | F | 26.17 | 6 | 5 | −16.667 | 1.56 |
| 012-0026 | QID | M | 5.73 | 3 | 6 | 100 | 1.39 |
| 012-0029 | BID | M | 5.25 | 7 | 7 | 0 | 1.41 |
| 012-0031 | QID | M | 2.78 | 7 | 6 | −14.286 | 3.18 |
| 012-0641 | BID | F | 2 | 10 | 4 | −60 | |
| 013-0001 | BID | M | 5.08 | 6 | 1 | −83.333 | 0.97 |
| 013-0008 | QID | M | 1.75 | 7 | 1 | −85.714 | 2.24 |
| 013-0109 | QID | M | 3.25 | 7 | 6 | −14.286 | 2.45 |
| 013-0565 | QID | F | 1.67 | 7 | 4 | −42.857 | 1.61 |
| 015-0095 | BID | M | 3 | 10 | 5 | −50 | 1.57 |
| 015-0572 | QID | F | 2.83 | 7 | 1 | −85.714 | |
| 015-0733 | BID | F | 2.5 | 7 | 4 | −42.857 | 2.09 |
| 016-0108 | BID | M | 3.92 | 8 | 2 | −75 | 1.86 |
| 016-0563 | BID | F | 4.42 | 8 | 4 | −50 | 9.54 |
| 016-0590 | BID | F | 2.83 | 10 | 4 | −60 | |
| 016-0620 | QID | F | 1.5 | 7 | 3 | −57.143 | 3.49 |
| 018-0047 | BID | M | 3 | 5 | 3 | −40 | |
| 018-0064 | QID | M | 1.33 | 5 | 2 | −60 | 2.63 |
| 018-0068 | QID | M | 2.28 | 7 | 6 | −14.286 | 3.13 |
| 018-0611 | QID | F | 2 | 9 | 1 | −88.889 | 1.05 |
| 018-0660 | BID | F | 3.42 | 10 | 6 | −40 | 2.75 |
| 019-0534 | BID | F | 1 | 7 | 3 | −57.143 | |
| 020-0021 | QID | M | 3.08 | 7 | 7 | 0 | 1.96 |
| 020-0537 | BID | F | 3.17 | 9 | 3 | −66.667 | 2.09 |
| 020-0538 | QID | F | 2.5 | 8 | 7 | −12.5 | 2.66 |
| 022-0053 | BID | M | 0.75 | 8 | 5 | −37.5 | 1.29 |
| 022-0511 | QID | F | 2 | 7 | 6 | −14.286 | 2.31 |
| 022-0585 | QID | F | 0.75 | 10 | 5 | −50 | 1.33 |
| 022-0725 | QID | F | 3 | 9 | 10 | 11.111 | |
| 022-0726 | BID | F | 2.5 | 10 | 7 | −30 | 3.25 |
| 022-0729 | BID | F | 3.75 | 7 | 6 | −14.286 | |
| 023-0079 | BID | M | 4.25 | 8 | 9 | 12.5 | 1.33 |
| 023-0101 | QID | M | 6.17 | 8 | 5 | −37.5 | 1.47 |
| 025-0564 | BID | F | 3.67 | 10 | 10 | 0 | 1.4 |
| 026-0004 | BID | M | 3.33 | 6 | 5 | −16.667 | 1.64 |
| 026-0036 | QID | M | 4.6 | 6 | 6 | 0 | 3.92 |
| 026-0075 | BID | M | 5.23 | 7 | 0 | −100 | 1.17 |
| 026-0076 | QID | M | 2.2 | 5 | 4 | −20 | 2.9 |
| 026-0604 | BID | F | 7.52 | 8 | 5 | −37.5 | 1.41 |
| 026-0656 | BID | F | 1.37 | 8 | 3 | −62.5 | |
| 029-0107 | QID | M | 2 | 7 | 4 | −42.857 | 1.6 |
| 029-0528 | BID | F | 6.58 | 9 | 2 | −77.778 | |
| 029-0584 | QID | F | 3.92 | 10 | 6 | −40 | 2.01 |
| 031-0634 | QID | F | 3 | 8 | 6 | −25 | 3.53 |
| 032-0055 | BID | M | 2.67 | 7 | 4 | −42.857 | 1.1 |
| 032-0086 | QID | M | 4 | 8 | 5 | −37.5 | 1.32 |
| 032-0543 | QID | F | 3.42 | 8 | 9 | 12.5 | 3.35 |
| 032-0709 | QID | F | 2.25 | 9 | 5 | −44.444 | 3.38 |
| 033-0012 | QID | M | 6.92 | 7 | 4 | −42.857 | 1.81 |
| 033-0546 | QID | F | 2.42 | 8 | 5 | −37.5 | 3.23 |
| 035-0063 | BID | M | 8 | 7 | 2 | −71.429 | 0.63 |
| 035-0072 | BID | M | 0.92 | 7 | 4 | −42.857 | |
| 035-0594 | BID | F | 2.13 | 9 | 3 | −66.667 | 2.33 |
| 037-0052 | QID | M | 1.17 | 8 | 4 | −50 | 2.5 |
| 037-0074 | QID | M | 7.08 | 5 | 6 | 20 | 1.4 |
| 037-0615 | QID | F | 3.75 | 10 | 9 | −10 | 1.65 |
| 037-0711 | QID | F | 4.75 | 10 | 3 | −70 | 3.48 |
| 038-0046 | QID | M | 5.17 | 7 | 3 | −57.143 | 1.72 |
| 040-0644 | BID | F | 4.75 | 8 | 7 | −12.5 | 1.06 |
| 041-0069 | BID | M | 3.08 | 8 | 6 | −25 | 1.3 |
| 041-0098 | QID | M | 2.35 | 8 | 3 | −62.5 | 2.4 |
| 041-0649 | QID | F | 2.42 | 9 | 10 | 11.111 | 4.75 |
| 042-0105 | BID | M | 9.17 | 8 | 8 | 0 | 2.09 |
| 043-0062 | BID | M | 6 | 8 | 2 | −75 | |
| 043-0081 | BID | M | 6 | 6 | 2 | −66.667 | |

As described in Example 2, the mean 6β-naltrexol plasma concentration in the oxycodone and naltrexone BID group was statistically different from that in the oxycodone and naltrexone QID group (p<0.001). There was also a significant difference between the BID group and the QID group in pain intensity reduction, with the BID group experiencing a significant reduction in pain intensity. It was unexpected that the QID group and BID group would differ in this manner. The plasma concentrations of 6β-naltrexol appear to be at steady state at the conclusion of each dosing interval. (See FIG. 7). Therefore, in addition to the above dose group differences, the plasma concentration-effect relationship between 6β-naltrexol concentrations and clinical outcomes (effects) can be observed, with lower steady state plasma concentrations of 6β-naltrexol corresponding to greater clinical efficacy (e.g., percent change in pain intensity).

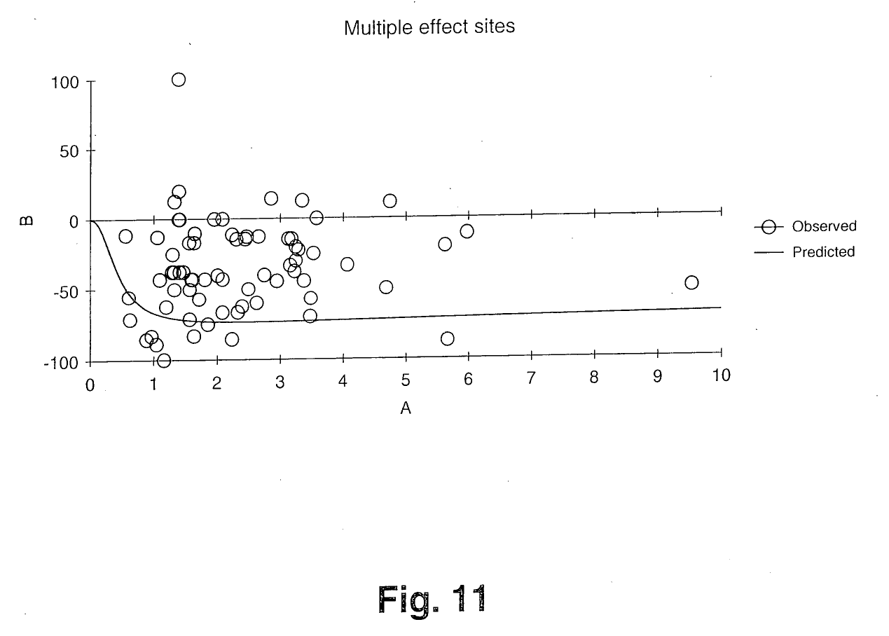

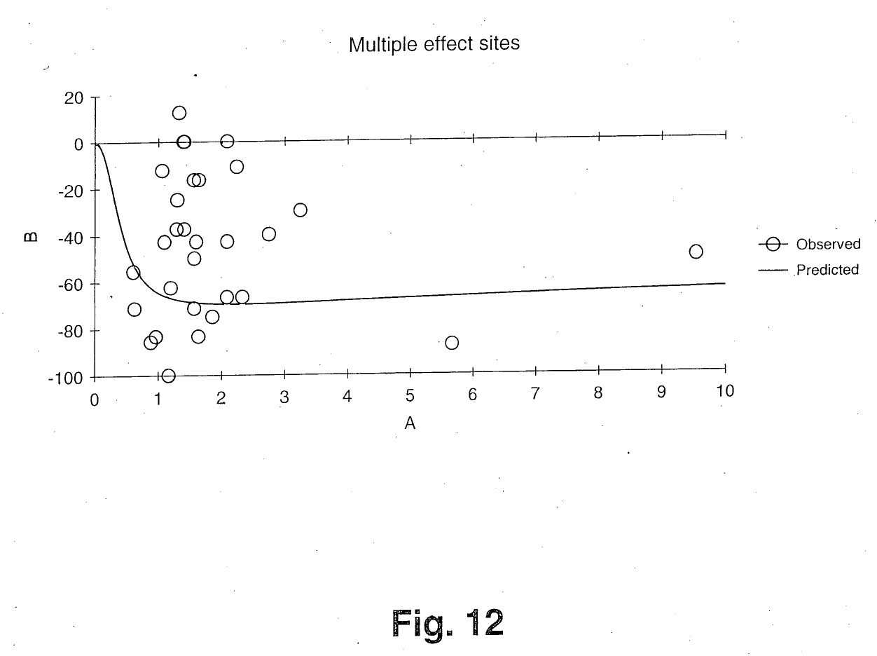

FIGS. 11 and 12 illustrate this plasma concentration-effect relationship. FIG. 11 plots the percent change in pain intensity reported by the subjects in Table 25 (y-axis) vs. 6β-naltrexol plasma concentrations measured for those subjects (x-axis). FIG. 11 includes data from subjects receiving the BID dosing regimen of the combination drug and subjects receiving the QID dosing regimen of the combination drug. The data as plotted in FIG. 11 describe a U shaped plasma concentration-effect relationship. FIG. 12 plots the percent change in pain intensity reported by the subjects receiving the BID dosing regimen of the combination drug vs. 6β-naltrexol plasma concentrations measured for those subjects.

For the first time, the plasma concentration-effect relationship of low dose of an opioid antagonist when administered with an opioid agonist has been represented by the Emax composite model:
E=[Emax1(Cpⁿ¹)/EC51ⁿ¹+Cpⁿ¹]+[Emax2(Cpⁿ²)/EC52ⁿ²+Cpⁿ²]
where the respective Emax values represent maximum effect for a given drug; EC51 and EC52 represent the potencies, for the drug notated as either 1 or 2, respectively (in other words, EC51 is not the concentration having 51% of the maximal effect, but rather EC51 is the concentration having a particular potency (e.g. 50% of the maximal effect for Effect No. 1); the respective values for C are the concentrations of drugs notated as 1 or 2, and the values of n1, and n2 that correspond to the sigmoidicity factors that are associated with particular EC values. In the Emax composite model, “+” is used to indicate absolute values; sometimes it is shown as a “−” which reflects a negative second term.

The Emax composite model is a recognized composite model for PK/PD data analysis set forth, for example, in Gabrielsson et al., PHARMACOKINETIC/PHARMACODYNAMIC DATA ANALYSIS: CONCEPTS AND APPLICATIONS, pp. 191-193 and 801-808 (2000), and the computer command files provided with the reference and described, including with examples of the computer printouts on pages 801-808, all of which is incorporated by reference herein. However, it is believed that the Emax composite model has not previously been utilized for the analysis of PK data from administering low doses of opioid antagonists such as naltrexone for enhancing the potency of opioid agonists such as oxycodone, as described herein. From the plasma concentration-effect data obtained in this Example, it is contemplated that the opioid antagonist, at lower plasma concentrations, is impacting the total effect (percent change in pain intensity), primarily as described by the terms of the equation denoted with a 2.

The recognition of the applicability and utility of a composite model as shown above enables the selection of preferred and/or suitable ranges for the combined use of an opioid antagonist with an opioid agonist as described herein. The composite model provides the relative contribution of an opioid antagonist with respect to enhancing pain relief, for example, as measured by a reduction in pain intensity. The effective percentage decrease in pain intensity, E, has been found to be described by a relatively wide scope of preferred plasma concentrations by the Emax composite model, as shown in the data and Figures described herein.

The plasma concentration-effect data were fit to the Emax composite model using the software program WinNonlin®(commercially available from Pharsight Corporation of Mountain View, Calif.) and the command files developed by Gabrielsson et al. The plasma concentration-effect data represented as circles in FIG. 11 were evaluated mathematically and the plasma concentration-effect curve shown in FIG. 11 was determined by the program and command files. Similarly, the program and command files were used to determine the plasma concentration-effect curve shown in FIG. 12 based on the data represented as circles in FIG. 12.

The computer output (printout) of this process included EC51 and EC52 parameters, as well as parameters reflecting statistical evaluation of the data, such as coefficient of variation (CV %). A variety of values, for example EC20 and EC90 (the concentrations at which 20% and 90%, respectively, of the maximum effect are obtained), may also be determined using the output from the WinNonlin® program and Gabrielsson et al command files (or similar programs and command files). Other values, for example EC0 and EC100 and all values in between, also may be determined graphically and/or using the values of N1 and N2 that correspond to the sigmoidicity factors.

Table 26 shows parameters based on the curve shown in FIG. 11. These parameters are based on the data for 6β-naltrexol plasma concentrations and reduction in pain from baseline pain intensity to final pain intensity from all subjects for whom plasma concentration data was obtained as described herein. These parameters are based on data from subjects receiving the BID dosing regimens and subjects receiving the QID dosing regimen. Estimate refers to the value estimated by the WinNonlin® program and command file for relating plasma concentrations to the pharmacodynamic effects such as percent reduction in pain intensity. Convergence of the model was easily achieved and the power of the condition number was acceptable.

TABLE 26
Parameters based on Total (BID and QID) data

| Parameter | Estimate | |
|---|---|---|
| Emax1 | 26.8 | units |
| EC51 | 14.4 | pg/ml |
| N1 (sigmoidicity factor) | 0.907 | units/pg/ml |
| Emax2 | 79.7 | units |
| EC52 | 0.439 | pg/ml |
| N2 (sigmoidicity factor) | 2.28 | units/pg/ml |

Table 27 shows parameters based on the curve shown in FIG. 12. These parameters are based on the data (6β-naltrexol plasma concentrations and percent reduction in pain intensity) for subjects receiving BID dosing regimens. Convergence of the model was easily achieved and the power of the condition number was acceptable.

TABLE 27
Parameters based on BID data

| Parameter | Estimate | |
|---|---|---|
| Emax1 | 27.0 | units |
| EC51 | 14.0 | pg/ml |
| N1 (sigmoidicity | 0.941 | units/pg/ml |
| factor) | | |
| Emax2 | 76.3 | units |
| EC52 | 0.422 | pg/ml |
| N2 (sigmoidicity | 2.16 | units/pg/ml |
| factor) | | |

As mentioned above, the BID dosing regimen of the combination drug comprising naltrexone and oxycodone resulted in statistically significant decreases in pain intensity. The Emax composite model provided the value of a EC52 plasma concentration of 6β-naltrexol based on that BID dosing regimen. Substantially the same EC52 result was obtained from the analysis of the total data set (comprising data from both the BID and QID dosing regimens). The fact that substantially the same EC52 result was obtained from the different data sets supports the strength of the Emax composite model for analysis of the data. It also supports the use of the Emax composite model in order to select desirable doses of naltrexone (or another opioid antagonist) in combination with oxycodone.

Tables 26 and 27 illustrate the use of the total set of clinical data and the subset associated with positive clinical results in the same Emax composite model to provide two sets of parameters. Either or both of the two sets of parameters can be used to identify plasma concentrations having a probability of attaining a desired reduction of pain intensity or other efficacy outcome (e.g., pharmacodynamic outcome) as described herein. From the plasma concentration-effect data and the Emax composite model, one can better assess what plasma concentrations of 6β-naltrexol provide desired reduction in pain intensity and, more generally, better pain treatment. Based on plasma concentration data (e.g., as shown in Table 25), presently preferred dosage forms for oral administration to a human subject comprise a dose amount of opioid antagonist that is based on a selected plasma concentration. Thus, naltrexone and/or 6β-naltrexol may be used to titrate a subject to the appropriate dose for that subject thus providing a convenient means for individualized dosing.

The Emax composite model can facilitate dose titration for a human subject. Dose titration refers to the process of employing different doses (usually escalating doses) in a subject until a dose effective to achieve a desired clinical outcome is found. Dose titration for the administration of an opioid antagonist and/or an opioid agonist according to the present disclosure may be facilitated by using plasma concentrations of 6β-naltrexol, naltrexone, or another marker of opioid antagonist. Dose titration may also be facilitated by using plasma concentrations of oxycodone, oxymorphone, or another marker of opioid agonist may be used alone or in combination with a marker of opioid antagonist for dose titration.

For dose titration of the administration of an opioid antagonist and an opioid agonist to a human subject, the subject's plasma concentration of 6β-naltrexol, naltrexone or another marker for opioid antagonist is analyzed, and one or more clinical outcomes (such as reduction in pain intensity) for the subject are analyzed. If a desired clinical outcome is not achieved (for example, if pain intensity is not reduced to a desired level), the administration of opioid antagonist and/or opioid agonist to the subject is adjusted. The composite model can be used to facilitate adjusting, or facilitate the decision to adjust, the administration of (a) the opioid antagonist or (b) the opioid agonist or (c) both.

In the present method of titrating a human subject, if a clinical outcome is not at a desired level, the plasma concentration of 6β-naltrexol is analyzed. If the 6-βnaltrexol plasma concentration is not at a desired level, then administration of the opioid antagonist to the subject is adjusted. The administration of the opioid antagonist may be adjusted by adjusting the dose amount and/or dosing regimen. However, if the 6β-naltrexol plasma concentration is already at a desired level, yet the clinical outcome is not at a desired level, then the administration of the opioid agonist to the subject is adjusted. The administration of the opioid agonist may be adjusted by adjusting the dose amount and/or dosing regimen.

For example, in a method of titrating the administration of an opioid agonist and an opioid antagonist a human subject to reduce pain intensity in the subject, if the reduction in pain intensity is not at a desired level, the plasma concentration of 6β-naltrexol is analyzed. If the 6β-naltrexol plasma concentration is below a desired level, then administration of the opioid antagonist to the subject is adjusted so that more opioid antagonist is administered to the subject. If the 6β-naltrexol plasma concentration is above a desired level, then administration of the opioid antagonist to the subject is adjusted so that less opioid antagonist is administered to the subject. However, if the 6β-naltrexol plasma concentration is already at a desired level, yet the reduction in pain intensity is not at a desired level, then the administration of the opioid agonist to the subject is adjusted so that more opioid agonist is administered to the subject.

The Emax composite model may be used to identify desired levels of the plasma concentration of opioid antagonist, for example a level indicated by the composite model as having a desired level of efficacy. For example, parameters, including but not limited to EC20, EC50 and EC90, identified by the composite model may be employed to select desirable levels of plasma concentrations of opioid antagonist (as measured directly or via a surrogate marker such as 6β-naltrexol).

Parameters provided by the composite model may be employed to select desirable doses of naltrexone from the plasma concentrations of 6β-naltrexol, based on the foregoing data, parameters and adjustments relating to 6β-naltrexol. As mentioned above, when naltrexone is administered to a human subject, the plasma concentration of 6β-naltrexol is useful as an indicator of the absorption of naltrexone, since 6β-naltrexol is generally present in plasma at concentrations much higher than those of naltrexone due to the rapid metabolism of naltrexone to yield 6β-naltrexol. For example, a 6β-naltrexol plasma concentration of about 0.4 μg/ml indicates a naltrexone plasma concentration of about 0.04 μg/ml in the plasma sample, and where a given 60β-naltrexol plasma concentration is provided herein, a naltrexone plasma concentration of about 1/10 of the given 6β-naltrexol plasma concentration is also contemplated.

The plasma concentration of 6β-naltrexol at steady state is generally proportional to the dose amount of naltrexone in a BID dosing regimen. It has been found that a dose of an opioid antagonist such as naltrexone given as a BID regimen that produces plasma concentrations of free 6β-naltrexol that are related by a proportionality factor to naltrexone correlated for a given dose of an opioid agonist statistically (p<0.001) with percent decreases in pain intensity from base line for moderate to severe pain.

Accordingly, a desirable dose amount of opioid antagonist, and optionally a desirable dose amount of opioid agonist, can be selected based on a steady state plasma concentration that exhibits a desired pharmacodynamic (PD) effect. Exemplary data for plasma concentrations and PD effects are shown in Table 25. Based on the proportional relationship between concentration and dose, a formula for converting between concentration and dose can be established by experimentally determining plasma concentrations that result from known dose amounts. This formula may be used to select dose amounts of opioid antagonists converted from plasma concentrations showing a desired PD effect. Furthermore, the dose of a co-administered opioid agonist may be adjusted, by increasing or decreasing the dose, relative to the opioid antagonist, to further optimize pain relief or other efficacy outcomes as described herein.

This linear relationship is true for the case where the daily dosing regimen results in a steady state plasma concentration. In the present Example, the greatest frequency of obtaining plasma concentrations associated with significant improvement in pain relief as reflected by the percentage change in the pain intensity score was obtained for the dose of naltrexone given BID. Naltrexone at the dose as described herein when given more frequently than BID resulted in a greater proportion of 6β-naltrexol concentrations increasing above those for the BID dosing regimen in a statistically significant (p<0.0001) proportion of the population. Stated differently, the plasma levels of naltrexone, as measured by its major metabolite 6β-natrexol were too high in the OID dosing regimen, thus a statistically significant increase in pain relief with the QID dosing regimen of naltrexone as described herein was not achieved. However, since individual patients in the QID dosing group did not achieve an increase in pain relief as shown in Table 25, a statistically significant increase in pain relief with a similar QID dosage regimen of the opioid antagonist (e.g., naltrexone) may be achieved when the dose of the opioid agonist (e.g., oxycodone) is increased relative to the amount of antagonist.

Parameters, including but not limited to EC20, EC50 and EC90, identified by the composite model may be employed to select desirable amounts of opioid antagonist in various dosage forms. A desired amount of opioid antagonist can be determined from a selected plasma concentration arising from a known amount of opioid antagonist, since the relationship between concentration and dose amount is generally linearly proportional. The plasma concentrations of 6β naltrexol from randomly selected samples from subjects receiving 1 μg of naltrexone and 20 mg of oxycodone in a BID dosing regimen were fit to the Emax composite model. The EC52 of 6β naltrexol in the plasma, as the surrogate marker for the active drug naltrexone in the plasma, corresponding to 1 μg of naltrexone from the BID dosing regimen was computed.

By way of example, but not as a limitation, parameters provided by a composite model are useful for predicting doses from desirable lower levels of plasma concentrations of 6p-naltrexol. More particularly, the EC52 parameter in Table 26 suggests that a 6β-naltrexol plasma concentration of about 0.439 μg/ml or more may be employed to attain better than a 50% reduction in pain intensity. Additional preferences may be selected; for example, if one wishes to attain better than 20% or better than 90% reduction in pain intensity, one may select the plasma concentrations indicated in FIG. 11 that correspond to the 20% or the 90% effectiveness levels, respectively.

As another example, but not as a limitation, parameters provided by the composite model are useful for selecting desirable higher levels of plasma concentrations of 6β-naltrexol. As one avenue, the EC51 parameter may be used in a fashion similar to the use of the EC52 parameter as described above.

A range of preferred dose amounts was calculated from the Emax composite model using EC20 derived from the graphic output and the sum of the EC52 plus the CV % obtained from the model. For example, a range of dose amounts is selected wherein the low point is the dose amount corresponding to the plasma concentration at EC20, and the high point is the dose amount corresponding to the plasma concentration that is the sum of the EC52 plus the CV % (133) obtained from the Emax composite model. By way of example, but not as a limitation, where the opioid antagonist naltrexone is provided in a dosing regimen that also includes 20 mg oxycodone, preferred dose amounts of opioid antagonist may comprise the range of from about 0.829 μg to about 2.37 μgs.

For a given opioid agonist that may be given in different dose amounts, it may be desirable to provide preferred concentrations or amounts of opioid antagonist. If the dosing regimen is to include 10 mg oxycodone (rather than 20 mg), an alternative preferred dose amounts may comprise the range of from about 0.415 μg to about 1.19 μgs. It is contemplated that, generally, a preferred dose amount may be adjusted in a proportionate manner to a change in oxycodone amount. If oxycodone amount is reduced or increased by a factor of 2, 4, or 8 (or other factor), the end points of the preferred range are each reduced or increased by a same factor (2, 4, or 8 or other factor)).

Accordingly, the plasma concentration-effect data set forth above for the subjects receiving the BID dosing regimen or the total plasma concentration-effect data (BID and QID) dosing regimens) can be employed to select dose amounts of opioid antagonist to be administered. For example, the plasma concentration-effect data in Table 27, which relate to the plasma concentration-effect data from subjects receiving the BID dosing regimen, and the mathematical evaluation of the data using the Emax composite model, as exemplified in FIG. 12, may be employed to select dose amounts of opioid antagonist to be administered in a BID dosing regimen. Employing that data and the composite model, for a BID dosing regimen that includes 20 mg oxycodone, presently preferred dose amounts of opioid antagonist comprise from about 0.829 μg to about 2.37 μgs. Where the BID dosing regimen comprises other amounts of oxycodone per dose, exemplary dose amounts of opioid antagonist are contemplated: 1 mg oxycodone per dose: from about 0.041 μg to about 0.119 μg opioid antagonist per dose 2.5 mg oxycodone per dose: from about 0.103 μg to about 0.297 μg opioid antagonist per dose 5 mg oxycodone per dose: from about 0.207 μg to about 0.593 μg opioid antagonist per dose 10 mg oxycodone per dose: from about 0.415 μg to about 1.19 μgs opioid antagonist per dose 40 mg oxycodone per dose: from about 1.66 μgs to about 4.74 μgs opioid antagonist per dose 80 mg oxycodone per dose: from about 3.32 μgs to about 9.48 μgs opioid antagonist per dose 160 mg oxycodone per dose: from about 6.64 μgs to about 18.96 μgs opioid antagonist per dose Thus, for a BID dosing regimen that includes an amount of oxycodone, presently preferred dose amounts of opioid antagonist may comprise from about 0.041 μg to about 18.96 μgs.

As another example, the plasma concentration-effect data in Table 26, which relate to the total plasma concentration-effect data from subjects receiving the BID dosing regimen and subject receiving the QID dosing regimen, and the mathematical evaluation of the data using the composite Emax/Imax model, as exemplified in FIG. 11, may be employed to select preferred dose amounts of opioid antagonist more generally. For a dosing regimen that includes 20 mg oxycodone, presently preferred dose amounts of opioid antagonist comprise from about 0.830 μg to about 5.02 μgs. Where the dosing regimen comprises other amounts of oxycodone per dose, exemplary dose amounts of opioid antagonist are contemplated:

1 mg oxycodone per dose: from about 0.041 μg to about 0.252 μg opioid antagonist per dose 2.5 mg oxycodone per dose: from about 0.104 μg to about 0.63 μg opioid antagonist per dose 5 mg oxycodone per dose: from about 0.208 μg to about 1.26 μg opioid antagonist per dose 10 mg oxycodone per dose: from about 0.415 μg to about 2.51 μgs opioid antagonist per dose 40 mg oxycodone per dose: from about 1.66 μgs to about 10.0 μgs opioid antagonist per dose 80 mg oxycodone per dose: from about 3.32 μgs to about 20.1 μgs opioid antagonist per dose 160 mg oxycodone per dose: from about 6.64 μgs to about 40.2 μgs opioid antagonist per dose Thus, for a BID dosing regimen that includes an amount of oxycodone, presently preferred dose amounts of opioid antagonist may comprise from about 0.041 μg to about 40.2 μgs.

Furthermore, any of the foregoing ranges may be broadened by substituting the foregoing lower ends with a lower end of about 0.0002 μg since dose amounts as low as about 0.0002 μg are presently contemplated. It was observed that the lower end of the ranges can approach zero based on the relatively low CV % s observed at the low end of the composite model (i.e., the values 132 and 151 for the BID and total (BID and QID) data sets, respectively). This indicates that even lower dose amounts of naltrexone and other opioid antagonists would be expected to be active, and dose amounts of about 0.0002 μg would be expected to be active albeit in a decreasing proportion of the population.

The present Example also provides preferred methods and materials comprising opioid antagonists other than naltrexone, such as naloxone and nalmefene. It is believed that, generally, the preferred dose amounts of naltrexone calculated above are useful for other opioid antagonists. Persons skilled in the field will recognize a particular opioid antagonist may have potency, bioavailability, metabolism, clearance, or other characteristics that suggest an adjustment to the dose amount, dosage form, or dosing regimen. For example, for opioid antagonists having reduce oral availability compared to naltrexone, it is contemplated that a higher oral dose amount will be provided, or that a more frequent dosing regimen will be employed, or that an intravenous dose will be provided, or some other adjustment will be made. Such adjustments are well within the ability of persons skill in the field.

As discussed above, methods and materials are provided for titrating an opioid antagonist administered to a human subject. By way of example, but not as a limitation, a suitable method comprises the steps of (a) administering an amount of an opioid antagonist and an amount of an opioid agonist to the subject, (b) measuring a plasma concentration in the subject of the opioid antagonist or a surrogate of the opioid antagonist, and (c) adjusting the amount of the opioid antagonist administered to the subject if the measured plasma concentration is outside a predetermined plasma concentration range. The predetermined plasma concentration range can be from concentrations predicted by a model of plasma concentration-effect relationship (e.g., the Emax composite model described above). The predetermined plasma concentration range can be the range predicted by the model to provide a reduction in pain intensity of about 20% or greater, alternatively about 50% or greater, alternatively about 90% or greater. The predetermined plasma concentration can be based on the plasma concentration-effect model shown in FIG. 11 or FIG. 12.

However, the present methods and materials for titrating an opioid antagonist administered to a human subject are not limited to the use of a composite model or to the use of predetermined plasma concentrations. By way of example, methods and materials of titrating an opioid antagonist administered to a human subject are provided, which comprise (a) administering an amount of an opioid antagonist and an amount of an opioid agonist to the subject, (b) assessing one or more symptoms or signs of an arthritic condition, inflammation associated with a chronic condition, or chronic pain, (c) measuring a plasma concentration in the subject of the opioid antagonist or a surrogate of the opioid antagonist, and (d) adjusting the amount of the opioid antagonist or the amount of the opioid agonist to the subject based on the measured plasma concentration. Step (d) may include comprises adjusting the amount of the opioid antagonist administered to the subject; alternatively or additionally, step (d) can comprises adjusting the amount of the opioid agonist administered to the subject.

As another example, methods and materials of titrating an opioid antagonist administered to a human subject are provided, which comprise (a) administering an amount of an opioid antagonist and an amount of an opioid agonist to the subject, (b) assessing one or more symptoms or signs of an arthritic condition, inflammation associated with a chronic condition, or chronic pain, and (c) adjusting the amount of the opioid antagonist administered to the subject if one or more of the assessed symptoms or signs are not alleviated to a desired extent. Step (c) can also comprise maintaining the amount of the opioid agonist administered to the subject. The method may also comprise the steps of (d) re-assessing one or more of the symptoms or signs after step (c), and (e) adjusting the amount of the opioid agonist if one or more of the assessed symptoms or signs are not alleviated to a desired extent.

In the titration methods and materials provided herein, it may be desirable to repeatedly administer the opioid antagonist such that a steady state is achieved before assessing one or more symptoms or signs of an arthritic condition, inflammation associated with a chronic condition, or chronic pain. The initial step of administering a first amount of an opioid antagonist and/or a first amount an opioid agonist can be repeated if the measured plasma concentration is within the predetermined plasma concentration range and/or if the assessed symptom(s) or sign(s) is alleviated to a desired extent.

In the titration methods and materials provided herein, it is contemplated that one or more of the assessed symptoms or signs may be pain, stiffness, and/or difficulty in physical function had by the subject, or measures of pain, stiffness and difficulty in physical function, such as the measures set forth in the WOMAC Osteoarthritis Index or one of its subscales. For example, a symptom or sign assessed for purposes of titration may be pain as measured as pain intensity. The pain intensity measurement may be attenuated as compared to a pain intensity baseline measurement of the subject. For example, the pain intensity measurement may be reduced by at least about 20%, alternatively at least about 50%, alternatively at least about 90%, compared to a pain intensity baseline measurement of the subject.

In the titration methods and materials provided herein, it is contemplated that a plasma concentration of the opioid antagonist or a surrogate of the opioid antagonist may be measured, and the amount of the opioid antagonist can be adjusted based in part on the measured plasma concentration. For example, the amount of the opioid antagonist administered to the subject is increased if the measured plasma concentration is lower than a predetermined plasma concentration value. As another example, the amount of the opioid antagonist administered to the subject is decreased in the measured plasma concentration is higher than a predetermined plasma concentration value. As yet another example, the amount of the opioid antagonist administered to the subject is maintained in the measured plasma concentration is within a predetermined plasma concentration range, and optionally the amount of the opioid agonist administered to the subject is increased.

While the foregoing generally preferred concentrations and amounts of opioid antagonists are contemplated for use with a wide variety of opioid agonists, it is contemplated that, for particular opioid agonists, particular concentrations and/or amounts may be selected based on the present disclosure. The foregoing generally preferred concentrations and amounts have been selected based on data from clinical studies employing the opioid antagonist naltrexone and the opioid agonist oxycodone, however they are also contemplated for use with a wide variety of opioid antagonists and opioid agonists.

### EXAMPLE 4

A clinical study was conducted as described in Example 1 (Part A) and data were obtained as described in Examples 1 and 2. Plasma samples from selected subjects in the clinical study were used to assay for the presence and concentration of selected cytokines.

Plasma samples were analyzed using a commercial cytokine assay from Pointilliste (www.pointilliste.com) to quantify the concentrations of IL2, IL4, IL5, IL6, IL10, IL13, GM-CSF, interferon and TNFα. Plasma samples were separately analyzed for IL1αand IL1β, which were quantitated using a conventional cytokine assay by Pointilliste. The cytokine assay for the quantitation of IL2, IL4, IL5, IL6, IL10, IL13, GM-CSF, interferon and TNFα employed, Pointilliste's Human Th1/Th2 Cytokine Canvas product which contains binding sites for each of these nine cytokines. The array pattern of cytokine antibodies printed in each well of a 96 well microtiter plate is shown in Table 28.

TABLE 28
Pattern of Human Th1/Th2 Cytokine Canvas

| | | | | |
|---|---|---|---|---|
| Alignment | Anti-IL2 | Anti-IL2 | Anti-IL13 | Anti-IL13 |
| marker | | | | |
| Negative | Anti-IL4 | Anti-IL4 | Anti-GM- | Anti-GM- |
| control | | | CSF | CSF |
| Negative | Anti-IL5 | Anti-IL5 | Anti-IFNγ | Anti-IFNγ |
| control | | | | |
| Reagent | Anti-IL6 | Anti-IL6 | Anti-TNFα | Anti-TNFα |
| Control 1 | | | | |
| Reagent | Anti-IL10 | Anti-IL10 | Negative | Alignment |
| Control 2 | | | control | marker |

Two measurements (duplicates) for each cytokine were possible for each plasma sample applied to a canvas, since the canvas has two binding sites for each cytokine.

Fifty-seven frozen human plasma samples containing EDTA as an anticoagulant were thawed on ice and transferred to a sterile 96-deep well polypropylene plate. The plate was centrifuged briefly at 4° C. to clarify the plasma. Aliquots of clarified plasma were removed for cytokine analysis, and the remaining samples in the 96-deep well polypropylene plate were stored at −80° C.

The aliquots of clarified plasma were transferred to sterile non-protein binding 96-well polypropylene plates to enable parallel processing of the samples. Each of the wells of these plates included a Pointilliste Human Th1/Th2 Cytokine Canvas as shown in Table 26. Two different 96 well plates were used, and each received a subsample of the aliquots at different dilutions. Two dilutions (1 in 1 and 1 in 10) of each sample were assayed on two separate human Th1/Th2 cytokine canvases. In order to quantify the cytokine concentrations, standard curves were generated for each dilution. A mixture of 9 cytokines was run on each of the canvases and used to calculate a standard curve, which was used to determine the amount of each cytokine in the samples. The standard curves were plotted with the signal intensity as a function of the cytokine concentration in ng/ml. A CCD camera was used with Pointilliste's Canvas Analysis Tools software to generate data corresponding to cytokine concentrations.

In addition to the assays using the Pointilliste Human Th1/Th2 Cytokine Canvas, conventional ELISA analysis was used to measure the concentrations of IL1αand IL1β in the plasma samples. The standard curves for IL1α and IL1β were also plotted with the signal intensity as a function of the cytokine concentration in ng/ml.

Tables 29 through 31 show measurements of cytokine concentrations (ng/ml) obtained as described herein and data calculated from those measured concentrations. In Tables 29 through 31, the following abbreviations are used: “OXY” refers to the treatment group receiving oxycodone QID as described in Example 1; “BID” refers to the treatment group receiving the combination drug of oxycodone and naltrexone BID as described in Example 1; “QID” refers to the treatment group receiving the combination drug of oxycodone and naltrexone QID as described in Example 1; “GM” refers to granulocyte/macrophage colony stimulating factor; “IFN” refers to interferon gamma; “TNF” refers to tumor necrosis factor alpha; “IL2” refers to interleukin 2; “IL4” refers to interleukin 4; “IL5” refers to interleukin 5; “IL6” refers to interleukin 6; “IL10” refers to interleukin 10; and “IL13” refers to interleukin 13.

Table 29 shows the individual cytokine measurements obtained from each sample as identified by sample identification number. Accordingly, Table 29 shows all the cytokine measurements that were obtained for each sample. Table 30 shows a compilation of the individual cytokine measurements obtained from the plasma samples. These measurements were used to determine the mean cytokine concentrations. The numbers of measurements for the various cytokines differ because different interferences affected samples and cytokine measurement within those samples differently.

TABLE 29
Individual Cytokine Concentrations (ng/ml)

| Requisition # | IL2 | IL2 | IL4 | IL4 | IL5 | IL5 |
|---|---|---|---|---|---|---|
| 21896190 | 0.20055269 | 0.11856273 | | | | |
| 21896054 | | | | | | |
| 22982837 | | | 0.09166477 | 0.07630626 | | |
| 21366538 | 0.2226875 | 0.1560676 | | | | |
| 20889685 | | | 0.15974633 | 0.15681396 | | |
| 20468981 | 0.26894192 | 0.16131945 | | | | |
| 20871516 | 0.36578316 | 0.15581297 | | | | |
| 20996662 | | | | | | |
| 20322246 | 0.72330313 | 0.55323963 | 0.26509831 | 0.2369057 | | |
| 20893086 | 0.17732026 | 0.08939692 | | | | |
| 20904434 | | | | | | |
| 20902230 | | | | | | |
| 20982936 | | | | | | |
| 21641580 | 2.05566036 | 2.02979174 | 0.66585024 | 0.58444767 | | |
| 21641624 | | | | | | |
| 20997134 | | | | | | |
| 20?91744 | 0.18491683 | 0.1726987 | | | | |
| 20904803 | | | | | | |
| 20704612 | 0.47551031 | 0.40121823 | 0.13785932 | 0.1410828 | | |
| 20903725 | | | | | | |
| 20894411 | | | | | | |
| 21970721 | 0.21063224 | 0.15920151 | | | | |
| 21970800 | | | | | | |
| 20868748 | | | | | | |
| 21060022 | | | | | | |
| 21060145 | 0.13903841 | 0.07079245 | | | | |
| 21641760 | | | | | | |
| 21641850 | 1.12078831 | 0.89434998 | 0.28574849 | 0.24575749 | | |
| 21641793 | | | | | | |
| 22029704 | | | | | | |
| 22029884 | 0.20803264 | 0.12020192 | | | | |
| 22029895 | | | | | | |
| 20712802 | | | | | | |
| 20895614 | | | | | | |
| 20895647 | | | | | | |
| 21970710 | | | | | | |
| 20895120 | 0.32586262 | 0.15010384 | | | | |
| 20895030 | 0.65247635 | 0.48797657 | 0.16596928 | 0.16197549 | | |
| 20895197 | 1.81737173 | 1.58252682 | 0.47248956 | 0.46554916 | | |
| 20884555 | 0.17843304 | 0.09934644 | | | | |
| 20892464 | | | | | | |
| 21013356 | 0.16723079 | 0.0920016 | | | | |
| 22927195 | | | | | | |
| 23366766 | | | | | | |
| 22043250 | | | | | | |
| 22016002 | 1.0243213 | 0.88010212 | 0.49134739 | 0.56386303 | 0.48660202 | 0.48831413 |
| 22015910 | 0.47709418 | 0.36350033 | | | | |
| 22464955 | | | | | | |
| 23018163 | 0.16338282 | 0.03667778 | | | | |
| 21720868 | | | 0.09131771 | 0.09070153 | | |
| 21721037 | | | | | | |
| 22121010 | | | | | | |
| Requisition # | IL6 | IL6 | IL10 | IL10 | IL13 | IL13 |
| 21896190 | 0.05665755 | 0.03555141 | | | 0.19392934 | 0.44115565 |
| 21896054 | 0.06451101 | 0.0692487 | | | | |
| 22982837 | | | | | | |
| 21366538 | 0.04990713 | 0.03994802 | | | 0.07605412 | 0.23363825 |
| 20889685 | 0.05213618 | 0.03663666 | | | 0.70581467 | 0.73173353 |
| 20468981 | | | | | | |
| 20871516 | | | | | 0.39807991 | 0.43517553 |
| 20996662 | | | | | | |
| 20322246 | 0.33537354 | 0.35986346 | 0.21636839 | 0.2438428 | 1.08994297 | 0.94101683 |
| 20893086 | | | | | 0.81325709 | 0.63358401 |
| 20904434 | | | | | 0.48282395 | 0.44626946 |
| 20902230 | | | | | | |
| 20982936 | | | | | 0.12980545 | 0.14265747 |
| 21641580 | 0.11742943 | 0.13925241 | 0.12089472 | 0.17918052 | 1.34132334 | 0.77623126 |
| 21641624 | | | 0.2371214 | 0.17262211 | 0.64922975 | 0.58858556 |
| 20997134 | | | | | | |
| 20?91744 | | | | | 0.47053745 | 0.33540459 |
| 20904803 | 0.09070808 | 0.00112223 | 0.12533194 | 0.37429311 | | |
| 20704612 | 0.07740341 | 0.07723647 | | | 0.62558119 | 0.33846604 |
| 20903725 | | | 0.07237012 | 0.22348702 | 0.1417683 | 0.10915494 |
| 20894411 | 0.0663709 | 0.06795795 | 0.0902624 | 0.22513845 | 0.1949292 | 0.17345874 |
| 21970721 | | | 0.04094618 | 0.08917843 | 0.50278268 | 0.45389581 |
| 21970800 | | | | | | |
| 20868748 | | | | | | |
| 21060022 | | | | | | |
| 21060145 | | | | | 0.48572734 | 0.46117658 |
| 21641760 | 0.14688034 | 0.13912545 | | | 0.064658 | 0.14512129 |
| 21641850 | 0.1582319 | 0.16709362 | | | 0.08465276 | 0.14293827 |
| 21641793 | | | | | 0.16898779 | 0.07300272 |
| 22029704 | 0.11310831 | 0.12393628 | | | 0.18947654 | 0.06004538 |
| 22029884 | 0.11153591 | 0.11069421 | 1.23635389 | 1.39363282 | 3.39229184 | 3.29071344 |
| 22029895 | 0.25877076 | 0.28127371 | | | 0.17262278 | 0.08433479 |
| 20712802 | | | | | 0.21364885 | 0.12981933 |
| 20895614 | | | | | | |
| 20895647 | 0.19964952 | 0.19868269 | | | 0.54685694 | 0.52753084 |
| 21970710 | | | | | 0.5223157 | 0.47200768 |
| 20895120 | | | 0.29585033 | 0.37571509 | 0.37002894 | 0.28470566 |
| 20895030 | 0.36904351 | 0.40419606 | 0.2361711 | 0.23885961 | 1.77426577 | 1.75789523 |
| 20895197 | 0.60404449 | 0.59568652 | 1.2602512 | 0.95982996 | 2.07889414 | 2.36158936 |
| 20884555 | | | | | 0.16535367 | 0.13963199 |
| 20892464 | | | | | | |
| 21013356 | | | | | 0.14153625 | 0.15088886 |
| 22927195 | 0.05790308 | 0.04453066 | | | | |
| 23366766 | 0.26943861 | 0.26152781 | | | | |
| 22043250 | 0.07944299 | 0.09620426 | | | | |
| 22016002 | 0.34897264 | 0.36998684 | 1.90278352 | 1.97097605 | 4.31719746 | 4.56515688 |
| 22015910 | 0.13710776 | 0.13450197 | 0.53936987 | 0.49835733 | 1.46693423 | 1.33230073 |
| 22464955 | 0.29919717 | 0.34984844 | 0.0579697 | 0.05035428 | 0.53207364 | 0.43591125 |
| 23018163 | 0.16754114 | 0.15867266 | 0.06037481 | 0.05236333 | 0.71059496 | 0.74330939 |
| 21720868 | 0.06949291 | 0.07330224 | | | | |
| 21721037 | | | | | | |
| 22121010 | 0.14418425 | 0.11755667 | 0.25846742 | 0.24059597 | 1.80730556 | 1.75018302 |
| Requisition # | GM-CSF | GM-CSF | IFN gamma | IFN gamma | TNF alpha | TNF alpha |
| 21896190 | | | | | | |
| 21896054 | | | | | | |
| 22982837 | | | | | | |
| 21366538 | | | | | | |
| 20889685 | | | | | 0.07988409 | 0.05482446 |
| 20468981 | | | | | | |
| 20871516 | | | | | 0.0855576 | 0.06890438 |
| 20996662 | | | | | | |
| 20322246 | | | 0.0722552 | 0.07442144 | 0.20004415 | 0.14688815 |
| 20893086 | | | | | | |
| 20904434 | | | | | 0.10294933 | 0.16723002 |
| 20902230 | | | | | | |
| 20982936 | | | | | | |
| 21641580 | 0.33378408 | 0.37383059 | 0.2443029 | 0.29138781 | 0.42359475 | 0.38327461 |
| 21641624 | | | | | | |
| 20997134 | | | | | | |
| 20?91744 | | | | | | |
| 20904803 | | | | | | |
| 20704612 | | | | | | |
| 20903725 | | | | | | |
| 20894411 | | | | | | |
| 21970721 | | | | | | |
| 21970800 | | | | | | |
| 20868748 | | | | | | |
| 21060022 | | | | | | |
| 21060145 | | | | | | |
| 21641760 | | | | | | |
| 21641850 | | | 0.07860321 | 0.06975249 | 0.14351714 | 0.15694653 |
| 21641793 | | | | | | |
| 22029704 | | | | | | |
| 22029884 | 0.67155886 | 0.66705429 | | | 0.053116 | 0.05331531 |
| 20888717 | | | | | | |
| 22029895 | | | | | | |
| 20712802 | | | | | | |
| 20895614 | | | | | | |
| 20895647 | | | | | 0.05856579 | 0.05757872 |
| 21970710 | | | | | | |
| 20895120 | | | | | | |
| 20895030 | | | | | 0.37967017 | 0.35271469 |
| 20895197 | 0.60203722 | 0.53138737 | 0.21970779 | 0.24065142 | 0.51531889 | 0.51520632 |
| 20884555 | | | | | | |
| 20892464 | | | | | | |
| 21013356 | | | | | 0.05315699 | 0.06095267 |
| 22927195 | | | | | | |
| 23366766 | | | | | | |
| 22043250 | | | | | | |
| 22016002 | 1.41686033 | 1.40590028 | 0.13459554 | 0.12790808 | 0.27045659 | 0.26908761 |
| 22015910 | 0.26198022 | 0.2071141 | | | 0.12529323 | 0.13392305 |
| 22464955 | | | | | 0.07389075 | 0.0611985 |
| 23018163 | | | | | 0.05621241 | 0.06228325 |
| 21720868 | | | | | | |
| 21721037 | | | | | 0.0695864 | 0.05220257 |
| 22121010 | 0.23366778 | 0.20236439 | | | 0.04704521 | 0.05611238 |

TABLE 30
Cytokine Concentration Measurements Obtained from
Plasma Samples from Clinical Study
Cytokine Values

| OXY | BID | QID |
|---|---|---|
| IL2 | IL2 | IL2 |
| 0.0920016 | 0.07079245 | 0.03667778 |
| 0.12020192 | 0.08939692 | 0.1560676 |
| 0.15010384 | 0.09934644 | 0.15920151 |
| 0.16131945 | 0.11856273 | 0.16338282 |
| 0.16723079 | 0.13903841 | 0.21063224 |
| 0.20803264 | 0.15581297 | 0.2226875 |
| 0.26894192 | 0.1726987 | 0.48797657 |
| 0.32586262 | 0.17732026 | 0.65247635 |
| 0.40121823 | 0.17843304 | 0.88010212 |
| 0.47551031 | 0.18491683 | 0.89434998 |
| 0.55323963 | 0.20055269 | 1.0243213 |
| 0.72330313 | 0.36350033 | 1.12078831 |
| 1.58252682 | 0.36578316 | 2.02979174 |
| 1.81737173 | 0.47709418 | 2.05566036 |
| IL4 | IL4 | IL4 |
| 0.09070153 | 0.07630626 | 0.16197549 |
| 0.09131771 | 0.09166477 | 0.16596928 |
| 0.13785932 | 0.15681396 | 0.24575749 |
| 0.1410828 | 0.15974633 | 0.28574849 |
| 0.2369057 | | 0.49134739 |
| 0.26509831 | | 0.56386303 |
| 0.46554916 | | 0.58444767 |
| 0.47248956 | | 0.66585024 |
| IL5 | IL5 | IL5 |
| | | 0.48660202 |
| | | 0.48831413 |
| IL6 | IL6 | IL6 |
| 0.04453066 | 0.03555141 | 0.00112223 |
| 0.05790308 | 0.03663666 | 0.03994802 |
| 0.06949291 | 0.05213618 | 0.04990713 |
| 0.07330224 | 0.05665755 | 0.06451101 |
| 0.07723647 | 0.0663709 | 0.0692487 |
| 0.07740341 | 0.06795795 | 0.09070808 |
| 0.11069421 | 0.07944299 | 0.11742943 |
| 0.11153591 | 0.09620426 | 0.13925241 |
| IL6 | IL6 | IL6 |
| 0.13912545 | 0.11310831 | 0.1582319 |
| 0.14688034 | 0.11755667 | 0.15867266 |
| 0.33537354 | 0.12393628 | 0.16709362 |
| 0.35986346 | 0.13450197 | 0.16754114 |
| 0.59568652 | 0.13710776 | 0.25877076 |
| 0.60404449 | 0.14418425 | 0.26152781 |
| | 0.19868269 | 0.26943861 |
| | 0.19964952 | 0.28127371 |
| | 0.29919717 | 0.34897264 |
| | 0.34984844 | 0.36904351 |
| | | 0.36998684 |
| | | 0.40419606 |
| IL10 | IL10 | IL10 |
| 0.07237012 | 0.05035428 | 0.04094618 |
| 0.21636839 | 0.0579697 | 0.05236333 |
| 0.22348702 | 0.0902624 | 0.06037481 |
| 0.2438428 | 0.22513845 | 0.08917843 |
| 0.29585033 | 0.24059597 | 0.12089472 |
| 0.37571509 | 0.25846742 | 0.12533194 |
| 0.95982996 | 0.49835733 | 0.17262211 |
| 1.23635389 | 0.53936987 | 0.17918052 |
| 1.2602512 | | 0.2361711 |
| 1.39363282 | | 0.2371214 |
| | | 0.23885961 |
| | | 0.37429311 |
| | | 1.90278352 |
| | | 1.97097605 |
| IL13 | IL13 | IL13 |
| 0.064658 | 0.06004538 | 0.07605412 |
| 0.07300272 | 0.12981933 | 0.08433479 |
| 0.10915494 | 0.13963199 | 0.08465276 |
| 0.14153625 | 0.16535367 | 0.12980545 |
| 0.1417683 | 0.17345874 | 0.14265747 |
| 0.14512129 | 0.18947654 | 0.14293827 |
| 0.15088886 | 0.19392934 | 0.17262278 |
| 0.16898779 | 0.1949292 | 0.23363825 |
| 0.28470566 | 0.21364885 | 0.44626946 |
| 0.33846604 | 0.33540459 | 0.45389581 |
| 0.37002894 | 0.39807991 | 0.48282395 |
| 0.62558119 | 0.43517553 | 0.50278268 |
| 0.94101683 | 0.43591125 | 0.58858556 |
| 1.08994297 | 0.44115565 | 0.64922975 |
| 2.07889414 | 0.46117658 | 0.71059496 |
| 2.36158936 | 0.47053745 | 0.74330939 |
| 3.29071344 | 0.47200768 | 0.77623126 |
| 3.39229184 | 0.48572734 | 1.34132334 |
| | 0.5223157 | 1.75789523 |
| | 0.52753084 | 1.77426577 |
| | 0.53207364 | 4.31719746 |
| | 0.54685694 | 4.56515688 |
| | 0.63358401 | |
| | 0.70581467 | |
| | 0.73173353 | |
| | 0.81325709 | |
| | 1.33230073 | |
| | 1.46693423 | |
| | 1.75018302 | |
| | 1.80730556 | |
| GM | GM | GM |
| 0.53138737 | 0.20236439 | 0.33378408 |
| 0.60203722 | 0.2071141 | 0.37383059 |
| 0.66705429 | 0.23366778 | 1.40590028 |
| 0.67155886 | 0.26198022 | 1.41686033 |
| IFN | IFN | IFN |
| 0.0722552 | | 0.06975249 |
| 0.07442144 | | 0.07860321 |
| 0.21970779 | | 0.12790808 |
| 0.24065142 | | 0.13459554 |
| | | 0.2443029 |
| | | 0.29138781 |
| TNF | TNF | TNF |
| 0.053116 | 0.04704521 | 0.05621241 |
| 0.05315699 | 0.05220257 | 0.06228325 |
| 0.05331531 | 0.05482446 | 0.10294933 |
| 0.06095267 | 0.05611238 | 0.14351714 |
| 0.14688815 | 0.05757872 | 0.15694653 |
| 0.20004415 | 0.05856579 | 0.16723002 |
| 0.51520632 | 0.0611985 | 0.26908761 |
| 0.51531889 | 0.06890438 | 0.27045659 |
| | 0.0695864 | 0.35271469 |
| | 0.07389075 | 0.37967017 |
| | 0.07988409 | 0.38327461 |
| | 0.0855576 | 0.42359475 |
| | 0.12529323 | |
| | 0.13392305 | |

| IL13 | IL13 | IL13 |
|---|---|---|
| 2.07889414 | 0.46117658 | 0.71059496 |
| 2.36158936 | 0.47053745 | 0.74330939 |
| 3.29071344 | 0.47200768 | 0.77623126 |
| 3.39229184 | 0.48572734 | 1.34132334 |
| | 0.5223157 | 1.75789523 |
| | 0.52753084 | 1.77426577 |
| | 0.53207364 | 4.31719746 |
| | 0.54685694 | 4.56515688 |
| | 0.63358401 | |
| | 0.70581467 | |
| | 0.73173353 | |
| | 0.81325709 | |
| | 1.33230073 | |
| | 1.46693423 | |
| | 1.75018302 | |
| | 1.80730556 | |
| GM | GM | GM |
| 0.53138737 | 0.20236439 | 0.33378408 |
| 0.60203722 | 0.2071141 | 0.37383059 |
| 0.66705429 | 0.23366778 | 1.40590028 |
| 0.67155886 | 0.26198022 | 1.41686033 |
| IFN | IFN | IFN |
| 0.0722552 | | 0.06975249 |
| 0.07442144 | | 0.07860321 |
| 0.21970779 | | 0.12790808 |
| 0.24065142 | | 0.13459554 |
| | | 0.2443029 |
| | | 0.29138781 |
| TNF | TNF | TNF |
| 0.053116 | 0.04704521 | 0.05621241 |
| 0.05315699 | 0.05220257 | 0.06228325 |
| 0.05331531 | 0.05482446 | 0.10294933 |
| 0.06095267 | 0.05611238 | 0.14351714 |
| 0.14688815 | 0.05757872 | 0.15694653 |
| 0.20004415 | 0.05856579 | 0.16723002 |
| 0.51520632 | 0.0611985 | 0.26908761 |
| 0.51531889 | 0.06890438 | 0.27045659 |
| | 0.0695864 | 0.35271469 |
| | 0.07389075 | 0.37967017 |
| | 0.07988409 | 0.38327461 |
| | 0.0855576 | 0.42359475 |
| | 0.12529323 | |
| | 0.13392305 | |

As indicated by the missing values in Table 29 and the different number of measurements in Table 30 for the various cytokines, the cytokine assay did not provide measurements of all nine cytokines for each sample. Many cytokine measurements were not obtained due to one or more interferences with the detection mechanism of the assay. The missing values are attributed to random occurrences of high background, excess heme, lipolysis, desiccation, and the lowest level of quantification (LLOQ) for IL1. However, the missing values for cytokine concentrations occurred randomly among the subjects, and the random occurrence of missing values is believed to not interfere with the accumulation of data. Accordingly the measurements which were obtained from the assay are believed to be meaningful.

Table 30 shows the differences in cytokine levels between the different treatment groups (OXY, BID and QID) in the clinical study. Table 31 the means for each treatment group of the plasma concentrations of the various cytokines, along with the standard deviation for the measurements within the treatment groups. The mean values for the cytokine concentrations detected for each plasma sample analyzed from the various treatment groups is set forth along with the standard deviation. The mean and standard deviation values were calculated using the duplicate values obtained from various plasma samples.

TABLE 31
Mean of Cytokine Concentrations
(ng/ml) from Random Sample of Subjects

| | OXY | BID | QID | | OXY | BID | QID | | OXY | BID | QID |
|---|---|---|---|---|---|---|---|---|---|---|---|
| | IL2 | IL2 | IL2 | | IL4 | IL4 | IL4 | | IL5 | IL5 | IL5 |
| N | 14 | 14 | 14 | N | 8 | 4 | 8 | N | 0 | 0 | 2 |
| Mean | 0.503 | 0.12 | 0.721 | Mean | 0.238 | 0.211 | 0.396 | Mean | | | 0.487 |
| Std Dev | 0.54 | 0.119 | 0.667 | Std Dev | 0.156 | 0.043 | 0.203 | Std Dev | | | 0.001 |
| | OXY | BID | QID | | OXY | BID | QID | | OXY | BID | QID |
| | IL6 | IL6 | IL6 | | IL10 | IL10 | IL10 | | IL13 | IL13 | IL13 |
| N | 14 | 18 | 20 | N | 10 | 8 | 14 | N | 18 | 30 | 22 |
| Mean | 0.2 | 0.128 | 0.189 | Mean | 0.628 | 0.245 | 0.414 | Mean | 0.876 | 0.559 | 0.917 |
| Std Dev | 0.194 | 0.087 | 0.123 | Std Dev | 0.52 | 0.188 | 0.652 | Std Dev | 1.121 | 0.459 | 1.244 |
| | OXY | BID | QID | | OXY | BID | QID | | OXY | BID | QID |
| | GM | GM | GM | | IFN | IFN | IFN | | TNF | TNF | TNF |
| N | 4 | 4 | 4 | N | 4 | 0 | 6 | N | 8 | 14 | 12 |
| Mean | 0.616 | 0.226 | 0.883 | Mean | 0.152 | | 0.158 | Mean | 0.2 | 0.073 | 0.231 |
| Std Dev | 0.066 | 0.027 | 0.611 | Std Dev | 0.091 | | 0.09 | Std Dev | 0.2-2 | 0.026 | 0.132 |

These data indicate that methods and materials as described herein for the treatment of arthritic conditions, inflammation associated with a chronic condition, and/or chronic pain, including pain in conjunction or associated with arthritic conditions or inflammation, are useful to decrease the plasma concentration of various proinflammatory cytokines. These data also indicate that cytokines are appropriate biomarkers, including for the monitoring, detection, diagnosis and/or treatment of arthritic conditions, inflammation associated with a chronic condition and/or chronic pain. Such biomarkers are useful to detect anti-inflammatory activity or other effects of the present methods and materials. Biomarkers, such as cytokines, are of interest to the pharmaceutical industry for various uses, including, for example, to determine potential activity of drugs in clinical development.

### EXAMPLE 5

Solid oral dosage forms comprising opioid agonists and/or opioid antagonists can be prepared by a variety of processes well-known to those skilled in the art. For example, methods and materials as described in U.S. Patent Application Publication No. 2003/0191147 (previously incorporated by reference herein) and WO 01/85257 (PCT/US01/14377) are useful in preparing dosage forms comprising opioid agonists and/or opioid antagonists, including wherein the dosage form comprises amounts of opioid antagonists of 1 mg or less. As another example, solid oral dosage forms comprising oxycodone hydrochloride (OXY) and naltrexone hydrochloride (NTX) are prepared as described herein. For clinical studies as described in Example 1, tablets having different amounts of oxycodone were manufactured, though the amount of naltrexone was the same (0.001 mg) among the tablets of different strength.

Tablet formulations containing oxycodone HCl at various dose levels (2.5, 5, 7.5, 10, 15 and 20 mg/tablet) and low-dose naltrexone HCl (0.001 mg) were prepared. Four matching active controls of oxycodone HCl tablets at various strengths (2.5, 5, 7.5, and 10 mg/tablet) and a matching placebo tablet were also prepared.

A constant weight series based on a common formulation is followed in the manufacture of oxycodone HCl/naltrexone HCl tablets, oxycodcone HCl tablets, and placebo tablets. Differences in the mass of the active pharmaceutical ingredient (API) in the various tablet dosage strengths (in this case oxycodone) are compensated for by adjusting the amount of lactose monohydrate to achieve a consistent mass among all active and placebo tablets.

The components, pharmaceutical grade, and function of each component used to make oxycodone HCl/naltrexone HCl tablets and oxycodone HCl tablets are provided in Table 32 below. Except for the Opadry® film coatings, the components used in the tablet dosage forms are compendial in the current USP/NF.

TABLE 32
Components for Oxycodone HCl/Naltrexone HCl Tablets
and Oxycodone HCl Tablets

| Component | Function |
|---|---|
| Oxycodone HCl, USP | Active pharmaceutical ingredient |
| Naltrexone HCl, USP* | Active pharmaceutical ingredient |
| Lactose, Monohydrate, NF | Diluent |
| Hydroxypropyl Methylcellulose, USP | Binder |
| Citric Acid, Anhydrous, USP | Acidifier for pH adjustment |
| Sodium Hydroxide, NF | Alkalizer for pH adjustment |
| Low-Substituted Hydroxypropyl | Disintegrant |
| Cellulose, NF | |
| Magnesium Stearate, NF | Lubricant |
| Talc, USP (Hydrous Magnesium | Glidant |
| Silicate) | |
| Water** for Injection, USP | Processing Solvent |
| Opadry ® Clear | Base Coat |
| Opadry ® II Yellow | Aesthetic Color Coat |

*Naltrexone HCl not present in oxycodone HCl tablets.
**Removed during processing

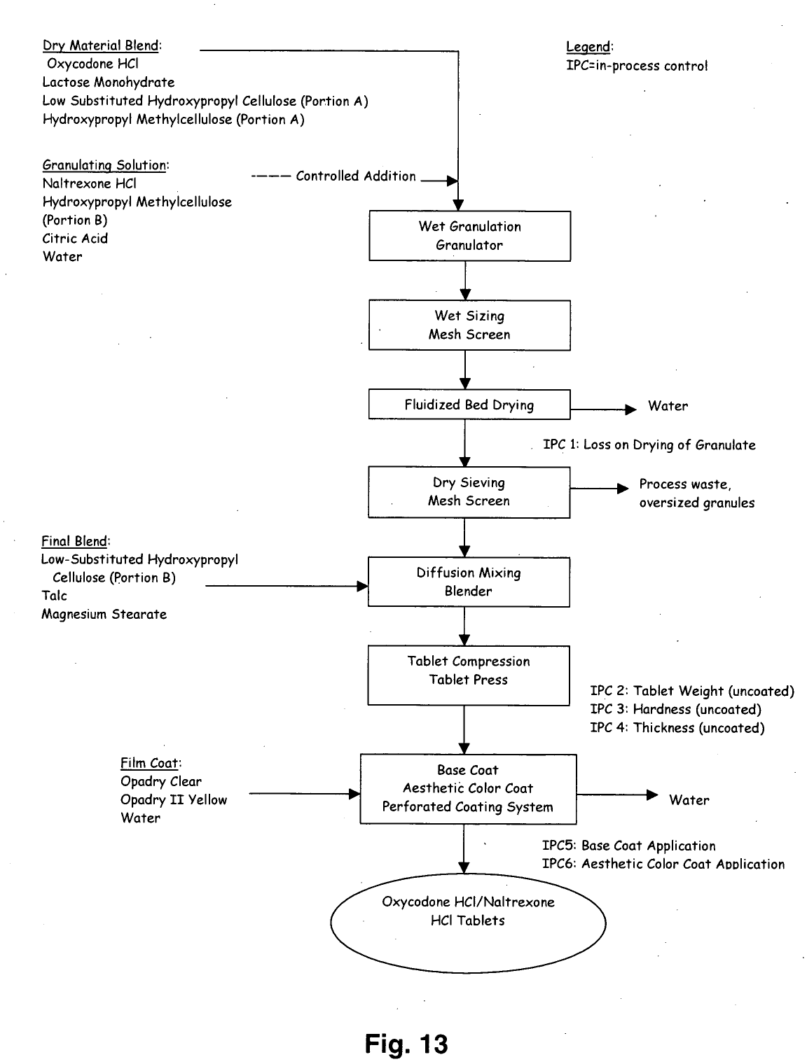

The following steps were used to prepare tablets comprising oxycodone and naltrexone. These steps as well as in-process controls (IPC) are summarized in the flowchart of FIG. 13.

Oxycodone HCl, lactose monohydrate, low-substituted hydroxypropyl cellulose (Portion A), and hydroxypropyl methylcellulose (Portion A) were dry blended in a granulator. This dry material blend was granulated in a wet granulation step with an aqueous solution of naltrexone HCl, citric acid, and hydroxypropyl methylcellulose solution (pH at 3.5) (Portion B). More water was added if needed to obtain a satisfactory granulation. The wet granulation was sieved in a wet sizing step through a mesh screen and dried in a fluidized bed to an endpoint moisture content of not more than 3 percent determined by a Loss on Drying (LOD) measurement.

The dried granulation was sieved through a mesh screen in a dry sieving step. A portion of the dried granulate approximately equal to the balance of formulation components was reserved. The remaining granulate was added to a V-blender.

Each of the three components (low-substituted hydroxypropyl cellulose (Portion B), talc, and magnesium stearate) were combined with an approximately equal portion of the reserved dry granulation to form intermediate mixtures. Each intermediate mixture was sequentially added through a mesh screen and into the V-blender. The granulation was blended after each addition to achieve uniformity.

The blended granulation was compressed into tablets on a rotary tablet press. Tablets had a mean weight of about 200 mg. (approximate range 190 mg to 210 mg), mean hardness in the range of about 5 kp to 8 kp (approximate range 4 kp to 10 kp) and mean thickness of 4.3 to 4.7 mm.

Tablets were film coated in a perforated pan that included application of a clear base coating followed by an aesthetic color coating. A commercially available clear coating (Colorcon-Opadry Clear) was applied to achieve an average coating weight of 2±0.4 mg per tablet. A commercially available color coating (Colorcon-Opadry II Yellow) was applied to achieve an average coating weight of approximately 8±1 mg per tablet.

The amounts of active ingredients and excipients in various tablets of different strengths are set forth in Tables 33 through 38.

Table 33 sets forth the composition of exemplary 2.5 mg strength tablets (tablets comprising 2.5 mg oxycodone HCl and 0.001 mg naltrexone hydrochloride).

TABLE 33
Oxycodone HCl 2.5 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 2.50 | 8.3 |
| Naltrexone HCl, USP | 0.001 | 0.0033** |
| Lactose, Monohydrate, NF | 169.80 | 560.3 |
| Hydroxypropyl Methylcellulose, USP | 2.82 | 9.3** |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium Silicate) | 6.00 | 19.8 |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic Color Coat) | 8.00 | 26.4** |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Table 34 sets forth the composition of exemplary 5 mg strength tablets (tablets comprising 5 mg oxycodone HCl and 0.001 mg naltrexone hydrochloride).

TABLE 34
Oxycodone HCl 5 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 5.00 | 16.5 |
| Naltrexone HCl, USP | 0.001 | 0.0033** |
| Lactose, Monohydrate, NF | 167.30 | 552.1 |
| Hydroxypropyl Methylcellulose, USP | 2.82 | 9.3** |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium Silicate) | 6.00 | 19.8 |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic Color Coat) | 8.00 | 26.4** |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Table 35 sets forth the composition of exemplary 7.5 mg strength tablets (tablets comprising 7.5 mg oxycodone HCl and 0.001 mg naltrexone hydrochloride).

TABLE 35
Oxycodone HCl 7.50 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 7.50 | 24.8 |
| Naltrexone HCl, USP | 0.001 | 0.0033** |
| Lactose, Monohydrate, NF | 164.80 | 543.8 |
| Hydroxypropyl Methylcellulose, USP | 2.82 | 9.3** |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium Silicate) | 6.00 | 19.8 |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic Color Coat) | 8.00 | 26.4** |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Table 36 sets forth the composition of exemplary 10 mg strength tablets (tablets comprising 10 mg oxycodone HCl and 0.001 mg naltrexone hydrochloride).

TABLE 36
Oxycodone HCl 10 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 10.00 | 33.0 |
| Naltrexone HCl, USP | 0.001 | 0.0033** |
| Lactose, Monohydrate, NF | 162.30 | 535.6 |
| Hydroxypropyl Methylcellulose, USP | 2.82 | 9.3** |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium Silicate) | 6.00 | 19.8 |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic Color Coat) | 8.00 | 26.4** |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Table 37 sets forth the composition of exemplary 15 mg strength tablets (tablets comprising 15 mg oxycodone HCl and 0.001 mg naltrexone hydrochloride).

TABLE 37
Oxycodone HCl 15 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 15.00 | 49.5 |
| Naltrexone HCl, USP | 0.001 | 0.0033** |
| Lactose, Monohydrate, NF | 157.30 | 519.1 |
| Hydroxypropyl Methylcellulose, USP | 2.82 | 9.3** |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium Silicate) | 6.00 | 19.8 |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic Color Coat) | 8.00 | 26.4** |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Table 38 sets forth the composition of exemplary 20 mg strength tablets (tablets comprising 20 mg oxycodone HCl and 0.001 mg naltrexone hydrochloride).

TABLE 38
Oxycodone HCl 20 mg/Naltrexone HCl 0.001 mg Tablets

| | Quantity per | Quantity per |
|---|---|---|
| Component | Tablet (mg) | Batch (g) |
| Tablet Core: | | |
| Oxycodone HCl, USP | 20.00 | 66.0 |
| Naltrexone HCl, USP | 0.001 | 0.0033** |
| Lactose, Monohydrate, NF | 152.30 | 502.6 |
| Hydroxypropyl Methylcellulose, USP | 2.82 | 9.3** |
| Citric Acid, Anhydrous, USP | 1.00 | 3.3 |
| Sodium Hydroxide, NF | q.s. to pH* | q.s. to pH* |
| Low-Substituted Hydroxypropyl | 16.88 | 55.7 |
| Cellulose, NF | | |
| Magnesium Stearate, NF | 1.00 | 3.3 |
| Talc, USP (Hydrous Magnesium Silicate) | 6.00 | 19.8 |
| Water for Injection, USP | † | 73.7** |
| Tablet Core Total | 200.00 | 660.0 |
| Color Coating: | | |
| Opadry Clear (Base Coat) | 2.00 | 6.6** |
| Opadry II Yellow (Aesthetic Color Coat) | 8.00 | 26.4** |
| Water for Injection, USP | † | 281.3** |
| Coated Tablet Total | 210.00 | 693.0 |

*For pH adjustment of granulation fluid to pH 3.5 ± 0.2
**Theoretical quantities per batch are tabulated. An overage is prepared in manufacturing to compensate for processing losses (e.g., fluid retention in transport lines and vessels, etc.).
† Removed during processing

Clinical supplies of oxycodone HCl/naltrexone HCl tablets, oxycodone HCl tablets, or placebo tablets were packaged in plastic film blister packs with foil backing. The blister packs were placed inside a sealed foil pouch with a silica gel desiccant to assure that products conform to specifications while in use.

An advantage of dosage forms prepared as referenced and described in this example, including tablets made by the procedure described above and summarized in FIG. 13 is that undesirable binding of the opioid antagonist to the excipients is essentially avoided. It was previously noted that some opioid antagonists undesirably bind significantly to certain pharmaceutical excipients in an environment of use (see, e.g., WO 01/85257 (PCT/US01/14377) and U.S. Patent Application Publication No. 2003/0191147). Undesirable binding generally causes an incomplete amount of the opioid antagonist to be released from a dosage form, within a particular time allotted for release in a dissolution test or in clinical use. For example, as described herein, the use of an acidic pH during the wet granulating step was advantageous with respect to avoiding undesirable binding. As described above, the wet granulation step employed a granulation solution having a pH adjusted to 3.5 with citric acid. The tablets manufactured by this manufacturing process did not exhibit undesirable binding of the opioid antagonist and the excipients to a significant degree. Accordingly, some embodiments of the present methods and materials include steps or excipients which reduce or minimize undesirable binding of opioid antagonist and one or more pharmaceutical excipients, so that such excipients do not bind the opioid antagonist to a significant degree in an environment of use.

While the invention will be described in connection with one or more embodiments, it will be understood that the invention is not limited to those embodiments. On the contrary, the invention includes all alternatives, modification, and equivalents as may be included within the spirit and scope of the appended claims.

### EXAMPLE 6

The effects of an opioid agonist and an opioid antagonist were evaluated in subjects (e.g., patients) with back pain. A clinical study was conducted in patients with moderate to severe chronic low back pain using an exemplary opioid agonist oxycodone alone or with an exemplary opioid antagonist naltrexone, including to evaluate BID and QID dosing combinations of oxycodone and naltrexone, in comparison to oxycodone alone and placebo.

A clinical study was designed to evaluate the efficacy and/or safety of oxycodone and naltrexone when compared to oxycodone alone, to evaluate the efficacy and/or safety of oxycodone and naltrexone when compared to placebo, to compare quality of life measures in patients who received oxycodone and naltrexone to those who received oxycodone alone or placebo, to compare the durability of analgesia of oxycodone and naltrexone compared to oxycodone alone or to placebo, and/or to compare opioid withdrawal rates upon study drug cessation in patients who received combinations and naltrexone to those who received oxycodone alone or placebo.

A multicenter, randomized, double-blind, active- and placebo-controlled study was designed and conducted. The study evaluated the efficacy and/or safety of exemplary oral formulations of oxycodone (OXY) and naltrexone (NTX) compared to oxycodone (OXY) and placebo after a 1- to 6-week titration period with a 12-week fixed-dose period in patients with moderate to severe chronic low back pain. The study was designed to enroll a total of 700 patients: 200 patients each in the oxycodone (OXY) and naltrexone (NTX) BID, oxycodone (OXY) and naltrexone (NTX) QID and oxycodone (OXY) QID treatment groups, and 100 patients in the placebo group.

At the screening visit, patients began a minimum 4-day washout period (maximum 10 days). During the washout period, patients stopped taking all of their pain medications other than acetaminophen (500 mg every 4-6 hours PRN [a maximum of 3000 mg/day]). A daily diary was to be utilized to record overall pain intensity (PI) and the amount of acetaminophen taken each day.

Patients who had taken a daily opioid dose equivalent of oxycodone (>20 mg and ≦160 mg) or tramadol >200 mg during the previous week had an opioid taper. A daily opioid dose equivalent of oxycodone is readily calculated by those skilled in the art. For example, The Drug Conversion Calculator Version 2.0 on American Pain Society's website (http://www.talaria.org/calculatorJ20.html) and/or the PDR Electronic Library, 2002) and/or Goodman and Gilman, supra (see, e.g., Tables 23.6 at page 606 of 10^{th} Edition. An exemplary opioid conversion chart is shown in Table 39.

TABLE 39

| | | | | Daily Quantity | Daily Quantity |
|---|---|---|---|---|---|
| | Daily Oral Dose | | Opioid Content | Equivalent to | Equivalent to |
| | Equivalent to OXY 20; | | Per Unit of | Oral OXY 20 mg/ | Oral OXY 80 mg/ |
| Opioid Compound | 80; 160 mg/day | US Brand Name | Preparation | day | day |
| Codeine Phosphate | 120 mg; 480 mg; 960 mg | Tylenol No. 3 | 30 mg/tablet | 4 tablets | 16 tablets |
| | | Tylenol No. 4 | 60 mg/tablet | 2 tablets | 8 tablets |
| | Other codeine-containing combinations: Tylenol with Codeine Elixir, Phenergan w/Codeine syrup, | | | | |
| | Phenergan VC w/Codeine Syrup, Robitussin A-C syrup, Robitussin-DAC Syrup, Soma Compound w/ | | | | |
| | Codeine Tablets, Tussi-Organdin NR Liquid, Tussi-Organidin-S NR Liquid. | | | | |
| Hydrocodone Bitartrate | 20 mg; 80 mg; 160 mg | Hycodan Syrup | 5 mg/5 mL | 20 mL | 80 mL |
| | | Hycodan Tablet | 5 mL/tablet | 4 tablets | 16 tablets |
| | | Hycomine | 5 mL/tablet | 4 tablets | 16 tablets |
| | | Lortab 2.5/500 | 2.5 mg/tablet | 8 tablets | 32 tablets |
| | | Lortab 5.0/500 | 5.0 mg/tablet | 4 tablets | 16 tablets |
| | | Lortab 7.5/500 | 7.5 mg/tablet | 2.5 tablets | 10 tablets |
| | | Lortab 10.5/500 | 10 mg/tablet | 2 tablets | 8 tablets |
| | | Lortab Elixir | 7.5 mg/15 mL | 40 mL | 160 mL |
| | | Maxidone | 10 mg/tablet | 2 tablets | 8 tablets |
| | | Norco 5/325 | 5 mg/tablet | 4 tablets | 16 tablets |
| | | Norco 7.5/325 | 7.5 mg/tablet | 2.5 tablets | 10 tablets |
| | | Norco 10/325 | 10 mg/tablet | 2 tablets | 8 tablets |
| | | Tussionex | 10 mg/5 mL | 10 mL | 40 mL |
| | | Pennkinetic E-R | | | |
| | | Suspension | | | |
| | | Vicodin | 5 mg/tablet | 4 tablets | 16 tablets |
| | | Vicodin ES | 7.5 mg/tablet | 2.5 tablets | 10 tablets |
| | | Vicodin HP | 10 mg/tablet | 2 tablets | 8 tablets |
| | | Vicodin Tuss | 5 mg/5 mL | 20 mL | 80 mL |
| | | Expectorant | | | |
| | | Vicoprofen | 7.5 mg/tablet | 2.5 tablets | 10 tablets |
| | | Zydone 5/400 | 5 mg/tablet | 4 tablets | 16 tablets |
| | | Zydone 7.5/400 | 7.5 mg/tablet | 2.5 tablets | 10 tablets |
| | | Zydone 10/400 | 10 mg/tablet | 2 tablets | 8 tablets |
| Propoxyphene HCl | 180 mg; 720 mg; | Darvon Pulvule | 65 mg/tablet | 3 tablets | 12 tablets |
| | 1440 mg | | | | |
| | | Darvon | 65 mg/tablet | 3 tablets | 12 tablets |
| | | Compound 65 | | | |
| Propoxyphene Napsylate | 300 mg; 1200 mg; 2400 mg | Darvocet-N 50 | 50 mg/tablet | 6 tablets | 24 tablets |
| | | Darvocet-N 100 | 100 mg/tablet | 3 tablets | 12 tablets |
| | | Darvon-N 100 | 100 mg/tablet | 3 tablets | 12 tablets |
| Oxycodone HCl | 20 mg; 80 mg; 160 mg | OxyContin 10 mg | 10 mg/tablet | 2 tablets | 8 tablets |
| | | OxyContin 20 mg | 20 mg/tablet | 1 tablet | 4 tablets |
| | | OxyContin 40 mg | 40 mg/tablet | Exceeds Dose | 2 tablets |
| | | | | Limit | |
| | | OxyContin 80 mg | 80 mg/tablet | Exceeds Dose | 1 tablet |
| | | | | Limit | |
| | | OxyContin 160 mg | 160 mg/tablet | Exceeds Dose | Exceeds Dose |
| | | | | Limit | Limit |
| | | OxyFast Oral | 20 mg/mL | 1 mL | 4 mL |
| | | Conc Solution | | | |
| | | OxyIR Capsule | 5 mg/capsule | 4 capsules | 16 capsules |
| | | Percocet 2.5/325 | 2.5 mg/tablet | 8 tablets | 32 tablets |
| | | Percocet 5.0/325 | 5.0 mg/tablet | 4 tablets | 16 tablets |
| | | Percocet 7.5/500 | 7.5 mg/tablet | 2.5 tablets | 10 tablets |
| | | Percocet 10/650 | 10 mg/tablet | 2 tablets | 8 tablets |
| | | Percodan | 5 mg/tablet | 4 tablets | 16 tablets |
| | | Percolone | 5 mg/tablet | 4 tablets | 16 tablets |
| | | Roxicodone Oral | 10 mg/10 mL | 20 mL | 80 mL |
| | | Solution | | | |
| | | Roxicodone 15 | 15 mg/tablet | 1 tablet | 5 tablets |
| | | (green) | | | |
| | | Roxicodone 30 | 30 mg/tablet | Exceeds Dose | 2 tablets |
| | | (blue) | | Limit | |
| | | Tylox | 5 mg/capsule | 4 tablets | 16 tablets |
| Meperidine HCl | 300 mg; 1200 mg; 2400 mg | Demerol Syrup | 50 mg/5 mL | 30 mL | 120 mL |
| | | Demoral 50 mg | 50 mg/tablet | 6 tablets | 24 tablets |
| | | Demoral 100 mg | 100 mg/tablet | 3 tablets | 12 tablets |
| Morphine Sulfate | 20 mg; 80 mg; 160 mg | Kadian 20 | 20 mg/tablet | 1 tablets | 4 tablets |
| | | Kadian 20 | 30 mg/tablet | Exceeds Dose | 2 tablets |
| | | | | Limit | |
| | | Kadian 20 | 50 mg/tablet | Exceeds Dose | 1.5 tablets |
| | | | | Limit | |
| | | Kadian 20 | 60 mg/tablet | Exceeds Dose | 1 tablet |
| | | | | Limit | |
| | | Kadian 100 | 100 mg/tablet | Exceeds Dose | Exceeds Dose |
| | | | | Limit | Limit |
| | | MS Contin 15 | 15 mg/tablet | 1 tablets | 5 tablets |
| | | MS Contin 30 | 30 mg/tablet | 1 tablets | 2.5 tablets |
| | | MS Contin 60 | 60 mg/tablet | 1 tablets | 1 tablet |
| | | MS Contin 100, | 100, 200 mg/ | Exceeds Dose | Exceeds Dose |
| | | 200 | tablet | Limit | Limit |
| | | MS IR Oral | 10 mg/5 mL | 10 mL | 40 mL |
| | | Solution | | | |
| | | MS IR Oral | 20 mg/5 mL | 5 mL | 20 mL |
| | | Solution | | | |
| | | MS IR Oral | 20 mg/5 mL | 5 mL | 20 mL |
| | | Solution Conc. | | | |
| | | MS IR 15 | 15 mg/tablet | 1 tablets | 5 tablets/capsule |
| | | | or capsule | | |
| | | MS IR 30 | 30 mg/tablet | Exceeds Dose | 2 tablets/capsule |
| | | | or capsule | Limit | |
| | | Oramorph SR | 15 mg/tablet | 1 tablet | 5 tablets |
| | | tablet 15 | | | |
| | | Oramorph SR | 30 mg/tablet | Exceeds Dose | 2.5 tablets |
| | | tablet 30 | | Limit | |
| | | Oramorph SR | 60 mg/tablet | Exceeds Dose | 1 tablet |
| | | tablet 30 | | Limit | |
| | | Oramorph SR | 100 mg/tablet | Exceeds Dose | Exceeds Dose |
| | | tablet 100 | | Limit | Limit |
| | | Roxanol 100 | 100 mg/5 mL | 1 mL | 4 mL |
| | | Conc Oral | | | |
| | | Solution | | | |
| | | Roxanol Conc | 20 mg/mL | 1 mL | 4 mL |
| | | Oral Solution | | | |
| | | Roxanol-T Oral | 20 mg/mL | 1 mL | 4 mL |
| | | Solution | | | |
| Methadone HCl | 6.7 mg; 26.8 mg; 53.3 mg | Dolophine HCl 5 | 5 mg | 1 tablets | 5 tablets |
| | | Dolophine HCl | 10 mg | Exceeds Dose | 2.5 tablets |
| | | 10 | | Limit | |
| Levorphanol Tatrate | 1.3 mg; 5.2 mg; 10.7 mg | Levo-Dromoran | 2 mg | Exceeds Dose | 2.5 tablets |
| | | | | Limit | |
| Hydromorphone HCl | 5 mg; 20 mg; 40 mg | Dilaudid Oral | 5 mg/5 mL | 1 mL | 4 mL |
| | | Liquid | | | |
| | | Dilaudid Rectal | 3 mg/supp | 1 supp | 6 supp |
| | | Supp | | | |
| | | Dilaudid 2 | 2 mg/tablet | 2 tablets | 10 tablets |
| | | Dilaudid 4 | 4 mg/tablet | 1 tablets | 5 tablets |
| | | (yellow) | | | |
| | | Dilaudid 8 | 8 mg/tablet | Exceeds Dose | 2.5 tablets |
| | | (white) | | Limit | |
| Butorphanol tartrate | 2 mg; 8 mg; 16 mg | Stadol | 1 mg/spray | 2 sprays | 8 sprays |
| Pentazocine HCl | 167 mg; 668 mg; 1336 mg | Talacen | 25 mg/caplet | 6 caplets | 24 caplets |
| | | Talwin Nx | 50 mg/caplet | 3 caplets | 12 caplets |
| Fentanyl | | Duragesic 25 | 25 mcg/h | Exceeds Dose | 1 patch |
| | | | | Limit | |
| | | Duragesic 50, | 50, 75, 100 mcg/h | Exceeds Dose | Exceeds Dose |
| | | 75, 100 | | Limit | Limit |

Reference:
1. Drug Conversion Calculator Version 2.0 on American Pain Society's website: http://www.talaria.org/calculatorJ20.html.
2. PDR Electronic Library, 2002.

Patients were tapered off of their opioid or tramadol medication for a sufficient amount of time to prevent withdrawal according to the investigator's clinical judgment. If necessary, patients could take non-opioid analgesic medication during the opioid taper.

A copy of the “Principles of Analgesic Use in the Treatment of Acute Pain and Cancer Pain”, Fourth Edition, by the American Pain Society (APS) was provided as a reference for the taper procedure. The following is an excerpt from the booklet regarding physical dependence on opioids and a guideline on the use of an opioid taper when discontinuing opioids (page 33):
“Physical dependence is revealed in patients taking chronic opioids when the abrupt discontinuation of an opioid or the administration of an opioid antagonist produces an abstinence syndrome. This syndrome is characterized by anxiety, irritability, chills alternating with hot flashes, salivation, lacrimation, rhinorrhea, diaphoresis, piloerection, nausea, vomiting, abdominal cramps, and insomnia. The time course of this abstinence syndrome is a function of the half-life of the opioid. With short half-life drugs such as morphine or hydromorphone, the symptoms may appear in 6-12 hours and peak at 24-72 hours; with the long half-life drugs methadone and levorphanol, the symptoms may be delayed for several days, and are typically less florid. The abstinence syndrome can be avoided by slowly withdrawing chronically used opioid. Weaning a patient from opioids may proceed as follows: one-half of the previous daily dose may be given in q6 hr doses for the first 2 days and reduced by 25% every 2 days. This schedule is continued until a total daily dose of 30 mg/day of oral morphine in the adult is reached. After 2 days at this minimum dose, the analgesic may be discontinued. An alternative withdrawal schedule consists of switching to oral methadone, using one-fourth of the equianalgesic dose as the initial weaning dose and proceeding as described above. Withdrawal symptoms usually can be avoided by a slow opioid tapering schedule. Anxiety, tachycardia, sweating, and other autonomic symptoms that persist may be lessened by using transdermal clonidine (Catapress TTS) in a patch size delivering 0.1-0.2 mg/day.”

The rate of tapering in patients' opioid medication(s) was to be no faster than the recommendation of the APS guideline. The opioid taper schedule was to be tailored to the individual patient's overall clinical condition and responses to optimize patient safety. Methadone was not to be used for the opioid taper due its long half-life and potential interference with the urine drug screen at the Baseline Visit.

The following assessments were to be conducted prior to the taper: (1) written informed consent; (2) clinic pain intensity (PI); (3) review inclusion and exclusion criteria; (4) detailed medical history including concomitant medications taken one month prior to the Screening Visit; (5) complete physical examination including height, weight and vital signs; and (6) EKG (QTc interval only).

Upon completion of the opioid taper, the following assessments were to be conducted: (1) blood samples for clinical laboratory tests (hematology tests, including hemoglobin, hematocrit, RBC, WBC, neutrophils, lymphocytes, monocytes, esoinophils, basophils and/or platelet count; chemistry tests, including bilirubin, AST, ALT, alkaline phosphatase, GGT, LDH, total protein, albumein, urea, creatinine, calcium, inorganic phosphorus, glucose and/or uric acid); (2) urine sample for urinalysis ((e.g., urine dipstick) including color/appearance, specific gravity, pH, protein, sugar, occult blood, and/or ketones); (3) urine pregnancy test for all women of childbearing potential; (4) obtain up to two acetaminophen bottle numbers and dispense acetaminophen, take-home diary and provide an appointment card for the next visit. The study nurse thoroughly reviewed each section of the diary with the patient. The diary issued at the Screening Visit was to be used to record the following information at bedtime immediately before a dose of acetaminophen was taken; (5) overall pain intensity (PI) in the past 24 hours; and (6) date and number of acetaminophen doses.

Patients who had taken lower daily opioid dose equivalents of oxycodone or tramadol could begin the washout period at the Screening Visit.

At the conclusion of the washout period, patients were randomly assigned to one of the following four treatment groups, if the required PI scores as specified by the study were met, as shown in Table 40.

TABLE 40

| Treatment | |
|---|---|
| Group | Study Drug |
| A | oxycodone (OXY) and naltrexone (NTX) QID |
| B | oxycodone (OXY) QID |
| C | Placebo QID |
| D | oxycodone (OXY) and naltrexone (NTX) BID |

The demographics of the four groups was balanced across the groups as shown in Table 41.

TABLE 41

| | | | Oxycodone | |
|---|---|---|---|---|
| | | | and | |
| | | Oxycodone | Naltrexone | Oxycodone and |
| | Placebo | QID | QID | Naltrexone BID |
| ITT Population | 101 | 206 | 206 | 206 |
| Female | 61.2% | 61.7% | 61.7% | 61.5% |
| Male | 38.8% | 38.3% | 38.3% | 38.5% |
| Age (Mean) | 48.7% | 47.9% | 47.8% | 48.0% |
| Weight (kg) | 84.4 | 85.4 | 90.1 | 85.6 |

Of the 719 subject, 315 had opioid use within one month (30 days) of first dose of study medication: 43 in the placebo group; 99 in the oxycodone QID treatment group; 85 in the oxycodone and naltrexone BID treatment group.

All treatment groups were scheduled for QID dosing to protect the double-blind study design as shown in Table 42.

TABLE 42

| Treatment | QID Daily Dosing Scheme | | | |
|---|---|---|---|---|
| Group | Upon Waking | Noon | Afternoon | Bedtime |
| A | OXY + NTX | OXY + NTX | OXY + NTX | OXY + NTX |
| B | OXY | OXY | OXY | OXY |
| C | Placebo | Placebo | Placebo | Placebo |
| D | OXY + NTX | Placebo | OXY + NTX | Placebo |

The number (percentage) of subjects with opioid use within one month (30 days) of first dose of study medication was in the placebo group 43 of 101 (42.6%), in the oxycodone QID group 99 of 206 (48.1%), in the oxycodone and naltrexone QID group 85 of 206 (41.3%) and in the oxycodone and naltrexone BID group 88 of 206 (42.7%). On a weekly basis, dose was to be escalated according to the titration schedule shown in 43.

TABLE 43

| | Total Daily | | Group B | Group C | |
|---|---|---|---|---|---|
| | Oxycodone | Group A | (OXY) | (Placebo) | Group D |
| Week | Dose (mg) | (OXY + NTX) QID | QID | QID | (OXY + NTX) BID |
| 1 | 10 | OXY 2.5 mg + NTX | 2.5 mg | 0 | OXY 5 mg + NTX 0.001 mg |
| | | 0.001 mg | | | |
| 2 | 20 | OXY 5 mg + NTX 0.001 mg | 5 mg | 0 | OXY 10 mg + NTX |
| | | | | | 0.001 mg |
| 3 | 30 | OXY 7.5 mg + NTX | 7.5 mg | 0 | OXY 15 mg + NTX |
| | | 0.001 mg | | | 0.001 mg |
| 4 | 40 | OXY 10 mg + NTX 0.001 mg | 10 mg | 0 | OXY 20 mg + NTX |
| | | | | | 0.001 mg |
| 5 | 60 | OXY 15 mg + NTX 0.001 mg | 15 mg | 0 | OXY 30 mg + NTX |
| | | | | | 0.001 mg |
| 6 | 80 | OXY 20 mg + NTX 0.001 mg | 20 mg | 0 | OXY 40 mg + NTX |
| | | | | | 0.001 mg |

Dose and adverse events were to be evaluated at each visit. The dose was to be increased to the next dose level if the average daily bedtime PI during the last 3 days of each preceding week was >2 and no unacceptable adverse events were reported. The dose was to be fixed, if the average daily bedtime PI during the last 3 days of each preceding week was ≦2 with no unacceptable adverse events, and the patient was to enter the 12-week fixed-dose period of the study. For these patients, the last week of the titration period was to serve as the first week of the fixed dose period. Therefore, only an additional 11 weeks of treatment were carried out. The same procedure was to occur if a patient had reached 80 mg at Week 6 of the titration period with no unacceptable adverse events.

If any unacceptable study drug related adverse events were encountered, the dose was to be reduced by one level. No more than one dose reduction was to occur. The patient was to remain at this reduced dose for a total of 12 weeks.

For patients experiencing unacceptable adverse events or intolerable pain in between scheduled clinic visits, the dose level was to be decreased or increased during the treatment week with the Sponsor's approval.

Patients were to record their PI every 24 hours in their daily diary immediately before their bedtime dose. In addition, adverse events and opioid-related adverse events were to be recorded in the daily diary.

At the weekly visits (±1 day) during the 1- to 6-week titration period, quality of analgesia and a global assessment of study medication were to be collected. The SF-12 as shown in Table 1 and Oswestry Disability Index (ODI) as shown in Table 44 were to be completed at the end of the titration period (if the titration period was ≧4 weeks).

TABLE 44
Oswestry Disability Index Version 2

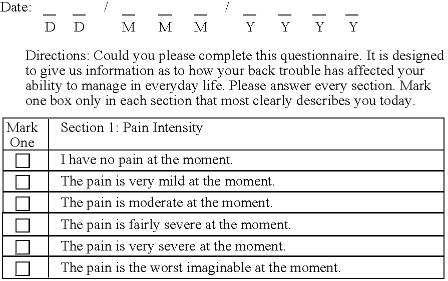

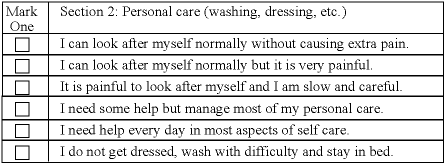

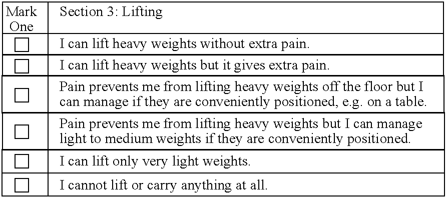

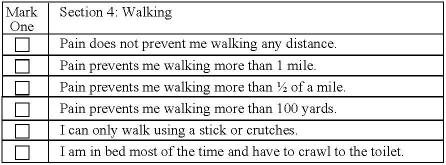

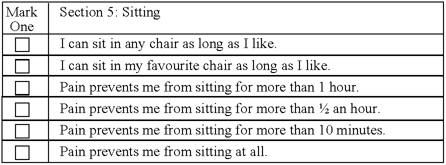

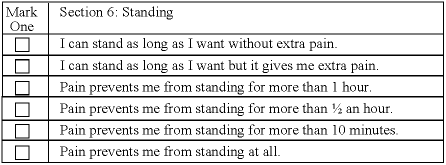

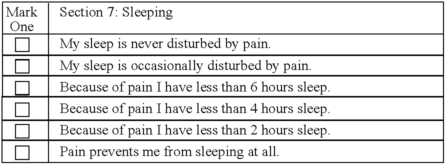

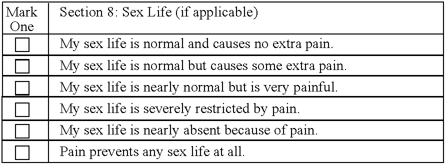

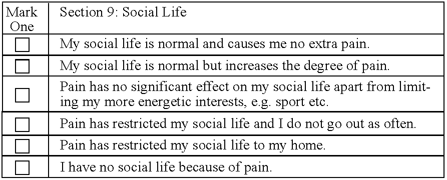

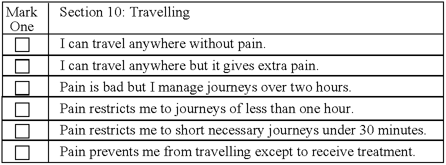

During the fixed-dose period, patients were to return to the clinic each week (±1 day) during the first two weeks and then at 2-week (±2 days) intervals for the remainder of the study. At each clinic visit, quality of analgesia and a global assessment of study medication was to be collected. The SF-12 and ODI were to be performed once every 4 weeks of the fixed-dose period.

Patients were to return for a post-treatment follow-up visit approximately one week (±1 day) after the final dose of study medication. If a patient required additional analgesic medication during the post-treatment follow-up period, he/she was to be allowed to take acetaminophen, NSAIDs, or COX-2 inhibitors. No opioids (or tramadol) were to be allowed during this period.

Safety was evaluated by vital signs (blood pressure, heart rate, respiratory rate and temperature), physical examinations, EKGs, clinical laboratory tests, adverse events including opioid-related adverse events, opioid toxicity assessments and the assessment of opiate withdrawal symptoms using the Short Opiate Withdrawal Scale (SOWS, see Table 6 above).

Approximately seven hundred patients with moderate to severe chronic low back pain were to be enrolled in this study. Inclusion criteria for subjects were as follows:
(1) males and females who are ≧18 and ≦70 years of age;
(2) patient had persistent moderate to severe low back pain for the previous 6 months;
(3) patient had taken one of the following analgesic medications daily during the past 30 days prior to screening: NSAID, COX-2 inhibitor, acetaminophen, tramadol, or opioid;
(4) patient had received either: (a) no opioids or a daily opioid dose equivalent of oxycodone ≦20 mg or tramadol ≦200 mg within one week prior to enrollment into the study; or (b) a daily opioid dose equivalent of oxycodone (>20 mg and ≦160 mg) or tramadol >200 mg within one week prior to enrollment into the study and had undergone an opioid taper period (see above);
(5) patient had a pain intensity score of ≧5 on an 11-point numerical scale at the Screening Visit;
(6) patient had a mean daily diary overall pain intensity (taken immediately before the bedtime dose of acetaminophen) of ≧5 on an 11-point numerical scale during the last 3 days of the washout period and a confirmatory pain intensity level of ≧5 on an 11-point numerical scale measured at the clinic at the Baseline Visit;
(7) patient was able to ambulate at least 100 meters;
(8) females who were postmenopausal, physically incapable of childbearing, or practicing an acceptable method of birth control. Acceptable methods of birth control include hormonal contraceptives or double-barrier methods (condom or diaphragm with a spermicidal agent or IUD). If practicing an acceptable method of birth control, a negative urine pregnancy test result had been obtained at the Screening Visit;
(9) patient agreed to refrain from taking any pain medications other than study drug during the washout period and the entire treatment period. [Aspirin (up to 325 mg/day) was permitted for cardiovascular prophylaxis if at a stable dose one month prior to the Screening Visit.]; and
(10) patient was able to understand and cooperate with study procedures, and has signed an informed consent form prior to entering the study.

Exclusion Criteria for subjects were as follows:
(1) patient had a positive urine drug screen at the Baseline Visit (Start of Titration Period, Week 1, Day 1) NOT caused by any therapeutic medication permitted during the study;
(2) patient had low back pain secondary to malignancy, inflammatory disease (rheumatic arthritis, ankylosing spondylitis, Reiter's syndrome), fibromyalgia, vertebral fracture, or infection;
(3) patient had major surgery within three months prior to the Screening Visit or had surgery planned during the proposed study period;
(4) patient had received oral or parenteral corticosteroid therapy within one month prior to the Screening Visit;
(5) patient had received intraspinal infusion of any medication or implanted spinal cord stimulator within one month prior to the Screening Visit or had plans for such treatment during proposed study period;
(6) patient had undergone any percutaneous (i.e. discogram, epidural injection, discectomy, IDET, etc.) or open (laminectomy, discectomy, fusion, instrumentation, etc.) procedures involving the lumbosacral spine within four months prior to the Screening Visit or had any such procedures planned during the proposed study period;
(7) patient was involved in an unsettled claim such as automobile accident, civil lawsuit, or worker's compensation pertaining to a low back injury, however, patients with settled claims were able to participate;
(8) patient weighed more than 300 lbs or less than 100 lbs;
(9) patient was pregnant or breast-feeding;
(10) patient had a history of severe hepatic or renal impairment;
(11) patient had a history of cardiac disease (such as coronary artery disease, cardiomyopathy, congestive heart disease, valvular disease, arrythmia, etc.), angina, cerebral aneurysm, cerebral vascular accident (CVA), transient ischemic event (TIA) or inadequately controlled hypertension;
(12) patient had a known allergy or significant reaction to any of the study medications: oxycodone, naltrexone, or acetaminophen;
(13) patient had severe impairment of pulmonary function, hypercarbia, hypoxia, significant chronic obstructive airways disease or cor pulmonale, acute or severe bronchial asthma, sleep apnea syndrome or respiratory depression;
(14) patient had or was suspected of having paralytic ileus, acute abdomen (serious abdominal pain requiring emergency surgery), or delayed gastric emptying;
(15) patient had a history of gastric, biliary, or small intestine surgery, or any other diseases that cause clinical malabsorption;
(16) patient has chronic biliary tract disease, chronic pancreatitis, or inflammatory bowel disorders;
(17) patient had untreated myxedema, untreated hypothyroidism, elevated intracranial pressure, severe anemia, adrenocortical insufficiency, hypotension or hypovolemia;
(18) patient had started or stopped monoamine oxidase inhibitors, tricyclic antidepressant drugs, serotonin reuptake inhibitors, glucosamine/chondroitin, or St. John's Wort within four weeks prior to receiving study medication, however, a constant dose for longer than four weeks was acceptable;
(19) patient had started or stopped physical therapy, transcutaneous electrical nerve stimulation, chiropractic, osteopathic, acupuncture, or other complementary treatment within the past four weeks or was expected to undergo any changes in these therapies in the duration of the study;
(20) patient was receiving high doses of sedatives, hypnotics or tranquilizers;
(21) patient was receiving phenothiazines or other agents that compromise vasomotor tone;
(22) patient had a history of alcohol or drug abuse within the past 5 years;
(23) patient had a psychiatric illness or medical illness/condition that, in the opinion of the investigator, would interfere with the completion of study, confound the results of the study, or pose risk to the patients;
(24) patient had a history of the following neoplastic disease: leukemia, lymphoma, or myeloproliferative disease, metastatic cancer, however, in patients with treated localized malignancies, the decision to exclude was made by the Sponsor on individual cases;
(25) patient had AST, ALT, GGT, or alkaline phosphatase >2 times the upper limit of normal; hematocrit <30%; or creatinine >1.8 at the Screening Visit;
(26) patient had previously received the oxycodone and naltrexone study drug;
(27) patient had participated in another investigational drug trial or therapeutic trial within 30 days of the Screening Visit; or
(28) patient had taken analgesic medication (other than acetaminophen—a maximum of 3000 mg/day) during the minimum 4-day washout period prior to randomization.

The physical descriptions of the drugs used for the study were as follows. Up to 2 containers of acetaminophen were dispensed at the Screening Visit for the minimum 4-day washout period (maximum 10 days). A commercially available source of acetaminophen (500 mg) was supplied. The investigational drug supplies were available in tablet dosage forms containing oxycodone HCl and naltrexone HCl, oxycodone HCl, or placebo. All of the tablet dosage forms were indistinguishable from one another to facilitate blinding.

The study procedures were as follows. Prior to any study-related activities, the patient provided written informed consent. The Investigator explained the study (characteristics of the drug substances, required procedures, potential hazards, and possible adverse reactions) to all prospective participants.

At the first visit (Screening Visit), pre-enrollment screening was performed. Patients who had taken a daily opioid dose equivalent of oxycodone (>20 mg and ≦160 mg) or tramadol >200 mg during the previous week were to have an opioid taper as described above. Patients were tapered off of their opioid or tramadol medication for a sufficient amount of time to prevent withdrawal according to the investigator's clinical judgment and assessments were performed as described above. Patients who had taken lower daily opioid dose equivalents of oxycodone or tramadol did not require a taper and could enroll in the study at the Screening Visit.

The following assessments were conducted at the Screening Visit: (1) written informed consent; (2) clinic pain intensity (PI); (3) review inclusion and exclusion criteria; (4) detailed medical history including concomitant medications taken one month prior to the Screening Visit; (5) complete physical examination including height, weight and vital signs; (6) EKG (QTc interval only); (7) blood samples for clinical laboratory tests (hematology and chemistry tests as described above); (8) urine sample for urinalysis; (9) urine pregnancy test for all women of childbearing potential; and (10) obtain up to two acetaminophen bottle numbers and dispense acetaminophen and take-home diary. The study nurse thoroughly reviewed each section of the diary with the patient. The diary issued at the Screening Visit was to be used by the patient to record the following information at bedtime immediately before a dose of acetaminophen is taken: (1) overall PI in the past 24 hours and (2) date and number of acetaminophen doses.

The second visit was to establish a baseline and the start of the titration period (Week 1, day 1). The patient returned to the study center a minimum of four days (maximum of ten days) after the Screening Visit for completion of the pre-dose assessments. These included: (1) urine sample for drug screening using a rapid drug screen kit (e.g., BioChek iCup™ Drug Screen), if this was positive for any drugs not caused by any therapeutic medication permitted during the study, no further assessments were done and the patient did not continue in the study); (2) review of take-home diary; (3) collect bottle(s) of acetaminophen and perform accountability; (4) baseline clinic PI rating; and (5) review inclusion and exclusion criteria and verify that: (a) the mean daily overall pain intensity score collected in the diary during the last 3 days of the washout period was ≧5 (on a scale of 0 to 10) while off all analgesic medications (except acetaminophen as directed); and (b) the clinic PI at this visit measured ≧5 (on a scale of 0 to 10); and (c) from the Screening Visit, clinical laboratory tests results were without significant clinical abnormalities; the urine pregnancy test was negative (if required).

Patients meeting the study entry criteria were randomly assigned to one of the four treatment groups, as described in Table 40 above, and were assigned a randomization number and study medication kit number. The following assessments were then conducted: (1) interim medical history (to identify any changes from the Screening Visit); (2) vital signs; (3) SF-12 Health Survey; (4) Oswestry Disability Index (ODI); and (5) review and record concomitant medications. Once these assessments and procedures were completed, study medication (one blister card) was dispensed for Week 1 of the titration period. A take-home daily diary was dispensed and an appointment card for the next visit was provided. The study nurse reviewed each section of the diary with the patient. The daily diaries issued at each visit were used to record the following information: (1) overall PI in the past 24 hours at bedtime immediately prior to dosing; and (2) adverse events (including an assessment of opioid-related adverse events).

During the titration period (Weeks 2-6, Day 1), patients returned to the study center at weekly intervals (±1 day) for up to six weeks for the following assessments: (1) opioid toxicity assessment; (2) review take-home diary (including overall daily bedtime PI and opioid-related adverse events); (3) record new/changed adverse events and concomitant medications; (4) collect study medication from previous week and account for used/unused supplies; (5) vital signs; (6) quality of analgesia; (7) global assessment of study medication; (8) dispense take-home daily diary; and (8) dispense study medication (one blister card). Dose and adverse events were evaluated at each visit. The dose was to be increased to the next dose level if the average daily bedtime PI during the last 3 days of each preceding week was >2 and no unacceptable adverse events were reported. The dose was to be fixed, if the average daily bedtime PI during the last 3 days of each preceding week was ≦2 with no unacceptable adverse events, and the patient entered the fixed-dose period of the study. For these patients, the last week of the titration period served as the first week of the fixed dose period. Therefore, only an additional 11 weeks of treatment were required. The same procedure occurred if a patient had reached 80 mg at Week 6 of the titration period with no unacceptable adverse events. If any unacceptable study drug related adverse events were encountered, the dose was to be reduced by one level. No more than one dose reduction can occur. The patient was to remain at this reduced dose for a total of 12 weeks. For patients experiencing unacceptable adverse events or intolerable pain in between scheduled clinic visits, the dose level was to be decreased or increased during the treatment week with the Sponsor's approval.

At the end of the titration period (if the titration period was ≧4 weeks), the following assessments were performed in addition to the above-described weekly assessments: (1) blood samples for clinical laboratory tests (hematology and chemistry tests as described above); (2) urine sample for urinalysis as described above; (3) SF-12 Health Survey; and (4) ODI. Patients were to remain at the last titrated dose for a total of 12 weeks. No dose adjustments were allowed during this period of the study.

Patients returned to the study center at Weeks 1 (if applicable) and 2 (±1 day) of the fixed-dose period for the following assessments: (1) opioid toxicity assessment; (2) review take-home diary (including overall daily bedtime PI and opioid-related adverse events); (3) record new/changed adverse events and concomitant medications; (4) collect study medication from previous visit and account for used/unused supplies; (5) vital signs; (6) quality of analgesia; (7) global assessment of study medication; (8) dispense take-home daily diary; (9) dispense study medication (two blister cards were dispensed on Week 2, Day 8 of the fixed-dose period and patients instructed to switch to the second blister card after taking 7 days of study drug from the first card).

Patients returned to the study center at Weeks 4, 6, 8, and 10 (±2 days) of the fixed-dose period for the following assessments: (1) opioid toxicity assessment; (2) review take-home diary (including overall daily bedtime PI and opioid-related adverse events); (3) record new/changed adverse events and concomitant medications; (4) collect study medication from previous visit and account for used/unused supplies; (5) vital signs; (6) quality of analgesia; (7) global assessment of study medication; (8) SF-12 Health Survey (only at Weeks 4 & 8 of the fixed-dose period); (9) ODI (only at Weeks 4 & 8 of the fixed-dose period); (10) dispense take-home daily diary; and dispense study medication (two blister cards dispensed and patients instructed to switch to the second blister card after taking 7 days of study drug from the first card).

Patients returned to the study center at the end of Week 12 (±2 days) of the fixed-dose period (end-of-treatment) for the following assessments: (1) review take-home diary (including overall daily bedtime PI and opioid-related adverse events); (2) record new/changed adverse events and concomitant medications; (3) collect study medication and account for used/unused supplies; (4) complete physical examination and vital signs; (5) EKG (QTc interval only); (6) blood samples for clinical laboratory tests (hematology and chemistry tests as described above); (7) urine sample for urinalysis as described above; (8) quality of analgesia; (9) global assessment of study medication; (10) SF-12 Health Survey; (11) ODI; (12) SOWS; and (13) dispense take-home daily diary (SOWS and adverse event log) for follow-up period and instruct patients to contact the study center immediately if they experience severe signs and symptoms of opioid withdrawal.

The study center contacted patients (telephone visits) before noon once daily (for four days after the last dose of study medication) to monitor for symptoms of opioid withdrawal. On each telephone call, the study center was to verify that the SOWS have been completed each day (in the morning) by the patients. In addition, there was to be a check for adverse events and concomitant medications. If necessary, a clinic visit could be required for those patients with clinically significant withdrawal symptoms.

Patients returned to the study center approximately one week (±1 day) after the last dose of study medication for a post-treatment follow-up visit. At this visit, the following assessments were completed: (1) review take-home diary; and (2) record new/changed adverse events and concomitant medications.

Patients could choose to discontinue study drug or study participation at any time, for any reason, and without prejudice. If a patient chose to discontinue study drug early, the investigator would request that the patient return to the clinic within 24 hours of stopping the study medication and complete the end-of-treatment assessments described above. For patients who had been on study medication for ≧4 weeks, Day 1 of the opioid withdrawal monitoring period was to begin 24 hours after the last dose of study medication. The investigator also requested that the patient remain in the study for the post-treatment follow-up visit. Study drug assigned to patients who discontinued the study early was not reassigned to another patient.

Efficacy assessments included: (1) pain intensity (PI), (2) quality of analgesia and/or (3) global assessment of study medication.

For the pain intensity (PI) scale assessment, the patient was prompted with the question “How would you rate your overall pain intensity at this time?” and the PI score was recorded in the clinic at the Screening and Baseline Visits. Pain intensity was also assessed by prompting the patient with the questions “How would you rate your overall pain intensity during the past 24 hours?” and a daily PI diary score was recorded at bedtime. For both Pain Intensity prompts, the response was scored on an 11-point numerical scale (0=no pain and 10=severe pain).

Quality of analgesia was assessed at clinic visits during the titration and fixed-dose periods. The patient was prompted with the question, “How would you rate the quality of your pain relief at this time?” and responses were selected from poor, fair, good, very good, and excellent.

Global assessment of study medication was also assessed at clinic visits during the titration and fixed-dose periods. The patient was prompted with the question, “How would you rate the study medication you received during the past week/past two weeks? (Please consider the quality of your pain relief, your side effects, your activity level, your mood and sense of well-being, etc. in this evaluation.)” Responses were selected from poor, fair, good, very good, and excellent.

Additionally, functional assessments (at baseline, at the end of the titration period (if the titration period is ≧4 weeks), at Weeks 4 and 8 of the fixed-dose period, and at Week 12 of the fixed-dose period (i.e., End-of-Treatment)) were conducted with SF-12 (Table 1) and ODI (Table 44).

Clinical examinations consisted of the standard-of-care evaluations that are routinely performed as part of ongoing care for patients with moderate to severe low back pain. Safety assessments included: (1) vital signs (blood pressure, heart rate, respiratory rate, and temperature); (2) physical examinations; (3) EKGs; (4) clinical laboratory tests; (5) adverse events; (6) assessment of opiate withdrawal symptoms using the SOWS; and (7) opioid toxicity assessments. Opioid toxicity assessments were performed during the titration period to evaluate dose escalation and the fixed-dose period to evaluate continuation of the study medication at the current dose level. The assessments included a review of the following: (A) CNS review by assessing for (1) confusion, altered mental state, (2) excessive drowsiness, lethargy, stupor, and (3) slurred speech (new onset); (B) respiratory review by assessing for (1) hypoventilation, shortness of breath, apnea and (2) decreased respiratory rate (<8) or cyanosis; and (C) cardiac review by assessing for heart rate <60, hypotension. If patients were terminated from the study, the end-of-treatment assessments and opioid withdrawal monitoring (as needed) were completed.

The randomization schedule was computer generated using a permuted block algorithm and randomly allocated study medication to randomization numbers. The randomization numbers were assigned sequentially through a central system as patients were entered into the study. The randomization schedule was stratified by patient sex. Investigative site was not a blocking factor in the randomization schedule. When three treatment groups were under study, patients were assigned to the groups in a ratio of 2:2:1. When four treatment groups were under study, after ˜50 patients had been enrolled in the three groups studied initially (˜20 patients each in the oxycodone and naltrexone QID and oxycodone QID arms and ˜10 patients in the placebo arm), patients were assigned to the groups in a ratio of 2:2:1:3 for some blocks and a ratio of 2:2:1:2 for other blocks. The larger size blocks were spread evenly throughout the smaller size blocks. Varying the treatment assignment ratio and block sizes throughout the randomization schedule accommodated for 20 oxycodone and naltrexone BID patients not enrolled initially and ensured that approximately equal numbers of patients were enrolled in the oxycodone and naltrexone BID, oxycodone and naltrexone QID and oxycodone QID groups at the conclusion of the enrollment period.

The primary analysis population was the intent-to-treat (ITT) population. The ITT population consisted of all patients who take study medication and was used for both efficacy and safety analyses. In the event that a patient was randomized incorrectly or was administered the incorrect study medication, the patient was analyzed according to the study drug actually received.

Demographic variables and baseline patient characteristics were summarized descriptively by treatment group and overall. Demographic variables included age, weight, height, gender, and ethnicity while baseline patient characteristics included baseline pain intensity, baseline SF-12, and baseline Oswestry Disability Index. Study drug administration, prior medications and concomitant medications were also summarized.

The following endpoints were summarized and analyzed within both dosing periods (titration and fixed-dose): (1) daily pain intensity ratings; (2) quality of analgesia; (3) global assessment of study medication; (4) SF-12; and (5) Oswestry Disability Index.

The baseline average daily pain intensity score for each patient was calculated as the average of the daily bedtime pain intensity scores recorded during the 3 days immediately prior to baseline/start of titration period visit. The post-baseline average daily pain intensity scores were calculated as the average of the daily bedtime pain intensity scores recorded during the last 3 days of dosing prior to each visit. Missing data was imputed using the last-observation-carried-forward (LOCF) approach for efficacy variables in the fixed-dose period. If the first visit in the fixed-dose period was missing, the last value in the titration period was imputed forward. Unless indicated, all testing of statistical significance was two-sided, and a difference resulting in a p-value of equal to or less than 0.05 was considered statistically significant.

A primary efficacy endpoint was the percent change from baseline to the end of the fixed-dose period (Week 12) in average daily pain intensity. The percent change from baseline was calculated as [PI(baseline)−PI(fixed-dose)]/PI(baseline)*100. Data was summarized descriptively by treatment group. An analysis of covariance (ANCOVA) model including treatment and center as effects and baseline pain intensity as the covariate was used for global and pairwise inferences. Separate models were used to evaluate both the treatment by center and treatment by baseline pain intensity interaction terms.

A primary comparison of interest was oxycodone and naltrexone (e.g., BID or QID) versus oxycodone (e.g., QID). Secondary analyses included actual and percent change from baseline values of daily pain index, SF-12, and Oswestry disability index summarized descriptively by treatment group for both the titration and fixed-dose periods. Treatments were compared globally and in pairwise fashion at each visit in the fixed-dose period using an ANCOVA model that included treatment and center as effects and baseline pain intensity as the covariate. Separate models were used to evaluate both the treatment by center and treatment by baseline pain intensity interaction terms.

Global assessment and quality of analgesia ratings were summarized descriptively by treatment group for both the titration and fixed-dose periods. Treatments were compared at each visit in the fixed-dose period, globally and in pairwise fashion, using the Cochran-Mantel-Haenszel row mean scores (CMH-RMS) test, using equally spaced scores.

Adverse events reported on case report forms were mapped to preferred terms and body systems using the MedDRA coding dictionary. The number and percent of patients reporting each event were summarized by treatment group and dosing period. Incidence of adverse events by maximum reported severity was also tabulated.

Treatment groups were examined for differences in the incidence (e.g., frequency), severity and/or duration (e.g., daily or over a period of drug treatment) of selected opioid-associated adverse events including constipation, dizziness, somnolence, pruritis, nausea and vomiting.

Change from baseline (either the Screening Visit or the Start of Titration Period Visit, whichever occurs later and has data present) were summarized descriptively for vital signs and QTc interval. Laboratory data were summarized descriptively on the original scale, change from baseline (Screening Visit), and in terms of the normal range.

Each assessment of opiate withdrawal symptoms using SOWS on Days 1 through 4 of opioid withdrawal monitoring were reduced to an average system score and were summarized in terms of changes from baseline, which was defined as the in-clinic assessment at the end of treatment visit (Week 12 of the fixed-dose period).

A primary efficacy endpoint was the percent change from baseline to the end of the fixed-dose period (Week 12) in average daily pain intensity. A primary comparison of interest was oxycodone and naltrexone (e.g., BID or QID) versus oxycodone (e.g., QID).

It was expected that there would be at least a 40% drop out rate in the study before the 12 week fixed-dose period concluded. With this high drop out rate, along with clinical considerations, it was estimated that enrolling 200 patients each in the oxycodone and naltrexone BID, oxycodone and naltrexone QID and oxycodone treatment groups, and 100 patients in the placebo group would be sufficient to detect clinically meaningful differences between treatments and to provide adequate safety and exposure data.

From this clinical study, various assessments were made, including a pain assessment (e.g., pain intensity), a quality of analgesia assessment, a global assessment, a functional assessment (e.g., using the Oswestry Disability Index (ODI) and/or SF-12 Health Survey), an assessment of withdrawal symptoms (e.g., using the Short Opioid Withdrawal Scales (SOWS)), as well as assessments of average titration doses (e.g., for the titration period), average fixed doses (e.g., for the fixed-dose period), and average study doses (e.g., for the entire study period including titration and fixed-dose period) for the treatment groups.

One efficacy assessment was a pain assessment. Pain intensity scores or ratings were measured for this study as described above and reported as actual values, as well as changes from baseline, and percent changes from baseline to fixed dose, for baseline as shown in Table 45A and each week of Weeks 1-12 of the fixed-dose period as shown in Tables 45B through -45KK. For example, Tables 45B and 45JJ show the actual values for pain intensity at Week 1 and Week 12, respectively, of the fixed-dose period; Tables 45C and 45JJ show the changes in pain intensity from baseline to Week 1 and Week 12, respectively, of the fixed-dose period; and Tables 45D and 45KK show the percent changes in pain intensity from baseline to Week 1 and Week 12, respectively, of the fixed-dose period. An efficacy endpoint for this study was percent change from baseline to the end of the fixed-dose period (Week 12) in average daily pain intensity as shown in Table 45KK. Reductions in mean PI occurred at each week of the fixed-dose period in all active treatment groups as compared to placebo, and the difference in pain intensity of each active treatment group versus placebo was statistically significant at each of Weeks 1-12 of the fixed-dose period. There were no statistically significant differences between active treatment groups (e.g., when one active treatment group was compared to another active treatment group).

TABLE 45A
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| BASELINE | | | | | |
| MEAN (SD) | 7.7 (1.44) | 7.6 (1.36) | 7.3 (1.36) | 7.6 (1.33) | 7.5 (1.37) |
| MEDIAN | 7.7 | 7.7 | 7.3 | 7.7 | 7.3 |
| MINIMUM (MAXIMUM) | 5.0 (10.0) | 5.0 (10.0) | 4.7 (10.0) | 4.7 (10.0) | 4.7 (10.0) |
| N | 101 | 206 | 206 | 206 | 719 |
| MODEL P-VALUES | | | | | |
| TREATMENT [1] | 0.020 | | | | |
| SEX [1] | <0.001 | | | | |
| TREATMENT*SEX [2] | 0.805 | | | | |
| PAIRWISE COMPARISON P-VALUES [1] | | | | | |
| PLACEBO QID | — | 0.444 | 0.008 | 0.510 | |
| OXY QID | — | — | 0.021 | 0.894 | |
| OXY + NTX QID | — | — | — | 0.015 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT AND SEX AS A BLOCKING FACTOR.
[2] P-VALUES FROM ANCOVA MODEL DEFINED IN [1] INCLUDING TREATMENT*SEX INTERACTION TERMS.

TABLE 45B
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE | | | | | |
| WEEK 1 | | | | | |
| MEAN (SD) | 5.4 (2.87) | 3.9 (2.53) | 4.1 (2.51) | 4.2 (2.55) | 4.3 (2.62) |
| MEDIAN | 5.5 | 3.7 | 4.0 | 3.7 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | <0.001 | | | | |
| SEX [1] | 0.620 | | | | |
| BASELINE PI [1] | <0.001 | | | | |
| TREATMENT*BASELINE PI [2] | 0.068 | | | | |
| TREATMENT*SEX [2] | 0.319 | | | | |
| PAIRWISE COMPARISON P-VALUES [1] | | | | | |
| PLACEBO QID | — | <0.001 | <0.001 | <0.001 | |
| OXY QID | — | — | 0.189 | 0.337 | |
| OXY + NTX QID | — | — | — | 0.716 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45C
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 1 | | | | | |
| MEAN (SD) | −2.3 (2.60) | −3.6 (2.60) | −3.1 (2.61) | −3.4 (2.65) | −3.2 (2.65) |
| MEDIAN | −1.7 | −3.7 | −3.3 | −3.3 | −3.3 |
| MINIMUM (MAXIMUM) | −9.0 (4.3) | −9.7 (3.0) | −8.3 (2.3) | −8.7 (2.3) | −9.7 (4.3) |
| N | 101 | 205 | 199 | 204 | 709 |
| MODEL P-VALUES | | | | | |
| TREATMENT [1] | <0.001 | | | | |
| SEX [1] | 0.620 | | | | |
| BASELINE PI [1] | <0.001 | | | | |
| TREATMENT*BASELINE PI [2] | 0.068 | | | | |
| TREATMENT*SEX [2] | 0.319 | | | | |
| PAIRWISE COMPARISON P-VALUES [1] | | | | | |
| PLACEBO QID | — | <0.001 | <0.001 | <0.001 | |
| OXY QID | — | — | 0.189 | 0.337 | |
| OXY + NTX QID | — | — | — | 0.716 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45D
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED | | | | | |
| DOSE WEEK 1 | | | | | |
| MEAN (SD) | −29.8 (35.84) | −47.5 (33.12) | −42.4 (34.71) | −44.1 (33.32) | −42.5 (34.40) |
| MEDIAN | −23.8 | −52.4 | −47.8 | −52.5 | −47.6 |
| MINIMUM (MAXIMUM) | −100.0 (86.7) | −100.0 (42.9) | −100.0 (41.2) | −100.0 (35.0) | −100.0 (86.7) |
| N | 101 | 205 | 199 | 204 | 709 |
| MODEL P-VALUES | | | | | |
| TREATMENT [1] | <0.001 | | | | |
| SEX [1] | 0.891 | | | | |
| BASELINE PI [1] | 0.026 | | | | |
| TREATMENT*BASELINE PI [2] | 0.611 | | | | |
| TREATMENT*SEX [2] | 0.238 | | | | |
| PAIRWISE COMPARISON | | | | | |
| P-VALUES [1] | | | | | |
| PLACEBO QID | — | <0.001 | 0.001 | <0.001 | |
| OXY QID | — | — | 0.198 | 0.305 | |
| OXY + NTX QID | — | — | — | 0.786 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45E
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE WEEK 2 | | | | | |
| MEAN (SD) | 5.4 (2.87) | 3.9 (2.47) | 4.1 (2.45) | 4.3 (2.60) | 4.3 (2.60) |
| MEDIAN | 5.0 | 3.7 | 3.7 | 4.0 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | <0.001 | | | | |
| SEX [1] | 0.804 | | | | |
| BASELINE PI [1] | <0.001 | | | | |
| TREATMENT*BASELINE PI [2] | 0.035 | | | | |
| TREATMENT*SEX [2] | 0.656 | | | | |
| PAIRWISE COMPARISON P-VALUES [1] | | | | | |
| PLACEBO QID | — | <0.001 | <0.001 | 0.001 | |
| OXY QID | — | — | 0.317 | 0.133 | |
| OXY + NTX QID | — | — | — | 0.627 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45F
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 2 | | | | | |
| MEAN (SD) | −2.3 (2.60) | −3.6 (2.59) | −3.2 (2.60) | −3.3 (2.71) | −3.2 (2.65) |
| MEDIAN | −2.0 | −3.7 | −3.3 | −3.3 | −3.3 |
| MINIMUM (MAXIMUM) | −8.7 (4.3) | −9.7 (3.0) | 10.0 (2.3) | −8.7 (2.3) | −10.0 (4.3) |
| N | 101 | 205 | 199 | 204 | 709 |
| MODEL P-VALUES | | | | | |
| TREATMENT [1] | <0.001 | | | | |
| SEX [1] | 0.804 | | | | |
| BASELINE PI [1] | <0.001 | | | | |
| TREATMENT*BASELINE PI [2] | 0.035 | | | | |
| TREATMENT*SEX [2] | 0.656 | | | | |
| PAIRWISE COMPARISON P-VALUES [1] | | | | | |
| PLACEBO QID | — | <0.001 | <0.001 | 0.001 | |
| OXY QID | — | — | 0.317 | 0.133 | |
| OXY + NTX QID | — | — | — | 0.627 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45G
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED | | | | | |
| DOSE WEEK 2 | | | | | |
| MEAN (SD) | −30.5 (35.85) | −47.1 (32.56) | −42.9 (33.92) | −42.0 (34.16) | −42.1 (34.21) |
| MEDIAN | −25.0 | −50.0 | −50.0 | −47.0 | −47.1 |
| MINIMUM (MAXIMUM) | −100.0 (86.7) | −100.0 (42.9) | −100.0 (41.2) | −100.0 (35.0) | −100.0 (86.7) |
| N | 101 | 205 | 199 | 204 | 709 |
| MODEL P-VALUES | | | | | |
| TREATMENT [1] | <0.001 | | | | |
| SEX [1] | 0.976 | | | | |
| BASELINE PI [1] | 0.004 | | | | |
| TREATMENT*BASELINE PI [2] | 0.458 | | | | |
| TREATMENT*SEX [2] | 0.507 | | | | |
| PAIRWISE COMPARISON | | | | | |
| P-VALUES [1] | | | | | |
| PLACEBO QID | — | <0.001 | <0.001 | 0.004 | |
| OXY QID | — | — | 0.337 | 0.126 | |
| OXY + NTX QID | — | — | — | 0.580 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45H
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE WEEK 3 | | | | | |
| MEAN (SD) | 5.3 (2.91) | 3.9 (2.52) | 4.1 (2.49) | 4.3 (2.59) | 4.3 (2.62) |
| MEDIAN | 5.0 | 3.3 | 3.7 | 4.0 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | <0.001 | | | | |
| SEX [1] | 0.539 | | | | |
| BASELINE PI [1] | <0.001 | | | | |
| TREATMENT*BASELINE PI [2] | 0.040 | | | | |
| TREATMENT*SEX [2] | 0.612 | | | | |
| PAIRWISE COMPARISON P-VALUES [1] | | | | | |
| PLACEBO QID | — | <0.001 | <0.001 | 0.001 | |
| OXY QID | — | — | 0.182 | 0.128 | |
| OXY + NTX QID | — | — | — | 0.864 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45I
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 3 | | | | | |
| MEAN (SD) | −2.4 (2.66) | −3.7 (2.64) | −3.1 (2.64) | −3.3 (2.73) | −3.2 (2.70) |
| MEDIAN | −2.0 | −4.0 | −3.3 | −3.3 | −3.3 |
| MINIMUM (MAXIMUM) | −9.0 (4.3) | −9.7 (3.0) | −10.0 (2.3) | −9.0 (2.3) | −10.0 (4.3) |
| N | 101 | 205 | 199 | 204 | 709 |
| MODEL P-VALUES | | | | | |
| TREATMENT [1] | <0.001 | | | | |
| SEX [1] | 0.539 | | | | |
| BASELINE PI [1] | <0.001 | | | | |
| TREATMENT*BASELINE PI [2] | 0.040 | | | | |
| TREATMENT*SEX [2] | 0.612 | | | | |
| PAIRWISE COMPARISON P-VALUES [1] | | | | | |
| PLACEBO QID | — | <0.001 | <0.001 | 0.001 | |
| OXY QID | — | — | 0.182 | 0.128 | |
| OXY + NTX QID | — | — | — | 0.864 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45J
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM BASELINE | | | | | |
| TO FIXED DOSE WEEK 3 | | | | | |
| MEAN (SD) | −30.8 (36.34) | −47.9 (33.29) | −42.4 (34.42) | −42.6 (34.14) | −42.14 (34.63) |
| MEDIAN | −25.0 | −54.2 | −47.6 | −47.6 | −47.6 |
| MINIMUM (MAXIMUM) | −100.0 (86.7) | −100.0 (42.9) | −100.0 (41.2) | −100.0 (35.0) | −100.0 (86.7) |
| N | 101 | 205 | 199 | 204 | 709 |
| MODEL P-VALUES | | | | | |
| TREATMENT [1] | <0.001 | | | | |
| SEX [1] | 0.756 | | | | |
| BASELINE PI [1] | 0.003 | | | | |
| TREATMENT*BASELINE PI [2] | 0.424 | | | | |
| TREATMENT*SEX [2] | 0.471 | | | | |
| PAIRWISE COMPARISON | | | | | |
| P-VALUES [1] | | | | | |
| PLACEBO QID | — | <0.001 | 0.002 | 0.004 | |
| OXY QID | — | — | 0.180 | 0.112 | |
| OXY + NTX QID | — | — | — | 0.819 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45K
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXEDDOSE WEEK 4 | | | | | |
| MEAN (SD) | 5.3 (2.94) | 3.9 (2.55) | 4.1 (2.53) | 4.2 (2.55) | 4.3 (2.63) |
| MEDIAN | 5.3 | 3.3 | 3.7 | 4.0 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | <0.001 | | | | |
| SEX [1] | 0.444 | | | | |
| BASELINE PI [1] | <0.001 | | | | |
| TREATMENT*BASELINE PI [2] | 0.051 | | | | |
| TREATMENT*SEX [2] | 0.355 | | | | |
| PAIRWISE COMPARISON P-VALUES [1] | | | | | |
| PLACEBO QID | — | <0.001 | 0.003 | 0.002 | |
| OXY QID | — | — | 0.158 | 0.211 | |
| OXY + NTX QID | — | — | — | 0.863 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45L
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 4 | | | | | |
| MEAN (SD) | −2.4 (2.70) | −3.6 (2.68) | −3.1 (2.67) | −3.3 (2.69) | −3.2 (2.71) |
| MEDIAN | −2.0 | −4.0 | −3.0 | −3.3 | −3.3 |
| MINIMUM (MAXIMUM) | −8.3 (4.3) | −9.7 (3.0) | −10.0 (3.0) | −8.7 (2.3) | −10.0 (4.3) |
| N | 101 | 205 | 199 | 204 | 709 |
| MODEL P-VALUES | | | | | |
| TREATMENT [1] | <0.001 | | | | |
| SEX [1] | 0.444 | | | | |
| BASELINE PI [1] | <0.001 | | | | |
| TREATMENT*BASELINE PI [2] | 0.051 | | | | |
| TREATMENT*SEX [2] | 0.355 | | | | |
| PAIRWISE COMPARISON P-VALUES [1] | | | | | |
| PLACEBO QID | — | <0.001 | 0.003 | 0.002 | |
| OXY QID | — | — | 0.158 | 0.211 | |
| OXY + NTX QID | — | — | — | 0.863 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45M
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED | | | | | |
| DOSE WEEK 4 | | | | | |
| MEAN (SD) | −31.6 (37.07) | −47.4 (33.84) | −41.6 (34.91) | −43.1 (33.65) | −42.3 (34.84) |
| MEDIAN | −25.0 | −53.3 | −45.5 | −48.9 | −47.1 |
| MINIMUM (MAXIMUM) | −100.0 (86.7) | −100.0 (42.9) | −100.0 (42.9) | −100.0 (35.0) | −100.0 (86.7) |
| N | 101 | 205 | 199 | 204 | 709 |
| MODEL P-VALUES | | | | | |
| TREATMENT [1] | 0.002 | | | | |
| SEX [1] | 0.613 | | | | |
| BASELINE PI [1] | 0.003 | | | | |
| TREATMENT*BASELINE PI [2] | 0.506 | | | | |
| TREATMENT*SEX [2] | 0.262 | | | | |
| PAIRWISE COMPARISON | | | | | |
| P-VALUES [1] | | | | | |
| PLACEBO QID | — | <0.001 | 0.007 | 0.005 | |
| OXY QID | — | — | 0.160 | 0.193 | |
| OXY + NTX QID | — | — | — | 0.908 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45N
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUALVALUE AT FIXED DOSE WEEK 5 | | | | | |
| MEAN (SD) | 5.3 (2.91) | 3.9 (2.59) | 4.1 (2.54) | 4.1 (2.55) | 4.2 (2.64) |
| MEDIAN | 5.0 | 3.3 | 4.0 | 3.7 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | <0.001 | | | | |
| SEX [1] | 0.244 | | | | |
| BASELINE PI [1] | <0.001 | | | | |
| TREATMENT*BASELINE PI [2] | 0.040 | | | | |
| TREATMENT*SEX [2] | 0.286 | | | | |
| PAIRWISE COMPARISON P-VALUES [1] | | | | | |
| PLACEBO QID | — | <0.001 | 0.002 | <0.001 | |
| OXY QID | — | — | 0.217 | 0.393 | |
| OXY + NTX QID | — | — | — | 0.697 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45O
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 5 | | | | | |
| MEAN (SD) | −2.4 (2.66) | −3.6 (2.72) | −3.1 (2.70) | −3.4 (2.68) | −3.3 (2.71) |
| MEDIAN | −2.0 | −4.0 | −3.0 | −3.3 | −3.3 |
| MINIMUM (MAXIMUM) | −7.3 (4.3) | −9.7 (3.0) | −10.0 (2.3) | −8.7 (2.3) | −10.0 (4.3) |
| N | 101 | 205 | 199 | 204 | 709 |
| MODEL P-VALUES | | | | | |
| TREATMENT [1] | <0.001 | | | | |
| SEX [1] | 0.244 | | | | |
| BASELINE PI [1] | <0.001 | | | | |
| TREATMENT*BASELINE PI [2] | 0.040 | | | | |
| TREATMENT*SEX [2] | 0.286 | | | | |
| PAIRWISE COMPARISON P-VALUES [1] | | | | | |
| PLACEBO QID | — | <0.001 | 0.002 | <0.001 | |
| OXY QID | — | — | 0.217 | 0.393 | |
| OXY + NTX QID | — | — | — | 0.697 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45P
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED | | | | | |
| DOSE WEEK 5 | | | | | |
| MEAN (SD) | −31.6 (36.63) | −47.4 (34.44) | −42.2 (34.87) | −44.4 (33.49) | −42.8 (34.90) |
| MEDIAN | −25.0 | −52.4 | −45.5 | −51.0 | −47.4 |
| MINIMUM (MAXIMUM) | −100.0 (86.7) | −100.0 (42.9) | −100.0 (41.2) | −100.0 (35.0) | −100.0 (86.7) |
| N | 101 | 205 | 199 | 204 | 709 |
| MODEL P-VALUES | | | | | |
| TREATMENT [1] | 0.002 | | | | |
| SEX [1] | 0.411 | | | | |
| BASELINE PI [1] | 0.004 | | | | |
| TREATMENT*BASELINE PI [2] | 0.459 | | | | |
| TREATMENT*SEX [2] | 0.186 | | | | |
| PAIRWISE COMPARISON | | | | | |
| P-VALUES [1] | | | | | |
| PLACEBO QID | — | <0.001 | 0.005 | 0.002 | |
| OXY QID | — | — | 0.219 | 0.373 | |
| OXY + NTX QID | — | — | — | 0.728 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45Q
PAIN INTENSITY - BY WEEK
LAST OBSERVATION CARRIED FORWARD IMPUTATION
ANALYSIS POPULATION: INTENT TO TREAT POPULATION

| | | | | OXY + NTX | |
|---|---|---|---|---|---|
| | PLACEBO QID | OXY QID | OXY + NTX QID | BID | TOTAL |
| | (N = 101) | (N = 206) | (N = 206) | (N = 206) | (N = 719) |
| ACTUAL VALUE AT FIXED DOSE | | | | | |
| WEEK 6 | | | | | |
| MEAN (SD) | 5.2 (3.03) | 3.9 (2.58) | 4.1 (2.57) | 4.1 (2.56) | 4.2 (2.67) |
| MEDIAN | 5.0 | 3.3 | 4.0 | 3.8 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | 0.002 | | | | |
| SEX [1] | 0.354 | | | | |
| BASELINE PI [1] | <0.001 | | | | |
| TREATMENT*BASELINE PI [2] | 0.032 | | | | |
| TREATMENT*SEX [2] | 0.427 | | | | |
| PAIRWISE COMPARISON P-VALUES [1] | | | | | |
| PLACEBO QID | — | <0.001 | 0.007 | 0.002 | |
| OXY QID | — | — | 0.174 | 0.404 | |
| OXY + NTX QID | — | — | — | 0.594 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45R
PAIN INTENSITY - BY WEEK
LAST OBSERVATION CARRIED FORWARD IMPUTATION
ANALYSIS POPULATION: INTENT TO TREAT POPULATION

| | | | | OXY + NTX | |
|---|---|---|---|---|---|
| | PLACEBO QID | OXY QID | OXY + NTX QID | BID | TOTAL |
| | (N = 101) | (N = 206) | (N = 206) | (N = 206) | (N = 719) |
| CHANGE FROM BASELINE TO FIXED | | | | | |
| DOSE WEEK 6 | | | | | |
| MEAN (SD) | −2.5 (2.79) | −3.6 (2.70) | −3.1 (2.76) | −3.4 (2.70) | −3.3 (2.75) |
| MEDIAN | −1.7 | −4.0 | −3.3 | −3.6 | −3.3 |
| MINIMUM (MAXIMUM) | −8.7 (4.3) | −9.7 (3.0) | −10.0 (2.3) | −8.7 (2.7) | −10.0 (4.3) |
| N | 101 | 205 | 199 | 204 | 709 |
| MODEL P-VALUES | | | | | |
| TREATMENT [1] | 0.002 | | | | |
| SEX [1] | 0.354 | | | | |
| BASELINE PI [1] | <0.001 | | | | |
| TREATMENT*BASELINE PI [2] | 0.032 | | | | |
| TREATMENT*SEX [2] | 0.427 | | | | |
| PAIRWISE COMPARISON P-VALUES [1] | | | | | |
| PLACEBO QID | — | <0.001 | 0.007 | 0.002 | |
| OXY QID | — | — | 0.174 | 0.404 | |
| OXY + NTX QID | — | — | — | 0.594 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45S
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED | | | | | |
| DOSE WEEK 6 | | | | | |
| MEAN (SD) | −32.8 (37.98) | −47.5 (34.20) | −41.6 (35.81) | −44.6 (33.82) | −42.9 (35.34) |
| MEDIAN | −25.0 | −51.9 | −45.5 | −50.0 | −47.6 |
| MINIMUM (MAXIMUM) | −100.0 (86.7) | −100.0 (42.9) | −100.0 (41.2) | −100.0 (36.4) | −100.0 (86.7) |
| N | 101 | 205 | 199 | 204 | 709 |
| MODEL P-VALUES | | | | | |
| TREATMENT [1] | 0.005 | | | | |
| SEX [1] | 0.488 | | | | |
| BASELINE PI [1] | 0.003 | | | | |
| TREATMENT*BASELINE PI [2] | 0.370 | | | | |
| TREATMENT*SEX [2] | 0.362 | | | | |
| PAIRWISE COMPARISON | | | | | |
| P-VALUES [1] | | | | | |
| PLACEBO QID | — | <0.001 | 0.018 | 0.005 | |
| OXY QID | — | — | 0.159 | 0.380 | |
| OXY + NTX QID | — | — | — | 0.590 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45T
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE | | | | | |
| WEEK 7 | | | | | |
| MEAN (SD) | 5.2 (2.99) | 4.0 (2.54) | 4.1 (2.53) | 4.2 (2.53) | 4.2 (2.63) |
| MEDIAN | 5.0 | 3.3 | 3.7 | 4.0 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | 0.001 | | | | |
| SEX [1] | 0.305 | | | | |
| BASELINE PI [1] | <0.001 | | | | |
| TREATMENT*BASELINE PI [2] | 0.043 | | | | |
| TREATMENT*SEX [2] | 0.461 | | | | |
| PAIRWISE COMPARISON P-VALUES [1] | | | | | |
| PLACEBO QID | — | <0.001 | 0.003 | 0.002 | |
| OXY QID | — | — | 0.325 | 0.385 | |
| OXY + NTX QID | — | — | — | 0.899 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45U
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 7 | | | | | |
| MEAN (SD) | −2.5 (2.77) | −3.6 (2.69) | −3.1 (2.71) | −3.4 (2.67) | −3.2 (2.72) |
| MEDIAN | −2.0 | −4.0 | −3.3 | −3.3 | −3.3 |
| MINIMUM (MAXIMUM) | −9.0 (4.3) | −9.7 (3.0) | −10.0 (2.3) | −8.7 (2.3) | −10.0 (4.3) |
| N | 101 | 205 | 199 | 204 | 709 |
| MODEL P-VALUES | | | | | |
| TREATMENT [1] | 0.001 | | | | |
| SEX [1] | 0.305 | | | | |
| BASELINE PI [1] | <0.001 | | | | |
| TREATMENT*BASELINE PI [2] | 0.043 | | | | |
| TREATMENT*SEX [2] | 0.461 | | | | |
| PAIRWISE COMPARISON P-VALUES [1] | | | | | |
| PLACEBO QID | — | <0.001 | 0.003 | 0.002 | |
| OXY QID | — | — | 0.325 | 0.385 | |
| OXY + NTX QID | — | — | — | 0.899 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45V
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED DOSE | | | | | |
| WEEK 7 | | | | | |
| MEAN (SD) | −32.2 (37.42) | −46.8 (34.05) | −42.4 (35.02) | −43.9 (33.48) | −42.6 (34.89) |
| MEDIAN | −25.0 | −52.9 | −46.4 | −50.0 | −48.0 |
| MINIMUM (MAXIMUM) | −100.0 (86.7) | −100.0 (42.9) | −100.0 (41.2) | −100.0 (35.0) | −100.0 (86.7) |
| N | 101 | 205 | 199 | 204 | 709 |
| MODEL P-VALUES | | | | | |
| TREATMENT [1] | 0.004 | | | | |
| SEX [1] | 0.460 | | | | |
| BASELINE PI [1] | 0.001 | | | | |
| TREATMENT*BASELINE PI [2] | 0.451 | | | | |
| TREATMENT*SEX [2] | 0.384 | | | | |
| PAIRWISE COMPARISON | | | | | |
| P-VALUES [1] | | | | | |
| PLACEBO QID | — | <0.001 | 0.006 | 0.004 | |
| OXY QID | — | — | 0.331 | 0.380 | |
| OXY + NTX QID | — | — | — | 0.919 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45W
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE | | | | | |
| WEEK 8 | | | | | |
| MEAN (SD) | 5.2 (3.01) | 4.0 (2.57) | 4.1 (2.56) | 4.2 (2.52) | 4.3 (2.64) |
| MEDIAN | 5.0 | 3.3 | 3.7 | 4.0 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | 0.002 | | | | |
| SEX [1] | 0.319 | | | | |
| BASELINE PI [1] | <0.001 | | | | |
| TREATMENT*BASELINE PI [2] | 0.057 | | | | |
| TREATMENT*SEX [2] | 0.535 | | | | |
| PAIRWISE COMPARISON P-VALUES [1] | | | | | |
| PLACEBO QID | — | <0.001 | 0.003 | 0.002 | |
| OXY QID | — | — | 0.339 | 0.388 | |
| OXY + NTX QID | — | — | — | 0.919 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS ACOVARIATE.
[2] P-VALUES FROM ANCOVA MODEL DEFINED IN [1] INCLUDING TREATMENT*BASELINE PI AND TREATMENT*SEX INTERACTION TERMS.

TABLE 45X
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 8 | | | | | |
| MEAN (SD) | −2.5 (2.80) | −3.6 (2.67) | −3.1 (2.76) | −3.4 (2.65) | −3.2 (2.73) |
| MEDIAN | −1.7 | −3.7 | −3.0 | −3.3 | −3.3 |
| MINIMUM (MAXIMUM) | −9.0 (4.3) | −9.7 (3.0) | −10.0 (2.3) | −9.0 (2.7) | −10.0 (4.3) |
| N | 101 | 205 | 199 | 204 | 709 |
| MODEL P-VALUES | | | | | |
| TREATMENT [1] | 0.002 | | | | |
| SEX [1] | 0.319 | | | | |
| BASELINE PI [1] | <0.001 | | | | |
| TREATMENT*BASELINE PI [2] | 0.057 | | | | |
| TREATMENT*SEX [2] | 0.535 | | | | |
| PAIRWISE COMPARISON P-VALUES [1] | | | | | |
| PLACEBO QID | — | <0.001 | 0.003 | 0.002 | |
| OXY QID | — | — | 0.339 | 0.388 | |
| OXY + NTX QID | — | — | — | 0.919 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45Y
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED DOSE | | | | | |
| WEEK 8 | | | | | |
| MEAN (SD) | −31.9 (37.78) | −46.5 (34.05) | −41.9 (35.56) | −43.5 (33.50) | −42.3 (35.10) |
| MEDIAN | −25.0 | −52.4 | −45.5 | −49.1 | −47.1 |
| MINIMUM (MAXIMUM) | −100.0 (86.7) | −100.0 (42.9) | −100.0 (41.2) | −100.0 (36.4) | −100.0 (86.7) |
| N | 101 | 205 | 199 | 204 | 709 |
| MODEL P-VALUES | | | | | |
| TREATMENT [1] | 0.005 | | | | |
| SEX [1] | 0.461 | | | | |
| BASELINE PI [1] | 0.001 | | | | |
| TREATMENT*BASELINE PI [2] | 0.429 | | | | |
| TREATMENT*SEX [2] | 0.429 | | | | |
| PAIRWISE COMPARISON | | | | | |
| P-VALUES [1] | | | | | |
| PLACEBO QID | — | <0.001 | 0.007 | 0.005 | |
| OXY QID | — | — | 0.301 | 0.365 | |
| OXY + NTX QID | — | — | — | 0.890 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45Z
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE | | | | | |
| WEEK 9 | | | | | |
| MEAN (SD) | 5.3 (2.95) | 3.9 (2.57) | 4.1 (2.52) | 4.2 (2.55) | 4.3 (2.64) |
| MEDIAN | 5.3 | 3.3 | 3.7 | 4.0 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | <0.001 | | | | |
| SEX [1] | 0.301 | | | | |
| BASELINE PI [1] | <0.001 | | | | |
| TREATMENT*BASELINE PI [2] | 0.093 | | | | |
| TREATMENT*SEX [2] | 0.719 | | | | |
| PAIRWISE COMPARISON P-VALUES [1] | | | | | |
| PLACEBO QID | — | <0.001 | 0.002 | <0.001 | |
| OXY QID | — | — | 0.185 | 0.260 | |
| OXY + NTX QID | — | — | — | 0.834 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45AA
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 9 | | | | | |
| MEAN (SD) | −2.4 (2.75) | −3.6 (2.70) | −3.1 (2.68) | −3.4 (2.69) | −3.2 (2.72) |
| MEDIAN | −1.7 | −4.0 | −3.0 | −3.3 | −3.3 |
| MINIMUM (MAXIMUM) | −8.7 (4.3) | −9.7 (3.0) | −10.0 (2.3) | −9.7 (2.3) | −10.0 (4.3) |
| N | 101 | 205 | 199 | 204 | 709 |
| MODEL P-VALUES | | | | | |
| TREATMENT [1] | <0.001 | | | | |
| SEX [1] | 0.301 | | | | |
| BASELINE PI [1] | <0.001 | | | | |
| TREATMENT*BASELINE PI [2] | 0.093 | | | | |
| TREATMENT*SEX [2] | 0.719 | | | | |
| PAIRWISE COMPARISON P-VALUES [1] | | | | | |
| PLACEBO QID | — | <0.001 | 0.002 | <0.001 | |
| OXY QID | — | — | 0.185 | 0.260 | |
| OXY + NTX QID | — | — | — | 0.834 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45BB
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED DOSE | | | | | |
| WEEK 9 | | | | | |
| MEAN (SD) | −30.8 (37.26) | −47.4 (34.17) | −41.8 (34.59) | −43.5 (33.71) | −42.3 (34.92) |
| MEDIAN | −22.2 | −53.3 | −46.7 | −50.0 | −47.4 |
| MINIMUM (MAXIMUM) | −100.0 (86.7) | −100.0 (42.9) | −100.0 (41.2) | −100.0 (35.0) | −100.0 (86.7) |
| N | 101 | 205 | 199 | 204 | 709 |
| MODEL P-VALUES | | | | | |
| TREATMENT [1] | <0.001 | | | | |
| SEX [1] | 0.441 | | | | |
| BASELINE PI [1] | 0.002 | | | | |
| TREATMENT*BASELINE PI [2] | 0.667 | | | | |
| TREATMENT*SEX [2] | 0.630 | | | | |
| PAIRWISE COMPARISON | | | | | |
| P-VALUES [1] | | | | | |
| PLACEBO QID | — | <0.001 | 0.003 | 0.002 | |
| OXY QID | — | — | 0.188 | 0.248 | |
| OXY + NTX QID | — | — | — | 0.863 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45CC
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N2 = 206) | (N = 206) | (N = 206) | (N = 719) |
| ACTUAL VALUE AT FIXED DOSE | | | | | |
| WEEK 10 | | | | | |
| MEAN (SD) | 5.2 (3.01) | 4.0 (2.54) | 4.2 (2.54) | 4.3 (2.57) | 4.3 (2.65) |
| MEDIAN | 5.3 | 3.7 | 3.7 | 4.0 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | 0.002 | | | | |
| SEX [1] | 0.310 | | | | |
| BASELINE PI [1] | <0.001 | | | | |
| TREATMENT*BASELINE PI [2] | 0.076 | | | | |
| TREATMENT*SEX [2] | 0.671 | | | | |
| PAIRWISE COMPARISON P-VALUES [1] | | | | | |
| PLACEBO QID | — | <0.001 | 0.005 | 0.004 | |
| OXY QID | — | — | 0.198 | 0.232 | |
| OXY+ NTX QID | — | — | — | 0.917 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS A COVARIATE.
[2] P-VALUES FROM ANCOVA MODEL DEFINED IN [1] INCLUDING TREATMENT*BASELINE PI AND TREATMENT*SEX INTERACTION TERMS.

TABLE 45DD
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 10 | | | | | |
| MEAN (SD) | −2.5 (2.79) | −3.6 (2.66) | −3.1 (2.70) | −3.3 (2.68) | −3.2 (2.71) |
| MEDIAN | −1.7 | −3.7 | −3.0 | −3.2 | −3.3 |
| MINIMUM (MAXIMUM) | −9.0 (4.3) | −9.7 (3.0) | −10.0 (2.3) | −9.7 (2.7) | −10.0 (4.3) |
| N | 101 | 205 | 199 | 204 | 709 |
| MODEL P-VALUES | | | | | |
| TREATMENT [1] | 0.002 | | | | |
| SEX [1] | 0.310 | | | | |
| BASELINE PI [1] | <0.001 | | | | |
| TREATMENT*BASELINE PI [2] | 0.076 | | | | |
| TREATMENT*SEX [2] | 0.671 | | | | |
| PAIRWISE COMPARISON P-VALUES [1] | | | | | |
| PLACEBO QID | — | <0.001 | 0.005 | 0.004 | |
| OXY QID | — | — | 0.198 | 0.232 | |
| OXY + NTX QID | — | — | — | 0.917 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS ACOVARIATE.
[2] P-VALUES FROM ANCOVA MODEL DEFINED IN [1] INCLUDING TREATMENT*BASELINE PI AND TREATMENT*SEX INTERACTION TERMS.

TABLE 45EE
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED DOSE | | | | | |
| WEEK 10 | | | | | |
| MEAN (SD) | −32.0 (37.74) | −46.8 (33.72) | −41.4 (34.86) | −42.8 (33.81) | −42.0 (34.90) |
| MEDIAN | −25.0 | −52.0 | −45.8 | −46.9 | −46.2 |
| MINIMUM (MAXIMUM) | −100.0 (86.7) | −100.0 (42.9) | −100.0 (41.2) | −100.0 (36.4) | −100.0 (86.7) |
| N | 101 | 205 | 199 | 204 | 709 |
| MODEL P-VALUES | | | | | |
| TREATMENT [1] | 0.004 | | | | |
| SEX [1] | 0.461 | | | | |
| BASELINE PI [1] | 0.004 | | | | |
| TREATMENT*BASELINE PI [2] | 0.535 | | | | |
| TREATMENT*SEX [2] | 0.604 | | | | |
| PAIRWISE COMPARISON | | | | | |
| P-VALUES [1] | | | | | |
| PLACEBO QID | — | <0.001 | 0.012 | 0.009 | |
| OXY QID | — | — | 0.186 | 0.222 | |
| OXY + NTX QID | — | — | — | 0.910 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS ACOVARIATE.
[2] P-VALUES FROM ANCOVA MODEL DEFINED IN [1] INCLUDING TREATMENT*BASELINE PI AND TREATMENT*SEX INTERACTION TERMS.

TABLE 45FF
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE | | | | | |
| WEEK 11 | | | | | |
| MEAN (SD) | 5.3 (3.01) | 3.9 (2.57) | 4.2 (2.57) | 4.4 (2.59) | 4.3 (2.68) |
| MEDIAN | 5.3 | 3.3 | 4.0 | 4.0 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | <0.001 | | | | |
| SEX [1] | 0.490 | | | | |
| BASELINE PI [1] | <0.001 | | | | |
| TREATMENT*BASELINE PI [2] | 0.061 | | | | |
| TREATMENT*SEX [2] | 0.749 | | | | |
| PAIRWISE COMPARISON P-VALUES [1] | | | | | |
| PLACEBO QID | — | <0.001 | 0.004 | 0.005 | |
| OXY QID | — | — | 0.085 | 0.055 | |
| OXY + NTX QID | — | — | — | 0.858 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS ACOVARIATE.
[2] P-VALUES FROM ANCOVA MODEL DEFINED IN [1] INCLUDING TREATMENT*BASELINE PI AND TREATMENT*SEX INTERACTION TERMS.

TABLE 45GG
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 11 | | | | | |
| MEAN (SD) | −2.4 (2.76) | −3.7 (2.70) | −3.1 (2.72) | −3.2 (2.69) | −3.2 (2.74) |
| MEDIAN | −1.7 | −4.0 | −3.0 | −3.0 | −3.0 |
| MINIMUM (MAXIMUM) | −8.7 (4.3) | −9.7 (3.0) | −10.0 (2.3) | −8.7 (2.7) | −10.0 (4.3) |
| N | 101 | 205 | 199 | 204 | 709 |
| MODEL P-VALUES | | | | | |
| TREATMENT [1] | <0.001 | | | | |
| SEX [1] | 0.490 | | | | |
| BASELINE PI [1] | <0.001 | | | | |
| TREATMENT*BASELINE PI [2] | 0.061 | | | | |
| TREATMENT*SEX [2] | 0.749 | | | | |
| PAIRWISE COMPARISON P-VALUES [1] | | | | | |
| PLACEBO QID | — | <0.001 | 0.004 | 0.005 | |
| OXY QID | — | — | 0.085 | 0.055 | |
| OXY + NTX QID | — | — | — | 0.858 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS ACOVARIATE.
[2] P-VALUES FROM ANCOVA MODEL DEFINED IN [1] INCLUDING TREATMENT*BASELINE PI AND TREATMENT*SEX INTERACTION TERMS.

TABLE 45HH
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED DOSE | | | | | |
| WEEK 11 | | | | | |
| MEAN (SD) | −31.3 (37.74) | −48.2 (34.19) | −41.4 (35.33) | −41.8 (34.11) | −42.0 (35.33) |
| MEDIAN | −24.1 | −55.6 | −43.8 | −43.9 | −45.0 |
| MINIMUM (MAXIMUM) | −100.0 (86.7) | −100.0 (42.9) | −100.0 (41.2) | −100.0 (40.0) | −100.0 (86.7) |
| N | 101 | 205 | 199 | 204 | 709 |
| MODEL P-VALUES | | | | | |
| TREATMENT [1] | <0.001 | | | | |
| SEX [1] | 0.693 | | | | |
| BASELINE PI [1] | 0.005 | | | | |
| TREATMENT*BASELINE PI [2] | 0.557 | | | | |
| TREATMENT*SEX [2] | 0.697 | | | | |
| PAIRWISE COMPARISON | | | | | |
| P-VALUES [1] | | | | | |
| PLACEBO QID | — | <0.001 | 0.008 | 0.012 | |
| OXY QID | — | — | 0.089 | 0.058 | |
| OXY + NTX QID | — | — | — | 0.862 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS ACOVARIATE.
[2] P-VALUES FROM ANCOVA MODEL DEFINED IN [1] INCLUDING TREATMENT*BASELINE PI AND TREATMENT*SEX INTERACTION TERMS.

TABLE 45II
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE | | | | | |
| WEEK 12 | | | | | |
| MEAN (SD) | 5.2 (3.05) | 4.0 (2.53) | 4.2 (2.56) | 4.3 (2.55) | 4.3 (2.65) |
| MEDIAN | 5.3 | 3.7 | 4.0 | 4.0 | 4.0 |
| MINIMUM (MAXIMUM) | 0.0 (10.0) | 0.0 (10.0) | 0.0 (10.0) | 0.0 (10.0) | 0.0 (10.0) |
| N | 101 | 205 | 199 | 204 | 709 |
| MODEL P-VALUES | | | | | |
| TREATMENT [1] | 0.003 | | | | |
| SEX [1] | 0.350 | | | | |
| BASELINE PI [1] | <0.001 | | | | |
| TREATMENT*BASELINE PI [2] | 0.049 | | | | |
| TREATMENT*SEX [2] | 0.831 | | | | |
| PAIRWISE COMPARISON P-VALUES [1] | | | | | |
| PLACEBO QID | — | <0.001 | 0.007 | 0.004 | |
| OXY QID | — | — | 0.249 | 0.316 | |
| OXY + NTX QID | — | — | — | 0.873 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS ACOVARIATE.
[2] P-VALUES FROM ANCOVA MODEL DEFINED IN [1] INCLUDING TREATMENT*BASELINE PI AND TREATMENT*SEX INTERACTION TERMS.

TABLE 45JJ
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO FIXED | | | | | |
| DOSE WEEK 12 | | | | | |
| MEAN (SD) | −2.5 (2.80) | −3.5 (2.65) | −3.0 (2.71) | −3.3 (2.69) | −3.2 (2.72) |
| MEDIAN | −1.7 | −3.7 | −3.0 | −3.0 | −3.0 |
| MINIMUM (MAXIMUM) | −8.7 (4.3) | −9.7 (3.0) | −10.0 (2.3) | −8.7 (4.0) | −10.0 (4.3) |
| N | 101 | 205 | 199 | 204 | 709 |
| MODEL P-VALUES | | | | | |
| TREATMENT [1] | 0.003 | | | | |
| SEX [1] | 0.350 | | | | |
| BASELINE PI [1] | <0.001 | | | | |
| TREATMENT*BASELINE PI [2] | 0.049 | | | | |
| TREATMENT*SEX [2] | 0.831 | | | | |
| PAIRWISE COMPARISON P-VALUES [1] | | | | | |
| PLACEBO QID | — | <0.001 | 0.007 | 0.004 | |
| OXY QID | — | — | 0.249 | 0.316 | |
| OXY + NTX QID | — | — | — | 0.873 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS ACOVARIATE.
[2] P-VALUES FROM ANCOVA MODEL DEFINED IN [1] INCLUDING TREATMENT*BASELINE PI AND TREATMENT*SEX INTERACTION TERMS.

TABLE 45KK
PAIN INTENSITY - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| PERCENT CHANGE FROM | | | | | |
| BASELINE TO FIXED DOSE | | | | | |
| WEEK 12 | | | | | |
| MEAN (SD) | −32.2 (38.04) | −46.2 (33.60) | −41.2 (35.15) | −42.6 (34.46) | −41.8 (35.14) |
| MEDIAN | −21.1 | −52.0 | −44.4 | −45.2 | −45.0 |
| MINIMUM (MAXIMUM) | −100.0 (86.7) | −100.0 (42.9) | −100.0 (41.2) | −100.0 (80.0) | −100.0 (86.7) |
| N | 101 | 205 | 199 | 204 | 709 |
| MODEL P-VALUES | | | | | |
| TREATMENT [1] | 0.009 | | | | |
| SEX [1] | 0.483 | | | | |
| BASELINE PI [1] | 0.003 | | | | |
| TREATMENT*BASELINE PI [2] | 0.441 | | | | |
| TREATMENT*SEX [2] | 0.731 | | | | |
| PAIRWISE COMPARISON | | | | | |
| P-VALUES [1] | | | | | |
| PLACEBO QID | — | <0.001 | 0.015 | 0.011 | |
| OXY QID | — | — | 0.254 | 0.291 | |
| OXY + NTX QID | — | — | — | 0.922 | |

NOTE:
USING AVERAGE PAIN INTENSITY OF LAST THREE DAYS WITHIN EACH DOSING WEEK.
[1] P-VALUES FROM ANCOVA MODEL INCLUDING TREATMENT AS THE MAIN EFFECT, SEX AS A BLOCKING FACTOR, AND BASELINE PAIN INTENSITY AS ACOVARIATE.
[2] P-VALUES FROM ANCOVA MODEL DEFINED IN [1] INCLUDING TREATMENT*BASELINE PI AND TREATMENT*SEX INTERACTION TERMS.

Another efficacy assessment was quality of analgesia and the results for this study are shown in Table 46 for Weeks 1, 2, 4, 6, 8, 10 and 12 of the fixed-dose period. The active treatment groups showed greater improvement in the quality of analgesia at each of the weeks shown of the fixed-dose period as compared with placebo, and the differences between each active treatment group versus placebo were statistically significant. There were no statistically significant differences between active treatment groups (e.g., when one active treatment group was compared to another active treatment group).

TABLE 46
QUALITY OF ANALGESIA - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| FIXED DOSE PERIOD: WEEK 1 | | | | | |
| EXCELLENT (4) | 5 (5.0%) | 25 (12.1%) | 22 (10.7%) | 14 (6.8%) | 66 (9.2%) |
| VERY GOOD (3) | 19 (18.8%) | 52 (25.2%) | 44 (21.4%) | 52 (25.2%) | 167 (23.2%) |
| GOOD (2) | 20 (19.8%) | 69 (33.5%) | 70 (34.0%) | 63 (30.6%) | 222 (30.9%) |
| FAIR (1) | 16 (15.8%) | 26 (12.6%) | 26 (12.6%) | 46 (22.3%) | 114 (15.9%) |
| POOR (0) | 38 (37.6%) | 33 (16.0%) | 37 (18.0%) | 26 (12.6%) | 134 (18.6%) |
| OVERALL P-VALUE [1] | <0.00 | | | | |
| PAIRWISE COMPARISON P-VALUES [2] | | | | | |
| PLACEBO QID | — | <0.001 | <0.001 | <0.00 | |
| OXY QID | — | — | 0.375 | 0.239 | |
| OXY + NTX QID | — | — | — | 0.805 | |
| FIXED DOSE PERIOD: WEEK 2 | | | | | |
| EXCELLENT (4) | 8 (7.9%) | 24 (11.7%) | 20 (9.7%) | 12 (5.8%) | 64 (8.9%) |
| VERY GOOD (3) | 12 (11.9%) | 52 (25.2%) | 49 (23.8%) | 51 (24.8%) | 164 (22.8%) |
| GOOD (2) | 23 (22.8%) | 70 (34.0%) | 67 (32.5%) | 58 (28.2%) | 218 (30.3%) |
| FAIR (1) | 14 (13.9%) | 27 (13.1%) | 28 (13.6%) | 50 (24.3%) | 119 (16.6%) |
| POOR (0) | 41 (40.6%) | 32 (15.5%) | 35 (17.0%) | 30 (14.6%) | 138 (19.2%) |
| OVERALL P-VALUE [1] | <0.001 | | | | |
| PAIRWISE COMPARISON P-VALUES [2] | | | | | |
| PLACEBO QID | — | <0.001 | <0.001 | <0.001 | |
| OXY QID | — | — | 0.463 | 0.064 | |
| OXY + NTX QID | — | — | — | 0.277 | |
| FIXED DOSE PERIOD: WEEK 4 | | | | | |
| EXCELLENT (4) | 8 (7.9%) | 20 (9.7%) | 16 (7.8%) | 11 (5.3%) | 55 (7.6%) |
| VERY GOOD (3) | 17 (16.8%) | 59 (28.6%) | 47 (22.8%) | 55 (26.7%) | 178 (24.8%) |
| GOOD (2) | 15 (14.9%) | 63 (30.6%) | 67 (32.5%) | 60 (29.1%) | 205 (28.5%) |
| FAIR (1) | 15 (14.9%) | 28 (13.6%) | 33 (16.0%) | 44 (21.4%) | 120 (16.7%) |
| POOR (0) | 43 (42.6%) | 35 (17.0%) | 36 (17.5%) | 31 (15.0%) | 145 (20.2%) |
| OVERALL P-VALUE [1] | <0.001 | | | | |
| PAIRWISE COMPARISON P-VALUES [2] | | | | | |
| PLACEBO QID | — | <0.001 | <0.001 | <0.001 | |
| OXY QID | — | — | 0.262 | 0.206 | |
| OXY + NTX QID | — | — | — | 0.907 | |
| FIXED DOSE PERIOD: WEEK 6 | | | | | |
| EXCELLENT (4) | 8 (7.9%) | 24 (11.7%) | 16 (7.8%) | 17 (8.3%) | 65 (9.0%) |
| VERY GOOD (3) | 18 (17.8%) | 44 (21.4%) | 47 (22.8%) | 49 (23.8%) | 158 (22.0%) |
| GOOD (2) | 17 (16.8%) | 67 (32.5%) | 67 (32.5%) | 64 (31.1%) | 215 (29.9%) |
| FAIR (1) | 12 (11.9%) | 34 (16.5%) | 31 (15.0%) | 43 (20.9%) | 120 (16.7%) |
| POOR (0) | 43 (42.6%) | 36 (17.5%) | 38 (18.4%) | 28 (13.6%) | 145 (20.2%) |
| OVERALL P-VALUE [1] | <0.001 | | | | |
| PAIRWISE COMPARISON P-VALUES [2] | | | | | |
| PLACEBO QID | — | <0.001 | <0.001 | <0.001 | |
| OXY QID | — | — | 0.553 | 0.925 | |
| OXY + NTX QID | — | — | — | 0.606 | |
| FIXED DOSE PERIOD: WEEK 8 | | | | | |
| EXCELLENT (4) | 6 (5.9%) | 24 (11.7%) | 24 (11.7%) | 16 (7.8%) | 70 (9.7%) |
| VERY GOOD (3) | 18 (17.8%) | 41 (19.9%) | 42 (20.4%) | 45 (21.8%) | 146 (20.3%) |
| GOOD (2) | 20 (19.8%) | 72 (35.0%) | 65 (31.6%) | 64 (31.1%) | 221 (30.7%) |
| FAIR (1) | 12 (11.9%) | 35 (17.0%) | 31 (15.0%) | 46 (22.3%) | 124 (17.2%) |
| POOR (0) | 42 (41.6%) | 33 (16.0%) | 37 (18.0%) | 30 (14.6%) | 142 (19.7%) |
| OVERALL P-VALUE [1] | <0.001 | | | | |
| PAIRWISE COMPARISON P-VALUES [2] | | | | | |
| PLACEBO QID | — | <0.001 | <0.001 | <0.001 | |
| OXY QID | — | — | 0.891 | 0.468 | |
| OXY + NTX QID | — | — | — | 0.570 | |
| FIXED DOSE PERIOD: WEEK 10 | | | | | |
| EXCELLENT (4) | 6 (5.9%) | 24 (11.7%) | 21 (10.2%) | 18 (8.7%) | 69 (9.6%) |
| VERY GOOD (3) | 20 (19.8%) | 45 (21.8%) | 43 (20.9%) | 44 (21.4%) | 152 (21.1%) |
| GOOD (2) | 13 (12.9%) | 66 (32.0%) | 63 (30.6%) | 58 (28.2%) | 200 (27.8%) |
| FAIR (1) | 16 (15.8%) | 35 (17.0%) | 32 (15.5%) | 50 (24.3%) | 133 (18.5%) |
| POOR (0) | 43 (42.6%) | 35 (17.0%) | 40 (19.4%) | 31 (15.0%) | 149 (20.7%) |
| OVERALL P-VALUE [1] | <0.001 | | | | |
| PAIRWISE COMPARISON P-VALUES [2] | | | | | |
| PLACEBO QID | — | <0.001 | <0.001 | <0.001 | |
| OXY QID | — | — | 0.536 | 0.405 | |
| OXY + NTX QID | — | — | — | 0.848 | |
| FIXED DOSE PERIOD: WEEK 12 | | | | | |
| EXCELLENT (4) | 8 (7.9%) | 21 (10.2%) | 22 (10.7%) | 18 (8.7%) | 69 (9.6%) |
| VERY GOOD (3) | 16 (15.8%) | 43 (20.9%) | 45 (21.8%) | 47 (22.8%) | 151 (21.0%) |
| GOOD (2) | 14 (13.9%) | 64 (31.1%) | 60 (29.1%) | 52 (25.2%) | 190 (26.4%) |
| FAIR (1) | 16 (15.8%) | 40 (19.4%) | 34 (16.5%) | 52 (25.2%) | 142 (19.7%) |
| POOR (0) | 44 (43.6%) | 37 (18.0%) | 38 (18.4%) | 32 (15.5%) | 151 (21.0%) |
| OVERALL P-VALUE [1] | <0.001 | | | | |
| PAIRWISE COMPARISON P-VALUES [2] | | | | | |
| PLACEBO QID | — | <0.001 | <0.001 | <0.001 | |
| OXY QID | — | — | 0.772 | 0.851 | |
| OXY + NTX QID | — | — | — | 0.635 | |
| OXY + NTX QID | — | — | — | 0.277 | |

[1] COCHRAN-MANTEL-HAENSZEL (ROW MEAN SCORES) TEST ACROSS TREATMENT GROUPS USING EQUALLY SPACED SCORES.
[2] COCHRAN-MANTEL-HAENSZEL (ROW MEAN SCORES) TEST BETWEEN TREATMENT GROUPS USING EQUALLY SPACED SCORES.

Another efficacy assessment was a global assessment and the results for this study are shown in Table 47 for Weeks 1, 2, 4, 6, 8, 10 and 12 of the fixed-dose period. The active treatment groups were statistically significantly better than placebo (in pairwise comparisons) as shown in Table 47. Table 47 also shows the p value versus placebo calculated for the scores from the global assessment for the weeks shown of the fixed-dose period, which were determined using the Cochran-Mantel-Haenszel row mean scores (CMH-RMS) test, using equally spaced scores.

TABLE 47
GLOBAL ASSESSMENT - BY WEEK
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| FIXED DOSE PERIOD: WEEK 1 | | | | | |
| EXCELLENT (4) | 6 (5.9%) | 14 (6.8%) | 18 (8.7%) | 14 (6.8%) | 52 (7.2%) |
| VERY GOOD (3) | 19 (18.8%) | 63 (30.6%) | 45 (21.8%) | 52 (25.2%) | 179 (24.9%) |
| GOOD (2) | 22 (21.8%) | 72 (35.0%) | 71 (34.5%) | 63 (30.6%) | 228 (31.7%) |
| FAIR (1) | 15 (14.9%) | 23 (11.2%) | 25 (12.1%) | 42 (20.4%) | 105 (14.6%) |
| POOR (0) | 36 (35.6%) | 33 (16.0%) | 40 (19.4%) | 30 (14.6%) | 139 (19.3%) |
| OVERALL P-VALUE [1] | 0.001 | | | | |
| PAIRWISE COMPARISON P-VALUES [2] | | | | | |
| PLACEBO QID | — | <0.001 | 0.004 | 0.002 | |
| OXY QID | — | — | 0.273 | 0.300 | |
| OXY + NTX QID | — | — | — | 0.925 | |
| FIXED DOSE PERIOD: WEEK 2 | | | | | |
| EXCELLENT (4) | 9 (8.9%) | 21 (10.2%) | 17 (8.3%) | 10 (4.9%) | 57 (7.9%) |
| VERY GOOD (3) | 13 (12.9%) | 50 (24.3%) | 50 (24.3%) | 51 (24.8%) | 164 (22.8%) |
| GOOD (2) | 24 (23.8%) | 79 (38.3%) | 62 (30.1%) | 58 (28.2%) | 223 (31.0%) |
| FAIR (1) | 14 (13.9%) | 22 (10.7%) | 30 (14.6%) | 48 (23.3%) | 114 (15.9%) |
| POOR (0) | 38 (37.6%) | 33 (16.0%) | 40 (19.4%) | 34 (16.5%) | 145 (20.2%) |
| OVERALL P-VALUE [1] | <0.001 | | | | |
| PAIRWISE COMPARISON P-VALUES [2] | | | | | |
| PLACEBO QID | — | <0.001 | 0.003 | 0.013 | |
| OXY QID | — | — | 0.214 | 0.037 | |
| OXY + NTX QID | — | — | — | 0.436 | |
| FIXED DOSE PERIOD: WEEK 4 | | | | | |
| EXCELLENT (4) | 8 (7.9%) | 20 (9.7%) | 17 (8.3%) | 7 (3.4%) | 52 (7.2%) |
| VERY GOOD (3) | 17 (16.8%) | 53 (25.7%) | 42 (20.4%) | 53 (25.7%) | 165 (22.9%) |
| GOOD (2) | 20 (19.8%) | 69 (33.5%) | 67 (32.5%) | 64 (31.1%) | 220 (30.6%) |
| FAIR (1) | 13 (12.9%) | 26 (12.6%) | 31 (15.0%) | 40 (19.4%) | 110 (15.3%) |
| POOR (0) | 40 (39.6%) | 37 (18.0%) | 42 (20.4%) | 37 (18.0%) | 156 (21.7%) |
| OVERALL P-VALUE [1] | 0.002 | | | | |
| PAIRWISE COMPARISON P-VALUES [2] | | | | | |
| PLACEBO QID | — | <0.001 | 0.009 | 0.013 | |
| OXY QID | — | — | 0.187 | 0.090 | |
| OXY + NTX QID | — | — | — | 0.749 | |
| FIXED DOSE PERIOD: WEEK 6 | | | | | |
| EXCELLENT (4) | 9 (8.9%) | 23 (11.2%) | 16 (7.8%) | 11 (5.3%) | 59 (8.2%) |
| VERY GOOD (3) | 17 (16.8%) | 43 (20.9%) | 43 (20.9%) | 49 (23.8%) | 152 (21.1%) |
| GOOD (2) | 19 (18.8%) | 73 (35.4%) | 65 (31.6%) | 64 (31.1%) | 221 (30.7%) |
| FAIR (1) | 13 (12.9%) | 28 (13.6%) | 32 (15.5%) | 41 (19.9%) | 114 (15.9%) |
| POOR (0) | 40 (39.6%) | 38 (18.4%) | 43 (20.9%) | 36 (17.5%) | 157 (21.8%) |
| OVERALL P-VALUE [1] | 0.009 | | | | |
| PAIRWISE COMPARISON P-VALUES [2] | | | | | |
| PLACEBO QID | — | 0.001 | 0.019 | 0.013 | |
| OXY QID | — | — | 0.246 | 0.255 | |
| OXY + NTX QID | — | — | — | 0.953 | |
| FIXED DOSE PERIOD: WEEK 8 | | | | | |
| EXCELLENT (4) | 8 (7.9%) | 20 (9.7%) | 23 (11.2%) | 10 (4.9%) | 61 (8.5%) |
| VERY GOOD (3) | 16 (15.8%) | 41 (19.9%) | 40 (19.4%) | 47 (22.8%) | 144 (20.0%) |
| GOOD (2) | 21 (20.8%) | 74 (35.9%) | 61 (29.6%) | 63 (30.6%) | 219 (30.5%) |
| FAIR (1) | 14 (13.9%) | 34 (16.5%) | 32 (15.5%) | 45 (21.8%) | 125 (17.4%) |
| POOR (0) | 39 (38.6%) | 36 (17.5%) | 43 (20.9%) | 36 (17.5%) | 154 (21.4%) |
| OVERALL P-VALUE [1] | 0.010 | | | | |
| PAIRWISE COMPARISON P-VALUES [2] | | | | | |
| PLACEBO QID | — | 0.002 | 0.006 | 0.017 | |
| OXY QID | — | — | 0.754 | 0.278 | |
| OXY + NTX QID | — | — | — | 0.471 | |
| FIXED DOSE PERIOD: WEEK 10 | | | | | |
| EXCELLENT (4) | 10 (9.9%) | 23 (11.2%) | 24 (11.7%) | 13 (6.3%) | 70 (9.7%) |
| VERY GOOD (3) | 15 (14.9%) | 45 (21.8%) | 37 (18.0%) | 44 (21.4%) | 141 (19.6%) |
| GOOD (2) | 16 (15.8%) | 65 (31.6%) | 59 (28.6%) | 60 (29.1%) | 200 (27.8%) |
| FAIR (1) | 18 (17.8%) | 34 (16.5%) | 34 (16.5%) | 46 (22.3%) | 132 (18.4%) |
| POOR (0) | 39 (38.6%) | 38 (18.4%) | 45 (21.8%) | 38 (18.4%) | 160 (22.3%) |
| OVERALL P-VALUE [1] | 0.008 | | | | |
| PAIRWISE COMPARISON P-VALUES [2] | | | | | |
| PLACEBO QID | — | 0.001 | 0.011 | 0.020 | |
| OXY QID | — | — | 0.418 | 0.171 | |
| OXY + NTX QID | — | — | — | 0.615 | |
| FIXED DOSE PERIOD: WEEK 12 | | | | | |
| EXCELLENT (4) | 8 (7.9%) | 22 (10.7%) | 24 (11.7%) | 13 (6.3%) | 67 (9.3%) |
| VERY GOOD (3) | 16 (15.8%) | 37 (18.0%) | 39 (18.9%) | 44 (21.4%) | 136 (18.9%) |
| GOOD (2) | 16 (15.8%) | 75 (36.4%) | 58 (28.2%) | 52 (25.2%) | 201 (28.0%) |
| FAIR (1) | 17 (16.8%) | 33 (16.0%) | 33 (16.0%) | 52 (25.2%) | 135 (18.8%) |
| POOR (0) | 41 (40.6%) | 38 (18.4%) | 45 (21.8%) | 40 (19.4%) | 164 (22.8%) |
| OVERALL P-VALUE [1] | 0.003 | | | | |
| PAIRWISE COMPARISON P-VALUES [2] | | | | | |
| PLACEBO QID | — | <0.001 | 0.003 | 0.017 | |
| OXY QID | — | — | 0.725 | 0.154 | |
| OXY + NTX QID | — | — | — | 0.311 | |

[1] COCHRAN-MANTEL-HAENSZEL (ROW MEAN SCORES) TEST ACROSS TREATMENT GROUPS USING EQUALLY SPACED SCORES.
[2] COCHRAN-MANTEL-HAENSZEL (ROW MEAN SCORES) TEST BETWEEN TREATMENT GROUPS USING EQUALLY SPACED SCORES.

Another efficacy assessment was a functional assessment. For this study an Oswestry ity Index (ODI) was used and the results are shown in Tables 48A-48I. Table 48A shows Disability Index ODI values. Table 48B shows actual values at the end of the titration. Table 48C shows change from baseline to the end of titration. Table 48D shows actual values for Week 4 of the fixed-dose period. Table 48E shows change from baseline to Week 4 of the fixed-dose period. Table 48F shows actual values for Week 8 of the fixed-dose period. Table 48G shows change from baseline to Week 8 of the fixed-dose period. Table 48H shows actual values for Week 12 of the fixed-dose period. Table 481 shows change from baseline to Week 12 of the fixed-dose period. Greater improvement as shown by changes from baseline was observed with the active treatment groups versus placebo.

TABLE 48A
OSWESTRY DISABILITY INDEX
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| BASELINE | | | | | |
| MEAN (SD) | 40.8 (14.03) | 40.9 (13.98) | 41.0 (14.05) | 39.8 (14.78) | 40.6 (14.22) |
| MEDIAN | 42.0 | 40.0 | 40.0 | 38.0 | 40.0 |
| MINIMUM (MAXIMUM) | 13.3 (74.0) | 10.0 (72.0) | 6.0 (82.0) | 4.0 (78.0) | 4.0 (82.0) |
| N | 101 | 206 | 206 | 205 | 718 |
| MODEL P-VALUES | | | | | |
| TREATMENT [1] | 0.807 | | | | |
| SEX [1] | 0.008 | | | | |
| TREATMENT* SEX [2] | 0.923 | | | | |
| PAIRWISE COMPARISON P-VALUES [1] | | | | | |
| PLACEBO QID | — | 0.956 | 0.927 | 0.542 | |
| OXY QID | — | — | 0.963 | 0.413 | |
| OYX + NTX QID | — | — | — | 0.387 | |

NOTE:
LOWER VALUES CORRESPOND TO BETTER HEALTH OR FUNCTIONING.
[1] P-VALUES FROM ANOVA MODEL INCLUDING TREATMENT AS THE MAIN EFFECT AND SEX AS A BLOCKING FACTOR.
[2] P-VALUES FROM ANOVA MODEL DEFINED IN [1] INCLUDING TREATMENT* SEX INTERACTION TERMS.

TABLE 48B
OSWESTRY DISABILITY INDEX
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT END OF TITRATION | | | | | |
| MEAN (SD) | 36.2 (16.40) | 31.6 (15.63) | 32.9 (15.84) | 32.3 (17.01) | 32.8 (16.22) |
| MEDIAN | 36.9 | 31.1 | 32.0 | 32.0 | 32.0 |
| MINIMUM (MAXIMUM) | 0.0 (72.0) | 0.0 (93.3) | 0.0 (78.0) | 2.2 (80.0) | 0.0 (93.3) |
| N | 88 | 173 | 157 | 160 | 578 |
| MODEL P-VALUES | | | | | |
| TREATMENT [1] | 0.074 | | | | |
| SEX [1] | 0.451 | | | | |
| BASELINE ODI [1] | <0.001 | | | | |
| TREATMENT* BASELINE ODI [2] | 0.508 | | | | |
| TREATMENT* SEX [2] | 0.746 | | | | |
| PAIRWISE COMPARISON P-VALUES [1] | | | | | |
| PLACEBO QID | — | 0.013 | 0.027 | 0.106 | |
| OXY QID | — | — | 0.767 | 0.303 | |
| OXY + NTX QID | — | — | — | 0.475 | |

NOTE:
LOWER VALUES CORRESPOND TO BETTER HEALTH OR FUNCTIONING.
[1] P-VALUES FROM ANCOVA MODEL INCLUDING TREATMENT AS THE MAIN EFFECT, SEX AS A BLOCKING FACTOR, AND BASELINE ODI AS A COVARIATE.
[2] P-VALUES FROM ANCOVA MODEL DEFINED IN [1] INCLUDING TREATMENT* BASELINE ODI AND TREATMENT* SEX INTERACTION TERMS.

TABLE 48C
OSWESTRY DISABILITY INDEX
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE TO END OF | | | | | |
| TITRATION | | | | | |
| MEAN (SD) | −5.9 (11.84) | −9.7 (13.13) | −9.6 (12.06) | −8.1 (13.64) | −8.6 (12.84) |
| MEDIAN | −4.0 | −8.9 | −8.0 | −6.0 | −6.8 |
| MINIMUM (MAXIMUM) | −56.0 (26.0) | −56.0 (48.9) | −40.0 (32.9) | −52.0 (46.9) | −56.0 (48.9) |
| N | 88 | 173 | 157 | 160 | 578 |
| MODEL P-VALUES | | | | | |
| TREATMENT [1] | 0.074 | | | | |
| SEX [1] | 0.451 | | | | |
| BASELINE ODI [1] | <0.001 | | | | |
| TREATMENT* BASELINE ODI [2] | 0.508 | | | | |
| TREATMENT* SEX [2] | 0.746 | | | | |
| PAIRWISE COMPARISON P-VALUES [1] | | | | | |
| PLACEBO QID | — | 0.013 | 0.027 | 0.106 | |
| OXY QID | — | — | 0.767 | 0.303 | |
| OXY + NTX QID | — | — | — | 0.475 | |

NOTE:
LOWER VALUES CORRESPOND TO BETTER HEALTH OR FUNCTIONING.
[1] P-VALUES FROM ANCOVA MODEL INCLUDING TREATMENT AS THE MAIN EFFECT, SEX AS A BLOCKING FACTOR, AND BASELINE ODI AS A COVARIATE.
[2] P-VALUES FROM ANCOVA MODEL DEFINED IN [1] INCLUDING TREATMENT* BASELINE ODI AND TREATMENT* SEX INTERACTION TERMS.

TABLE 48D
OSWESTRY DISABILITY INDEX
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE WEEK 4 | | | | | |
| MEAN (SD) | 34.4 (17.73) | 31.3 (15.80) | 31.8 (16.32) | 30.2 (16.99) | 31.6 (16.58) |
| MEDIAN | 33.3 | 30.0 | 30.0 | 28.0 | 30.0 |
| MINIMUM (MAXIMUM) | 0.0 (73.3) | 0.0 (93.3) | 0.0 (78.0) | 0.0 (80.0) | 0.0 (93.3) |
| N | 95 | 197 | 191 | 196 | 679 |
| MODEL P-VALUES | | | | | |
| TREATMENT [1] | 0.267 | | | | |
| SEX [1] | 0.867 | | | | |
| BASELINE ODI [1] | <0.001 | | | | |
| TREATMENT* BASELINE ODI [2] | 0.075 | | | | |
| TREATMENT* SEX [2] | 0.955 | | | | |
| PAIRWISE COMPARISON P-VALUES [1] | | | | | |
| PLACEBO QID | — | 0.077 | 0.128 | 0.064 | |
| OXY QID | — | — | 0.769 | 0.909 | |
| OXY + NTX QID | — | — | — | 0.685 | |

NOTE:
LOWER VALUES CORRESPOND TO BETTER HEALTH OR FUNCTIONING.
[1] P-VALUES FROM ANCOVA MODEL INCLUDING TREATMENT AS THE MAIN EFFECT, SEX AS A BLOCKING FACTOR, AND BASELINE ODI AS A COVARIATE.
[2] P-VALUES FROM ANCOVA MODEL DEFINED IN [1] INCLUDING TREATMENT* BASELINE ODI AND TREATMENT* SEX INTERACTION TERMS.

TABLE 48E
OSWESTRY DISABILITY INDEX
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE FIXED | | | | | |
| DOSE WEEK 4 | | | | | |
| MEAN (SD) | −6.8 (12.01) | −9.5 (13.79) | −9.2 (12.59) | −9.4 (13.82) | −9.0 (13.23) |
| MEDIAN | −6.0 | −10.0 | −8.0 | −6.4 | −8.0 |
| MINIMUM (MAXIMUM) | −46.0 (26.0) | −66.0 (48.9) | −50.0 (32.9) | −58.0 (46.9) | −66.0 (48.9) |
| N | 95 | 197 | 191 | 195 | 678 |
| MODEL P-VALUES | | | | | |
| TREATMENT [1] | 0.267 | | | | |
| SEX [1] | 0.867 | | | | |
| BASELINE ODI [1] | <0.001 | | | | |
| TREATMENT* BASELINE ODI [2] | 0.075 | | | | |
| TREATMENT* SEX [2] | 0.955 | | | | |
| PAIRWISE COMPARISON P-VALUES [1] | | | | | |
| PLACEBO QID | — | 0.077 | 0.128 | 0.064 | |
| OXY QID | — | — | 0.769 | 0.909 | |
| OXY + NTX QID | — | — | — | 0.685 | |

NOTE:
LOWER VALUES CORRESPOND TO BETTER HEALTH OR FUNCTIONING.
[1] P-VALUES FROM ANCOVA MODEL INCLUDING TREATMENT AS THE MAIN EFFECT, SEX AS A BLOCKING FACTOR, AND BASELINE ODI AS A COVARIATE.
[2] P-VALUES FROM ANCOVA MODEL DEFINED IN [1] INCLUDING TREATMENT* BASELINE ODI AND TREATMENT* SEX INTERACTION TERMS.

TABLE 48F
OSWESTRY DISABILITY INDEX
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE WEEK 8 | | | | | |
| MEAN (SD) | 33.8 (17.42) | 30.8 (16.04) | 31.3 (16.71) | 30.1 (17.33) | 31.2 (16.81) |
| MEDIAN | 32.0 | 30.0 | 28.4 | 30.0 | 30.0 |
| MINIMUM (MAXIMUM) | 0.0 (73.3) | 0.0 (93.3) | 0.0 (78.0) | 0.0 (80.0) | 0.0 (93.3) |
| N | 95 | 197 | 192 | 196 | 680 |
| MODEL P-VALUES | | | | | |
| TREATMENT [1] | 0.359 | | | | |
| SEX [1] | 0.971 | | | | |
| BASELINE ODI [1] | <0.001 | | | | |
| TREATMENT* BASELINE ODI [2] | 0.401 | | | | |
| TREATMENT* SEX [2] | 0.997 | | | | |
| PAIRWISE COMPARISON P-VALUES [1] | | | | | |
| PLACEBO QID | — | 0.092 | 0.141 | 0.123 | |
| OXY QID | — | — | 0.797 | 0.863 | |
| OXY + NTX QID | — | — | — | 0.933 | |

NOTE:
LOWER VALUES CORRESPOND TO BETTER HEALTH OR FUNCTIONING.
[1] P-VALUES FROM ANCOVA MODEL INCLUDING TREATMENT AS THE MAIN EFFECT, SEX AS A BLOCKING FACTOR, AND BASELINE ODI AS A COVARIATE.
[2] P-VALUES FROM ANCOVA MODEL DEFINED IN [1] INCLUDING TREATMENT* BASELINE ODI AND TREATMENT* SEX INTERACTION TERMS.

TABLE 48G
OSWESTRY DISABILITY INDEX
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE | | | | | |
| FIXED DOSE WEEK 8 | | | | | |
| MEAN (SD) | −7.3 (12.58) | −10.0 (13.78) | −9.7 (13.76) | −9.5 (14.42) | −9.4 (13.80) |
| MEDIAN | −6.0 | −10.0 | −8.0 | −6.4 | −8.0 |
| MINIMUM (MAXIMUM) | −52.0 (26.0) | −64.5 (48.9) | −52.0 (32.9) | −70.0 (46.9) | −70.0 (48.9) |
| N | 95 | 197 | 192 | 195 | 679 |
| MODEL P-VALUES | | | | | |
| TREATMENT [1] | 0.359 | | | | |
| SEX [1] | 0.971 | | | | |
| BASELINE ODI [1] | <0.001 | | | | |
| TREATMENT* BASELINE ODI [2] | 0.401 | | | | |
| TREATMENT* SEX [2] | 0.997 | | | | |
| PAIRWISE COMPARISON P-VALUES [1] | | | | | |
| PLACEBO QID | — | 0.092 | 0.141 | 0.123 | |
| OXY QID | — | — | 0.797 | 0.863 | |
| OXY + NTX QID | — | — | — | 0.933 | |

NOTE:
LOWER VALUES CORRESPOND TO BETTER HEALTH OR FUNCTIONING.
[1] P-VALUES FROM ANCOVA MODEL INCLUDING TREATMENT AS THE MAIN EFFECT, SEX AS A BLOCKING FACTOR, AND BASELINE ODI AS A COVARIATE.
[2] P-VALUES FROM ANCOVA MODEL DEFINED IN [1] INCLUDING TREATMENT* BASELINE ODI AND TREATMENT* SEX INTERACTION TERMS.

TABLE 48H
OSWESTRY DISABILITY INDEX
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| ACTUAL VALUE AT FIXED DOSE | | | | | |
| WEEK 12 | | | | | |
| MEAN (SD) | 34.3 (17.70) | 31.3 (16.72) | 30.9 (16.51) | 30.8 (17.57) | 31.5 (17.05) |
| MEDIAN | 34.0 | 30.0 | 28.0 | 30.0 | 30.0 |
| MINIMUM (MAXIMUM) | 0.0 (73.3) | 0.0 (93.3) | 0.0 (78.0) | 0.0 (80.0) | 0.0 (93.3) |
| N | 95 | 197 | 192 | 196 | 680 |
| MODEL P-VALUES | | | | | |
| TREATMENT [1] | 0.256 | | | | |
| SEX [1] | 0.885 | | | | |
| BASELINE ODI [1] | <0.001 | | | | |
| TREATMENT* BASELINE ODI [2] | 0.310 | | | | |
| TREATMENT* SEX [2] | 0.958 | | | | |
| PAIRWISE COMPARISON P-VALUES [1] | | | | | |
| PLACEBO QID | — | 0.101 | 0.050 | 0.155 | |
| OXY QID | — | — | 0.683 | 0.791 | |
| OXY + NTX QID | — | — | — | 0.502 | |

NOTE:
LOWER VALUES CORRESPOND TO BETTER HEALTH OR FUNCTIONING.
[1] P-VALUES FROM ANCOVA MODEL INCLUDING TREATMENT AS THE MAIN EFFECT, SEX AS A BLOCKING FACTOR, AND BASELINE ODI AS A COVARIATE.
[2] P-VALUES FROM ANCOVA MODEL DEFINED IN [1] INCLUDING TREATMENT* BASELINE ODI AND TREATMENT* SEX INTERACTION TERMS.

TABLE 48I
OSWESTRY DISABILITY INDEX
LAST OBSERVATION CARRIED FORWARD IMPUTATION
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| CHANGE FROM BASELINE FIXED DOSE WEEK 12 | | | | | |
| MEAN (SD) | −6.8 (12.85) | −9.5 (14.80) | −10.1 (13.38) | −8.9 (14.51) | −9.1 (14.07) |
| MEDIAN | −6.0 | −10.0 | −8.0 | −6.0 | −8.0 |
| MINIMUM (MAXIMUM) | −52.0 (26.0) | −72.0 (48.9) | −52.0 (32.9) | −74.0 (46.9) | −74.0 (48.9) |
| N | 95 | 197 | 192 | 195 | 679 |
| MODEL P-VALUES | | | | | |
| TREATMENT [1] | 0.256 | | | | |
| SEX [1] | 0.885 | | | | |
| BASELINE ODI [1] | <0.001 | | | | |
| TREATMENT* BASELINE ODI [2] | 0.310 | | | | |
| TREATMENT* SEX [2] | 0.958 | | | | |
| PAIRWISE COMPARISON P-VALUES [1] | | | | | |
| PLACEBO QID | — | 0.101 | 0.050 | 0.155 | |
| OXY QID | — | — | 0.683 | 0.791 | |
| OXY + NTX QID | — | — | — | 0.502 | |

NOTE:
LOWER VALUES CORRESPOND TO BETTER HEALTH OR FUNCTIONING.
[1] P-VALUES FROM ANCOVA MODEL INCLUDING TREATMENT AS THE MAIN EFFECT, SEX AS A BLOCKING FACTOR, AND BASELINE ODI AS A COVARIATE.
[2] P-VALUES FROM ANCOVA MODEL DEFINED IN [1] INCLUDING TREATMENT* BASELINE ODI AND TREATMENT* SEX INTERACTION TERMS.

The overall incidences of adverse events in all three active treatment groups were compared, and the numerical differences observed between the oxycodone and naltrexone QID and oxycodone and naltrexone BID treatment groups versus the oxycodone QID treatment group for selected adverse effects are shown in the following tables. The most frequent adverse effects reported as numbers of adverse events (AEs) were those commonly associated with opioid medications (so-called opioid related or opioid associated adverse effects): constipation, nausea, vomiting, dizziness, somnolence, and pruritis.

Table 49 shows the Opioid Associated Adverse Event Score (OAAES) for the treatment groups. The OAAES is a derived measure using the maximum severity for each of six opioid-associated adverse events (constipation, nausea, vomiting, dizziness, somnolence, and pruritis). For each patient, a maximum severity score (mild=1, moderate=2, and severe=3) was summed for each of the six treatment emergent opioid-associated adverse events that were present. If the patient did not have one of the six opioid-associated adverse events, the score for that event was zero. The OAAES scores for the oxycodone and naltrexone BID and QID treatment groups were lower than the OAAES scores for the oxycodone treatment group.

Table 50A through 50D show opioid-related adverse effects (e.g., constipation, nausea, vomiting, dizziness, somnolence, and pruritus). Table 50A shows the number (and percentage) of patients reporting opioid-associated adverse effects. Table 50B shows the number of each adverse event (and percentage that each adverse effect and system organ class comprised of the total adverse events). Table 50C shows the number (and percentage) of patients reporting each adverse event (and percentage that each adverse effect and system organ class comprised of the total adverse events) by maximum severity. Table 50D shows the number of each adverse event (and percentage that each adverse effect and system organ class comprised of the total adverse events) by severity through the treatment period.

TABLE 49
OPIOID ASSOCIATED ADVERSE EVENT SCORE (OAAES)
THROUGH THE TREATMENT PERIOD [1]
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| OAAES | | | | | |
| MEAN (SD) | 2.5 (2.84) | 5.6 (3.95) | 5.0 (3.81) | 5.1 (3.59) | 4.9 (3.79) |
| MEDIAN | 2.0 | 5.0 | 5.0 | 5.0 | 4.0 |
| MINIMUM (MAXIMUM) | 0.0 (12.0) | 0.0 (16.0) | 0.0 (17.0) | 0.0 (14.0) | 0.0 (17.0) |
| N | 101 | 206 | 206 | 206 | 719 |
| MODEL P-VALUES | | | | | |
| TREATMENT [2] | <0.001 | | | | |
| PAIRWISE COMPARISON P-VALUES [2] | | | | | |
| PLACEBO QID | — | <0.001 | <0.001 | <0.001 | |
| OXY QID | — | — | 0.123 | 0.163 | |
| OXY + NTX QID | — | — | — | 0.883 | |

[1] ADVERSE EVENT START DATE IS BETWEEN THE FIRST DOSE OF DRUG IN THE TITRATION PERIOD THROUGH THE LAST DOSE DATE, INCLUSIVE.
[2] P-VALUES FROM ANOVA MODEL INCLUDING TREATMENT AS THE MAIN EFFECT.

TABLE 50A
OPIOID-ASSOCIATED ADVERSE EVENTS THROUGH THE TREATMENT PERIOD [1]
ANALYSIS POPULATION: INTENT TO TREAT POPULATION

| | NUMBER (%) OF PATIENTS REPORTING EVENTS | | | | |
|---|---|---|---|---|---|
| SYSTEM ORGAN CLASS | PLACEBO QID | OXY QID | OXY + NTX QID | OXY + NTX BID | TOTAL |
| ADVERSE EVENT | (N = 101) | (N = 206) | (N = 206) | (N = 206) | (N = 719) |
| GASTROINTESTINAL DISORDERS | 51 (50.5%) | 160 (77.7%) | 156 (75.7%) | 159 (77.2%) | 526 (73.2%) |
| CONSTIPATION | 27 (26.7%) | 122 (59.2%) | 121 (58.7%) | 112 (54.4%) | 382 (53.1%) |
| NAUSEA | 34 (33.7%) | 120 (58.3%) | 114 (55.3%) | 121 (58.7%) | 389 (54.1%) |
| VOMITING | 11 (10.9%) | 50 (24.3%) | 46 (22.3%) | 49 (23.8%) | 156 (21.7%) |
| NERVOUS SYSTEM DISORDERS | 52 (51.5%) | 163 (79.1%) | 152 (73.8%) | 154 (74.8%) | 521 (72.5%) |
| DIZZINESS | 29 (28.7%) | 107 (51.9%) | 108 (52.4%) | 114 (55.3%) | 358 (49.8%) |
| SOMNOLENCE | 45 (44.6%) | 140 (68.0%) | 137 (66.5%) | 135 (65.5%) | 457 (63.6%) |
| SKIN AND SUBCUTANEOUS TISSUE | 21 (20.8%) | 124 (60.2%) | 94 (45.6%) | 105 (51.0%) | 344 (47.8%) |
| DISORDERS | | | | | |
| PRURITUS | 21 (20.8%) | 124 (60.2%) | 94 (45.6%) | 105 (51.0%) | 344 (47.8%) |

[1] ADVERSE EVENT START DATE IS BETWEEN THE FIRST DOSE OF DRUG IN THE TITRATION PERIOD THROUGH THE LAST DOSE DATE, INCLUSIVE.

TABLE 50B
OPIOID-ASSOCIATED ADVERSE EVENTS THROUGH THE TREATMENT PERIOD [1]
EVENT TABLE [2]
ANALYSIS POPULATION: INTENT TO TREAT POPULATION

| | NUMBER (%) OF EVENTS | | | | |
|---|---|---|---|---|---|
| SYSTEM ORGAN CLASS | PLACEBO QID | OXY QID | OXY + NTX QID | OXY + NTX BID | TOTAL |
| ADVERSE EVENT | (N = 101) | (N = 206) | (N = 206) | (N = 206) | (N = 719) |
| ALL EVENTS | 559 | 2549 | 2106 | 2155 | 7369 |
| GASTROINTESTINAL DISORDERS | 226 (40.4%) | 1143 (44.8%) | 900 (42.7%) | 963 (44.7%) | 3232 (43.9%) |
| CONSTIPATION | 105 (18.8%) | 576 (22.6%) | 467 (22.2%) | 425 (19.7%) | 1573 (21.3%) |
| NAUSEA | 103 (18.4%) | 459 (18.0%) | 362 (17.2%) | 434 (20.1%) | 1358 (18.4%) |
| VOMITING | 18 (3.2%) | 108 (4.2%) | 71 (3.4%) | 104 (4.8%) | 301 (4.1%) |
| NERVOUS SYSTEM DISORDERS | 272 (48.7%) | 952 (37.3%) | 847 (40.2%) | 833 (38.7%) | 2904 (39.4%) |
| DIZZINESS | 80 (14.3%) | 390 (15.3%) | 358 (17.0%) | 388 (18.0%) | 1216 (16.5%) |
| SOMNOLENCE | 192 (34.3%) | 562 (22.0%) | 489 (23.2%) | 445 (20.6%) | 1688 (22.9%) |
| SKIN AND SUBCUTANEOUS TISSUE | 61 (10.9%) | 454 (17.8%) | 359 (17.0%) | 359 (16.7%) | 1233 (16.7%) |
| DISORDERS | | | | | |
| PRURITUS | 61 (10.9%) | 454 (17.8%) | 359 (17.0%) | 359 (16.7%) | 1233 (16.7%) |

[1] ADVERSE EVENT START DATE IS BETWEEN THE FIRST DOSE OF DRUG IN THE TITRATION PERIOD THROUGH THE LAST DOSE DATE, INCLUSIVE.
[2] FOR EACH SUBJECT, ALL OCCURRENCES OF THE SAME ADVERSE EVENT WILL BE INCLUDED IN THE TABLE. PERCENTAGES ARE BASED ON TOTAL NUMBER OF EVENTS LISTED IN ALL EVENT ROW, WHILE N IN HEADER REPRESENTS TOTAL NUMBER OF PATIENTS.

TABLE 50C
OPIOID-ASSOCIATED ADVERSE EVENTS BY MAXIMUM SEVERITY
THROUGH THE TREATMENT PERIOD [1]
ANALYSIS POPULATION: INTENT TO TREAT POPULATION
NUMBER (%) OF PATIENTS REPORTING EVENTS

| SYSTEM ORGAN CLASS | | | | | |
|---|---|---|---|---|---|
| ADVERSE EVENT | PLACEBO QID | OXY QID | OXY + NTX QID | OXY + NTX BID | TOTAL |
| (MAXIMUM SEVERITY) | (N = 101) | (N = 206) | (N = 206) | (N = 206) | (N = 719) |
| GASTROINTESTINAL DISORDERS | 51 (50.5%) | 160 (77.7%) | 156 (75.7%) | 159 (77.2%) | 526 (73.2%) |
| CONSTIPATION | | | | | |
| SEVERE | 3 (3.0%) | 24 (11.7%) | 20 (9.7%) | 15 (7.3%) | 62 (8.6%) |
| MODERATE | 7 (6.9%) | 43 (20.9%) | 40 (19.4%) | 35 (17.0%) | 125 (17.4%) |
| MILD | 17 (16.8%) | 55 (26.7%) | 61 (29.6%) | 62 (30.1%) | 195 (27.3%) |
| NAUSEA | | | | | |
| SEVERE | 2 (2.0%) | 24 (11.7%) | 16 (7.8%) | 28 (13.6%) | 70 (9.7%) |
| MODERATE | 14 (13.9%) | 46 (22.3%) | 48 (23.3%) | 41 (19.9%) | 149 (20.7%) |
| MILD | 18 (17.8%) | 50 (24.3%) | 50 (24.3%) | 52 (25.2%) | 170 (23.6%) |
| VOMITING | | | | | |
| SEVERE | 1 (1.0%) | 14 (6.8%) | 11 (5.3%) | 11 (5.3%) | 37 (5.1%) |
| MODERATE | 7 (6.9%) | 16 (7.8%) | 21 (10.2%) | 20 (9.7%) | 64 (8.9%) |
| MILD | 3 (3.0%) | 20 (9.7%) | 14 (6.8%) | 18 (8.7%) | 55 (7.6%) |
| NERVOUS SYSTEM DISORDERS | 52 (51.5%) | 163 (79.1%) | 152 (73.8%) | 354 (74.8%) | 521 (72.5%) |
| DIZZINESS | | | | | |
| SEVERE | 3 (3.0%) | 13 (6.3%) | 17 (8.3%) | 20 (9.7%) | 53 (7.4%) |
| MODERATE | 6 (5.9%) | 33 (16.0%) | 32 (15.5%) | 33 (16.0%) | 104 (14.5%) |
| MILD | 20 (19.8%) | 61 (29.6%) | 59 (28.6%) | 61 (29.6%) | 201 (28.0%) |
| SOMNOLENCE | | | | | |
| SEVERE | 6 (5.9%) | 32 (15.5%) | 25 (12.1%) | 19 (9.2%) | 82 (11.4%) |
| MODERATE | 21 (20.8%) | 60 (29.1%) | 51 (24.8%) | 56 (27.2%) | 188 (26.1%) |
| MILD | 18 (17.8%) | 48 (23.3%) | 61 (29.6%) | 60 (29.1%) | 187 (26.0%) |
| SKIN AND SUBCUTANEOUS TISSUE | 21 (20.8%) | 124 (60.2%) | 94 (45.6%) | 105 (51.0%) | 344 (47.8%) |
| DISORDERS | | | | | |
| PRURITUS | | | | | |
| SEVERE | 0 (0.0%) | 13 (6.3%) | 10 (4.9%) | 3 (1.5%) | 26 (3.6%) |
| MODERATE | 5 (5.0%) | 52 (25.2%) | 28 (13.6%) | 36 (17.5%) | 121 (16.8%) |
| MILD | 16 (15.8%) | 59 (28.6%) | 56 (27.2%) | 66 (32.0%) | 197 (27.4%) |

[1] ADVERSE EVENT START DATE IS BETWEEN THE FIRST DOSE OF DRUG IN THE TITRATION PERIOD THROUGH THE LAST DOSE DATE, INCLUSIVE.

TABLE 50D
OPIOID-ASSOCIATED ADVERSE EVENTS BY SEVERITY THROUGH THE TREATMENT PERIOD [1]
EVENT TABLE [2]
ANALYSIS POPULATION: INTENT TO TREAT POPULATION
NUMBER (%) OF EVENTS

| SYSTEM ORGAN CLASS | | | | | |
|---|---|---|---|---|---|
| ADVERSE EVENT | PLACEBO QID | OXY QID | OXY + NTX QID | OXY + NTX BID | TOTAL |
| (MAXIMUM SEVERITY) | (N = 101) | (N = 206) | (N = 206) | (N = 206) | (N = 719) |
| ALL EVENTS | 559 | 2549 | 2106 | 2155 | 7369 |
| GASTROINTESTINAL DISORDERS | 226 (40.4%) | 1143 (44.8%) | 900 (42.7%) | 963 (44.7%) | 3232 (43.9%) |
| CONSTIPATION | | | | | |
| SEVERE | 6 (1.1%) | 27 (1.1%) | 23 (1.1%) | 19 (0.9%) | 75 (1.0%) |
| MODERATE | 22 (3.9%) | 119 (4.7%) | 91 (4.3%) | 63 (2.9%) | 295 (4.0%) |
| MILD | 77 (13.8%) | 430 (16.9%) | 353 (16.8%) | 343 (15.9%) | 1203 (16.3%) |
| NAUSEA | | | | | |
| SEVERE | 2 (0.4%) | 30 (1.2%) | 18 (0.9%) | 29 (1.3%) | 79 (1.1%) |
| MODERATE | 19 (3.4%) | 93 (3.6%) | 91 (4.3%) | 78 (3.6%) | 281 (3.8%) |
| MILD | 82 (14.7%) | 336 (13.2%) | 253 (12.0%) | 327 (15.2%) | 998 (13.5%) |
| VOMITING | | | | | |
| SEVERE | 2 (0.4%) | 21 (0.8%) | 12 (0.6%) | 13 (0.6%) | 48 (0.7%) |
| MODERATE | 7 (1.3%) | 26 (1.0%) | 27 (1.3%) | 33 (1.5%) | 93 (1.3%) |
| MILD | 9 (1.6%) | 61 (2.4%) | 32 (1.5%) | 58 (2.7%) | 160 (2.2%) |
| NERVOUS SYSTEM DISORDERS | 272 (48.7%) | 952 (37.3%) | 847 (40.2%) | 833 (38.7%) | 2904 (39.4%) |
| DIZZINESS | | | | | |
| SEVERE | 3 (0.5%) | 13 (0.5%) | 18 (0.9%) | 21 (1.0%) | 55 (0.7%) |
| MODERATE | 10 (1.8%) | 63 (2.5%) | 47 (2.2%) | 51 (2.4%) | 171 (2.3%) |
| MILD | 67 (12.0%) | 314 (12.3%) | 293 (13.9%) | 316 (14.7%) | 990 (13.4%) |
| SOMNOLENCE | | | | | |
| SEVERE | 15 (2.7%) | 39 (1.5%) | 29 (1.4%) | 22 (1.0%) | 105 (1.4%) |
| MODERATE | 36 (6.4%) | 133 (5.2%) | 97 (4.6%) | 94 (4.4%) | 160 (4.9%) |
| MILD | 141 (25.2%) | 390 (15.3%) | 363 (17.2%) | 329 (15.3%) | 1223 (16.6%) |
| SKIN AND SUBCUTANEOUS TISSUE | 61 (10.9%) | 454 (17.8%) | 359 (17.0%) | 359 (16.7%) | 1233 (16.7%) |
| DISORDERS | | | | | |
| PRURITUS | | | | | |
| SEVERE | 0 (0.0%) | 13 (0.5%) | 10 (0.5%) | 3 (0.1%) | 26 (0.4%) |
| MODERATE | 5 (0.9%) | 93 (3.6%) | 48 (2.3%) | 49 (2.3%) | 195 (2.6%) |
| MILD | 56 (10.0%) | 348 (13.7%) | 301 (14.3%) | 307 (14.2%) | 1012 (13.7%) |

[1] ADVERSE EVENT START DATE IS BETWEEN THE FIRST DOSE OF DRUG IN THE TITRATION PERIOD THROUGH THE LAST DOSE DATE, INCLUSIVE.
[2] FOR EACH SUBJECT, ALL OCCURRENCES OF THE SAME ADVERSE EVENT WILL BE INCLUDED IN THE TABLE. PERCENTAGES ARE BASED ON TOTAL NUMBER OF EVENTS LISTED IN ALL EVENT ROW, WHILE N IN HEADER REPRESENTS TOTAL NUMBER OF PATIENTS.

Exemplary opioid-related adverse effects include constipation, somnolence, and pruritus. Tables 51A-F show “Average AE Day of OAAES,” which is the total number of days with an adverse event, divided by the number of days on the study drug, including for the opioid-related adverse effects constipation, dizziness, somnolence, pruritus, nausea, and vomiting. The “N” values inside the Tables (as opposed to the N values at the top of the columns) for each treatment group shows the number of subjects reporting the adverse effect. For example, Tables 51A, C and D show that constipation, somnolence or pruritus was attenuated in the oxycodone and naltrexone BID treatment group as compared to the treatment group receiving oxycodone QID.

TABLE 51A
AE ANALYSIS-AVERAGE AE DAYS OF OPIOID ASSOCIATED AES START DURING TREATMENT
ANALYSIS POPULATION: INTENT TO TREAT POPULATION

| | PLACEBO QID | | OXY QID | | OXY + NTX QID | | OXY + NTX BID | | TOTAL | |
|---|---|---|---|---|---|---|---|---|---|---|
| | (N = 101) | | (N = 206) | | (N = 206) | | (N = 206) | | (N = 719) | |
| AVERAGE DAY FOR | | | | | | | | | | |
| CONSTIPATION | | | | | | | | | | |
| MEAN (SD) | 0.07 | (0.190) | 0.24 | (0.326) | 0.25 | (0.343) | 0.21 | (0.324) | 0.21 | (0.320) |
| MEDIAN | 0.00 | | 0.05 | | 0.03 | | 0.03 | | 0.02 | |
| MINIMUM (MAXIMUM) | 0.00 | (0.95) | 0.00 | (1.13) | 0.00 | (1.06) | 0.00 | (1.29) | 0.00 | (1.29) |
| N | 101 | | 206 | | 206 | | 2206 | | 719 | |
| MODEL P-VALUES | | | | | | | | | | |

| | | | | |
|---|---|---|---|---|
| TREATMENT [1] | <0.001 | | | |
| PAIRWISE COMPARISON P-VALUES | | | | |
| [1] | | | | |
| PLACEBO QID | — | <0.001 | <0.001 | <0.001 |
| OXY QID | — | — | 0.753 | 0.301 |
| OXY + NTX QID | — | — | — | 0.178 |

NOTE: AVERAGE AE DAY = (TOTAL NUMBER OF DAYS AE WITH AE)/(NUMBER OF DAYS ON STUDY DRUG). FOR CONTINUING AES, LAST DOSE DATE WERE USED AS STOP DATE.
[1] P-VALUES FROM ANOVA MODEL INCLUDING TREATMENT AS THE MAIN EFFECT.

TABLE 51B
AE ANALYSIS - AVERAGE AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AVERAGE AE DAY FOR DIZZINESS | | | | | |
| MEAN (SD) | 0.09 (0.315) | 0.14 (0.281) | 0.17 (0.290) | 0.21 (0.430) | 0.16 (0.338) |
| MEDIAN | 0.00 | 0.01 | 0.01 | 0.01 | 0.00 |
| MINIMUM (MAXIMUM) | 0.00 (2.50) | 0.00 (1.73) | 0.00 (1.03) | 0.00 (3.82) | 0.00 (3.82) |
| N | 101 | 206 | 206 | 206 | 719 |
| MODEL P-VALUES | | | | | |
| TREATMENT [1] | 0.040 | | | | |
| PAIRWISE COMPARISON P- | | | | | |
| VALUES [1] | | | | | |
| PLACEBO QID | — | 0.218 | 0.055 | 0.006 | |
| OXY QID | — | — | 0.397 | 0.064 | |
| OXY + NTX QID | — | — | — | 0.315 | |

NOTE:
AVERAGE AE DAY = (TOTAL NUMBER OF DAYS WITH AE)/(NUMBER OF DAYS ON STUDY DRUG). FOR CONTINUING AES, LAST DOSE DATE WERE USED AS STOP DATE.
[1] P-VALUES FROM ANOVA MODEL INCLUDING TREATMENT AS THE MAIN EFFECT.

TABLE 51C
AE ANALYSIS - AVERAGE AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AVERAGE AE DAY FOR | | | | | |
| SOMNOLENCE | | | | | |
| MEAN (SD) | 0.15 (0.276) | 0.31 (0.392) | 0.31 (0.366) | 0.28 (0.382) | 0.28 (0.371) |
| MEDIAN | 0.00 | 0.11 | 0.10 | 0.07 | 0.07 |
| MINIMUM (MAXIMUM) | 0.00 (1.33) | 0.00 (2.00) | 0.00 (1.24) | 0.00 (1.57) | 0.00 (2.00) |
| N | 101 | 206 | 206 | 206 | 719 |
| MODEL P-VALUES | | | | | |
| TREATMENT [1] | 0.002 | | | | |
| PAIRWISE COMPARISON P- | | | | | |
| VALUES [1] | | | | | |
| PLACEBO QID | — | <0.001 | <0.001 | 0.004 | |
| OXY QID | — | — | 0.931 | 0.356 | |
| OXY + NTX QID | — | — | — | 0.403 | |

NOTE:
AVERAGE AE DAY = (TOTAL NUMBER OF DAYS WITH AE)/(NUMBER OF DAYS ON STUDY DRUG). FOR CONTINUING AES, LAST DOSE DATE WERE USED AS STOP DATE.
[1] P-VALUES FROM ANOVA MODEL INCLUDING TREATMENT AS THE MAIN EFFECT.

TABLE 51D
AE ANALYSIS - AVERAGE AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AVERAGE AE DAY FOR PRURITUS | | | | | |
| MEAN (SD) | 0.05 (0.149) | 0.23 (0.355) | 0.16 (0.294) | 0.16 (0.294) | 0.17 (0.303) |
| MEDIAN | 0.00 | 0.03 | 0.00 | 0.01 | 0.00 |
| MINIMUM (MAXIMUM) | 0.00 (1.00) | 0.00 (2.00) | 0.00 (1.50) | 0.00 (1.40) | 0.00 (2.00) |
| N | 101 | 206 | 206 | 206 | 719 |
| MODEL P-VALUES | | | | | |
| TREATMENT [1] | <0.001 | | | | |
| PAIRWISE COMPARISON P- | | | | | |
| VALUES [1] | | | | | |
| PLACEBO QID | — | <0.001 | 0.002 | 0.001 | |
| OXY QID | — | — | 0.009 | 0.016 | |
| OXY + NTX QID | — | — | — | 0.843 | |

NOTE:
AVERAGE AE DAY = (TOTAL NUMBER OF DAYS WITH AE)/(NUMBER OF DAYS ON STUDY DRUG). FOR CONTINUING AES, LAST DOSE DATE WERE USED AS STOP DATE.
[1] P-VALUES FROM ANOVA MODEL INCLUDING TREATMENT AS THE MAIN EFFECT.

TABLE 51E
AE ANALYSIS - AVERAGE AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AVERAGE AE DAY FOR NAUSEA | | | | | |
| MEAN (SD) | 0.07 (0.178) | 0.14 (0.266) | 0.13 (0.237) | 0.17 (0.335) | 0.14 (0.271) |
| MEDIAN | 0.00 | 0.03 | 0.01 | 0.02 | 0.01 |
| MINIMUM (MAXIMUM) | 0.00 (0.89) | 0.00 (2.00) | 0.00 (1.33) | 0.00 (2.14) | 0.00 (2.14) |
| N | 101 | 206 | 206 | 206 | 719 |
| MODEL P-VALUES | | | | | |
| TREATMENT [1] | 0.024 | | | | |
| PAIRWISE COMPARISON P- | | | | | |
| VALUES [1] | | | | | |
| PLACEBO QID | — | 0.039 | 0.064 | 0.002 | |
| OXY QID | — | — | 0.792 | 0.223 | |
| OXY + NTX QID | — | — | — | 0.138 | |

NOTE:
AVERAGE AE DAY = (TOTAL NUMBER OF DAYS WITH AE)/(NUMBER OF DAYS ON STUDY DRUG). FOR CONTINUING AES, LAST DOSE DATE WERE USED AS STOP DATE.
[1] P-VALUES FROM ANOVA MODEL INCLUDING TREATMENT AS THE MAIN EFFECT.

TABLE 51F
AE ANALYSIS - AVERAGE AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AVERAGE AE DAY FOR VOMITING | | | | | |
| MEAN (SD) | 0.00 (0.010) | 0.02 (0.087) | 0.01 (0.047) | 0.03 (0.121) | 0.02 (0.084) |
| MEDIAN | 0.00 | 0.00 | 0.00 | 0.00 | 0.00 |
| MINIMUM (MAXIMUM) | 0.00 (0.06) | 0.00 (1.00) | 0.00 (0.50) | 0.00 (1.00) | 0.00 (1.00) |
| N | 101 | 206 | 206 | 206 | 719 |
| MODEL P-VALUES | | | | | |
| TREATMENT [1] | 0.025 | | | | |
| PAIRWISE COMPARISON P- | | | | | |
| VALUES [1] | | | | | |
| PLACEBO QID | — | 0.045 | 0.268 | 0.005 | |
| OXY QID | — | — | 0.270 | 0.319 | |
| OXY + NTX QID | — | — | — | 0.036 | |

NOTE:
AVERAGE AE DAY = (TOTAL NUMBER OF DAYS WITH AE)/(NUMBER OF DAYS ON STUDY DRUG). FOR CONTINUING AES, LAST DOSE DATE WERE USED AS STOP DATE.
[1] P-VALUES FROM ANOVA MODEL INCLUDING TREATMENT AS THE MAIN EFFECT.

Exemplary opioid-related adverse effects include constipation, somnolence, and pruritus. Tables 52A-F show “AE Day of OAAES,” which is the stop date of an adverse event, minus the start date of the adverse event plus 1, including for the opioid-related adverse effects constipation, dizziness, somnolence, pruritus, nausea, and vomiting. The “N” values inside the Tables (as opposed to the N values at the top of the columns) for each treatment group shows the number of subjects reporting the adverse effect. For example, Tables 52A, 52C and D show that constipation, somnolence or pruritus was attenuated in the oxycodone and naltrexone BID treatment group as compared to the treatment group receiving oxycodone QID.

TABLE 52A
AE ANALYSIS - AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AE DAY FOR CONSTIPATION | | | | | |
| MEAN (SD) | 5.13 (14.994) | 17.59 (27.733) | 17.75 (30.914) | 14.04 (26.960) | 14.87 (27.374) |
| MEDIAN | 0.00 | 3.00 | 2.00 | 2.00 | 1.00 |
| MINIMUM (MAXIMUM) | 0.00 (102.00) | 0.00 (114.00) | 0.00 (128.00) | 0.00 (119.00) | 0.00 (128.00) |
| N | 101 | 206 | 206 | 206 | 719 |
| MODEL P-VALUES | | | | | |
| TREATMENT [1] | <0.001 | | | | |
| PAIRWISE COMPARISON P-VALUES [1] | | | | | |
| PLACEBO QID | — | <0.001 | <0.001 | 0.007 | |
| OXY QID | — | — | 0.951 | 0.185 | |
| OXY + NTX QID | — | — | — | 0.165 | |

NOTE:
AE DAY = AE STOP DATE − AE START DATE + 1. FOR CONTINUING AES, LAST DOSE DATE WERE USED AS STOP DATE.
[1] P-VALUES FROM ANOVA MODEL INCLUDING TREATMENT AS THE MAIN EFFECT.

TABLE 52B
AE ANALYSIS - AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AE DAY FOR DIZZINESS | | | | | |
| MEAN (SD) | 3.22 (12.773) | 8.59 (18.871) | 9.92 (22.423) | 8.63 (17.848) | 8.23 (19.062) |
| MEDIAN | 0.00 | 1.00 | 1.00 | 1.00 | 0.00 |
| MINIMUM (MAXIMUM) | 0.00 (120.00) | 0.00 (114.00) | 0.00 (117.00) | 0.00 (114.00) | 0.00 (120.00) |
| N | 101 | 206 | 206 | 206 | 719 |
| MODEL P-VALUES | | | | | |
| TREATMENT [1] | 0.032 | | | | |
| PAIRWISE COMPARISON P-VALUES [1] | | | | | |
| PLACEBO QID | — | 0.020 | 0.004 | 0.019 | |
| OXY QID | — | — | 0.476 | 0.983 | |
| OXY + NTX QID | — | — | — | 0.489 | |

NOTE:
AE DAY = AE STOP DATE − AE START DATE + 1. FOR CONTINUING AES, LAST DOSE DATE WERE USED AS STOP DATE.
[1] P-VALUES FROM ANOVA MODEL INCLUDING TREATMENT AS THE MAIN EFFECT.

TABLE 52C
AE ANALYSIS - AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AE DAY FOR SOMNOLENCE | | | | | |
| MEAN (SD) | 9.95 (20.878) | 21.53 (32.137) | 18.56 (29.642) | 15.86 (26.947) | 17.43 (28.767) |
| MEDIAN | 0.00 | 6.00 | 4.00 | 3.00 | 3.00 |
| MINIMUM (MAXIMUM) | 0.00 (132.00) | 0.00 (123.00) | 0.00 (119.00) | 0.00 (121.00) | 0.00 (123.00) |
| N | 101 | 206 | 206 | 206 | 719 |
| MODEL P-VALUES | | | | | |
| TREATMENT [1] | 0.007 | | | | |
| PAIRWISE COMPARISON P-VALUES [1] | | | | | |
| PLACEBO QID | — | <0.001 | 0.013 | 0.089 | |
| OXY QID | — | — | 0.291 | 0.044 | |
| OXY + NTX QID | — | — | — | 0.338 | |

NOTE:
AE DAY = AE STOP DATE − AE START DATE + 1. FOR CONTINUING AES, LAST DOSE DATE WERE USED AS STOP DATE.
[1] P-VALUES FROM ANOVA MODEL INCLUDING TREATMENT AS THE MAIN EFFECT.

TABLE 52D
AE ANALYSIS - AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AE DAY FOR PRURITUS | | | | | |
| MEAN (SD) | 2.60 (9.151) | 15.92 (28.817) | 11.37 (24.921) | 10.64 (23.555) | 11.23 (24.523) |
| MEDIAN | 0.00 | 2.00 | 0.00 | 1.00 | 0.00 |
| MINIMUM (MAXIMUM) | 0.00 (67.00) | 0.00 (124.00) | 0.00 (112.00) | 0.00 (137.00) | 0.00 (137.00) |
| N | 101 | 206 | 206 | 206 | 719 |
| MODEL P-VALUES | | | | | |
| TREATMENT [1] | <0.001 | | | | |
| PAIRWISE COMPARISON P-VALUES [1] | | | | | |
| PLACEBO QID | — | <0.001 | 0.003 | 0.006 | |
| OXY QID | — | — | 0.057 | 0.027 | |
| OXY + NTX QID | — | — | — | 0.760 | |

NOTE:
AE DAY = AE STOP DATE − AE START DATE + 1. FOR CONTINUING AES, LAST DOSE DATE WERE USED AS STOP DATE.
[1] P-VALUES FROM ANOVA MODEL INCLUDING TREATMENT AS THE MAIN EFFECT.

TABLE 52E
AE ANALYSIS - AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AE DAY FOR NAUSEA | | | | | |
| MEAN (SD) | 3.04 (10.028) | 8.16 (16.511) | 7.25 (16.866) | 7.54 (14.626) | 7.00 (15.389) |
| MEDIAN | 0.00 | 2.00 | 1.00 | 2.00 | 1.00 |
| MINIMUM (MAXIMUM) | 0.00 (87.00) | 0.00 (118.00) | 0.00 (108.00) | 0.00 (116.00) | 0.00 (118.00) |
| N | 101 | 206 | 206 | 206 | 719 |
| MODEL P-VALUES | | | | | |
| TREATMENT [1] | 0.043 | | | | |
| PAIRWISE COMPARISON P-VALUES [1] | | | | | |
| PLACEBO QID | — | 0.006 | 0.024 | 0.016 | |
| OXY QID | — | — | 0.548 | 0.683 | |
| OXY + NTX QID | — | — | — | 0.847 | |

NOTE:
AE DAY = AE STOP DATE − AE START DATE + 1. FOR CONTINUING AES, LAST DOSE DATE WERE USED AS STOP DATE.
[1] P-VALUES FROM ANOVA MODEL INCLUDING TREATMENT AS THE MAIN EFFECT.

TABLE 52F
AE ANALYSIS - AE DAYS OF OPIOID ASSOCIATED AES START DURING TREATMENT
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AE DAY FOR VOMITING | | | | | |
| MEAN (SD) | 0.23 (0.847) | 1.09 (2.839) | 0.76 (2.345) | 1.11 (2.815) | 0.88 (2.515) |
| MEDIAN | 0.00 | 0.00 | 0.00 | 0.00 | 0.00 |
| MINIMUM (MAXIMUM) | 0.00 (7.00) | 0.00 (18.00) | 0.00 (25.00) | 0.00 (18.00) | 0.00 (25.00) |
| N | 101 | 206 | 206 | 206 | 719 |
| MODEL P-VALUES | | | | | |
| TREATMENT [1] | 0.015 | | | | |
| PAIRWISE COMPARISON P-VALUES [1] | | | | | |
| PLACEBO QID | — | 0.005 | 0.079 | 0.004 | |
| OXY QID | — | — | 0.187 | 0.922 | |
| OXY + NTX QID | — | — | — | 0.157 | |

NOTE:
AE DAY = AE STOP DATE − AE START DATE + 1. FOR CONTINUING AES, LAST DOSE DATE WERE USED AS STOP DATE.
[1] P-VALUES FROM ANOVA MODEL INCLUDING TREATMENT AS THE MAIN EFFECT.

Another assessment was an assessment of opiate withdrawal symptoms at the end of treatment using for this study the Short Opioid Withdrawal Scales (SOWS) (see Table 6 above). The SOWS scores are shown in Table 53 of the subjects at baseline (last day of study drug administration) and Days 1, 2, 3 and 4 (after the last day of study drug administration), based on subjects with greater than or equal to four weeks of dosing. In general, a lower SOWS score indicates that symptoms of withdrawal are better and a higher SOWS score indicates that symptoms of withdrawal are worse. The SOWS scores in Table 53 show that withdrawal was attenuated in the treatment groups receiving oxycodone and naltrexone BID or oxycodone and naltrexone QID as compared to the treatment group receiving oxycodone QID. Moreover, the treatment group that received oxycodone and naltrexone BID had a change in SOWS scores at Days 1 and 3 that did not differ to a statistically significant degree from the treatment group that received placebo. The SOWS scores for the oxycodone and naltrexone BID treatment group were better than the SOWS scores for the oxycodone QID treatment group on all days (Days 1, 2, 3 and 4) of opioid withdrawal monitoring.

TABLE 53
SHORT OPIATE WITHDRAWAL SCORES[1]
PATIENTS WITH ≧4 WEEKS OF DOSING
ANALYSIS POPULATION: INTENT TO TREAT POPULATION

| | PLACEBO QID | OXY QID | OXY + NTX QID | OXY + NTX |
|---|---|---|---|---|
| | (N = 70) | (N = 138) | (N = 128) | BID (N = 131) |
| LAST DOSE DATE (BASELINE) | | | | |
| MEAN (SD) | 0.2 (0.25) | 0.3 (0.37) | 0.3 (0.35) | 0.3 (0.38) |
| MEDIAN | 0.0 | 0.2 | 0.2 | 0.2 |
| MINIMUM (MAXIMUM) | 0.0 (0.9) | 0.0 (1.5) | 0.0 (1.6) | 0.0 (2.4) |
| N | 61 | 110 | 103 | 99 |
| OPIOID WITHDRAWAL MONITORING - DAY 1 | | | | |
| MEAN (SD) | 0.2 (0.20) | 0.5 (0.53) | 0.5 (0.48) | 0.4 (0.44) |
| MEDIAN | 0.1 | 0.4 | 0.4 | 0.3 |
| MINIMUM (MAXIMUM) | 0.0 (0.9) | 0.0 (2.5) | 0.0 (2.0) | 0.0 (2.3) |
| N | 67 | 124 | 117 | 121 |
| CHANGE FROM BASELINE TO DAY 1 | | | | |
| MEAN (SD) | −0.0 (0.26) | 0.3 (0.48) | 0.2 (0.41) | 0.1 (0.41) |
| MEDIAN | 0.0 | 0.2 | 0.1 | 0.0 |
| MINIMUM (MAXIMUM) | −0.7 (0.9) | −0.7 (1.9) | −0.8 (1.8) | −0.9 (1.8) |
| N | 59 | 106 | 102 | 97 |
| TREATMENT P-VALUE [2]: | <0.001 | | | |
| PAIRWISE COMPARISON P-VALUES [2] | | | | |
| PLACEBO QID | — | <0.001 | <0.001 | 0.060 |
| OXY QID | — | — | 0.398 | 0.008 |
| OXY + NTX QID | — | — | — | 0.072 |
| OPIOID WITHDRAWAL MONITORING - DAY 2 | | | | |
| MEAN (SD) | 0.2 (0.30) | 0.6 (0.54) | 0.6 (0.51) | 0.5 (0.52) |
| MEDIAN | 0.1 | 0.4 | 0.5 | 0.4 |
| MINIMUM (MAXIMUM) | 0.0 (1.6) | 0.0 (2.5) | 0.0 (2.3) | 0.0 (2.6) |
| N | 67 | 126 | 120 | 125 |
| CHANGE FROM BASELINE TO DAY 2 | | | | |
| MEAN (SD) | 0.0 (0.35) | 0.3 (0.52) | 0.3 (0.46) | 0.2 (0.57) |
| MEDIAN | 0.0 | 0.2 | 0.2 | 0.1 |
| MINIMUM (MAXIMUM) | −0.7 (1.4) | −1.1 (2.4) | −1.0 (1.8) | −0.9 (2.6) |
| N | 59 | 105 | 101 | 96 |
| TREATMENT P-VALUE [2]: | 0.005 | | | |
| PAIRWISE COMPARISON P-VALUES [2] | | | | |
| PLACEBO QID | — | <0.001 | 0.002 | 0.012 |
| OXY QID | — | — | 0.768 | 0.330 |
| OXY + NTX QID | — | — | — | 0.499 |
| OPIOID WITHDRAWAL MONITORING - DAY 3 | | | | |
| MEAN (SD) | 0.2 (0.40) | 0.6 (0.53) | 0.5 (0.45) | 0.5 (0.50) |
| MEDIAN | 0.1 | 0.4 | 0.4 | 0.4 |
| MINIMUM (MAXIMUM) | 0.0 (2.4) | 0.0 (2.2) | 0.0 (2.1) | 0.0 (2.7) |
| N | 67 | 130 | 123 | 125 |
| CHANGE FROM BASELINE TO DAY 3 | | | | |
| MEAN (SD) | 0.1 (0.43) | 0.3 (0.48) | 0.2 (0.39) | 0.2 (0.56) |
| MEDIAN | 0.0 | 0.2 | 0.2 | 0.1 |
| MINIMUM (MAXIMUM) | −0.7 (2.2) | −1.1 (1.7) | −1.2 (1.5) | −1.1 (2.7) |
| N | 58 | 104 | 100 | 95 |
| TREATMENT P-VALUE [2]: | 0.117 | | | |
| PAIRWISE COMPARISON P-VALUES [2] | | | | |
| PLACEBO QID | — | 0.020 | 0.048 | 0.062 |
| OXY QID | — | — | 0.696 | 0.624 |
| OXY + NTX QID | — | — | — | 0.918 |
| OPIOID WITHDRAWAL MONITORING - DAY 4 | | | | |
| MEAN (SD) | 0.2 (0.34) | 0.5 (0.55) | 0.4 (0.40) | 0.4 (0.38) |
| MEDIAN | 0.1 | 0.3 | 0.3 | 0.3 |
| MINIMUM (MAXIMUM) | 0.0 (1.3) | 0.0 (2.7) | 0.0 (1.7) | 0.0 (1.9) |
| N | 61 | 118 | 117 | 116 |
| CHANGE FROM BASELINE TO DAY 4 | | | | |
| MEAN (SD) | 0.0 (0.38) | 0.2 (0.55) | 0.2 (0.42) | 0.1 (0.40) |
| MEDIAN | 0.0 | 0.1 | 0.1 | 0.1 |
| MINIMUM (MAXIMUM) | −0.7 (1.1) | −1.1 (2.7) | −1.4 (1.5) | −0.9 (1.1) |
| N | 52 | 94 | 93 | 86 |
| TREATMENT P-VALUE [2]: | 0.255 | | | |
| PAIRWISE COMPARISON P-VALUES [2] | | | | |
| PLACEBO QID | — | 0.066 | 0.106 | 0.362 |
| OXY QID | — | — | 0.792 | 0.288 |
| OXY + NTX QID | — | — | — | 0.422 |

SHORT OPIATE WITHDRAWAL SCORES[1]
PATIENTS WITH <4 WEEKS OP DOSING
ANALYSIS POPULATION: INTENT TO TREAT POPULATION

| | PLACEBO QID | OXY QID | PTI-801 QID | PTI-801 BID |
|---|---|---|---|---|
| | (N = 8) | (N = 16) | (N = 17) | (N = 15) |
| LAST DOSE DATE (BASELINE) | | | | |
| MEAN(SD) | 0.6 (0.23) | 0.3 (0.29) | 0.2 (0.21) | 0.5 (0.56) |
| MEDIAN | 0.7 | 0.2 | 0.3 | 0.4 |
| MIN, MAX | 0.2, 0.8 | 0.0, 0.7 | 0.0, 0.5 | 0.0, 1.2 |
| N | 5 | 6 | 11 | 6 |
| OPIOID WITHDRAWAL MONITORING - DAY 1 | | | | |
| MEAN(SD) | 0.2 (0.40) | 0.2 (0.27) | 0.3 (0.39) | 0.4 (0.45) |
| MEDIAN | 0.0 | 0.1 | 0.1 | 0.2 |
| MIN, MAX | 0.0, 0.8 | 0.0, 0.9 | 0.0, 1.0 | 0.0, 1.2 |
| N | 4 | 12 | 10 | 11 |
| CHANGE FROM BASELINE TO DAY 1 | | | | |
| MEAN(SD) | −0.53 (0.26) | 0.0 (0.16) | −0.1 (0.22) | 0.1 (0.20) |
| MEDIAN | 0.6 | 0.0 | −0.1 | 0.0 |
| MIN, MAX | −0.7, −0.2 | −0.2, 0.2 | −0.4, 0.2 | −0.1, 0.4 |
| N | 3 | 5 | 6 | 6 |
| TREATMENT P-VALUE [2]: | 0.009 | | | |
| PAIRWISE COMPARISON P-VALUES [2] | | | | |
| PLACEBO QID | — | 0.004 | 0.014 | 0.001 |
| OXY QID | — | — | 0.433 | 0.599 |
| PTI-801 QID | — | — | — | 0.178 |
| OPIOID WITHDRAWAL MONITORING - DAY 2 | | | | |
| MEAN(SD) | 0.2 (0.28) | 0.2 (0.38) | 0.3 (0.38) | 0.2 (0.31) |
| MEDIAN | 0.1 | 0.0 | 0.1 | 0.1 |
| MIN, MAX | 0.0, 0.7 | 0.0, 1.3 | 0.0, 1.1 | 0.0, 1.0 |
| N | 6 | 11 | 10 | 11 |
| CHANGE FROM BASELINE TO DAY 2 | | | | |
| MEAN(SD) | −0.5 (0.23) | 0.2 (0.66) | −0.1 (0.19) | −0.2 (0.31) |
| MEDIAN | −0.6 | 0.1 | −0.1 | −0.1 |
| MIN, MAX | −0.6, −0.2 | −0.5, 1.3 | −0.4, 0.1 | −0.7, 0.2 |
| N | 3 | 5 | 6 | 6 |
| TREATMENT P-VALUE [2]: | 0.180 | | | |
| PAIRWISE COMPARISON P-VALUES [2] | | | | |
| PLACEBO QID | — | 0.036 | 0.233 | 0.304 |
| OXY QID | — | — | 0.209 | 0.149 |
| PTI-801 QID | — | — | — | 0.831 |
| OPIOID WITHDRAWAL MONITORING - DAY 3 | | | | |
| MEAN(SD) | 0.1 (0.13) | 0.2 (0.39) | 0.2 (0.25) | 0.3 (0.35) |
| MEDIAN | 0.1 | 0.0 | 0.0 | 0.2 |
| MIN, MAX | 0.0, 0.3 | 0.0, 1.3 | 0.0, 0.7 | 0.0, 1.0 |
| N | 6 | 11 | 8 | 12 |
| CHANGE FROM BASELINE TO DAY 3 | | | | |
| MEAN(SD) | −0.5 (0.23) | 0.2 (0.67) | −0.1 (0.22) | −0.2 (0.36) |
| MEDIAN | −0.6 | 0.0 | −0.1 | −0.1 |
| MIN, MAX | −0.6, −0.2 | −0.5, 1.3 | −0.4, 0.2 | −0.8, 0.2 |
| N | 3 | 5 | 6 | 6 |
| TREATMENT P-VALUE [2]: | 0.250 | | | |
| PAIRWISE COMPARISON P-VALUES [2] | | | | |
| PLACEBO QID | — | 0.057 | 0.233 | 0.381 |
| OXY QID | — | — | 0.320 | 0.174 |
| PTI-801 QID | — | — | — | 0.684 |
| OPIOID WITHDRAWAL MONITORING - DAY 4 | | | | |
| MEAN(SD) | 0.1 (0.09) | 0.1 (0.26) | 0.1 (0.15) | 0.3 (0.42) |
| MEDIAN | 0.0 | 0.0 | 0.0 | 0.1 |
| MIN, MAX | 0.0, 0.2 | 0.0, 0.8 | 0.0, 0.4 | 0.0, 1.1 |
| N | 5 | 10 | 8 | 11 |
| CHANGE FROM BASELINE TO DAY 4 | | | | |
| MEAN(SD) | −0.7 (0.07) | 0.2 (0.41) | −0.1 (0.22) | 0.2 (0.24) |
| MEDIAN | −0.7 | 0.1 | −0.1 | 0.1 |
| MIN, MAX | −0.7, −0.6 | −0.1, 0.8 | −0.4, 0.2 | −0.1, 0.5 |
| N | 2 | 4 | 6 | 5 |
| TREATMENT P-VALUE [2]: | 0.013 | | | |
| PAIRWISE COMPARISON P-VALUES [2] | | | | |
| PLACEBO QID | — | 0.003 | 0.029 | 0.004 |
| OXY QID | — | — | 0.114 | 0.831 |
| PTI-801 QID | — | — | — | 0.141 |

SHORT OPIATE WITHDRAWAL SCORES[1]
ALL PATIENTS
ANALYSIS POPULATION: INTENT TO TREAT POPULATION

| | PLACEBO QID | OXY QID | PTI-801 QID | PTI-802 BID |
|---|---|---|---|---|
| | (N = 78) | (N = 154) | (N = 145) | (N = 146) |
| LAST DOSE DATE (BASELINE) | | | | |
| MEAN(SD) | 0.2 (0.27) | 0.3 (0.36) | 0.3 (0.34) | 0.3 (0.39) |
| MEDIAN | 0.1 | 0.2 | 0.2 | 0.2 |
| MIN, MAX | 0.0, 0.9 | 0.0, 1.5 | 0.0, 1.6 | 0.0, 2.4 |
| N | 66 | 116 | 114 | 105 |
| OPIOID WITHDRAWAL MONITORING - DAY 1 | | | | |
| MEAN(SD) | 0.2 (0.21) | 0.5 (0.52) | 0.5 (0.47) | 0.4 (0.44) |
| MEDIAN | 0.1 | 0.4 | 0.4 | 0.3 |
| MIN, MAX | 0.0, 0.9 | 0.0, 2.5 | 0.0, 2.0 | 0.0, 2.3 |
| N | 71 | 136 | 127 | 132 |
| CHANGE FROM BASELINE TO DAY 1 | | | | |
| MEAN(SD) | −0.0 (0.28) | 0.3 (0.47) | 0.2 (0.41) | 0.1 (0.40) |
| MEDIAN | 0.0 | 0.2 | 0.1 | 0.0 |
| MIN, MAX | −0.7, 0.9 | −0.7, 1.9 | −0.8, 1.8 | −0.9, 1.8 |
| N | 62 | 111 | 100 | 103 |
| TREATMENT P-VALUE [2]: | <0.001 | | | |
| PAIRWISE COMPARISON P-VALUES [2] | | | | |
| PLACEBO QID | — | <0.001 | <0.001 | 0.020 |
| OXY QID | — | — | 0.328 | 0.010 |
| PTI-801 QID | — | — | — | 0.109 |
| OPIOID WITHDRAWAL MONITORING - DAY 2 | | | | |
| MEAN(SD) | 0.2 (0.30) | 0.5 (0.54) | 0.6 (0.51) | 0.5 (0.51) |
| MEDIAN | 0.1 | 0.4 | 0.4 | 0.4 |
| MIN, MAX | 0.0, 1.6 | 0.0, 2.5 | 0.0, 2.3 | 0.0, 2.6 |
| N | 73 | 137 | 130 | 136 |
| CHANGE FROM BASELINE TO DAY 2 | | | | |
| MEAN(SD) | 0.0 (0.36) | 0.3 (0.53) | 0.3 (0.45) | 0.2 (0.56) |
| MEDIAN | 0.0 | 0.2 | 0.2 | 0.1 |
| MIN, MAX | −0.7, 1.4 | −1.1, 2.4 | −1.0, 1.8 | −0.9, 2.6 |
| N | 62 | 110 | 107 | 102 |
| TREATMENT P-VALUE [2]: | 0.002 | | | |
| PAIRWISE COMPARISON P-VALUES [2] | | | | |
| PLACEBO QID | — | <0.001 | 0.001 | 0.008 |
| OXY QID | — | — | 0.571 | 0.201 |
| PTI-801 QID | — | — | — | 0.475 |
| OPIOID WITHDRAWAL MONITORING - DAY 3 | | | | |
| MEAN(SD) | 0.2 (0.39) | 0.5 (0.53) | 0.5 (0.45) | 0.5 (0.49) |
| MEDIAN | 0.1 | 0.4 | 0.4 | 0.4 |
| MIN, MAX | 0.0, 2.4 | 0.0, 2.2 | 0.0, 2.1 | 0.0, 2.7 |
| N | 73 | 141 | 131 | 137 |
| CHANGE FROM BASELINE TO DAY 3 | | | | |
| MEAN(SD) | 0.0 (0.44) | 0.2 (0.48) | 0.2 (0.39) | 0.2 (0.56) |
| MEDIAN | 0.0 | 0.2 | 0.2 | 0.1 |
| MIN, MAX | −0.7, 2.2 | −1.1, 1.7 | −1.2, 1.5 | −1.1, 2.7 |
| N | 61 | 109 | 106 | 101 |
| TREATMENT P-VALUE [2]: | 0.051 | | | |
| PAIRWISE COMPARISON P-VALUES [2] | | | | |
| PLACEBO QID | — | 0.007 | 0.029 | 0.046 |
| OXY QID | — | — | 0.536 | 0.414 |
| PTI-801 QID | — | — | — | 0.839 |
| OPIOID WITHDRAWAL MONITORING - DAY 4 | | | | |
| MEAN(SD) | 0.2 (0.33) | 0.4 (0.54) | 0.4 (0.40) | 0.4 (0.38) |
| MEDIAN | 0.1 | 0.3 | 0.3 | 0.3 |
| MIN, MAX | 0.0, 1.3 | 0.0, 2.7 | 0.0, 1.7 | 0.0, 1.9 |
| N | 66 | 128 | 125 | 127 |
| CHANGE FROM BASELINE TO DAY 4 | | | | |
| MEAN(SD) | 0.0 (0.40) | 0.2 (0.54) | 0.2 (0.42) | 0.1 (0.39) |
| MEDIAN | 0.0 | 0.1 | 0.1 | 0.1 |
| MIN, MAX | −0.7, 1.1 | −1.1, 2.7 | −1.4, 1.5 | −0.9, 1.1 |
| N | 54 | 98 | 99 | 91 |
| TREATMENT P-VALUES [2]: | 0.149 | | | |
| PAIRWISE COMPARISON P-VALUES [2] | | | | |
| PLACEBO QID | — | 0.026 | 0.074 | 0.193 |
| OXY QID | — | — | 0.591 | 0.285 |
| PTI-801 QID | — | — | — | 0.585 |

[1] SOWS OBTAINED FIVE DAYS OR MORE AFTER THE LAST DOSE DATE ARE NOT ANALYZED. A HIGHER SCORE INDICATES WORSE WITHDRAW SYMPTOM.
[2] P-VALUES FROM ANOVA MODEL WITH TREATMENT AS THE EFFECT.

Another efficacy assessment was a dose analysis of the average titration dose for the treatment groups in this clinical study. Table 54 shows the average titration dose in mg of opioid agonist. Table 54 shows that the oxycodone and naltrexone BID or oxycodone and naltrexone QID treatment groups had lower mean titration doses of as oxycodone (22.2 mg for the oxycodone and naltrexone BID group and 22.0 mg for the oxycodone and naltrexone QID group) than the treatment group receiving oxycodone QID (24.2 mg). Table 54 shows that the opioid antagonist enhanced the potency of the opioid agonist for alleviating back pain. Lower doses of oxycodone in the oxycodone and naltrexone BID or oxycodone and naltrexone QID treatment groups achieved the desired effects than in the oxycodone QID treatment group.

TABLE 54
DOSE ANALYSIS
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AVERAGE TITRATION DOSE (MG) | | | | | |
| MEAN (SD) | 26.0 (8.34) | 24.2 (8.22) | 22.0 (9.72) | 22.2 (9.39) | 23.3 (9.12) |
| MEDIAN | 30.0 | 25.9 | 24.4 | 22.5 | 25.9 |
| MINIMUM (MAXIMUM) | 10.0 (38.0) | 0.0 (42.3) | 0.0 (44.4) | 0.0 (45.6) | 0.0 (45.6) |
| N | 101 | 206 | 206 | 206 | 719 |
| MODEL P-VALUES | | | | | |
| TREATMENT (1) | <0.001 | | | | |
| PAIRWISE COMPARISON P-VALUES [1] | | | | | |
| PLACEBO QID | — | 0.093 | <0.001 | <0.001 | |
| OXY QID | — | — | 0.013 | 0.028 | |
| OXY + NTX QID | — | — | — | 0.767 | |

[1] P-VALUES FROM ANOVA MODEL INCLUDING TREATMENT AS THE MAIN EFFECT.

Another efficacy assessment was a dose analysis of the average fixed dose for the treatment groups in this clinical study. Table 55 shows the average fixed dose in mg of opioid agonist. Table 55 shows lower mean fixed doses for the treatment groups receiving oxycodone and naltrexone BID (52.8 mg) and QID (52.0 mg) compared to the treatment group receiving oxycodone (57.2 mg). Table 55 shows that the opioid antagonist enhanced the potency of the opioid agonist for alleviating the back pain. Lower fixed doses of oxycodone in the oxycodone and naltrexone BID and QID treatment groups achieved the desired effect than in the oxycodone QID treatment group.

TABLE 55
DOSE ANALYSIS
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AVERAGE FIXED DOSE (MG) | | | | | |
| MEAN (SD) | 69.6 (18.89) | 57.2 (23.35) | 52.0 (27.01) | 52.8 (26.11) | 56.0 (25.32) |
| MEDIAN | 80.0 | 60.0 | 60.0 | 60.0 | 60.0 |
| MINIMUM (MAXIMUM) | 10.0 (80.0) | 10.0 (80.0) | 10.0 (80.0) | 10.0 (80.0) | 10.0 (80.0) |
| N | 57 | 142 | 135 | 131 | 465 |
| MODEL P-VALUES | <0.001 | | | | |
| TREATMENT (1) | | | | | |
| PAIRWISE COMPARISON P-VALUES [1] | | | | | |
| PLACEBO QID | — | 0.001 | <0.001 | <0.001 | |
| OXY QID | — | — | 0.083 | 0.147 | |
| OXY + NTX QID | — | — | — | 0.786 | |

[1] P-VALUES FROM ANOVA MODEL INCLUDING TREATMENT AS THE MAIN EFFECT.

Another efficacy assessment in this clinical study was a dose analysis of the average study drug dose for the treatment groups. Table 56 shows the average study dose in mg of opioid agonist. Table 56 shows that the oxycodone and naltrexone BID and QID treatment groups had lower mean study drug doses of as oxycodone (34.7 mg for the BID group and 34.5 mg for the QID group) compared to the treatment group receiving oxycodone (39.0 mg). Table 56 shows that the opioid antagonist enhanced the potency of the opioid agonist for alleviating the back pain. Lower study drug doses of oxycodone in the oxycodone and naltrexone BID and QID treatment groups achieved the desired effect than in the oxycodone QID treatment group.

TABLE 56
DOSE ANALYSIS
ANALYSIS POPULATION: INTENT TO TREAT POPULATION

| | PLACEBO QID | OXY QID | OXY + NTX QID | OXY + NTX BID | TOTAL |
|---|---|---|---|---|---|
| | (N = 101) | (N = 206) | (N = 206) | (N = 206) | (N = 719) |
| AVERAGE STUDY DRUG DOSE (MG) | | | | | |
| MEAN (SD) | 41.5 (20.57) | 39.0 (19.87) | 34.5 (20.44) | 34.7 (20.88) | 36.9 (20.56) |
| MEDIAN | 38.0 | 35.6 | 29.3 | 29.3 | 32.4 |
| MINIMUM (MAXIMUM) | 10.0 (66.4) | 10.0 (67.8) | 10.0 (68.1) | 10.0 (67.9) | 10.0 (68.1) |
| N | 101 | 206 | 206 | 206 | 719 |
| MODEL P-VALUES | | | | | |
| TREATMENT [1] | 0.006 | | | | |
| PAIRWISE COMPARISON P-VALUES [1] | | | | | |
| PLACEBO QID | — | 0.308 | 0.005 | 0.006 | |
| OXY QID | — | — | 0.027 | 0.035 | |
| OXY + NTX QID | — | — | — | 0.921 | |

[1] P-VALUES FROM ANOVA MODEL INCLUDING TREATMENT AS THE MAIN EFFECT.

In summary, in this study oxycodone and naltrexone BID and oxycodone and naltrexone QID were shown to be safe and effective treatments for patients with back pain. Patients taking oxycodone and naltrexone BID or oxycodone and naltrexone QID requires less opioid drug dose (approximately 35 mg per day) to achieve comparable pain relief compared to patients taking oxycodone QID (approximately 39 mg) (p<0.05). See, e.g., Table 56. Both oxycodone and naltrexone BID and oxycodone and naltrexone QID alleviated back pain (about 45% mean reduction in pain intensity from baseline) see, e.g., Table 45, and also provided better attenuation of adverse effects than oxycodone QID. See, e.g., Tables 49-52. For example, oxycodone and naltrexone BID and oxycodone and naltrexone QID provided alleviation of back pain, and the symptoms of withdrawal (as reflected in the SOWS scores above) were attenuated as compared to oxycodone QID. See, e.g., Table 53. Patients taking oxycodone and naltrexone BID reported substantially lower mean SOWS scores (e.g., 1.2) than patients taking oxycodone QID (e.g., 2.6) in the first 24 hours following drug discontinuation (p<0.01). See, e.g., Table 53. This large difference in scores (>50%) reflects attenuated (e.g., milder) withdrawal effects/events in the oxycodone and naltrexone BID group and more severe dependency and withdrawal effects/events in the oxycodone QID group. Moreover, the treatment group receiving oxycodone and naltrexone BID reported about 50% less signs or symptoms of physical dependence and withdrawal effects (p>0.01) after cessation of prolonged high-dose opioid therapy as compared to patients on oxycodone QID. See, e.g., Table 53. Patients taking oxycodone and naltrexone BID or oxycodone and naltrexone QID reported comparable pain relief (e.g., equivalent) to patients taking oxycodone QID yet reported about about 20% less overall opioid-related side effects during treatment, including somnolence and severe pruritis (p<0.05), see, e.g., Tables 50C, 50D, 52C and 52D, and about 44% less moderate and severe constipation. See, e.g., Table 50D. Also, oxycodone and naltrexone BID and oxycodone and naltrexone QID alleviated back pain at lower titration doses and lower study drug doses of oxycodone, indicating that the opioid antagonist naltrexone enhanced the potency of the opioid agonist oxycodone for alleviating back pain. See, e.g., Tables 54 and 56.

For the clinical studies as described in this Example, tablets having different amounts of oxycodone were manufactured as described in Example 5 and at larger scale. The amount of naltrexone was the same (0.001 mg) among the tablets of different strength. Tablet formulations containing oxycodone HCl at various dose levels (2.5, 5, 7.5, 10, 15, 20, 30 and 40 mg/tablet) and low-dose naltrexone HCl (0.001 mg) were prepared. Six matching active controls of oxycodone HCl tablets at various strengths (2.5, 5, 7.5, 10, 15 and 20 mg/tablet) and a matching placebo tablet were also prepared. The amounts of active ingredients and excipients in various tablets of different strengths are set forth in Tables 57 through 64. Tables 57 through 64 show the quantitative compositions of the tablets.

Table 57 sets forth the composition of exemplary 2.5 mg strength tablets (tablets comprising 2.5 mg oxycodone HCl and 0.001 mg naltrexone hydrochloride).

TABLE 57
Oxycodone HCl 2.5 mg/Naltrexone HCl 0.001 mg Tablets

| | Quantity | |
|---|---|---|
| | per | |
| | Tablet | Quantity per |
| Component | (mg) | Batch (kg) |
| Tablet Core: | | |
| Oxycodone HCl, USP | 2.5 | 0.625 |
| Naltrexone HCl, USP | 0.001 | 0.25 |
| Lactose, Monohydrate, NF | 169.80 | 42.45 |
| Hydroxypropyl Methylcellulose, USP | 2.82 | 0.71 |
| Portion A (added to the Dry Material Blend) | — | 0.47 |
| Portion B (added to the Granulating Solution) | — | 0.24 |
| Low-Substituted Hydroxypropyl Cellulose, NF | 16.88 | 4.22 |
| Portion A (added to the Dry Material Blend) | — | 0.47 |
| Portion B (added to the Final Blend) | — | 3.75 |
| Magnesium Stearate, NF | 1.00 | 0.25 |
| Talc, USP (Hydrous Magnesium Silicate) | 6.00 | 1.5 |
| Citric Acid, Anhydrous, USP | 1.00 | 0.25 |
| 2 N Sodium Hydroxide Solution* | q.s. to pH | q.s. to pH* |
| Water for Injection, USP | — | 5.0** |
| Tablet Core Total | 200.00 | 50.0 |
| Color Coating: | | |
| Opadry Clear (Base Coat) | 2.00 | 0.5 |
| Opadry II Yellow (Aesthetic Color Coat) | 8.00 | 2.0 |
| Water for Injection, USP | — | 21.31** |
| Coated Tablet Total | 210.00 | 52.5 |

*For pH adjustment of granulation fluid to pH 3.5 ± 0.2
**Removed during processing

Table 58 sets forth the composition of exemplary 5 mg strength tablets (tablets comprising 5 mg oxycodone HCl and 0.001 mg naltrexone hydrochloride).

TABLE 58
Oxycodone HCl 5 mg/Naltrexone HCl 0.001 mg Tablets

| | Quantity | |
|---|---|---|
| | per | |
| | Tablet | Quantity per |
| Component | (mg) | Batch (kg) |
| Tablet Core: | | |
| Oxycodone HCl, USP | 5.00 | 1.25 |
| Naltrexone HCl, USP | 0.001 | 0.25 |
| Lactose, Monohydrate, NF | 167.30 | 41.83 |
| Hydroxypropyl Methylcellulose, USP | 2.82 | 0.71 |
| Portion A (added to the Dry Material Blend) | — | 0.47 |
| Portion B (added to the Granulating Solution) | — | 0.24 |
| Low-Substituted Hydroxypropyl Cellulose, NF | 16.88 | 4.22 |
| Portion A (added to the Dry Material Blend) | — | 0.47 |
| Portion B (added to the Final Blend) | — | 3.75 |
| Magnesium Stearate, NF | 1.00 | 0.25 |
| Talc, USP (Hydrous Magnesium Silicate) | 6.00 | 1.5 |
| Citric Acid, Anhydrous, USP | 1.00 | 0.25 |
| 2 N Sodium Hydroxide Solution* | q.s. to pH | q.s. to pH* |
| Water for Injection, USP | — | 5.0** |
| Tablet Core Total | 200.00 | 50.0 |
| Color Coating: | | |
| Opadry Clear (Base Coat) | 2.00 | 0.5 |
| Opadry II Yellow (Aesthetic Color Coat) | 8.00 | 2.0 |
| Water for Injection, USP | — | 21.31** |
| Coated Tablet Total | 210.00 | 52.5 |

*For pH adjustment of granulation fluid to pH 3.5 ± 0.2
**Removed during processing

Table 59 sets forth the composition of exemplary 7.5 mg strength tablets (tablets comprising 7.5 mg oxycodone HCl and 0.001 mg naltrexone hydrochloride).

TABLE 59
Oxycodone HCl 7.5 mg/Naltrexone HCl 0.001 mg Tablets

| | Quantity | |
|---|---|---|
| | per | |
| | Tablet | Quantity per |
| Component | (mg) | Batch (kg) |
| Tablet Core: | | |
| Oxycodone HCl, USP | 7.50 | 1.88 |
| Naltrexone HCl, USP | 0.001 | 0.25 |
| Lactose, Monohydrate, NF | 164.80 | 41.20 |
| Hydroxypropyl Methylcellulose, USP | 2.82 | 0.71 |
| Portion A (added to the Dry Material Blend) | — | 0.47 |
| Portion B (added to the Granulating Solution) | — | 0.24 |
| Low-Substituted Hydroxypropyl Cellulose, NF | 16.88 | 4.22 |
| Portion A (added to the Dry Material Blend) | — | 0.47 |
| Portion B (added to the Final Blend) | — | 3.75 |
| Magnesium Stearate, NF | 1.00 | 0.25 |
| Talc, USP (Hydrous Magnesium Silicate) | 6.00 | 1.5 |
| Citric Acid, Anhydrous, USP | 1.00 | 0.25 |
| 2 N Sodium Hydroxide Solution* | q.s. to pH | q.s. to pH* |
| Water for Injection, USP | — | 5.0** |
| Tablet Core Total | 200.00 | 50.0 |
| Color Coating: | | |
| Opadry Clear (Base Coat) | 2.00 | 0.5 |
| Opadry II Yellow (Aesthetic Color Coat) | 8.00 | 2.0 |
| Water for Injection, USP | — | 21.31** |
| Coated Tablet Total | 210.00 | 52.5 |

*For pH adjustment of granulation fluid to pH 3.5 ± 0.2
**Removed during processing

Table 60 sets forth the composition of exemplary 10 mg strength tablets (tablets comprising 10 mg oxycodone HCl and 0.001 mg naltrexone hydrochloride).

TABLE 60
Oxycodone HCl 10 mg/Naltrexone HCl 0.001 mg Tablets

| | Quantity | |
|---|---|---|
| | per | |
| | Tablet | Quantity per |
| Component | (mg) | Batch (kg) |
| Tablet Core: | | |
| Oxycodone HCl, USP | 10.00 | 2.5 |
| Naltrexone HCl, USP | 0.001 | 0.25 |
| Lactose, Monohydrate, NF | 162.30 | 40.58 |
| Hydroxypropyl Methylcellulose, USP | 2.82 | 0.71 |
| Portion A (added to the Dry Material Blend) | — | 0.47 |
| Portion B (added to the Granulating Solution) | — | 0.24 |
| Low-Substituted Hydroxypropyl Cellulose, NF | 16.88 | 4.22 |
| Portion A (added to the Dry Material Blend) | — | 0.47 |
| Portion B (added to the Final Blend) | — | 3.75 |
| Magnesium Stearate, NF | 1.00 | 0.25 |
| Talc, USP (Hydrous Magnesium Silicate) | 6.00 | 1.5 |
| Citric Acid, Anhydrous, USP | 1.00 | 0.25 |
| 2 N Sodium Hydroxide Solution* | q.s. to pH | q.s. to pH* |
| Water for Injection, USP | — | 5.0** |
| Tablet Core Total | 200.00 | 50.0 |
| Color Coating: | | |
| Opadry Clear (Base Coat) | 2.00 | 0.5 |
| Opadry II Yellow (Aesthetic Color Coat) | 8.00 | 2.0 |
| Water for Injection, USP | — | 21.31** |
| Coated Tablet Total | 210.00 | 52.5 |

*For pH adjustment of granulation fluid to pH 3.5 ± 0.2
**Removed during processing

Table 61 sets forth the composition of exemplary 15 mg strength tablets (tablets comprising 15 mg oxycodone HCl and 0.001 mg naltrexone hydrochloride).

TABLE 61
Oxycodone HCl 15 mg/Naltrexone HCl 0.001 mg Tablets

| | Quantity | |
|---|---|---|
| | per | |
| | Tablet | Quantity per |
| Component | (mg) | Batch (kg) |
| Tablet Core: | | |
| Oxycodone HCl, USP | 15.00 | 3.75 |
| Naltrexone HCl, USP | 0.001 | 0.25 |
| Lactose, Monohydrate, NF | 157.30 | 39.33 |
| Hydroxypropyl Methylcellulose, USP | 2.82 | 0.71 |
| Portion A (added to the Dry Material Blend) | — | 0.47 |
| Portion B (added to the Granulating Solution) | — | 0.24 |
| Low-Substituted Hydroxypropyl Cellulose, NF | 16.88 | 4.22 |
| Portion A (added to the Dry Material Blend) | — | 0.47 |
| Portion B (added to the Final Blend) | — | 3.75 |
| Magnesium Stearate, NF | 1.00 | 0.25 |
| Talc, USP (Hydrous Magnesium Silicate) | 6.00 | 1.5 |
| Citric Acid, Anhydrous, USP | 1.00 | 0.25 |
| 2 N Sodium Hydroxide Solution* | q.s. to pH | q.s. to pH* |
| Water for Injection, USP | — | 5.0** |
| Tablet Core Total | 200.00 | 50.0 |
| Color Coating: | | |
| Opadry Clear (Base Coat) | 2.00 | 0.5 |
| Opadry II Yellow (Aesthetic Color Coat) | 8.00 | 2.0 |
| Water for Injection, USP | — | 21.31** |
| Coated Tablet Total | 210.00 | 52.5 |

*For pH adjustment of granulation fluid to pH 3.5 ± 0.2
**Removed during processing

Table 62 sets forth the composition of exemplary 20 mg strength tablets (tablets comprising 20 mg oxycodone HCl and 0.001 mg naltrexone hydrochloride).

TABLE 62
Oxycodone HCl 20 mg/Naltrexone HCl 0.001 mg Tablets

| | Quantity | |
|---|---|---|
| | per | |
| | Tablet | Quantity per |
| Component | (mg) | Batch (kg) |
| Tablet Core: | | |
| Oxycodone HCl, USP | 20.00 | 5.00 |
| Naltrexone HCl, USP | 0.001 | 0.25 |
| Lactose, Monohydrate, NF | 152.30 | 38.08 |
| Hydroxypropyl Methylcellulose, USP | 2.82 | 0.71 |
| Portion A (added to the Dry Material Blend) | — | 0.47 |
| Portion B (added to the Granulating Solution) | — | 0.24 |
| Low-Substituted Hydroxypropyl Cellulose, NF | 16.88 | 4.22 |
| Portion A (added to the Dry Material Blend) | — | 0.47 |
| Portion B (added to the Final Blend) | — | 3.75 |
| Magnesium Stearate, NF | 1.00 | 0.25 |
| Talc, USP (Hydrous Magnesium Silicate) | 6.00 | 1.5 |
| Citric Acid, Anhydrous, USP | 1.00 | 0.25 |
| 2 N Sodium Hydroxide Solution* | q.s. to pH | q.s. to pH* |
| Water for Injection, USP | — | 5.0** |
| Tablet Core Total | 200.00 | 50.0 |
| Color Coating: | | |
| Opadry Clear (Base Coat) | 2.00 | 0.5 |
| Opadry II Yellow (Aesthetic Color Coat) | 8.00 | 2.0 |
| Water for Injection, USP | — | 21.31** |
| Coated Tablet Total | 210.00 | 52.5 |

*For pH adjustment of granulation fluid to pH 3.5 ± 0.2
**Removed during processing

Table 63 sets forth the composition of exemplary 30 mg strength tablets (tablets comprising 30 mg oxycodone HCl and 0.001 mg naltrexone hydrochloride).

TABLE 63
Oxycodone HCl 30 mg/Naltrexone HCl 0.001 mg Tablets

| | Quantity | |
|---|---|---|
| | per | |
| | Tablet | Quantity per |
| Component | (mg) | Batch (kg) |
| Tablet Core: | | |
| Oxycodone HCl, USP | 30.00 | 7.5 |
| Naltrexone HCl, USP | 0.001 | 0.25 |
| Lactose, Monohydrate, NF | 142.30 | 35.58 |
| Hydroxypropyl Methylcellulose, USP | 2.82 | 0.71 |
| Portion A (added to the Dry Material Blend) | — | 0.47 |
| Portion B (added to the Granulating Solution) | — | 0.24 |
| Low-Substituted Hydroxypropyl Cellulose, NF | 16.88 | 4.22 |
| Portion A (added to the Dry Material Blend) | — | 0.47 |
| Portion B (added to the Final Blend) | — | 3.75 |
| Magnesium Stearate, NF | 1.00 | 0.25 |
| Talc, USP (Hydrous Magnesium Silicate) | 6.00 | 1.5 |
| Citric Acid, Anhydrous, USP | 1.00 | 0.25 |
| 2 N Sodium Hydroxide Solution* | q.s. to pH | q.s. to pH* |
| Water for Injection, USP | — | 5.0** |
| Tablet Core Total | 200.00 | 50.0 |
| Color Coating: | | |
| Opadry Clear (Base Coat) | 2.00 | 0.5 |
| Opadry II Yellow (Aesthetic Color Coat) | 8.00 | 2.0 |
| Water for Injection, USP | — | 21.31** |
| Coated Tablet Total | 210.00 | 52.5 |

*For pH adjustment of granulation fluid to pH 3.5 ± 0.2
**Removed during processing

Table 64 sets forth the composition of exemplary 40 mg strength tablets (tablets comprising 40 mg oxycodone HCl and 0.001 mg naltrexone hydrochloride).

TABLE 64
Oxycodone HCl 40 mg/Naltrexone HCl 0.001 mg Tablets

| | Quantity | |
|---|---|---|
| | per | |
| | Tablet | Quantity per |
| Component | (mg) | Batch (kg) |
| Tablet Core: | | |
| Oxycodone HCl, USP | 40.00 | 10.0 |
| Naltrexone HCl, USP | 0.001 | 0.25 |
| Lactose, Monohydrate, NF | 132.30 | 33.08 |
| Hydroxypropyl Methylcellulose, USP | 2.82 | 0.71 |
| Portion A (added to the Dry Material Blend) | — | 0.47 |
| Portion B (added to the Granulating Solution) | — | 0.24 |
| Low-Substituted Hydroxypropyl Cellulose, NF | 16.88 | 4.22 |
| Portion A (added to the Dry Material Blend) | — | 0.47 |
| Portion B (added to the Final Blend) | — | 3.75 |
| Magnesium Stearate, NF | 1.00 | 0.25 |
| Talc, USP (Hydrous Magnesium Silicate) | 6.00 | 1.5 |
| Citric Acid, Anhydrous, USP | 1.00 | 0.25 |
| 2 N Sodium Hydroxide Solution* | q.s. to pH | q.s. to pH* |
| Water for Injection, USP | — | 5.0** |
| Tablet Core Total | 200.00 | 50.0 |
| Color Coating: | | |
| Opadry Clear (Base Coat) | 2.00 | 0.5 |
| Opadry II Yellow (Aesthetic Color Coat) | 8.00 | 2.0 |
| Water for Injection, USP | — | 21.31** |
| Coated Tablet Total | 210.00 | 52.5 |

*For pH adjustment of granulation fluid to pH 3.5 ± 0.2
**Removed during processing

An advantage of dosage forms prepared as referenced and described in Example 5 and this Example, is that undesirable binding of the opioid antagonist to the excipients is minimized.

What is claimed is:

1. A method for treating back pain in a human subject comprising administering to the subject an opioid agonist and an opioid antagonist, wherein back pain is alleviated and wherein the amount of the antagonist is effective to attenuate withdrawal.

2. The method of claim 1, wherein the incidence, severity or duration of withdrawal is attenuated.

3. The method of claim 1, wherein one or more symptoms or signs of withdrawal are attenuated.

4. The method of claim 3, wherein the symptom or sign of withdrawal is feeling sick, stomach cramps, muscle spasms/twitching, a feeling of coldness, heart pounding, muscular tension, aches and pains, yawning, funny eyes, or insomnia/problems sleeping.

5. The method of claim 3, wherein the symptom or sign of withdrawal is hyperalgesia, increased heart rate, increased respiratory rate, increased pupilary diameter, muscle cramps, yawning, upset stomach, runny nose, watery eyes, abdominal cramps, clammy/damp skin, or chills/gooseflesh.

6. The method of claim 1, wherein the withdrawal is measured on the Short Opiate Withdrawal Scale (SOWS).

7. A method for treating back pain in a human subject comprising administering to the subject an opioid agonist and an opioid antagonist, wherein back pain is alleviated and wherein the amount of the antagonist is effective to attenuate one or more opioid-related adverse effects.

8. The method of claim 1, wherein the incidence, severity or duration of one or more of the opioid-related adverse effects is attenuated.

9. The method of claim 7, wherein the adverse effect is constipation, somnolence, or pruritus.

10. A method for treating back pain in a human subject comprising administering to the subject an opioid agonist and an opioid antagonist, wherein back pain is alleviated and wherein the amount of the antagonist is effective to attenuate one or more adverse effects of dependence, tolerance, or addiction.

11. The method of claim 10, wherein the incidence, severity or duration of the adverse effect is attenuated.

12. The method of claim 10, wherein the adverse effect is dependence, and one or more symptoms or signs of dependence is attenuated.

13. The method of claim 12, wherein the symptom or sign of dependence is anxiety, irritability, hot flashes, joint pain, salivation, lacrimation, rhinorrhea, diaphoresis, piloerection, nausea, vomiting, abdominal cramps, diarrhea, or sleep disturbances/insomnia.

14. The method of claim 10, wherein the adverse effect is tolerance, and one or more symptoms or signs of tolerance is attenuated.

15. The method of claim 14, wherein the symptom or sign of tolerance is a need to increase dose to maintain pain relief, a decrease in duration of pain relief for a given dose, anxiety, or sweating.

16. The method of claim 10, wherein the adverse effect is addiction, and one or more symptoms or signs of addiction is attenuated.

17. The method of claim 10, wherein the symptom or sign of addition is impaired control over opioid agonist, compulsive use of opioid agonist, continued use of opioid agonist despite harm to the subject, or craving of opioid agonist.

18. The method of claim 1, 7 or 10, wherein the back pain is associated with a neck, upper back, middle back or lower back of the subject.

19. The method of claim 1, 7 or 10, wherein the back pain is chronic.

20. The method of claim 1, 7 or 10, wherein the back pain is moderate or severe.

21. The method of claim 1, 7 or 10, wherein the back pain is low back pain.

22. The method of claim 1, 7 or 10, wherein the pain is measured as pain intensity.

23. The method of claim 22, wherein the pain intensity is measured as percent change from a baseline measurement of the subject.

24. The method of claim 23, wherein the pain intensity is reduced from the baseline measurement.

25. The method of claim 1, 7 or 10, wherein the pain is measured on an 11-point numerical scale.

26. The method of claim 25, wherein the pain is reduced in points, as compared to a baseline measurement of the subject.

27. The method of claim 25, wherein the pain is reduced by at least 1 point, as compared to a baseline measurement of the subject.

28. The method of claim 1, 7 or 10 further comprising administering to the subject an additional therapeutic agent that is a non-steroidal anti-inflammatory drug, muscle relaxant, cytokine inhibitor, corticosteroid, anti-rheumatic drug, anticonvulsant agent, tricyclic antidepressant agent, anti-dynorphin agent, or glutamate receptor antagonist agent.

29. The method of claim 28, wherein the additional therapeutic agent is a TNF-α inhibitor, corticosteroid, anti-rheumatic drug, non-steroidal anti-inflammatory drug, celecoxib, ropecoxib, valdecoxib, etanercept, infiximab, anti-TNF-α, D2E7 human Mab, CDP-870, CDP-571, humicade, PEGylated soluble TNF-α Receptor-1, TBP-1, PASSTNF-alpha, AGT-1, ienercept, CytoTAB, TACE, small molecule TNF mRNA synthesis inhibitor, PEGylated p75TNFR Fc mutein (Immunex), TNF-α antisense inhibitor, methotrexate, leflunomide, D-Penicillamine, sulfasalazine, a gold composition, minocycline, azathioprine, hydroxychloroquine, an antimalarial drug, cyclosporine, or a biologic agent that designed to either inhibit or supplement a cytokine.

30. The method of claims 1, 7 or 10, wherein the antagonist, the agonist, or both the antagonist and the agonist is administered chronically.

31. The method of claims 1, 7 or 10, wherein the antagonist, the agonist, or both the antagonist and the agonist is administered for at least six weeks.

32. The method of claims 1, 7 or 10, wherein the antagonist, the agonist, or both the antagonist and the agonist is administered for at least twelve weeks.

33. The method of claim 1, 7 or 10 wherein the subject is physically dependent on the same or a different opioid agonist prior to administration of the administration of the opioid antagonist.

34. The method of claim 1, 7 or 10 wherein the subject is tolerant to an opioid agonist prior to administration of the administration of the opioid antagonist.

35. The method of claim 1, 7 or 10 wherein the subject is addicted to an opioid agonist prior to administration of the administration of the opioid antagonist.

36. The method of claim 1, 7 or 10 wherein the amount of the opioid antagonist is 0.002 mg, less than 0.002 mg, about 0.001 mg, less than 0.001 mg, more than 0.0001 mg, about 0.0001 mg, 0.00001 mg, less than 0.00001 mg, more than 0.00001 mg, about 0.000001 mg, less than 0.000001 mg, or more than 0.000001 mg.

37. The method of claim 36, wherein the agonist, the antagonist, or both the agonist and the antagonist is administered twice a day.

38. The method of claims 36, wherein the agonist, the antagonist, or both the agonist and the antagonist is administered once a day.

39. The method of claim 1, 7 or 10, wherein the agonist, the antagonist, or both the agonist and the antagonist is administered no more than twice in a 24-hour period.

40. The method of claims 1, 7 or 10, wherein the agonist, the antagonist, or both the agonist and the antagonist is administered no more than once in a 24-hour period.

41. The method of claims 1, 7 or 10, wherein the amount of the antagonist is 0.004 mg or less in a 24-hour period.

42. The method of claims 1, 7 or 10, wherein the amount of the antagonist is 0.002 mg or less in a 24-hour period.

43. The method of claims 1, 7 or 10, wherein the antagonist, the agonist, or both the antagonist and the agonist is administered in an oral dosage form.

44. The method of claim 43, wherein the oral dosage form is a solid oral dosage form or a liquid oral dosage form.

45. The method of claims 1, 7 or 10, wherein the agonist is codeine, hydromorphone, meperidine, morphine, oxycodone, oxymorphone, propoxyphene, hydrocodone, pentazocine, fentanyl, sufentanyl, methadone, tramadol, or dihydrocodeine.

46. The method of claims 1, 7 or 10, wherein the antagonist is naltrexone, nalmefene, or naloxone.

47. The method of claims 1, 7 or 10, wherein the mode of administration of the agonist, the antagonist, or both, is oral, intravenous, intrathecal, epidural, intramuscular, subcutaneous, perineural, intradermal, topical, or transcutaneous.

48. The method of claims 1, 7 or 10, wherein the agonist is oxycodone and the antagonist is naltrexone.

49. The method of claims 1, 7 or 10, wherein the amount of the agonist is from about 2.5 mg to about 160 mg.

50. The method of claims 1, 7 or 10, wherein the amount of the antagonist is from about 0.0001 mg to about 0.004 mg.

51. The method of claims 1, 7 or 10, wherein the amount 6f the agonist is about 2.5 mg, about 5 mg, about 10 mg, about 15 mg, about 20 mg, about 30 mg, about 40 mg, about 80 mg, about 160 mg, or about 320 mg.

52. The method of claims 1, 7 or 10, wherein the amount of the antagonist administered is at least about 1250 fold less than the amount of the agonist administered.

53. The method of claims 1, 7 or 10, wherein the amount of the antagonist administered is at most about 1,600,000 fold less than the amount of the agonist administered.

54. The method of claim 1, 7 or 10, wherein the amount of the opioid antagonist administered to the subject is no more than about 80.4 μgs, about 40.2 μgs, about 20 μgs, about 10 μgs, about 5 μgs, about 2.5 μgs, about 1.2 μgs, about 0.6 μg about 0.3 μg or about 0.12 μg.

55. The method of claim 1, 7 or 10, wherein the amount of the opioid antagonist administered to the subject is at least about 0.0002 μg about 0.1 μg about 0.2 μg about 0.4 μg about 0.8 μg about 1.6 μg about 3.3 μg or about 6.6 μg of the opioid antagonist.

56. The method of claim 1, 7 or 10, wherein the amount of the antagonist administered is effective to enhance the potency of the agonist for alleviating back pain.

57. The method of claim 56, wherein the enhanced potency is measured by administering a lower dose to achieve alleviation of back pain.

58. A method for treating back pain in a human subject being administered an opioid agonist, the method comprising administering to the subject an opioid antagonist, wherein back pain is alleviated, and wherein the amount of the antagonist is effective to attenuate withdrawal.

59. A method for treating back pain in a human subject being administered an opioid agonist, the method comprising administering to the subject an opioid antagonist, wherein back pain is alleviated, and wherein the amount of the antagonist is effective to attenuate one or more opioid-related adverse effects.

60. A method for treating back pain in a human subject being administered an opioid agonist, the method comprising administering to the subject an opioid antagonist, wherein back pain is alleviated, and wherein the amount of the antagonist is effective to attenuate one or more adverse effects of dependence, tolerance, or addiction.

61. The method of claims 58, 59 or 60, wherein the subject is physically dependent on an opioid agonist prior to administration of the administration of the opioid antagonist.

62. The method of claims 58, 59 or 60, wherein the subject is tolerant to an opioid agonist prior to administration of the administration of the opioid antagonist.

63. The method of claims 58, 59 or 60, wherein the subject is addicted to an opioid agonist prior to administration of the administration of the opioid antagonist.

64. A method of dosing an opioid antagonist administered to a human subject having back pain, the method comprising the steps of:
(a) administering an amount of an opioid antagonist and an amount of an opioid agonist to the subject;
(b) assessing back pain;
(c) measuring a level of the opioid antagonist or a surrogate of the opioid antagonist in a sample from the subject; and
(d) adjusting the amount of the opioid antagonist or the amount of the opioid agonist to the subject based on the measured level.

65. The method of claim 64, wherein step (d) comprises adjusting the amount of the opioid antagonist administered to the subject.

66. The method of claim 64, wherein step (d) comprises adjusting the amount of the opioid agonist administered to the subject.

67. The method of claim 64, wherein the amount of the opioid antagonist administered to the subject is increased if the measured level is lower than a predetermined value.

68. The method of claim 64, wherein the amount of the opioid antagonist administered to the subject is decreased in the measured level is higher than a predetermined value.

69. The method of claim 64, wherein the amount of the opioid antagonist administered to the subject is maintained in the measured level is within a predetermined range.

70. The method of claim 64, further comprising the step of increasing the amount of the opioid agonist administered to the subject.

71. The method of claim 64, wherein the level of 6β-naltrexol is measured as a surrogate.

72. The method of claim 64, wherein the 6β-naltrexol is a surrogate marker for assessing one or more symptoms or signs of an arthritic condition, inflammation associated with a chronic condition, or chronic pain.

73. The method of claim 64, wherein the sample is a plasma sample.

74. A method of dosing an opioid antagonist to a human subject having back pain, the method comprising the steps of:
(a) administering an amount of an opioid antagonist and an amount of an opioid agonist to the subject;
(b) assessing back pain; and
(c) adjusting the amount of the opioid antagonist administered to the subject the back pain is not alleviated to a desired extent.

75. The method of claim 74, wherein step (a) comprises repeatedly administering the opioid antagonist such that a steady state is achieved.

76. The method of claim 74, wherein step (c) also comprises maintaining the amount of the opioid agonist administered to the subject.

77. The method of claim 74, further comprising the steps of:
(d) re-assessing the back pain after step (c);
(e) adjusting the amount of the opioid agonist if the back pain is not alleviated to a desired extent.

78. The method of claim 74, wherein the back pain is low back pain.

79. The method of claim 78, wherein the back pain is measured as pain intensity.

80. The method of claim 79, wherein the pain intensity measurement is attenuated as compared to a pain intensity baseline measurement of the subject.

81. The method of claim 79, wherein the pain intensity measurement is reduced by at least about 20%, at least about 50%, or at least about 90% compared to a pain intensity baseline measurement of the subject.

82. The method of claim 74, further comprising the steps of:
(d) measuring a level of the opioid antagonist or a surrogate of the opioid antagonist in a sample from the subject, and
(e) further adjusting the amount of the opioid antagonist based on the measured level of the antagonist or the surrogate.

83. The method of claim 74, wherein the sample is a plasma sample.

84. A method of dosing an opioid antagonist to a human subject, the method comprising the steps of:
(a) administering an amount of an opioid antagonist and an amount of an opioid agonist to the subject;
(b) measuring a level of the opioid antagonist or a surrogate of the opioid antagonist in a sample from the subject; and
(c) adjusting the amount of the opioid antagonist administered to the subject if the measured level is outside a predetermined range.

85. The method of claim 84, further comprising repeating step (a) if the measured level is within the predetermined range.

86. The method of claim 85, wherein the sample is a plasma sample.

87. The method of claim 86, wherein the predetermined range is a range of plasma concentrations predicted by a model of plasma concentration-effect relationship as the range which provides about 20% or greater reduction in pain intensity, or about 50% or greater reduction in pain intensity, or about 90% or greater reduction in pain intensity.

88. The method of claim 87, wherein the predetermined plasma concentration is based on the plasma concentration-effect relationship shown in FIG. 11 or the plasma concentration-effect relationship shown in FIG. 12.

89. A method of determining the amount of an opioid antagonist or opioid agonist to be administered to a human subject having back pain, the method comprising the steps of:
(a) assessing back pain in a human subject being administered an opioid antagonist and an opioid agonist:
(b) measuring a level of the opioid antagonist or a surrogate of the opioid antagonist in a sample obtained from the human subject;
(c) on the basis of the measured level, adjusting the amount of the opioid antagonist or the amount of the opioid agonist for administration to the human subject.

90. The method of claim 89, wherein the amount of the opioid antagonist administered to the subject is increased if the measured level is lower than a predetermined value.

91. The method of claim 89, wherein the amount of the opioid antagonist administered to the subject is decreased in the measured level is higher than a predetermined value.

92. The method of claim 89, wherein the amount of the opioid antagonist administered to the subject is maintained in the measured level is within a predetermined range.

93. A method of reducing the level of a biomarker in a human subject having back pain, comprising administering to the subject a composition comprising an opioid antagonist and optionally an opioid agonist.

94. The method of claim 93, further comprising administering an opioid agonist to the subject.

95. The method of claim 93, wherein the biomarker is a cytokine.

96. The method of claim 93, wherein the biomarker is IL1a, IL1b, IL2, IL4, IL5, IL6, IL13, GM-CSF, interferon-γ, or TNFα.

97. The method of claim 93, wherein the biomarker is TNFα, IL6, or IL4.

98. The method of claim 93, wherein the biomarker is TNFα, and the level is reduced to about 0.2 ng/ml or lower.

99. The method of claim 93, wherein the biomarker is IL6, and the level is reduced to about 0.18 ng/ml or lower.

100. The method of claim 93, wherein the biomarker is IL4, and the level is reduced to about 0.23 ng/ml or lower.

101. A method to monitor the response of a human subject being treated for back pain, by administering an opioid antagonist, comprising the steps of:
(a) determining the level of one or more one biomarker(s) in a first sample from the subject prior to treatment with the opioid antagonist;
(b) determining the level of the biomarker in at least a second sample from the subject subsequent to the initial treatment with the opioid antagonist; and
(c) comparing the level of the biomarker in the second sample with the level of the biomarker in the first sample;
wherein a change in the level of the biomarker in the second sample compared to the level of the biomarker in the first sample indicates the effectiveness of the treatment.

102. A method to monitor the response of a human subject being treated for back pain, by administering an opioid antagonist and an opioid agonist, comprising the steps of:
(a) determining the level of one or more one biomarker(s) in a first sample from the subject prior to treatment with the opioid antagonist and the opioid agonist;
(b) determining the level of the biomarker in at least a second sample from the subject subsequent to the initial treatment with the opioid antagonist and the opioid agonist; and
(c) comparing the level of the biomarker in the second sample with the level of the biomarker in the first sample;
wherein a change in the level of the biomarker in the second sample compared to the level of the biomarker in the first sample indicates the effectiveness of the treatment.

103. The method of claims 101 or 102, further comprising the step of adjusting the amount of the opioid antagonist administered to the subject after comparing the levels of the biomarker.

104. The method of claim 103, further comprising the step of adjusting the amount of the opioid agonist administered to the subject after comparing the levels of the biomarker.

105. The method of claims 101 or 102, wherein the biomarker is a cytokine.

106. The method of claims 101 or 102, wherein the biomarker is IL1a, IL1b, IL2, IL4, IL5, IL6, IL13, GM-CSF, interferon-γ, or TNFα.

107. The method of claims 101 or 102, wherein the biomarker is TNFα, IL6, or IL4.

108. The method of claims 101 or 102, wherein the biomarker is TNFα, and the level is reduced to about 0.2 ng/ml or lower.

109. The method of claims 101 or 102, wherein the biomarker is IL6, and the level is reduced to about 0.18 ng/ml or lower.

110. The method of claims 101 or 102, wherein the biomarker is IL4, and the level is reduced to about 0.23 ng/ml or lower.
